(19) United States
(12) Patent Application Publication
(10) Pub. No.: US 2024/0325459 A1
(43) Pub. Date: Oct. 3, 2024

(54) METHODS AND COMPOSITIONS FOR TREATING DISORDERS ASSOCIATED WITH BILE ACIDS

(71) Applicant: Novome Biotechnologies, Inc., South San Francisco, CA (US)

(72) Inventors: Peter Spanogiannopoulos, South San Francisco, CA (US); Weston Robert Whitaker, South San Francisco, CA (US); Kathy Lam, South San Francisco, CA (US); Jessica Tan, South San Francisco, CA (US); Zachary Nicholas Russ, South San Francisco, CA (US); William Cain DeLoache, South San Francisco, CA (US); Lauren Popov, South San Francisco, CA (US); Lane Weaver, South San Francisco, CA (US)

(21) Appl. No.: 18/228,870

(22) Filed: Aug. 1, 2023

Related U.S. Application Data
(63) Continuation of application No. PCT/US2022/014991, filed on Feb. 2, 2022.
(60) Provisional application No. 63/144,715, filed on Feb. 2, 2021.

(51) Int. Cl. A61K 35/74; C12N 9/04

(52) U.S. Cl. CPC A61K 35/74; C12N 9/0006; C12Y 101/01145

(57) ABSTRACT

The disclosure relates generally to bacteria that have been modified to metabolize a bile acid, pharmaceutical compositions including the bacteria, and methods of using the bacteria and pharmaceutical compositions to treat disorders associated with an elevated amount of bile acid, e.g., bile acid diarrhea.

### RELATED APPLICATIONS

This application is a continuation of International Application No. PCT/US2022/014991, filed on Feb. 2, 2022, which claims the benefit of, and priority to, U.S. Provisional Patent Application No. 63/144,715, filed on Feb. 2, 2021, the contents of each of which are hereby incorporated by reference in their entirety for any and all purposes.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Jun. 19, 2024, is named “NVM-007WO_SL_v2.xml” and is 259,853 bytes in size.

### BACKGROUND

Bile acids are planar, amphipathic detergent-like molecules that form micelles and possess a myriad of biological functions. In humans these steroid acids are synthesized in the liver by hepatocytes from cholesterol and are conjugated with taurine or glycine to yield anions called bile salts or conjugated bile acids. These molecules are collected in the bile and stored in the gallbladder. From the gallbladder, conjugated bile acids are secreted into the gastrointestinal tract (GIT) where they aid in digestion. In addition to facilitating the absorption of nutrients, dietary fats, steroids, and vitamins, bile salts and acids also act as signaling molecules and metabolic integrators via interactions with host receptors to regulate their own biosynthesis and multiple aspects of host metabolism and physiology. Additionally, bile acids have been shown to play an important role in host immunity and prevention of intestinal pathogen expansion.

Bile acids synthesized in the liver are called primary bile acids. The bile acid pool in humans is tightly regulated. In healthy individuals, approximately 95% of bile acids are reabsorbed at the terminal ileum and recycled to the liver via enterohepatic circulation. The remaining 5% of primary bile acids transit through the colon. Throughout the gastrointestinal tract (GIT), bile acids encounter gut bacteria, which are capable of metabolizing bile acids to numerous products with altered bioactivity and bioavailability. Bile acids that have been modified by gut bacteria are called secondary bile acids. Gut bacteria are capable of deconjugating bile salts through enzymes called bile salt hydrolases (BSHs). Following deconjugation, the bile acids cholic acid (CA) and chenodeoxycholic acid (CDCA) are dehydroxylated at the 7-hydroxyl residue by select gut bacteria generating the secondary bile acids deoxycholic acid (DCA) and lithocholic acid (LCA), respectively. In the colon, bile acids can be absorbed by passive diffusion and are directed to the liver for conjugation and then moved to the gallbladder for storage.

Bile acids are associated with multiple diseases including but not limited to Irritable Bowel Syndrome (IBS), Bile Acid Diarrhea (BAD), Inflammatory Bowel Disease (IBD), Crohn's Disease (CD), Ulcerative Colitis (UC), Cholestasis, Intrahepatic Cholestasis of Pregnancy (ICP), Cancer, Colorectal Cancer (CRC), Nonalcoholic Fatty Liver Disease (NAFLD), Steatosis, Non-alcoholic Steatohepatitis (NASH), In-born Errors of Bile Acid Metabolism, Progressive Familial Intrahepatic Cholestasis (PFIC), Primary Biliary Cirrhosis (PBC), Primary Sclerosing Cholangitis (PSC), Metabolic Syndrome, Type 2 Diabetes (T2D), Obesity, hypercholesteremia, dyslipidemia, or atherosclerosis.

Bile acid diarrhea (BAD) is chronic diarrhea caused by bile acid dysfunction in the GIT. BAD is likely an underappreciated cause of chronic diarrhea, with some estimates that 1% of the global population may experience bile acid driven diarrheal dysfunction. Studies suggest that between 25-50% of individuals diagnosed with diarrhea-predominant irritable bowel syndrome (IBS-D) or functional chronic diarrhea may suffer from BAD.

Despite a paucity of prospective clinical studies demonstrating their efficacy for diarrheal symptoms, off-label use of bile acid sequestrants (BAS) are currently the treatment of choice for BAD. Nevertheless, BAS have several limitations as an intervention for BAD, including poor patient compliance due to low palatability and adverse events, and their interference with the absorption of other medications.

Accordingly, there is an ongoing need for effective therapies for treating and managing diseases or disorders associated with bile acids and bile salts.

### SUMMARY

The disclosure relates generally to bacteria that have been modified to metabolize a bile acid and/or bile salt. Disclosed bacteria are useful for preventing or treating diseases and/or disorders that are associated with bile acids, e.g., an elevated amount of bile acid in a subject. Disclosed bacteria are further useful for generating bile acids that have therapeutic use.

For example, in certain aspects, the gut of a subject receiving a bacterium provided by the present disclosure comprises a complex-native microbiota. As used herein, the term “complex-native microbiota” refers to the microbiota naturally present in the gastrointestinal tract of a subject. In certain embodiments, a complex-native microbiota comprises at least 10 bacterial species, at least 50 bacterial species, at least 100 bacterial species, at least 500 bacterial species, or at least 1000 bacterial species. For example, a complex-native microbiota can comprise between about 10 and about 50, between about 10 and about 100, between about 10 and about 500, between about 10 and about 1000, between about 10 and about 1500, between about 10 and about 2000, between about 100 and about 500, between about 100 and about 1000, between about 100 and about 2000, between about 500 and about 1000, between about 500 and about 2000, or between about 1000 and about 2000 bacterial species. In certain embodiments, a subject that does not have a complex-native microbiota can include, for example, a germ-free mouse, a gnotobiotic mouse, or a germ-free or gnotobiotic mouse experimentally colonized with 1, 2, 3, 4, 5, 6, 7, 8, or 9 strains of bacteria.

In some aspects, provided herein is a bacterium (e.g., a commensal and/or anaerobic bacterium) comprising one or more transgenes that increase the bacterium's ability to metabolize one or more bile acids or bile salts. In some aspects, provided herein is a bacterium (e.g., a commensal and/or anaerobic bacterium) comprising one or more transgenes that enable the bacterium's ability to metabolize one or more bile acids or bile salts. In some aspects, one or more transgenes comprise: (i) a transgene encoding a 3α-hydroxysteroid dehydrogenase (3α-HSDH), or a functional fragment or variant thereof, having at least 80% identity to an amino sequence encoded by SEQ ID NO: 18; and/or (ii) a transgene encoding a 3β-hydroxysteroid dehydrogenase (3β-HSDH), or a functional fragment or variant thereof, having at least 80% identity to an amino sequence encoded by any one of SEQ ID NOs: 28, 30, 32, or 36. In some aspects, a 3α-HSDH, or functional fragment or variant thereof, comprises an amino sequence encoded by SEQ ID NO: 18. In some aspects, a 3β-HSDH, or a functional fragment or variant thereof, comprises an amino sequence encoded by any one of SEQ ID NOs: 28, 30, 32, or 36.

In some aspects, a bacterium described herein is capable of achieving a rate of metabolism of the one or more bile acids or bile salts of greater than 0.5 mM/hour, greater than 0.6 mM/hour, greater than 0.7 mM/hour, greater than 0.8 mM/hour, greater than 0.9 mM/hour, or greater than 1.0 mM/hour. In some aspects, a bacterium described herein is capable of achieving a rate of metabolism of the one or more bile acids or bile salts of about 0.5 mM/hour, about 0.6 mM/hour, about 0.7 mM/hour, about 0.8 mM/hour, about 0.9 mM/hour, or about 1.0 mM/hour. In some aspects, a bacterium described herein is capable of achieving a rate of metabolism of the one or more bile acids or bile salts of greater than 0.5 mM/hour. In some aspects, a bacterium described herein is capable of achieving a rate of metabolism of the one or more bile acids or bile salts of greater than 0.8 mM/hour. In some aspects, rate of metabolism is maintained for at least 6 hours, at least 12 hours, at least 24 hours, at least 36 hours, at least 48 hours, at least 60 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

In some aspects, a bacterium described herein is capable of achieving a rate of metabolism of the one or more bile acids or bile salts of greater than 0.5 mM/hour, greater than 0.6 mM/hour, greater than 0.7 mM/hour, greater than 0.8 mM/hour, greater than 0.9 mM/hour, or greater than 1.0 mM/hour in a subject's gut. In some aspects, a bacterium described herein is capable of achieving a rate of metabolism of the one or more bile acids or bile salts of about 0.5 mM/hour, about 0.6 mM/hour, about 0.7 mM/hour, about 0.8 mM/hour, about 0.9 mM/hour, or about 1.0 mM/hour in a subject's gut. In some aspects, a bacterium described herein is capable of achieving a rate of metabolism of the one or more bile acids or bile salts of greater than 0.5 mM/hour in a subject's gut. In some aspects, a bacterium described herein is capable of achieving a rate of metabolism of the one or more bile acids or bile salts of greater than 0.8 mM/hour in a subject's gut. In some aspects, rate of metabolism is maintained for at least 6 hours, at least 12 hours, at least 24 hours, at least 36 hours, at least 48 hours, at least 60 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

In some aspects, a bacterium described herein is capable of converting at least 10%, at least 20%, at least 30%, at least 40%, at least 50%, at least 60%, at least 70%, at least 80%, or at least 90% of the one or more bile acids or bile salts to one or more different bile acid or bile salt products in a subject's gut. In some aspects, a bacterium described herein converts at least 70% of the one or more bile acids or bile salts to one or more different bile acid or bile salt products in a subject's gut. In some aspects, conversion is maintained for at least 6 hours, at least 12 hours, at least 24 hours, at least 36 hours, at least 48 hours, at least 60 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

In some aspects, the gut of a subject receiving a bacterium provided by the present disclosure comprises a complex-native microbiota. In some aspects, a complex-native microbiota comprises at least 10 bacterial species. In some aspects, a complex-native microbiota comprises greater than 10 bacterial species.

In someone aspects, provided herein is a bacterium (e.g., a commensal and/or anaerobic bacterium) comprising one or more transgenes that increase the bacterium's ability to metabolize one or more bile acids or bile salts (for example, relative to a similar or otherwise identical bacterium that does not comprise the one or more transgenes). In some embodiments, provided herein is a bacterium (e.g., a commensal and/or anaerobic bacterium) comprising one or more transgenes that enable the bacterium to metabolize one or more bile acids or bile salts (for example, relative to a similar or otherwise identical bacterium that does not comprise the one or more transgenes). In some embodiments, a bacterium provided by the present disclosure metabolizes one or more bile acids or bile salts substrates to one or more different bile acid or bile salt products with an altered property (e.g., bioactivity, and/or bioavailability) relative to the bile acid or bile salt substrate. In some embodiments of the present disclosure it is contemplated that the one or more transgenes described herein may, e.g., be on a plasmid, bacterial artificial chromosome, or be genomically integrated.

In certain embodiments, a contemplated bacterium is of a genus selected from the group consisting of Bacteroides, Alistipes, Faecalibacterium, Faecalicatena, Parabacteroides, Prevotella, Roseburia, Ruminococcus, Clostridium, Oscillibacter, Gemmiger, Barnesiella, Dialister, Parasutterella, Phascolarctobacterium, Propionibacterium, Sutterella, Blautia, Paraprevotella, Coprococcus, Odoribacter, Spiroplasma, Anaerostipes, and Akkermansia. For example, a contemplated bacterium may be of the Bacteroides genus, i.e., may be a Bacteroides species bacterium.

Exemplary bile acids include cholic acid (CA), chenodeoxycholic acid (CDCA), deoxycholic acid (DCA), lithocholic acid (LCA), isocholic acid (isoCA), isochenodeoxycholic acid (isoCDCA), isodeoxycholic acid (isoDCA), isolithocholic acid (isoLCA), ursocholic acid (UCA), ursodeoxycholic acid (UDCA), lagocholic acid (lagoCA), lagodeoxycholic acid (lagoDCA), β-muricholic acid (β-MCA), α-muricholic acid (α-MCA), γ-muricholic acid (γ-MCA), and ω-muricholic acid (ω-MCA). Exemplary bile salts include taurocholic acid (TCA), glycocholic acid (GCA), taurochenodeoxycholic acid (TCDCA), glycochenodeoxycholic (GCDCA), taurodeoxycholic acid (TDCA), glycodeoxycholic acid (GDCA), taurolithocholic acid (TLCA), and glycolithocholic acid (GLCA).

In certain embodiments, a contemplated bacterium metabolizes a bile acid or bile salt substrate to a different bile acid or bile salt product that has an altered property, e.g., altered bioactivity (e.g., affinity for a receptor, detergent effects), compared to the bile acid or bile salt substrate. In some embodiments, a bile acid or bile salt product has reduced affinity for a receptor (e.g., a TGR5 receptor) relative to the bile acid or bile salt substrate. In some embodiments, a bile acid or bile salt product has increased affinity for a receptor (e.g., a TGR5 receptor) relative to the bile acid or bile salt substrate. In some embodiments, a bile acid or bile salt product has an altered property, e.g., affinity for a receptor selected from the group consisting of. Farnesoid X receptor (FXR), G protein-couple bile acid receptor 1 (GPBAR1, also known as GPCR19, M-BAR, and TGR5), Pregnane X receptor (PXR), Vitamin D receptor (VDR), Constitutive Androstane receptor (CAR), Sphingosine-1-Phosphate receptor 2 (S1PR2), Muscarinic Acetylcholine receptor M3 (M3R), Epidermal Growth Factor receptor (EGFR), Liver X receptors (LXR), and glucocorticoid receptor. In some embodiments, a bile acid or bile salt product has an altered property, e.g., affinity for a TGR5 receptor. In certain embodiments, a contemplated bacterium metabolizes a bile acid or bile salt substrate to a different bile acid or bile salt product that has altered detergent effects, e.g., increased or decreased detergent effects.

In certain embodiments, a contemplated bacterium metabolizes a bile acid or bile salt substrate to a different bile acid or bile salt product that has altered bioavailability, compared to the bile acid or bile salt substrate.

In certain embodiments, a contemplated bacterium metabolizes chenodeoxycholic acid (CDCA), or is capable of metabolizing CDCA. In some embodiments, CDCA is metabolized to a product with altered bioactivity (e.g., affinity for a receptor). In some embodiments, a contemplated bacterium may increase the affinity of CDCA for a human receptor (e.g., by metabolizing CDCA to a different molecule with increased affinity for a human receptor), or be capable of increasing the affinity of CDCA for a human receptor. In some embodiments, a contemplated bacterium may reduce the affinity of CDCA for a human receptor (e.g., by metabolizing CDCA to a different molecule with reduced affinity for a human receptor), or be capable of reducing the affinity of CDCA for a human receptor. For example, a contemplated bacterium may reduce the affinity of CDCA for human TGR5 (e.g., by metabolizing CDCA to a different molecule with reduced affinity for TGR5), or be capable of reducing the affinity of CDCA for human TGR5. Other non-limiting examples of receptors include the Farnesoid X receptor (FXR), G protein-couple bile acid receptor 1 (GPBAR1, also known as GPCR19, M-BAR, and TGR5), Pregnane X receptor (PXR), Vitamin D receptor (VDR), Constitutive Androstane receptor (CAR), Sphingosine-1-Phosphate receptor 2 (S1PR2), Muscarinic Acetylcholine receptor M3 (M3R), Epidermal Growth Factor receptor (EGFR), Liver X receptors (LXR), and glucocorticoid receptors.

In certain embodiments, a contemplated bacterium metabolizes CDCA to ursodeoxycholic acid (UDCA), or is capable of metabolizing CDCA to ursodeoxycholic acid (UDCA). For example, a contemplated bacterium may comprise a transgene encoding a 7α-hydroxysteroid dehydrogenase (7α-HSDH), or a functional fragment or variant thereof, for example, a Bacteroides fragilis, Escherichia coli, Paeniclostridium sordellii, Clostridium absonum, or Brucella melitensis 7α-HSDH, for example, a 7α-HSDH comprising an amino sequence encoded by any one of SEQ ID NOs: 1-6, or having at least 80% identity to an amino sequence encoded by any one of SEQ ID NOs: 1-6. In certain embodiments, a contemplated bacterium may comprise a transgene encoding a 7α-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 1-6. In certain embodiments, a contemplated bacterium comprises a nucleic acid comprising the nucleotide sequence of any one of SEQ ID NOs: 1-6, or a nucleotide sequence having at least 80% identity to any one of SEQ ID NOs: 1-6. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 1-6. Alternatively, or in addition, a contemplated bacterium may comprise a transgene encoding a 7β-hydroxysteroid dehydrogenase (7β-HSDH), or a functional fragment or variant thereof, for example, a Ruminococcus gnavus, Colinsella aerofaciens, Clostridium absonum, or Ruminococcus torques 7β-HSDH, for example, a 7β-HSDH comprising an amino sequence encoded by any one of SEQ ID NOs: 7-11, or having at least 80% identity to an amino sequence encoded by any one of SEQ ID NOs: 7-11. In certain embodiments, a contemplated bacterium may comprise a transgene encoding a 7β-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 7-11. In certain embodiments, a contemplated bacterium comprises a nucleic acid comprising the nucleotide sequence of any one of SEQ ID NOs: 7-11, or a nucleotide sequence having at least 80% identity to any one of SEQ ID NOs: 7-11. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 7-11.

In certain embodiments, a contemplated bacterium metabolizes CDCA to allo-chenodeoxycholic acid (alloCDCA) or isoallo-chenodeoxycholic acid (isoalloCDCA), or is capable of metabolizing CDCA to alloCDCA or isoalloCDCA. For example, a contemplated bacterium may comprise a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23. In certain embodiments, a contemplated bacterium may comprise a transgene encoding a 3α-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 12-23. In certain embodiments, a contemplated bacterium comprises a nucleic acid comprising the nucleotide sequence of any one of SEQ ID NOs: 12-23, or a nucleotide sequence having at least 80% identity to any one of SEQ ID NOs: 12-23. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 12-23. Alternatively, or in addition, a contemplated bacterium may comprise a transgene encoding a 5α-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, for example, a 5α-reductase comprising an amino sequence encoded by any one of SEQ ID NOs: 61-67, or having at least 80% identity to an amino sequence encoded by any one of SEQ ID NOs: 61-67. In certain embodiments, a contemplated bacterium may comprise a transgene encoding a 5α-reductase, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 61-67. In certain embodiments, a contemplated bacterium comprises a nucleic acid comprising the nucleotide sequence of any one of SEQ ID NOs: 61-67, or a nucleotide sequence having at least 80% identity to any one of SEQ ID NOs: 61-67. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 61-67. Alternatively, or in addition, a contemplated bacterium may comprise a transgene encoding a 5β-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, for example, a 5β-reductase comprising an amino sequence encoded by any one of SEQ ID NOs: 68-74, or having at least 80% identity to an amino sequence encoded by any one of SEQ ID NOs: 68-74. In certain embodiments, a contemplated bacterium may comprise a transgene encoding a 5β-reductase, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 68-74. In certain embodiments, a contemplated bacterium comprises a nucleic acid comprising the nucleotide sequence of any one of SEQ ID NOs: 68-74, or a nucleotide sequence having at least 80% identity to any one of SEQ ID NOs: 68-74. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 68-74. Alternatively, or in addition, a contemplated bacterium may comprise a transgene encoding a 3β-hydroxysteroid dehydrogenase (3β-HSDH), or a functional fragment or variant thereof, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis3β-HSDH, for example, a 3β-HSDH comprising an amino sequence encoded by any one of SEQ ID NOs: 24-47, or having at least 80% identity to an amino sequence encoded by any one of SEQ ID NOs: 24-47. In certain embodiments, a contemplated bacterium may comprise a transgene encoding a 3β-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 24-47. In certain embodiments, a contemplated bacterium comprises a nucleic acid comprising the nucleotide sequence of any one of SEQ ID NOs: 24-47, or a nucleotide sequence having at least 80% identity to any one of SEQ ID NOs: 24-47. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 24-47.

In certain embodiments, a contemplated bacterium metabolizes deoxycholic acid (DCA), or is capable of metabolizing DCA. In some embodiments, DCA is metabolized to a bile acid product with altered bioactivity (e.g., affinity for a receptor). In some embodiments, a contemplated bacterium may increase the affinity of DCA for human receptors (e.g., by metabolizing DCA to a different molecule with increased affinity for a human receptor). In some embodiments, a contemplated bacterium may reduce the affinity of DCA for human receptors (e.g., by metabolizing DCA to a different molecule with reduced affinity for a human receptor). For example, a contemplated bacterium may reduce the affinity of DCA for human TGR5 (e.g., by metabolizing DCA to a different molecule with reduced affinity for TGR5), or be capable of reducing the affinity of DCA for human TGR5.

In certain embodiments, a contemplated bacterium metabolizes DCA to isodeoxycholic acid (isoDCA), or is capable of metabolizing DCA or isoDCA. For example, a contemplated bacterium may comprise a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23. In certain embodiments, a contemplated bacterium may comprise a transgene encoding a 3α-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 12-23. In certain embodiments, a contemplated bacterium comprises a nucleic acid comprising the nucleotide sequence of any one of SEQ ID NOs: 12-23, or a nucleotide sequence having at least 80% identity to any one of SEQ ID NOs: 12-23. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 12-23. Alternatively, or in addition, a contemplated bacterium may comprise a transgene encoding a 3β-hydroxysteroid dehydrogenase (3β-HSDH), or a functional fragment or variant thereof, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis3β-HSDH, for example, a 3β-HSDH comprising an amino sequence encoded by any one of SEQ ID NOs: 24-47, or having at least 80% identity to an amino sequence encoded by any one of SEQ ID NOs: 24-47. In certain embodiments, a contemplated bacterium may comprise a transgene encoding a 3β-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 24-47. In certain embodiments, a contemplated bacterium comprises a nucleic acid comprising the nucleotide sequence of any one of SEQ ID NOs: 24-47, or a nucleotide sequence having at least 80% identity to any one of SEQ ID NOs: 24-47. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 24-47.

In certain embodiments, a contemplated bacterium metabolizes DCA to lagodeoxycholic acid (lagoDCA), or is capable of metabolizing DCA or lagoDCA. For example, a contemplated bacterium may comprise a transgene encoding a 12α-hydroxysteroid dehydrogenase (12α-HSDH), or a functional fragment or variant thereof, for example, a Eggerthella lenta, Eggerthella sp. CAG:298, Clostridium sp. ATCC29733, Clostridium hylemonae, Clostridium scindens, or Clostridium hiranonis 12α-HSDH, for example, a 12α-HSDH comprising an amino sequence encoded by any one of SEQ ID NOs: 48-54, or having at least 80% identity to an amino sequence encoded by any one of SEQ ID NOs: 48-54. In certain embodiments, a contemplated bacterium may comprise a transgene encoding a 12α-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 48-54. In certain embodiments, a contemplated bacterium comprises a nucleic acid comprising the nucleotide sequence of any one of SEQ ID NOs: 48-54, or a nucleotide sequence having at least 80% identity to any one of SEQ ID NOs: 48-54. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 48-54. Alternatively, or in addition, a contemplated bacterium may comprise a transgene encoding a 12β-hydroxysteroid dehydrogenase (12β-HSDH), or a functional fragment or variant thereof, for example, a Clostridium paraputrificum, Eisenbergiella sp. OF01-20, Olsenella sp. GAM18, Collinsella tanakaei, Ruminococcus sp. AF14-10, or Ruminococcus lactaris 12β-HSDH, for example, a 12β-HSDH comprising an amino sequence encoded by any one of SEQ ID NOs: 55-60, or having at least 80% identity to an amino sequence encoded by any one of SEQ ID NOs: 55-60. In certain embodiments, a contemplated bacterium may comprise a transgene encoding a 12α-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 55-60. In certain embodiments, a contemplated bacterium comprises a nucleic acid comprising the nucleotide sequence of any one of SEQ ID NOs: 55-60, or a nucleotide sequence having at least 80% identity to any one of SEQ ID NOs: 55-60. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 55-60.

In certain embodiments, a contemplated bacterium metabolizes DCA to allo-deoxycholic acid (alloDCA) or isoallo-deoxycholic acid (isoalloDCA), or is capable of metabolizing DCA to alloDCA or isoalloDCA. For example, a contemplated bacterium may comprise a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23. In certain embodiments, a contemplated bacterium may comprise a transgene encoding a 3α-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 12-23. In certain embodiments, a contemplated bacterium comprises a nucleic acid comprising the nucleotide sequence of any one of SEQ ID NOs: 12-23, or a nucleotide sequence having at least 80% identity to any one of SEQ ID NOs: 12-23. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 12-23. Alternatively, or in addition, a contemplated bacterium may comprise a transgene encoding a 5α-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, for example, a 5α-reductase comprising an amino sequence encoded by any one of SEQ ID NOs: 61-67, or having at least 80% identity to an amino sequence encoded by any one of SEQ ID NOs: 61-67. In certain embodiments, a contemplated bacterium may comprise a transgene encoding a 5α-reductase, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 61-67. In certain embodiments, a contemplated bacterium comprises a nucleic acid comprising the nucleotide sequence of any one of SEQ ID NOs: 61-67, or a nucleotide sequence having at least 80% identity to any one of SEQ ID NOs: 61-67. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 61-67. Alternatively, or in addition, a contemplated bacterium may comprise a transgene encoding a 5β-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, for example, a 5β-reductase comprising an amino sequence encoded by any one of SEQ ID NOs: 68-74, or having at least 80% identity to an amino sequence encoded by any one of SEQ ID NOs: 68-74. In certain embodiments, a contemplated bacterium may comprise a transgene encoding a 5β-reductase, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 68-74. In certain embodiments, a contemplated bacterium comprises a nucleic acid comprising the nucleotide sequence of any one of SEQ ID NOs: 68-74, or a nucleotide sequence having at least 80% identity to any one of SEQ ID NOs: 68-74. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 68-74. Alternatively, or in addition, a contemplated bacterium may comprise a transgene encoding a 3β-hydroxysteroid dehydrogenase (3β-HSDH), or a functional fragment or variant thereof, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, for example, a 3β-HSDH comprising an amino sequence encoded by any one of SEQ ID NOs: 24-47, or having at least 80% identity to an amino sequence encoded by any one of SEQ ID NOs: 24-47. In certain embodiments, a contemplated bacterium may comprise a transgene encoding a 3β-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 24-47. In certain embodiments, a contemplated bacterium comprises a nucleic acid comprising the nucleotide sequence of any one of SEQ ID NOs: 24-47, or a nucleotide sequence having at least 80% identity to any one of SEQ ID NOs: 24-47. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 24-47.

In certain embodiments, a contemplated bacterium metabolizes LCA to isoallolithocholic acid (isoalloLCA), or is capable of metabolizing LCA to isoalloLCA. For example, a contemplated bacterium may comprise a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, and Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23. In certain embodiments, a contemplated bacterium may comprise a transgene encoding a 3α-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 12-23. In certain embodiments, a contemplated bacterium comprises a nucleic acid comprising the nucleotide sequence of any one of SEQ ID NOs: 12-23, or a nucleotide sequence having at least 80% identity to any one of SEQ ID NOs: 12-23. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 12-23. Alternatively, or in addition, a contemplated bacterium may comprise a transgene encoding a 5α-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, for example, a 5α-reductase comprising an amino sequence encoded by any one of SEQ ID NOs: 61-67, or having at least 80% identity to an amino sequence encoded by any one of SEQ ID NOs: 61-67. In certain embodiments, a contemplated bacterium may comprise a transgene encoding a 5α-reductase, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 61-67. In certain embodiments, a contemplated bacterium comprises a nucleic acid comprising the nucleotide sequence of any one of SEQ ID NOs: 61-67, or a nucleotide sequence having at least 80% identity to any one of SEQ ID NOs: 61-67. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 61-67. Alternatively, or in addition, a contemplated bacterium may comprise a transgene encoding a 5β-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, for example, a 5β-reductase comprising an amino sequence encoded by any one of SEQ ID NOs: 68-74, or having at least 80% identity to an amino sequence encoded by any one of SEQ ID NOs: 68-74. In certain embodiments, a contemplated bacterium may comprise a transgene encoding a 5β-reductase, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 68-74. In certain embodiments, a contemplated bacterium comprises a nucleic acid comprising the nucleotide sequence of any one of SEQ ID NOs: 68-74, or a nucleotide sequence having at least 80% identity to any one of SEQ ID NOs: 68-74. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 68-74. Alternatively, or in addition, a contemplated bacterium may comprise a transgene encoding a 3β-hydroxysteroid dehydrogenase (3β-HSDH), or a functional fragment or variant thereof, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, for example, a 3-HSDH comprising an amino sequence encoded by any one of SEQ ID NOs: 24-47, or having at least 80% identity to an amino sequence encoded by any one of SEQ ID NOs: 24-47. In certain embodiments, a contemplated bacterium may comprise a transgene encoding a 3β-HSDH, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 24-47. In certain embodiments, a contemplated bacterium comprises a nucleic acid comprising the nucleotide sequence of any one of SEQ ID NOs: 24-47, or a nucleotide sequence having at least 80% identity to any one of SEQ ID NOs: 24-47. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 24-47.

In certain embodiments, a contemplated bacterium metabolizes a bile acid or bile salt to a sulfated product, or is capable of metabolizing a bile acid or bile salt to a sulfated product. For example, a contemplated bacterium may comprise a transgene encoding a sulfotransferase (SULT), or a functional fragment variant thereof, for example a Homo sapiens or Mus musculus, for example, a SULT comprising an amino sequence encoded by any one of SEQ ID NOs: 75-76, or having at least 80% identity to an amino sequence encoded by any one of SEQ ID NOs: 75-76. In certain embodiments, a contemplated bacterium may comprise a transgene encoding a SULT, or a functional fragment or variant thereof, comprising an amino sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to an amino sequence encoded by any one of SEQ ID NOs: 75-76. In certain embodiments, a contemplated bacterium comprises a nucleic acid comprising the nucleotide sequence of any one of SEQ ID NOs: 75-76, or a nucleotide sequence having at least 80% identity to any one of SEQ ID NOs: 75-76. In certain embodiments, a contemplated bacterium comprises a nucleic acid comprising a nucleic acid sequence having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to any one of SEQ ID NOs: 75-76. In some embodiments, a contemplated bacterium may comprise a transgene encoding SULT2A1, or a functional fragment variant thereof. In some embodiments, a contemplated bacterium may comprise a transgene encoding a modified SULT2A1, or a functional fragment variant thereof. In some embodiments, an encoded SULT2A1 is from Homo sapiens. In some embodiments, a contemplated bacterium may comprise a transgene encoding SULT2A8, or a functional fragment variant thereof. In some embodiments, a contemplated bacterium may comprise a transgene encoding a modified SULT2A8, or a functional fragment variant thereof. In some embodiments, an encoded SULT2A1 is from Mus musculus.

In some embodiments, a contemplated bacterium metabolizes, or is capable of metabolizing, CA to isoCA. In some embodiments, a contemplated bacterium comprises: (i) a transgene encoding a 3α-hydroxysteroid dehydrogenase (3α-HSDH), or a functional fragment or variant thereof, and/or (ii) a transgene encoding a 3β-hydroxysteroid dehydrogenase (3β-HSDH), or a functional fragment or variant thereof.

In some embodiments a contemplated bacterium metabolizes, or is capable of metabolizing, CA to UCA. In some embodiments, a contemplated bacterium comprises: (i) a transgene encoding a 7α-hydroxysteroid dehydrogenase (7α-HSDH), or a functional fragment or variant thereof, and/or (ii) a transgene encoding a 7β-hydroxysteroid dehydrogenase (7β-HSDH), or a functional fragment or variant thereof.

In some embodiments, a contemplated bacterium metabolizes, or is capable of metabolizing, CA to lagoCA. In some embodiments, a contemplated bacterium comprises: (i) a transgene encoding a 12α-hydroxysteroid dehydrogenase (12α-HSDH), or a functional fragment or variant thereof, and/or (ii) a transgene encoding a 12β-hydroxysteroid dehydrogenase (12β-HSDH), or a functional fragment or variant thereof.

In some embodiments, a contemplated bacterium metabolizes, or is capable of metabolizing, CA to isoUCA. In some embodiments, a contemplated bacterium comprises: (i) a transgene encoding a 3α-hydroxysteroid dehydrogenase (3α-HSDH), or a functional fragment or variant thereof, (ii) a transgene encoding a 3β-hydroxysteroid dehydrogenase (3β-HSDH), or a functional fragment or variant thereof, (iii) a transgene encoding a 7α-hydroxysteroid dehydrogenase (7α-HSDH), or a functional fragment or variant thereof, and/or (iv) a transgene encoding a 7β-hydroxysteroid dehydrogenase (7β-HSDH), or a functional fragment or variant thereof.

In some embodiments, a contemplated bacterium metabolizes, or is capable of metabolizing, CA to iso-lagoCA. In some embodiments, a contemplated bacterium comprises: (i) a transgene encoding a 3α-hydroxysteroid dehydrogenase (3α-HSDH), or a functional fragment or variant thereof, (ii) a transgene encoding a 3β-hydroxysteroid dehydrogenase (3β-HSDH), or a functional fragment or variant thereof, (iii) a transgene encoding a 12α-hydroxysteroid dehydrogenase (12α-HSDH), or a functional fragment or variant thereof, and/or (iv) a transgene encoding a 12β-hydroxysteroid dehydrogenase (12β-HSDH), or a functional fragment or variant thereof.

In some embodiments, a contemplated bacterium metabolizes, or is capable of metabolizing, CA to lagoUCA. In some embodiments, a contemplated bacterium comprises: (i) a transgene encoding a 7α-hydroxysteroid dehydrogenase (7α-HSDH), or a functional fragment or variant thereof, (ii) a transgene encoding a 7β-hydroxysteroid dehydrogenase (7β-HSDH), or a functional fragment or variant thereof, (iii) a transgene encoding a 12α-hydroxysteroid dehydrogenase (12α-HSDH), or a functional fragment or variant thereof, and/or (iv) a transgene encoding a 12β-hydroxysteroid dehydrogenase (12β-HSDH), or a functional fragment or variant thereof.

In some embodiments, a contemplated bacterium metabolizes, or is capable of metabolizing, CA to iso-lagoCA. In some embodiments, a contemplated bacterium comprises: (i) a transgene encoding a 3α-hydroxysteroid dehydrogenase (3α-HSDH), or a functional fragment or variant thereof, (ii) a transgene encoding a 3β-hydroxysteroid dehydrogenase (3β-HSDH), or a functional fragment or variant thereof, (iii) a transgene encoding a 7α-hydroxysteroid dehydrogenase (7α-HSDH), or a functional fragment or variant thereof, (iv) a transgene encoding a 7β-hydroxysteroid dehydrogenase (7β-HSDH), or a functional fragment or variant thereof, (v) a transgene encoding a 12α-hydroxysteroid dehydrogenase (12α-HSDH), or a functional fragment or variant thereof, and/or (vi) a transgene encoding a 12β-hydroxysteroid dehydrogenase (12β-HSDH), or a functional fragment or variant thereof.

In some embodiments, a contemplated bacterium metabolizes, or is capable of metabolizing, CDCA to isoCDCA. In some embodiments, a contemplated bacterium comprises: (i) a transgene encoding a 3α-hydroxysteroid dehydrogenase (3α-HSDH), or a functional fragment or variant thereof, and/or (ii) a transgene encoding a 3 O-hydroxysteroid dehydrogenase (3β-HSDH), or a functional fragment or variant thereof.

In some embodiments, a contemplated bacterium metabolizes, or is capable of metabolizing, CDCA to isoUDCA. In some embodiments, a contemplated bacterium comprises: (i) a transgene encoding a 3α-hydroxysteroid dehydrogenase (3α-HSDH), or a functional fragment or variant thereof, (ii) a transgene encoding a 3β-hydroxysteroid dehydrogenase (3β-HSDH), or a functional fragment or variant thereof, (iii) a transgene encoding a 7α-hydroxysteroid dehydrogenase (7α-HSDH), or a functional fragment or variant thereof, and/or (iv) a transgene encoding a 7β-hydroxysteroid dehydrogenase (7β-HSDH), or a functional fragment or variant thereof.

In some embodiments, a contemplated bacterium metabolizes, or is capable of metabolizing, LCA to isoLCA. In some embodiments, a contemplated bacterium comprises: (i) a transgene encoding a 3α-hydroxysteroid dehydrogenase (3α-HSDH), or a functional fragment or variant thereof, and/or (ii) a transgene encoding a 3β-hydroxysteroid dehydrogenase (3β-HSDH), or a functional fragment or variant thereof.

In some embodiments, a contemplated bacterium metabolizes, or is capable of metabolizing, CA to isoalloCA. In some embodiments, a contemplated bacterium comprises: (i) a transgene encoding a 3α-hydroxysteroid dehydrogenase (3α-HSDH), or a functional fragment or variant thereof, (ii) a transgene encoding a 5α-reductase, or a functional fragment or variant thereof, (iii) a transgene encoding a 5β-reductase, or a functional fragment or variant thereof, and/or (iv) a transgene encoding a 3β-hydroxysteroid dehydrogenase (3β-HSDH), or a functional fragment or variant thereof.

In certain embodiments, at least one transgene or nucleic acid is operably linked to at least one promoter, e.g., a constitutive or inducible promoter, e.g., a phage-derived promoter. Exemplary promoters include those comprising the consensus sequence GTTAA(n)₄₋₇GTTAA(n)₃₄₋₃₈TA(n)₂TTTG (SEQ ID: 79), or comprising SEQ ID NO: 77, SEQ ID NO: 78, or SEQ ID NO: 79.

In certain embodiments, a contemplated bacterium has been modified to colonize the human gut with increased abundance, stability, predictability, or ease of initial colonization (for example, relative to a similar or otherwise identical bacterium that has not been modified). For example, a contemplated bacterium may be modified to increase its ability to utilize a privileged nutrient as carbon source. For example, a contemplated bacterium may comprise one or more transgenes that increase its ability to utilize a privileged nutrient as carbon source. Exemplary privileged nutrients include, e.g., a marine polysaccharide, e.g., a porphyran. A disclosed bacterium may, e.g., upon administration to a human subject, result in an abundance of greater than 10¹², greater than 10¹¹, greater than 10¹⁰, greater than 10⁹, greater than 10⁸, or greater than 10⁷ colony-forming units (CFUs) per gram of fecal content. A disclosed bacterium may, e.g., upon administration to a human subject, result in an abundance of at least 10¹², at least 10¹¹, at least 10¹⁰, at least 10⁹, at least 10⁸, or at least 10⁷ CFUs per gram of fecal content. A disclosed bacterium may, e.g., upon administration to a human subject, result in an abundance of less than 10¹², less than 10¹¹, less than 10¹⁰, less than 10⁹, less than 10⁸, or less than 10⁷ CFUs per gram of fecal content. In some embodiments, a disclosed bacterium may result in an abundance of about 10¹² to about 10¹⁰, about 10¹² to about 10⁹, about 10¹² to about 10⁸, about 10¹¹ to about 10¹⁰, about 10¹¹ to about 10⁹, about 10¹¹ to about 10⁸, about 10¹⁰ to about 10⁹, about 10¹⁰ to about 10⁸, or about 10⁹ to about 10⁸ CFUs per gram of fecal content. In some aspects, an abundance is achieved about 1 hour, about 2 hours, about 3 hours, about 4 hours, about 6 hours, about 7 hours, about 8 hours, about 9 hours, about 10 hours, about 11 hours, or about 12 hours after administering a bacterium as described herein. In some embodiments, an abundance is maintained for at least 6 hours, at least 12 hours, at least 24 hours, at least 36 hours, at least 48 hours, at least 72 hours, at least 1 week, at least 2 weeks, at least 3 weeks, at least 4 weeks, at least 2 months, at least 3 months, at least 4 months, at least 5 months, at least 6 months, or at least 1 year. In some embodiments, an abundance is maintained at least 10¹², at least 10¹¹, at least 10¹⁰, at least 10⁹, at least 10⁸, or at least 10⁷ CFUs per gram of fecal content for at least 6 hours, at least 12 hours, at least 24 hours, at least 36 hours, at least 48 hours, at least 72 hours, at least 1 week, at least 2 weeks, at least 3 weeks, at least 4 weeks, at least 2 months, at least 3 months, at least 4 months, at least 5 months, at least 6 months, or at least 1 year. In some embodiments, an abundance is maintained at about 10¹², at about 10¹¹, at about 10¹⁰, at about 10⁹, at about 10⁸, or at about 10⁷ CFUs per gram of fecal content for at least 6 hours, at least 12 hours, at least 24 hours, at least 36 hours, at least 48 hours, at least 72 hours, at least 1 week, at least 2 weeks, at least 3 weeks, at least 4 weeks, at least 2 months, at least 3 months, at least 4 months, at least 5 months, at least 6 months, or at least 1 year. In some embodiments, an abundance is maintained at about 10¹² to about 10¹⁰, about 10¹² to about 10⁹, about 10¹² to about 10⁸, about 10¹¹ to about 10¹⁰, about 10¹¹ to about 10⁹, about 10¹¹ to about 10⁸, about 10¹⁰ to about 10⁹, about 10¹⁰ to about 10⁸, or about 10⁹ to about 10⁸ CFUs per gram of fecal content for at least 6 hours, at least 12 hours, at least 24 hours, at least 36 hours, at least 48 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

In another aspect, provided herein is a pharmaceutical composition comprising a disclosed bacterium and a pharmaceutically acceptable excipient. In certain embodiments, a contemplated pharmaceutical composition is formulated as a capsule or tablet, e.g., an enteric coated capsule. In certain embodiments, a contemplated pharmaceutical composition further comprises a privileged nutrient, e.g., a marine polysaccharide, e.g., a porphyran.

In another aspect, provided herein is a method of reducing a level of a bile acid or bile salt (e.g., CDCA or DCA) in a subject. In certain embodiments, a contemplated method comprises administering to the subject an effective amount of a bacterium comprising one or more transgenes that increase the bacterium's ability to metabolize the bile acid or bile salt. In certain embodiments, a contemplated method comprises administering to the subject an effective amount of a bacterium comprising one or more transgenes that enable the bacterium to metabolize the bile acid or bile salt. In certain embodiments, a contemplated method comprises administering to the subject an effective amount of a disclosed bacterium or pharmaceutical composition. In certain embodiments, a subject has a disease and/or disorder associated with bile acids or bile salts. For example, in some embodiments, a subject has a bile acid disorder, e.g., bile acid diarrhea. In some embodiments, a bacterium is of a genus selected from Escherichia (e.g., E. coli), Clostridia, Rhodococcus, Corynebacterium, Pseudomonas, Acidobacteria, Streptomyces, Bacillus, and Paenibacillus.

In another aspect, provided herein is a method of treating a disease and/or disorder associated with bile acids or bile salts in a subject in need thereof. In some aspects, provided herein is a method of treating a bile acid disorder in a subject in need thereof. In certain embodiments, a contemplated method comprises administering to the subject an effective amount of a bacterium comprising one or more transgenes that increase the bacterium's ability to metabolize the bile acid or bile salt. In certain embodiments, a contemplated method comprises administering to the subject an effective amount of a bacterium comprising one or more transgenes that enable the bacterium to metabolize the bile acid or bile salt. In certain embodiments, a contemplated method comprises administering to the subject an effective amount of a disclosed bacterium or pharmaceutical composition. In some embodiments, a bacterium is of a genus selected from Escherichia (e.g., E. coli), Clostridia, Rhodococcus, Corynebacterium, Pseudomonas, Acidobacteria, Streptomyces, Bacillus, and Paenibacillus. Exemplary diseases and/or disorders associated with bile acids or bile salts include bile acid diarrhea (e.g., type 1 or type 2 (idiopathic) bile acid diarrhea), a metabolic disorder (e.g., obesity, type 2 diabetes, hyperlipidemia, or atherosclerosis), cholelithiasis (e.g., intrahepatic cholestasis of pregnancy, or cholelithiasis associated with primary sclerosing cholangitis or primary biliary cholangitis), liver disease (e.g., cystic liver disease or non-alcoholic fatty liver disease), cancer (e.g., colon cancer or gastrointestinal cancer), an autoimmune or inflammatory disorder (e.g., inflammatory bowel disease (IBS)), or a bacterial infection (e.g., a Clostridiodes difficile infection). In some embodiments, a bile acid disorder is bile acid diarrhea. In some embodiments, bile acid diarrhea is type 1 or type 2 (idiopathic) bile acid diarrhea.

In another aspect, provided herein is a method of altering the hydrophobicity of a bile acid pool in a subject. In some aspects, provided herein is a method of altering the hydrophobicity of a bile acid pool in a subject where said method comprises administering to the subject an effective amount of a disclosed bacterium or pharmaceutical composition. In some aspects, methods provided herein further comprise administering a privileged nutrient to the subject. In some aspects, a privileged nutrient is administered to a subject prior to, at the same time as, or after a bacterium described in the present disclosure. In some embodiments, a privileged nutrient is a marine polysaccharide. In some aspects, a marine polysaccharide is a porphyran. In some aspects, an administered bacterium alters the hydrophobicity of a bile acid pool in a subject by increasing the hydrophobicity of said bile acid pool. In some aspects, an administered bacterium alters the hydrophobicity of a bile acid pool in a subject by decreasing the hydrophobicity of said bile acid pool. Methods of determining the hydrophobicity of a bile acid pool are known in the art, and include calculating a bile acid hydrophobicity index by taking the sum of the following formula for each bile acid: (Heuman index value of a given bile acid multiplied by its proportion). (See, Heuman (1989) J Lipid Res 30(5):719-730). In certain embodiments, a bacterium disclosed can increase this cumulative index (make a bile acid pool more hydrophilic) or decrease this cumulative index (make a bile acid pool more hydrophobic).

Contemplated methods may comprise administration of a disclosed bacterium or pharmaceutical composition to a subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In certain embodiments, the time between consecutive administrations of a disclosed bacterium or pharmaceutical composition to a subject is greater than 48 hours.

Contemplated methods may further comprise administrating a privileged nutrient to the subject, e.g., a marine polysaccharide, e.g., a porphyran. For example, a disclosed privileged nutrient may be administered to the subject prior to, at the same time as, or after a disclosed bacterium.

In many embodiments, a subject receiving compositions and methods of the present disclosure is an animal. In some embodiments, the subject is a human.

These and other aspects and features of the disclosure are described in the following detailed description and claims.

### DESCRIPTION OF THE DRAWINGS

The disclosure can be more completely understood with reference to the following drawings.

FIG. 1 Panels A and B present a schematic overview of bile acid metabolism by engineered bacteria to be used therapeutically. FIG. 1A is an exemplary schematic of engineered bacteria (e.g., an engineered Bacteroides strain) that are engineered to selectively metabolize bile acids or bile salts that are associated with causing, promoting, and/or increasing disease to products that are generally considered not to promote disease. Exemplary bacteria include those engineered to have transgenes encoding hydroxysteroid dehydrogenases (HSDHs). FIG. 1B is an exemplary schematic of engineered bacteria (e.g., an engineered Bacteroides strain) that are engineered to selectively metabolize bile acid or bile salt substrates to products that possess therapeutic activity. Exemplary bacteria include those engineered to have transgenes encoding hydroxysteroid dehydrogenases (HSDHs).

FIG. 2 Panel A, B, C, D, and E present exemplary bile acid enzyme-mediated metabolic pathways that may be carried out by engineered bacteria (e.g., engineered Bacteroides strains) engineered to have transgenes encoding various hydroxysteroid dehydrogenases (HSDHs) that can metabolize specific bile acids. FIG. 2A shows a conversion pathway of chenodeoxycholic acid (CDCA) to ursodeoxycholic acid (UDCA) by bacterial 7-hydroxysteroid dehydrogenases (HSDHs). FIG. 2B shows a conversion pathway of deoxycholic acid (DCA) to isodeoxycholic acid (isoDCA) by bacterial 3-HSDHs. FIG. 2C shows a conversion pathway of DCA to lagodeoxycholic acid (lagoDCA) by bacterial 12-HSDHs. FIG. 2D shows examples of predominate bile acid species that are found in humans and highlights the 3-OH, 7-OH, and 12-OH residues of each molecule that can be targeted with transgenes encoding various HSDHs. This demonstrates that other than LCA, there are multiple hydroxyl residues per bile acid substrate that can be targeted with various transgenes encoding HSDHs. FIG. 2E shows examples of predominate bile acid species that are found in humans and resulting bile acid products that are generated by targeting a single hydroxyl residue or targeting multiple hydroxyl residues using engineered bacteria expressing HSDHs. With this approach multiple bile acid products can be generated from a single bile acid substrate, other than LCA. These modifications using HSDHs generate bile acid products that have decreased hydrophobicity (or increased hydrophilicity).

FIG. 3 presents exemplary bile acid enzyme-mediated metabolic pathways carried out by engineered bacteria (e.g., engineered Bacteroides strains) engineered to have transgenes encoding various hydroxysteroid dehydrogenases (HSDHs) to generate cholic acid (CA) isomers. Engineered Bacteroides strains expressing various site-specific HSDHs enzymes targeting one of the three hydroxyl residues of CA were incubated in BHIS media supplemented with 100 μM CA under anaerobic conditions at 37° C. for 24 hours either alone or in various combinations with one another. Shown are the chromatogram traces for m/z=407.2783-407.2823 following UHPLC-HRMS analysis of conditioned media samples. The text over the chromatograms indicated the identity of the bile acid metabolite. SC, sterile control; NB144, non-metabolizing control Bacteroides strain. This figure demonstrates various site-specific HSDHs pathways can be combined to generate a collection of various CA isomers.

FIG. 4 presents exemplary bile acid enzyme-mediated metabolic pathways carried out by engineered bacteria (e.g., engineered Bacteroides strains) engineered to have transgenes encoding various hydroxysteroid dehydrogenases (HSDHs) to generate chenodeoxycholic acid (CDCA) isomers. Engineered Bacteroides strains expressing various site-specific HSDHs enzymes targeting one of the two hydroxyl residues of CDCA were incubated in BHIS media supplemented with 100 μM CDCA under anaerobic conditions at 37° C. for 24 hours either alone or in various combinations with one another. Shown are the chromatogram traces for m/z=391.2834-391.2874 following UHPLC-HRMS analysis of conditioned media samples. The text over the chromatograms indicated the identity of the bile acid metabolite. SC, sterile control; NB144, non-metabolizing control Bacteroides strain. This figure demonstrates various site-specific HSDHs pathways can be combined to generate a collection of various CDCA isomers.

FIG. 5 presents exemplary bile acid enzyme-mediated metabolic pathways carried out by engineered bacteria (e.g., engineered Bacteroides strains) engineered to have transgenes encoding various hydroxysteroid dehydrogenases (HSDHs) to generate deoxycholic acid (DCA) isomers. Engineered Bacteroides strains expressing various site-specific HSDHs enzymes targeting one of the two hydroxyl residues of DCA were incubated in BHIS media supplemented with 100 μM DCA under anaerobic conditions at 37° C. for 24 hours. Shown are the chromatogram traces for m/z=391.2834-391.2874 following UHPLC-HRMS analysis of conditioned media samples. The text over the chromatograms indicated the identity of the bile acid metabolite. SC, sterile control; NB144, non-metabolizing control Bacteroides strain. This figure demonstrates various site-specific HSDHs pathways can be used to generate a collection of various DCA isomers.

FIG. 6 presents exemplary bile acid enzyme-mediated metabolic pathways carried out by engineered bacteria (e.g., engineered Bacteroides strains) engineered to have transgenes encoding various hydroxysteroid dehydrogenases (HSDHs) to generate a lithocholic acid (LCA) isomer. Engineered Bacteroides strains expressing site-specific HSDHs enzymes targeting the one hydroxyl residues of LCA was incubated in BHIS media supplemented with 100 μM LCA under anaerobic conditions at 37° C. for 24 hours. Shown are the chromatogram traces for m/z=375.2886-375.2924 following UHPLC-HRMS analysis of conditioned media samples. The text over the chromatograms indicated the identity of the bile acid metabolite. SC, sterile control; NB144, non-metabolizing control Bacteroides strain. This figure demonstrates that site-specific HSDHs pathways can be used to generate a LCA isomer.

FIG. 7 Panels A, B, C, and D present exemplary bile acid enzyme-mediated metabolic pathways carried out by engineered bacteria (e.g., engineered Bacteroides strains) engineered to have transgenes encoding various bile acid metabolizing enzymes to generate the isoallo-bile acids. Four enzymes generally involved in the metabolism are a 3α-hydroxysteroid dehydrogenase (HSDH), a 5α-reductase, a 5β-reductase, and a 3β-HSDH producing isoallo bile acid products. FIG. 7A shows a conversion pathway of CA to isoalloCA. FIG. 7B shows a conversion pathway of CDCA to isoalloCDCA. FIG. 7C shows a conversion pathway of DCA to isoalloDCA. FIG. 7D shows a conversion pathway of LCA to isoalloLCA.

FIG. 8 Panels A and B present exemplary bile acid enzyme-mediated metabolic pathways carried out by engineered bacteria (e.g., engineered Bacteroides strains) engineered to have transgenes encoding various bile acid metabolizing enzymes to generate the bile acid metabolite isoallochenodeoxycholic acid. FIG. 8A shows a four-enzyme pathway involved in the biosynthesis of isoalloCDCA, which includes 3β-hydroxysteroid dehydrogenase (3β-HSDH), 5β-reductase (5BR), 5α-reductase (5AR), and 3α-hydroxysteroid dehydrogenase (3α-HSDH), FIG. 8B shows various engineered Bacteroides strains expressing enzymes in the isoallo-bile acid enzymes incubated in BHIS media supplemented with 100 μM CDCA under anaerobic conditions at 37° C. for 24 hours. Shown are the chromatogram traces for the various metabolites in the isoalloCDCA pathway following UHPLC-HRMS analysis of conditioned media samples. The text over the chromatograms indicated the identity of the bile acid metabolite. This data demonstrates that engineered Bacteroides can be used to generate isoallo-bile acid metabolites.

FIG. 9 Panels A and B present exemplary bile acid enzyme-mediated metabolic pathways carried out by engineered bacteria (e.g., engineered Bacteroides strains) engineered to have transgenes encoding sulfotransferases to generate sulfated bile acid metabolites. FIG. 9A shows a metabolic pathway engineered in Bacteroides to metabolize cholic acid (CA) to sulfated cholic acid (CA-S). FIG. 9B shows various engineered Bacteroides strains expressing sulfotransferase enzymes incubated in BHIS media supplemented with 100 μM bile acid under anaerobic conditions at 37° C. for 24 hours. SC, sterile control; NB144, non-metabolizing control Bacteroides strain. sZR0393 expresses a modified version of the SULT2A1 from Homo sapiens and sZR0394 expresses a modified version of the SULT2A8 from Mus musculus. Shown are the chromatogram traces for the various sulfated bile acid metabolites following UHPLC-HRMS analysis of conditioned media samples. Shown are the chromatogram traces for CA-S [m/z=487.2347-487.2395], CDCA-S [m/z=471.2398-471.2446], DCA-S [m/z=471.2398-471.2446], and LCA-S [m/z=455.2450-455.2496] following UHPLC-HRMS analysis of conditioned media samples with strains grown in the presence of each bile acid. This data demonstrates that engineered Bacteroides strain expressing various sulfotransferases are capable of generated sulfated-bile acid products,

FIG. 10 Panels A, B, C, and D show in vitro conversion of CDCA to UDCA using an engineered Bacteroides strain. Bacteroides strains were incubated in BHIS media supplemented with 100 μM CDCA under anaerobic conditions at 37° C. Conditioned media was sampled over time and analyzed for CDCA depletion and UDCA production using UHPLC-HRMS. FIG. 10A shows an exemplary schematic of a reaction for microbial conversion of CDCA to UDCA using 7α-HSDH and 7β-HSDH enzymes. FIG. 10B shows levels of CDCA (circles) and UDCA (squares) over time following incubation with a control non-metabolizing Bacteroides strain NB144 that is not engineered to metabolize CDCA. FIG. 10C shows levels of CDCA (circles) and UDCA (squares) over time following incubation with a engineered UDCA-producing Bacteroides strain sPS049. Strain sPS049 completely converted 100 μM of CDCA into UDCA within 60 minutes of incubation. FIG. 10D shows rate of bile acid metabolism expected in the human GIT for CDCA (left column for all groups) and UDCA (right column for all groups). Rate of metabolism within a human gut was extrapolated based on linear rate of bile acid metabolism observed in this in vitro experiment (FIG. 10B and FIG. 10C) and expected colonization level of a Bacteroides strain in a human GIT. The engineered UDCA-producing Bacteroides strain sPS049 was modeled to deplete >3 mM of CDCA per hour. Experiments were performed in triplicate.

FIG. 11 Panels A and B show ex vivo conversion of CDCA to UDCA using an engineered Bacteroides strain in the presence of a complex community of human fecal bacteria. Human fecal slurries from five healthy individuals (A-E) were mixed with NB144, a non-metabolizing control Bacteroides strain, or sPS049, a UDCA-producing Bacteroides strain, and incubated in BHIS media supplemented with 100 μM CDCA under anaerobic conditions at 37° C. Conditioned media was sampled over time and analyzed for CDCA depletion (left column for all groups) and UDCA production (right column for all groups) using UHPLC-HRMS. FIG. 11A shows rate of bile acid metabolism expected in the human gut environment following ex vivo incubation of Bacteroides strains with human fecal bacteria from five healthy individuals. FIG. 11B shows rate of bile acid metabolism expected in a human gut environment following ex vivo incubation of Bacteroides strains with human fecal bacteria averaged across five healthy individuals (from FIG. 11A). Rate of metabolism within a human gut was extrapolated based on linear rate of bile acid metabolism observed in this ex vivo experiment and expected colonization level of a Bacteroides strain in a human GIT. The engineered UDCA-producing Bacteroides strain sPS049 was modeled to deplete ˜2 mM of CDCA per hour in the presence of a complex human gut microbial community. Rate determinations were performed in triplicate.

FIG. 12 Panels A and B show ex vivo conversion of CDCA to UDCA from complex microbial community samples of conventionally-raised mice colonized with an engineered Bacteroides strain. Conventionally-raised mice were colonized with sPS064, a non-metabolizing control Bacteroides strain, or sPS049, a UDCA-producing engineered Bacteroides strain. Mice were colonized by Bacteroides strains by gavage and then transferred on a porphyran-containing chow diet for strain maintenance. Following one week of colonization, mice were sacrificed and cecal and fecal samples were collected. FIG. 12A shows cecal and fecal sample resuspensions performed with BHIS media supplemented with CDCA at 0.1 mM final concentrations and incubated under anaerobic conditions at 37° C.

FIG. 12B shows cecal and fecal sample resuspensions performed with BHIS media supplemented with CDCA at 1 mM final concentrations and incubated under anaerobic conditions at 37° C. Conditioned media was sampled over time and analyzed for CDCA depletion (left column for all groups) and UDCA production (right column for all groups) using UHPLC-HRMS. Rate of metabolism within the gut was extrapolated based on a linear rate of bile acid metabolism observed in this ex vivo experiment and expected colonization level of a Bacteroides strain in a GIT. At an assay concentration of 1 mM CDCA, engineered UDCA-producing Bacteroides strain sPS049 was capable of depleting ˜5-6 mM of CDCA per hour in the presence of a complex gut microbial community under porphyran-colonization conditions. Rate determinations were calculated from n=3 mice per group.

FIG. 13 Panels A and B show in vivo conversion of CDCA to UDCA by an engineered Bacteroides strain in mice. FIG. 13A shows fecal bile acid levels of CDCA and UDCA from gnotobiotic mice colonized with various Bacteroides strains following oral delivery of CDCA by gavage. Germ-free mice were colonized with NB144, a non-metabolizing control Bacteroides strain, or sPS049, a UDCA-producing engineered Bacteroides strain by gavage and maintained on a porphyran-supplemented chow. Following one week of colonization, mice were gavaged with a 500 mg/kg dose of CDCA. Mice were then singly housed for 24 hours, and then fecal samples were collected and pooled for analysis of CDCA depletion (left column for all groups) and UDCA production (right column for all groups) using UHPLC-HRMS. The UDCA-producing Bacteroides strain sPS049 was able to reduce the levels of CDCA and produce a corresponding increase amount of UDCA, in the feces compared to the control strain NB144. Experimental groups included n=3 mice. FIG. 13B shows fecal bile acid levels of CDCA and UDCA from conventionally-raised mice colonized with various Bacteroides strains following oral delivery of CDCA by gavage. Conventionally-raised mice were colonized with sPS064, a non-metabolizing control Bacteroides strain, or sPS049, a UDCA-producing engineered Bacteroides strain by gavage and maintained on a porphyran-supplemented chow. Following one week of colonization, mice were gavaged with a 200 mg/kg dose of CDCA. Mice were then singly housed for 24 hours, and then fecal samples were collected and pooled for analysis of CDCA depletion (left column for all groups) and UDCA production (right column for all groups) using UHPLC-HRMS. The UDCA-producing Bacteroides strain sPS049 was able to reduce levels of CDCA and produce a corresponding increase amount of UDCA, in the feces compared to the control strain sPS064. Experimental groups included n=5 mice.

FIG. 14 Panels A, B, C, D and E show in vitro conversion of DCA to isoDCA using an engineered Bacteroides strain. Bacteroides strains were incubated in BHIS media supplemented with 100 μM DCA under anaerobic conditions at 37° C. Conditioned media was sampled over time and analyzed for DCA depletion (circles or left column for all groups) and isoDCA production (squares or right column for all groups) using UHPLC-HRMS. FIG. 14A shows a reaction for microbial conversion of DCA to isoDCA using 3α-HSDH and 3β-HSDH enzymes. FIG. 14B shows levels of DCA and isoDCA over time following incubation with a control non-metabolizing Bacteroides strain NB144. NB144 does not metabolize DCA. FIG. 14C and FIG. 14D show levels of DCA and isoDCA over time following incubation with the engineered isoDCA-producing Bacteroides strain sPS235 (expressing 3α-HSDH from Eggerthella lenta SEQ ID NO: 13 and 3β-HSDH from Ruminococcus gnavus SEQ ID NO: 24) and sJT0025 (expressing 3α-HSDH from a compost metagenome SEQ ID NO: 18 and 3β-HSDH from Holdemania filiformis SEQ ID NO: 32). Strains sPS235 and sJT0025 can completely convert 100 μM of DCA into isoDCA within 240 minutes of incubation. FIG. 14E shows rate of bile acid metabolism expected in a human gut environment. Rate of metabolism within a human gut was extrapolated based on a linear rate of bile acid metabolism observed in this in vitro experiment and expected colonization level of a Bacteroides strain in a human GIT. Engineered isoDCA-producing Bacteroides strain sPS235 was modeled to deplete ˜0.15 mM of DCA per hour while superior strains sJT0022 (SEQ ID NO: 18 and 28), sJT0023 (SEQ ID NO: 18 and 30)), sJT0025 (SEQ ID NO: 18 and 32), and sJT0026(SEQ ID NO: 18 and 36) show metabolism rates ˜1 mM/hour or greater.

FIG. 15 shows in vivo conversion of DCA to isoDCA by an engineered Bacteroides strain in mice. FIG. 15 shows fecal bile acid levels of DCA and isoDCA from conventionally-raised mice colonized with various Bacteroides strains following oral delivery of DCA by gavage. Conventionally-raised mice were colonized with sPS064, a non-metabolizing control Bacteroides strain, or multiple isoDCA-producing engineered Bacteroides strains by gavage and maintained on a porphyran-supplemented chow. IsoDCA producting strains used were sPS235 (SEQ ID NO: 13 and 24), sJT0022 (SEQ ID NO: 18 and 28), sJT0023 (SEQ ID NO: 18 and 30), sJT0025 (SEQ ID NO: 18 and 32), and sJT0026 (SEQ ID NO: 18 and 36). Following a minimum of one week of colonization, mice were singly housed for 24 hours, and then fecal samples were collected and pooled for analysis of DCA depletion (left column for all groups) and isoDCA production (right column for all groups) using UHPLC-HRMS. The control strain sPS064 as well at the isoDCA strain sPS235 did not reduce DCA levels in feces or generate substantial isoDCA. Compared to the control strain, sPS235 showed a 26% increase (1.63 moles/gram increase) in DCA levels. In comparison, isoDCA-producing Bacteroides strain sJT0022, sJT0023, JT0025, and sJT0026 were able to reduce levels of DCA and produce an increase of isoDCA, in the feces. Compared to the control strain, sJT0022, sJT0023, JT0025, and sJT0026 showed an average of a 77% decrease (4.3 μmoles/gram decrease) in DCA levels. Note the reduction of DCA levels in the feces only occurred with animals that were colonized with the best metabolizing strains as determined by in vitro experiments (FIG. 14E), which were strains sJT0022, sJT0023, JT0025, and sJT0026. Experimental groups included n=3-4 mice.

FIG. 16 Panels A, B, C, and D show in vitro conversion of DCA to lagoDCA using an engineered Bacteroides strain. Bacteroides strains were incubated in BHIS media supplemented with 100 μM DCA under anaerobic conditions at 37° C. Conditioned media was sampled over time and analyzed for DCA depletion (circles) and isoDCA production (squares) using UHPLC-HRMS. FIG. 16A shows a reaction for the microbial conversion of DCA to lagoDCA using 12α-HSDH and 12β-HSDH enzymes. FIG. 16B shows levels of DCA and lagoDCA over time following incubation with a control non-metabolizing Bacteroides strain NB144. NB144 does not metabolize DCA. FIG. 16C shows the levels of DCA and lagoDCA over time following incubation with engineered lagoDCA-producing Bacteroides strain sPS385. Strain sPS385 can almost completely convert 100 μM of DCA into lagoDCA within 10 minutes of incubation. FIG. 16D shows rate of bile acid metabolism expected in a human GIT for DCA (left column for all groups) and lagoDCA (right column for all groups). Rate of metabolism within a human gut was extrapolated based on a linear rate of bile acid metabolism observed in this in vitro experiment (FIG. 16B and FIG. 16C) and expected colonization level of a Bacteroides strain in a human GIT. The engineered lagoDCA-producing Bacteroides strain sPS385 was modeled to deplete >1 mM of DCA per hour. Experiments were performed in triplicate.

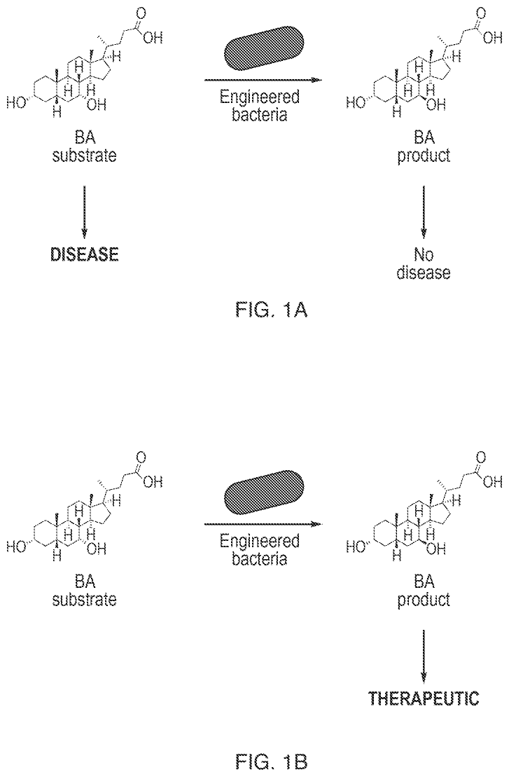

### DETAILED DESCRIPTION

The disclosure relates generally to bacteria that have been modified to metabolize a bile acid or bile salt. For example, in one aspect, provided herein is a bacterium or bacteria (e.g., a commensal and/or anaerobic bacterium) comprising one or more transgenes that increase the bacterium's or bacteria's ability to metabolize one or more bile acids (for example, relative to a similar or otherwise identical bacterium that does not comprise the one or more transgenes).

It is contemplated that disclosed bacteria may, upon administration to a subject, metabolize a bile acid or bile salt in the subject, and therefore be useful for treating a disease or disorder associated with bile acids or bile salts in the subject, e.g., bile acid diarrhea. Accordingly, the disclosure further relates to pharmaceutical compositions or units and methods of using disclosed bacteria to treat diseases or disorders associated with bile acids or bile salts, e.g., bile acid diarrhea.

It is contemplated that disclosed bacteria may, upon administration to a subject, metabolize a bile acid or bile salt in the subject to bile acid or bile salt product that displays therapeutic properties. Accordingly, the disclosure further relates to pharmaceutical compositions or units and methods of using disclosed bacteria to treat disorders or diseases with such bile acid or bile salt products.

A contemplated modified bacterium may additionally have the ability to utilize a carbon source, such as the marine polysaccharide porphyran, that other bacteria in the gut of a subject to be treated are largely unable to utilize. As a result, the proliferation, abundance, or stability of the modified bacteria in the gut of the subject may be maintained by supplying it with the carbon source.

### I. Modified Bacteria

The disclosure relates generally to bacteria that have been modified to metabolize a bile acid or bile salt. For example, a contemplated bacterium may be modified to comprise one or more transgenes that increase the bacterium's ability to metabolize one or more bile acid or bile salts (for example, relative to a similar or otherwise identical bacterium that does not comprise the one or more transgenes). It is contemplated that the one or more transgenes may, e.g., be on a plasmid, bacterial artificial chromosome, or be genomically integrated. When a bacterium comprises one or more transgenes encoding multiple proteins, it is contemplated that the open reading frames encoding two or more of the proteins may, e.g., be present in a single operon.

Exemplary bile acids include cholic acid (CA), chenodeoxycholic acid (CDCA), deoxycholic acid (DCA), lithocholic acid (LCA), isocholic acid (isoCA), isochenodeoxycholic acid (isoCDCA), isodeoxycholic acid (isoDCA), isolithocholic acid (isoLCA), ursocholic acid (UCA), ursodeoxycholic acid (UDCA), lagocholic acid (lagoCA), lagodeoxycholic acid (lagoDCA), β-muricholic acid (β-MCA), α-muricholic acid (α-MCA), γ-muricholic acid (γ-MCA), and ω-muricholic acid (ω-MCA). Exemplary bile salts include taurocholic acid (TCA), glycocholic acid (GCA), taurochenodeoxycholic acid (TCDCA), glycochenodeoxycholic (GCDCA), taurodeoxycholic acid (TDCA), glycodeoxycholic acid (GDCA), taurolithocholic acid (TLCA), and glycolithocholic acid (GLCA). It is understood that reference herein to one or more “bile acids” may also include one or more “bile salts,” and that reference to one or more “bile salts” may also include one or more “bile acids.”

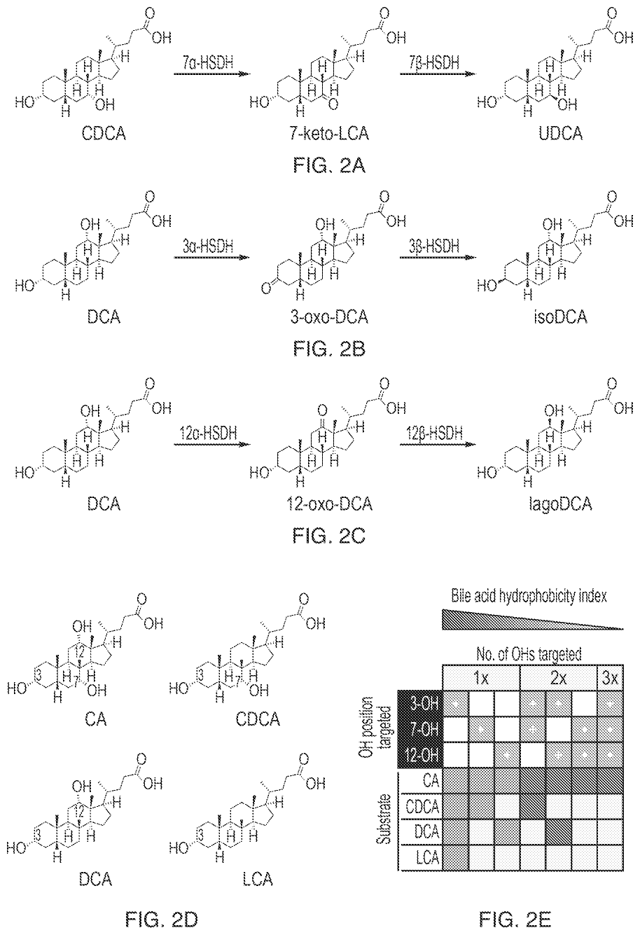

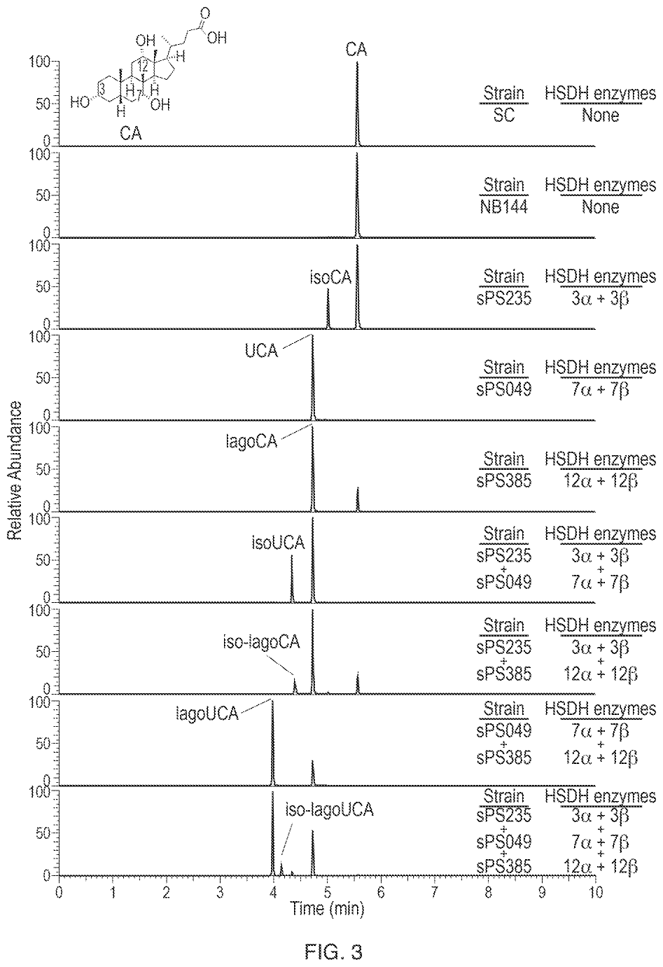

Potential pathways for bile acid or bile salt metabolism and related products, including genes related to bile acid metabolism, are depicted in FIG. 2 and FIG. 3.

Exemplary bile acids which may be metabolized by the bacteria, compositions, and methods disclosed herein include cholic acid (CA), chenodeoxycholic acid (CDCA), deoxycholic acid (DCA), and lithocholic acid (LCA). In certain embodiments, a contemplated bacterium metabolizes CA, and/or CDCA, and/or DCA, and/or LCA to a product so as to alter the bioactivity of the substrate (e.g., by metabolizing a bile acid or bile salt to a different molecule with altered affinity for a human receptor).

In certain embodiments, a contemplated bacterium metabolizes CDCA to ursodeoxycholic acid (UDCA). UDCA is a secondary bile acid found in humans and other mammals. UDCA can be, for example, generated from CDCA by the sequential action of two microbial enzymes: 7α-HSDH and 7β-HSDH. UDCA is a 7β-hydroxy epimer of CDCA. Also known as ursodiol, UDCA has been used in pharmacotherapy for several bile acid diseases or disorders, such as gallstone disease and primary biliary cholangitis among others. UDCA is considered safe and conjugated UDCA is marketed as a supplement. UDCA shows less affinity for TGR5 and impacts colonic secretion to a lesser degree compared to other bile acids like CDCA.

In certain embodiments, a contemplated bacterium metabolizes DCA. The primary bile acid DCA can be, for example, converted to an epimer by gut microbial HSDHs. Epimerization of the 3α-hydroxyl of DCA yields isodeoxycholic acid (isoDCA). Epimerization of the 12α-hydroxyl yields lagodeoxycholic acid (lagoDCA). Both isoDCA and lagoDCA display less affinity for TGR5 compared to the substrate DCA. Furthermore, 3-oxo bile acid intermediates (e.g., 3-oxo-LCA) and iso bile acids (e.g., isoDCA and isoLCA) display immunomodulatory activity. Accordingly, in certain embodiments, a contemplated bacterium metabolizes DCA to isodeoxycholic acid (isoDCA) and/or lagodeoxycholic acid (lagoDCA).

Planar bile acids, also called allo or isoallo bile acids, can be, for example, synthesized by the action of four enzymes: a 3α-HSDH, a 5α-reductase, a 5β-reductase, and finally a 3α-HSDH, producing allo-bile acids, or a 3β-HSDH, producing isoallo bile acids. Allo bile acid products show decreased affinity for TGR5 compared to their substrates, and isoallo bile acids demonstrate immunomodulatory properties. Accordingly, in certain embodiments, a contemplated bacterium metabolizes CDCA to allo-chenodeoxycholic acid (alloCDCA), CDCA to isoallo-chenodeoxycholic acid (isoalloCDCA), DCA to allo-deoxycholic acid (alloDCA), and/or DCA isoallo-deoxycholic acid (isoalloDCA).

In certain embodiments, a contemplated bacterium may encode or one more transgenes encoding a SULT enzyme that metabolizes a bile acid or bile salt to sulfated product.

In certain embodiments, a contemplated bacterium may encode one or more transgenes that modify the 3-, 7,- or 12-hydroxy group of a bile acid. Commensal gut microbes encode hydroxysteroid dehydrogenase (HSDH) enzymes that can modify the 3, 7, and 12-hydroxy groups of bile acids. For example, a contemplated bacterium may comprise: (i) a first transgene encoding a 3,7, or 12α-HSDHs, which oxidizes a hydroxyl group from the α-configuration to a keto group; and (ii) a second transgene encoding a 3, 7, or 12β-HSDH, which reduces the keto group to a hydroxyl in the β configuration. Microbial HSDHs have been described in gut bacterial species spanning the major phyla found in the gut, including Bacteroidetes, Firmicutes, and Actinobacteria.

Exemplary genes related to bile acid metabolism, the expression of which in a bacterium may increase bile acid metabolism, include those encoding: a 7α-hydroxysteroid dehydrogenase (7α-HSDH), a 7β-hydroxysteroid dehydrogenase (7β-HSDH), a 3α-hydroxysteroid dehydrogenase (3α-HSDH), a 3β-hydroxysteroid dehydrogenase (3β-HSDH), a 5α-reductase, a 5β-reductase, a 12α-hydroxysteroid dehydrogenase (12α-HSDH), and a 12β-hydroxysteroid dehydrogenase (12β-HSDH). Accordingly, in certain embodiments, a contemplated bacterium comprises one or more transgenes encoding: a 7α-HSDH, or a functional fragment or variant thereof, a 7β-HSDH or a functional fragment or variant thereof, a 3α-HSDH or a functional fragment or variant thereof, a 3β-HSDH or a functional fragment or variant thereof, a 5α-reductase or a functional fragment or variant thereof, a 5β-reductase or a functional fragment or variant thereof, a 12α-HSDH or a functional fragment or variant thereof, a 12β-HSDH or a functional fragment or variant thereof, or any combination thereof.

As used herein, the term “functional fragment” of a biological entity (e.g., a gene, protein (e.g., 7α-HSDH, 7β-HSDH, 3α-HSDH, 3β-HSDH, 5α-reductase, 5β-reductase, 12α-HSDH or 12β-HSDH), promoter, or ribosome binding site) refers to a fragment of the full-length biological entity that retains, for example, at least 10%, at least 20%, at least 30%, at least 40%, at least 50%, at least 60%, at least 70%, at least 80%, at least 90%, or 100% of the biological activity of the corresponding full-length, naturally occurring biologically entity.

In certain embodiments, a contemplated bacterium may metabolize a substrate or generate a product selected from: 5β-Cholanic acid-3α,7α-diol, 5β-Cholanic acid-3α-ol-7-one, 5β-Cholanic acid-3α,7β-diol, 5β-Cholanic acid-3-one-7α-ol, 5β-Cholanic acid-3,7-dione, 5β-Cholanic acid-3-one-7β-ol, 5β-Cholanic acid-3β,7α-diol, 5β-Cholanic acid-3β-ol-7-one, 5β-Cholanic acid-3β,7β-diol, 3α-Sulfooxy-5β-Cholanic acid-7α-ol, 7α-Sulfooxy-5β-Cholanic acid-3α-ol, 5β-Cholanic acid-3α,12α-diol, 5β-Cholanic acid-3α-ol-12-one, 5β-Cholanic acid-3α,12β-diol, 5β-Cholanic acid-3-one-12α-ol, 5β-Cholanic acid-3,12-dione, 5β-Cholanic acid-3-one-12β-ol, 5β-Cholanic acid-3β,12α-diol, 5β-Cholanic acid-3β-ol-12-one, 5β-Cholanic acid-3β,12β-diol, 3α-Sulfooxy-5β-Cholanic acid-12α-ol, 12α-Sulfooxy-5β-Cholanic acid-3α-ol, 5β-Cholanic acid-3α-ol, 5β-Cholanic acid-3-one, 5β-Cholanic acid-3β-ol, 3α-Sulfooxy-5β-cholanic acid, 5β-Cholanic acid-3α,7α,12α-triol, 5β-Cholanic acid-3α,7α-diol-12-one, 5β-Cholanic acid-3α,7α,12β-triol, 5β-Cholanic acid-3α,12α-diol-7-one, 5β-Cholanic acid-3α-ol-7,12-dione, 5β-Cholanic acid-3α,12β-diol-7-one, 5β-Cholanic acid-3α,7β,12α-triol, 5β-Cholanic acid-3α,7β-12-one, 5β-Cholanic acid-3α,7β,12β-triol, 5β-Cholanic acid-3-one-7α,12α-diol, 5β-Cholanic acid-3,12-dione-7α-ol, 5β-Cholanic acid-3-one-7α,12β-diol, 5β-Cholanic acid-3,7-dione-12α-ol, 5β-Cholanic acid-3,7,12-trione, 5β-Cholanic acid-3,7-one-12β-ol, 5β-Cholanic acid-3-one-7β,12α-diol, 5β-Cholanic acid-3,12-dione-7β-ol, 5β-Cholanic acid-3-one-7β,12β-diol, 5β-Cholanic acid-3β,7α,12α-triol, 5β-Cholanic acid-3β,7α-diol-12-one, 5β-Cholanic acid-3β,7α,12β-triol, 5β-Cholanic acid-3β,12α-diol-7-one, 5β-Cholanic acid-3β-ol-7,12-dione, 5β-Cholanic acid-3β,12β-diol-7-one, 5β-Cholanic acid-3β,7β,12α-triol, 5β-Cholanic acid-3β,7β-diol-12-one, 5β-Cholanic acid-3β,7β-12β-triol, 3α-Sulfooxy-5β-Cholanic acid-7α,12α-diol, 7α-Sulfooxy-5β-Cholanic acid-3α,12α-diol, 12α-Sulfooxy-5β-Cholanic acid-3α,7α-diol, 5α-Cholanic acid-3α,7α-diol, 5α-Cholanic acid-3α-ol-7-one, 5α-Cholanic acid-3α,7β-diol, 5α-Cholanic acid-3-one-7α-ol, 5α-Cholanic acid-3,7-dione, 5α-Cholanic acid-3-one-7β-ol, 5α-Cholanic acid-3β,7α-diol, 5α-Cholanic acid-3β-ol-7-one, 5α-Cholanic acid-3β,7β-diol, 5α-Cholanic acid-3α,12α-diol, 5α-Cholanic acid-3α-ol-12-one, 5α-Cholanic acid-3α,12β-diol, 5α-Cholanic acid-3-one-12α-ol, 5α-Cholanic acid-3,12-dione, 5α-Cholanic acid-3-one-12β-ol, 5α-Cholanic acid-3β,12α-diol, 5α-Cholanic acid-3β-ol-12-one, 5α-Cholanic acid-3β,12β-diol, 5α-Cholanic acid-3α-ol, 5α-Cholanic acid-3-one, 5α-Cholanic acid-3β-ol, 5α-Cholanic acid-3α,7α,12α-triol, 5α-Cholanic acid-3α,7α-diol-12-one, 5α-Cholanic acid-3α,7α,12β-triol, 5α-Cholanic acid-3α,12α-diol-7-one, 5α-Cholanic acid-3α-ol-7,12-dione, 5α-Cholanic acid-3α,12β-diol-7-one, 5α-Cholanic acid-3α,7β,12α-triol, 5α-Cholanic acid-3α,7β-12-one, 5α-Cholanic acid-3α,7β,12β-triol, 5α-Cholanic acid-3-one-7α,12α-diol, 5α-Cholanic acid-3,12-dione-7α-ol, 5α-Cholanic acid-3-one-7α,12β-diol, 5α-Cholanic acid-3,7-dione-12α-ol, 5α-Cholanic acid-3,7,12-trione, 5α-Cholanic acid-3,7-one-12β-ol, 5α-Cholanic acid-3-one-7β,12α-diol, 5α-Cholanic acid-3,12-dione-7β-ol, 5α-Cholanic acid-3-one-7β,12β-diol, 5α-Cholanic acid-3β,7α,12α-triol, 5α-Cholanic acid-3β,7α-diol-12-one, 5α-Cholanic acid-3β,7α,12-triol, 5α-Cholanic acid-3β,12α-diol-7-one, 5α-Cholanic acid-3β-ol-7,12-dione, 5α-Cholanic acid-3β,12β-diol-7-one, 5α-Cholanic acid-3β,7β,12α-triol, 5α-Cholanic acid-3β,7β-diol-12-one, 5α-Cholanic acid-3β,7β-12β-triol, 5β-Cholanic acid-3α,7α,12α-triol N-(carboxymethyl)-amide, 5β-Cholanic acid-3α,7α-diol-12-one N-(carboxymethyl)-amide, 5β-Cholanic acid-3α,7α,12β-triol N-(carboxymethyl)-amide, 5β-Cholanic acid-3α,12α-diol-7-one N-(carboxymethyl)-amide, 5β-Cholanic acid-3α-ol-7,12-dione N-(carboxymethyl)-amide, 5β-Cholanic acid-3α,12β-diol-7-one N-(carboxymethyl)-amide, 5β-Cholanic acid-3α,7β,12α-triol N-(carboxymethyl)-amide, 5β-Cholanic acid-3α,7β-12-one N-(carboxymethyl)-amide, 5β-Cholanic acid-3α,7β,12β-triol N-(carboxymethyl)-amide, 5β-Cholanic acid-3-one-7α,12α-diol N-(carboxymethyl)-amide, 5β-Cholanic acid-3,12-dione-7α-ol N-(carboxymethyl)-amide, 5β-Cholanic acid-3-one-7α,12β-diol N-(carboxymethyl)-amide, 5β-Cholanic acid-3,7-dione-12α-ol N-(carboxymethyl)-amide, 5β-Cholanic acid-3,7,12-trione N-(carboxymethyl)-amide, 5β-Cholanic acid-3,7-one-12β-ol N-(carboxymethyl)-amide, 5β-Cholanic acid-3-one-7β,12α-diol N-(carboxymethyl)-amide, 5β-Cholanic acid-3,12-dione-7β-ol N-(carboxymethyl)-amide, 5β-Cholanic acid-3-one-7β,12β-diol N-(carboxymethyl)-amide, 5β-Cholanic acid-3β,7α,12α-triol N-(carboxymethyl)-amide, 5β-Cholanic acid-3β,7α-diol-12-one N-(carboxymethyl)-amide, 5β-Cholanic acid-3β,7α,12β-triol N-(carboxymethyl)-amide, 5β-Cholanic acid-3β,12α-diol-7-one N-(carboxymethyl)-amide, 5β-Cholanic acid-3β-ol-7,12-dione N-(carboxymethyl)-amide, 5β-Cholanic acid-3β,12β-diol-7-one N-(carboxymethyl)-amide, 5β-Cholanic acid-3β,7β,12α-triol N-(carboxymethyl)-amide, 5β-Cholanic acid-3α,7β-diol-12-one N-(carboxymethyl)-amide, 5β-Cholanic acid-3β,7β-12β-triol N-(carboxymethyl)-amide, 3α-Sulfooxy-5β-Cholanic acid-7α,12α-diol N-(carboxymethyl)-amide, 7α-Sulfooxy-5β-Cholanic acid-3α,12α-diol N-(carboxymethyl)-amide, 12α-Sulfooxy-5β-Cholanic acid-3α,7α-diol N-(carboxymethyl)-amide, 5β-Cholanic acid-3α,7α,12α-triol N-(2-sulphoethyl)-amide, 5β-Cholanic acid-3α,7α-diol-12-one N-(2-sulphoethyl)-amide, 5β-Cholanic acid-3α,7α,12β-triol N-(2-sulphoethyl)-amide, 5β-Cholanic acid-3α,12α-diol-7-one N-(2-sulphoethyl)-amide, 5β-Cholanic acid-3α-ol-7,12-dione N-(2-sulphoethyl)-amide, 5β-Cholanic acid-3α,12β-diol-7-one N-(2-sulphoethyl)-amide, 5β-Cholanic acid-3α,7β,12α-triol N-(2-sulphoethyl)-amide, 5β-Cholanic acid-3α,7β-12-one N-(2-sulphoethyl)-amide, 5β-Cholanic acid-3α,7β,123-triol N-(2-sulphoethyl)-amide, 5β-Cholanic acid-3-one-7α,12α-diol N-(2-sulphoethyl)-amide, 5β-Cholanic acid-3,12-dione-7α-ol N-(2-sulphoethyl)-amide, 5β-Cholanic acid-3-one-7α,12β-diol N-(2-sulphoethyl)-amide, 5β-Cholanic acid-3,7-dione-12α-ol N-(2-sulphoethyl)-amide, 5β-Cholanic acid-3,7,12-trione N-(2-sulphoethyl)-amide, 5β-Cholanic acid-3,7-one-12β-ol N-(2-sulphoethyl)-amide, 5β-Cholanic acid-3-one-7β,12α-diol N-(2-sulphoethyl)-amide, 5β-Cholanic acid-3,12-dione-7β-ol N-(2-sulphoethyl)-amide, 5β-Cholanic acid-3-one-7β,12β-diol N-(2-sulphoethyl)-amide, 5β-Cholanic acid-3β,7α,12α-triol N-(2-sulphoethyl)-amide, 5β-Cholanic acid-3β,7α-diol-12-one N-(2-sulphoethyl)-amide, 5β-Cholanic acid-3β,7α,12β-triol N-(2-sulphoethyl)-amide, 5β-Cholanic acid-3β,12α-diol-7-one N-(2-sulphoethyl)-amide, 5β-Cholanic acid-3β-ol-7,12-dione N-(2-sulphoethyl)-amide, 5β-Cholanic acid-3β,12β-diol-7-one N-(2-sulphoethyl)-amide, 5β-Cholanic acid-3β,7β,12α-triol N-(2-sulphoethyl)-amide, 5β-Cholanic acid-3β,7β-diol-12-one N-(2-sulphoethyl)-amide, 5β-Cholanic acid-3β,7β-12β-triol N-(2-sulphoethyl)-amide, 3α-Sulfooxy-5β-Cholanic acid-7α,12α-diol N-(2-sulphoethyl)-amide, 7α-Sulfooxy-5β-Cholanic acid-3α,12α-diol N-(2-sulphoethyl)-amide, 12α-Sulfooxy-5β-Cholanic acid-3α,7α-diol N-(2-sulphoethyl)-amide, 5β-Cholanic acid-3α,7α-diol N-(carboxymethyl)-amide, 5β-Cholanic acid-3α-ol-7-one N-(carboxymethyl)-amide, 5β-Cholanic acid-3α,7β-diol N-(carboxymethyl)-amide, 5β-Cholanic acid-3-one-7α-ol N-(carboxymethyl)-amide, 5β-Cholanic acid-3,7-dione N-(carboxymethyl)-amide, 5β-Cholanic acid-3-one-7β-ol N-(carboxymethyl)-amide, 5β-Cholanic acid-3β,7α-diol N-(carboxymethyl)-amide, 5β-Cholanic acid-3β-ol-7-one N-(carboxymethyl)-amide, 5β-Cholanic acid-3β,7β-diol N-(carboxymethyl)-amide, 3α-Sulfooxy-5β-Cholanic acid-7α-ol N-(carboxymethyl)-amide, 7α-Sulfooxy-5β-Cholanic acid-3α-ol N-(carboxymethyl)-amide, 5β-Cholanic acid-3α,7α-diol N-(2-sulphoethyl)-amide, 5-Cholanic acid-3α-ol-7-one N-(2-sulphoethyl)-amide, 5β-Cholanic acid-3α,7β-diol N-(2-sulphoethyl)-amide, 5β-Cholanic acid-3-one-7α-ol N-(2-sulphoethyl)-amide, 5β-Cholanic acid-3,7-dione N-(2-sulphoethyl)-amide, 5β-Cholanic acid-3-one-7β-ol N-(2-sulphoethyl)-amide, 5β-Cholanic acid-3β,7α-diol N-(2-sulphoethyl)-amide, 5β-Cholanic acid-3β-ol-7-one N-(2-sulphoethyl)-amide, 5β-Cholanic acid-3β,7β-diol N-(2-sulphoethyl)-amide, 3α-Sulfooxy-5β-Cholanic acid-7α-ol N-(2-sulphoethyl)-amide, 7α-Sulfooxy-5β-Cholanic acid-3α-ol N-(2-sulphoethyl)-amide, 5β-Cholanic acid-3α,12α-diol N-(carboxymethyl)-amide, 5β-Cholanic acid-3α-ol-12-one N-(carboxymethyl)-amide, 5β-Cholanic acid-3α,12β-diol N-(carboxymethyl)-amide, 5β-Cholanic acid-3-one-12α-ol N-(carboxymethyl)-amide, 5β-Cholanic acid-3,12-dione N-(carboxymethyl)-amide, 5β-Cholanic acid-3-one-12β-ol N-(carboxymethyl)-amide, 5β-Cholanic acid-3β,12α-diol N-(carboxymethyl)-amide, 5β-Cholanic acid-3β-ol-12-one N-(carboxymethyl)-amide, 5β-Cholanic acid-3β,12β-diol N-(carboxymethyl)-amide, 3α-Sulfooxy-5β-Cholanic acid-12α-ol N-(carboxymethyl)-amide, 12α-Sulfooxy-5β-Cholanic acid-3α-ol N-(carboxymethyl)-amide, 5β-Cholanic acid-3α,12α-diol N-(2-sulphoethyl)-amide, 5β-Cholanic acid-3α-ol-12-one N-(2-sulphoethyl)-amide, 5β-Cholanic acid-3α,12β-diol N-(2-sulphoethyl)-amide, 5β-Cholanic acid-3-one-12α-ol N-(2-sulphoethyl)-amide, 5β-Cholanic acid-3,12-dione N-(2-sulphoethyl)-amide, 5β-Cholanic acid-3-one-12β-ol N-(2-sulphoethyl)-amide, 5β-Cholanic acid-3β,12α-diol N-(2-sulphoethyl)-amide, 5β-Cholanic acid-3β-ol-12-one N-(2-sulphoethyl)-amide, 5β-Cholanic acid-3β,12β-diol N-(2-sulphoethyl)-amide, 3α-Sulfooxy-5β-Cholanic acid-12α-ol N-(2-sulphoethyl)-amide, 12α-Sulfooxy-5β-Cholanic acid-3α-ol N-(2-sulphoethyl)-amide, 5β-Cholanic acid-3α-ol N-(carboxymethyl)-amide, 5β-Cholanic acid-3-one N-(carboxymethyl)-amide, 5β-Cholanic acid-3β-ol N-(carboxymethyl)-amide, 3α-Sulfooxy-5β-cholanic acid N-(carboxymethyl)-amide, 5β-Cholanic acid-3α-ol N-(2-sulphoethyl)-amide, 5β-Cholanic acid-3-one N-(2-sulphoethyl)-amide, 5β-Cholanic acid-3β-ol N-(2-sulphoethyl)-amide, and 3α-Sulfooxy-5β-cholanic acid N-(2-sulphoethyl)-amide for example, by expressing one or more transgenes encoding 7α-HSDH, 7β-HSDH, 3α-HSDH, 3β-HSDH, 5α-reductase, 5β-reductase, 12α-HSDH or 12β-HSDH, SULT, or a combination thereof.

In certain embodiments, a contemplated bacterium comprises a transgene encoding a 7α-HSDH, for example, a Bacteroides, Escherichia, Paeniclostridium, Clostridium or Brucella 7α-HSDH, for example, a Bacteroides fragilis, Escherichia coli, Paeniclostridium sordellii, Clostridium absonum, or Brucella melitensis 7α-HSDH, or a functional fragment or variant of any of the foregoing proteins. For example, in certain embodiments, a contemplated bacterium comprises a transgene encoding a Bacteroides fragilis, Escherichia coli, Paeniclostridium sordellii, Clostridium absonum, or Brucella melitensis 7α-HSDH, or a protein having at least 80%, 85%, 86%, 87%, 88%, 89%, 90%, 91%, 92%, 93%, 94%, 95%, 96%, 97%, 98%, or 99% identity to a Bacteroides fragilis, Escherichia co/i, Paeniclostridium sordellii, Clostridium absonum, or Brucella melitensis 7α-HSDH. Exemplary 7α-HSDH coding sequences are depicted in SEQ ID NOs: 1-6. Accordingly, in certain embodiments, a bacterium has been modified to comprise a transgene encoding an amino sequence encoded by any one of SEQ ID NOs: 1-6, or having at least 80%, 85%, 86%, 87%, 88%, 89%, 90%, 91%, 92%, 93%, 94%, 95%, 96%, 97%, 98%, or 99% identity to an amino sequence encoded by any one of SEQ ID NOs: 1-6. In certain embodiments, a bacterium has been modified to comprise a nucleic acid comprising the nucleotide sequence of any one of SEQ ID NOs: 1-6, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 1-6.

In certain embodiments, a contemplated bacterium comprises a transgene encoding a 7β-HSDH, for example, a Ruminococcus, Colinsella, or Clostridium 7β-HSDH, for example, a Ruminococcus gnavus, Colinsella aerofaciens, Clostridium absonum, or Ruminococcus torques 7β-HSDH, or a functional fragment or variant of any of the foregoing proteins. For example, in certain embodiments, a contemplated bacterium comprises a transgene encoding a Ruminococcus gnavus, Colinsella aerofaciens, Clostridium absonum, or Ruminococcus torques 7β-HSDH, or a protein having at least 80%, 85%, 86%, 87%, 88%, 89%, 90%, 91%, 92%, 93%, 94%, 95%, 96%, 97%, 98%, or 99% identity to a Ruminococcus gnavus, Colinsella aerofaciens, Clostridium absonum, or Ruminococcus torques 7β-HSDH. Exemplary 7β-HSDH coding sequences are depicted in SEQ ID NOs: 7-11. Accordingly, in certain embodiments, a bacterium has been modified to comprise a transgene encoding an amino sequence encoded by any one of SEQ ID NOs: 7-11, or having at least 80%, 85%, 86%, 87%, 88%, 89%, 90%, 91%, 92%, 93%, 94%, 95%, 96%, 97%, 98%, or 99% identity to an amino sequence encoded by any one of SEQ ID NOs: 7-11. In certain embodiments, a bacterium has been modified to comprise a nucleic acid comprising the nucleotide sequence of any one of SEQ ID NOs: 7-11, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 7-11.

In certain embodiments, a contemplated bacterium comprises a transgene encoding a 3α-HSDH, for example, a Ruminococcus or Eggerthella 3α-HSDH, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, or a functional fragment or variant of any of the foregoing proteins. For example, in certain embodiments, a contemplated bacterium comprises a transgene encoding a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, or a protein having at least 80%, 85%, 86%, 87%, 88%, 89%, 90%, 91%, 92%, 93%, 94%, 95%, 96%, 97%, 98%, or 99% identity to a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH. Exemplary 3α-HSDH coding sequences are depicted in SEQ ID NOs: 12-23. Accordingly, in certain embodiments, a bacterium has been modified to comprise a transgene encoding an amino sequence encoded by any one of SEQ ID NOs: 12-23, or having at least 80%, 85%, 86%, 87%, 88%, 89%, 90%, 91%, 92%, 93%, 94%, 95%, 96%, 97%, 98%, or 99% identity to an amino sequence encoded by any one of SEQ ID NOs: 12-23. In certain embodiments, a bacterium has been modified to comprise a nucleic acid comprising the nucleotide sequence of any one of SEQ ID NOs: 12-23, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 12-23.

In certain embodiments, a contemplated bacterium comprises a transgene encoding a 3β-HSDH, for example, a Ruminococcus, Eggerthella, Parabacteroides, or Bacteroides 3β-HSDH, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, or a functional fragment or variant of any of the foregoing proteins. For example, in certain embodiments, a contemplated bacterium comprises a transgene encoding a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, or a protein having at least 80%, 85%, 86%, 87%, 88%, 89%, 90%, 91%, 92%, 93%, 94%, 95%, 96%, 97%, 98%, or 99% identity to a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH. Exemplary 3β-HSDH coding sequences are depicted in SEQ ID NOs: 24-47. Accordingly, in certain embodiments, a bacterium has been modified to comprise a transgene encoding an amino sequence encoded by any one of SEQ ID NOs: 24-47 or having at least 80%, 85%, 86%, 87%, 88%, 89%, 90%, 91%, 92%, 93% 94% 95% 96%, 97% 98%, or 99% identity to an amino sequence encoded by any one of SEQ ID NOs: 24-47. In certain embodiments, a bacterium has been modified to comprise a nucleic acid comprising the nucleotide sequence of any one of SEQ ID NOs: 24-47, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 24-47.

In certain embodiments, a contemplated bacterium comprises a transgene encoding a 5α-reductase, for example, a Parabacteroides or Bacteroides 5α-reductase, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, or a functional fragment or variant of any of the foregoing proteins. For example, in certain embodiments, a contemplated bacterium comprises a transgene encoding a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, or a protein having at least 80%, 85%, 86%, 87%, 88%, 89%, 90%, 91%, 92%, 93%, 94%, 95%, 96%, 97%, 98%, or 99% identity to a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase. Exemplary 5α-reductase coding sequences are depicted in SEQ ID NOs: 61-67. Accordingly, in certain embodiments, a bacterium has been modified to comprise a transgene encoding an amino sequence encoded by any one of SEQ ID NOs: 61-67, or having at least 80%, 85%, 86%, 87%, 88%, 89%, 90%, 91%, 92%, 93%, 94%, 95%, 96%, 97%, 98%, or 99% identity to an amino sequence encoded by any one of SEQ ID NOs: 61-67. In certain embodiments, a bacterium has been modified to comprise a nucleic acid comprising the nucleotide sequence of any one of SEQ ID NOs: 61-67, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 61-67.

In certain embodiments, a contemplated bacterium comprises a transgene encoding a 5β-reductase, for example, a Parabacteroides or Bacteroides 5β-reductase, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, or a functional fragment or variant of any of the foregoing proteins. For example, in certain embodiments, a contemplated bacterium comprises a transgene encoding a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, or a protein having at least 80%, 85%, 86%, 87%, 88%, 89%, 90%, 91%, 92%, 93%, 94%, 95%, 96%, 97%, 98%, or 99% identity to a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase. Exemplary 5β-reductase coding sequences are depicted in SEQ ID NOs: 68-74. Accordingly, in certain embodiments, a bacterium has been modified to comprise a transgene encoding an amino sequence encoded by any one of SEQ ID NOs: 68-74, or having at least 80%, 85%, 86%, 87%, 88%, 89%, 90%, 91%, 92%, 93%, 94%, 95%, 96%, 97%, 98%, or 99% identity to an amino sequence encoded by any one of SEQ ID NOs: 68-74. In certain embodiments, a bacterium has been modified to comprise a nucleic acid comprising the nucleotide sequence of any one of SEQ ID NOs: 68-74, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 68-74.

In certain embodiments, a contemplated bacterium comprises a transgene encoding a 12α-HSDH, for example, a Eggerthella or Clostridium 12α-HSDH, for example, a Eggerthella lenta, Eggerthella sp. CAG:298, Clostridium sp. ATCC29733, Clostridium hylemonae, Clostridium scindens, or Clostridium hiranonis 12α-HSDH, or a functional fragment or variant of any of the foregoing proteins. For example, in certain embodiments, a contemplated bacterium comprises a transgene encoding a Eggerthella lenta, Eggerthella sp. CAG:298, Clostridium sp. ATCC29733, Clostridium hylemonae, Clostridium scindens, or Clostridium hiranonis 12α-HSDH, or a protein having at least 80%, 85%, 86%, 87%, 88%, 89%, 90%, 91%, 92%, 93%, 94%, 95%, 96%, 97%, 98%, or 99% identity to a Eggerthella lenta, Eggerthella sp. CAG:298, Clostridium sp. ATCC29733, Clostridium hylemonae, Clostridium scindens, or Clostridium hiranonis 12α-HSDH. Exemplary 12α-HSDH coding sequences are depicted in SEQ ID NOs: 48-54. Accordingly, in certain embodiments, a bacterium has been modified to comprise a transgene encoding an amino sequence encoded by any one of SEQ ID NOs: 48-54, or having at least 80%, 85%, 86%, 87%, 88%, 89%, 90%, 91%, 92%, 93%, 94%, 95%, 96%, 97%, 98%, or 99% identity to an amino sequence encoded by any one of SEQ ID NOs: 48-54. In certain embodiments, a bacterium has been modified to comprise a nucleic acid comprising the nucleotide sequence of any one of SEQ ID NOs: 48-54, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 48-54.

In certain embodiments, a contemplated bacterium comprises a transgene encoding a 12β-HSDH, for example, a Clostridium, Eisenbergiella, Olsenella, Collinsella, or Ruminococcus 12β-HSDH, for example, a Clostridium paraputrificum, Eisenbergiella sp. OF01-20, Olsenella sp. GAM18, Collinsella tanakaei, Ruminococcus sp. AF14-10, or Ruminococcus lactaris 12β-HSDH, or a functional fragment or variant of any of the foregoing proteins. For example, in certain embodiments, a contemplated bacterium comprises a transgene encoding a Clostridium paraputrificum, Eisenbergiella sp. OF01-20, Olsenella sp. GAM18, Collinsella tanakaei, Ruminococcus sp. AF14-10, or Ruminococcus lactaris 12β-HSDH, or a protein having at least 80%, 85%, 86%, 87%, 88%, 89%, 90%, 91%, 92%, 93%, 94%, 95%, 96%, 97%, 98%, or 99% identity to a Clostridium paraputrificum, Eisenbergiella sp. OF01-20, Olsenella sp. GAM18, Collinsella tanakaei, Ruminococcus sp. AF14-10, or Ruminococcus lactaris 12β-HSDH. Exemplary 12β-HSDH coding sequences are depicted in SEQ ID NOs: 55-60. Accordingly, in certain embodiments, a bacterium has been modified to comprise a transgene encoding an amino sequence encoded by any one of SEQ ID NOs: 55-60, or having at least 80%, 85%, 86%, 87%, 88%, 89%, 90%, 91%, 92%, 93%, 94%, 95%, 96%, 97%, 98%, or 99% identity to an amino sequence encoded by any one of SEQ ID NOs: 55-60. In certain embodiments, a bacterium has been modified to comprise a nucleic acid comprising the nucleotide sequence of any one of SEQ ID NOs: 55-60, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 55-60.

In certain embodiments (for example, so as to metabolize CDCA to UDCA) a contemplated bacterium may comprise: (i) a transgene encoding a 7α-hydroxysteroid dehydrogenase (7α-HSDH), or a functional fragment or variant thereof, for example, a Bacteroides fragilis, Escherichia coli, Paeniclostridium sordellii, Clostridium absonum, or Brucella melitensis 7α-HSDH, for example, a 7α-HSDH comprising an amino sequence encoded by any one of SEQ ID NOs: 1-6, or having at least 80% identity to an amino sequence encoded by any one of SEQ ID NOs: 1-6; and (ii) a transgene encoding a 7β-hydroxysteroid dehydrogenase (7β-HSDH), or a functional fragment or variant thereof, for example, a Ruminococcus gnavus, Colinsella aerofaciens, Clostridium absonum, or Ruminococcus torques 7β-HSDH, for example, a 7β-HSDH comprising an amino sequence encoded by any one of SEQ ID NOs: 7-11, or having at least 80% identity to an amino sequence encoded by any one of SEQ ID NOs: 7-11.

In certain embodiments (for example, so as to metabolize LCA to isoalloLCA) a contemplated bacterium may comprise: (i) a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23; (ii) a transgene encoding a 5α-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, for example, a 5α-reductase comprising an amino sequence encoded by any one of SEQ ID NOs: 61-67, or having at least 80% identity to an amino sequence encoded by any one of SEQ ID NOs: 61-67; and (iii) a transgene encoding a 5β-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, for example, a 5β-reductase comprising an amino sequence encoded by any one of SEQ ID NOs: 68-74, or having at least 80% identity to an amino sequence encoded by any one of SEQ ID NOs: 68-74; and a transgene encoding a 3β-hydroxysteroid dehydrogenase, or a functional fragment or variant thereof, for example, Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, for example, 3β-hydroxysteroid dehydrogenase comprising an amino sequence encoded by any one of SEQ ID NOs: 24-47, or having at least 80% identity to an amino sequence encoded by any one of SEQ ID NOs: 24-47.

In certain embodiments (for example, so as to metabolize CDCA to isoalloCDCA) a contemplated bacterium may comprise: (i) a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23; (ii) a transgene encoding a 5α-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, for example, a 5α-reductase comprising an amino sequence encoded by any one of SEQ ID NOs: 61-67, or having at least 80% identity to an amino sequence encoded by any one of SEQ ID NOs: 61-67; (iii) a transgene encoding a 5β-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, for example, a 5β-reductase comprising an amino sequence encoded by any one of SEQ ID NOs: 68-74, or having at least 80% identity to an amino sequence encoded by any one of SEQ ID NOs: 68-74; and (iv) a transgene encoding a 3β-hydroxysteroid dehydrogenase (3β-HSDH), or a functional fragment or variant thereof, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, for example, a 3β-HSDH comprising an amino sequence encoded by any one of SEQ ID NOs: 24-47, or having at least 80% identity to an amino sequence encoded by any one of SEQ ID NOs: 24-47.

In certain embodiments (for example, so as to metabolize DCA to isoDCA) a contemplated bacterium may comprise: (i) a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23; and (ii) a transgene encoding a 3β-hydroxysteroid dehydrogenase (3β-HSDH), or a functional fragment or variant thereof, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, for example, a 3β-HSDH comprising an amino sequence encoded by any one of SEQ ID NOs: 24-47 or having at least 80% identity to an amino sequence encoded by any one of SEQ ID NOs: 24-47.

In certain embodiments (for example, so as to metabolize DCA lagoDCA) a contemplated bacterium may comprise: (i) a transgene encoding a 12α-hydroxysteroid dehydrogenase (12α-HSDH), or a functional fragment or variant thereof, for example, a Eggerthella lenta, Eggerthella sp. CAG:298, Clostridium sp. ATCC29733, Clostridium hylemonae, Clostridium scindens, or Clostridium hiranonis 12α-HSDH, for example, a 12α-HSDH comprising an amino sequence encoded by any one of SEQ ID NOs: 48-54, or having at least 80% identity to an amino sequence encoded by any one of SEQ ID NOs: 48-54; and (ii) a transgene encoding a 12β-hydroxysteroid dehydrogenase (12β-HSDH), or a functional fragment or variant thereof, for example, a Clostridium paraputrificum, Eisenbergiella sp. OF01-20, Olsenella sp. GAM18, Collinsella tanakaei, Ruminococcus sp. AF14-10, or Ruminococcus lactaris 12β-HSDH, for example, a 12β-HSDH comprising an amino sequence encoded by any one of SEQ ID NOs: 55-60, or having at least 80% identity to an amino sequence encoded by any one of SEQ ID NOs: 55-60.

In certain embodiments (for example, so as to metabolize DCA to alloDCA) a contemplated bacterium may comprise: (i) a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23; (ii) a transgene encoding a 5α-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, for example, a 5α-reductase comprising an amino sequence encoded by any one of SEQ ID NOs: 61-67, or having at least 80% identity to an amino sequence encoded by any one of SEQ ID NOs: 61-67; and (iii) a transgene encoding a 5β-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, for example, a 5β-reductase comprising an amino sequence encoded by any one of SEQ ID NOs: 68-74, or having at least 80% identity to an amino sequence encoded by any one of SEQ ID NOs: 68-74.

In certain embodiments (for example, so as to metabolize DCA to isoalloDCA) a contemplated bacterium may comprise: (i) a transgene encoding a 3α-hydroxysteroid dehydrogenase (3α-HSDH), or a functional fragment or variant thereof, for example, a Ruminococcus gnavus, Eggerthella lenta, Eggerthella sp. CAG298, Gordonibacter massiliensis, Raoultibacter timonensis, Lachnospiraceae sp., Paraeggerthella hongkongensis, Eggerthella sinensis, Eggerthella guodeyinii, Gordonibacter pamelaeae, or Raoultibacter massiliensis 3α-HSDH, for example, a 3α-HSDH comprising an amino sequence encoded by any one of SEQ ID NOs: 12-23, or having at least 80% identity to an amino sequence encoded by any one of SEQ ID NOs: 12-23; (ii) a transgene encoding a 5α-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5α-reductase, for example, a 5α-reductase comprising an amino sequence encoded by any one of SEQ ID NOs: 61-67, or having at least 80% identity to an amino sequence encoded by any one of SEQ ID NOs: 61-67; (iii) a transgene encoding a 5β-reductase, or a functional fragment or variant thereof, for example, a Parabacteroides merdae, Bacteroides dorei, Bacteroides vulgatus, Bacteroides thetaiotaomicron, Bacteroides caccae, Bacteroides finegoldii, or Bacteroides uniformis 5β-reductase, for example, a 5β-reductase comprising an amino sequence encoded by any one of SEQ ID NOs: 68-74, or having at least 80% identity to an amino sequence encoded by any one of SEQ ID NOs: 68-74; and (iv) a transgene encoding a 3β-hydroxysteroid dehydrogenase (3β-HSDH), or a functional fragment or variant thereof, for example, a Ruminococcus gnavus, Eggerthella lenta, Eggerthella sp. CAG298, Lachnospiraceae sp. 2_1_46FAA, Absiella sp. AM29-15, Clostridium cadaveris, Holdemania filiformis, Clostridium disporicum, Clostridium sp. CL-6, Erysipelotrichia sp., Holdemania sp. 1001302B_160321_E10, Clostridium innocuum, Erysipelotrichaceae sp. 66202529, Clostridium sp. NSJ-6, Parabacteroides merdae, Bacteroides dorei, Bacteroides vulgatus, Bacteroides thetaiotaomicron, Bacteroides caccae, Bacteroides finegoldii, or Bacteroides uniformis 3β-HSDH, for example, a 3β-HSDH comprising an amino sequence encoded by any one of SEQ ID NOs: 24-47, or having at least 80% identity to an amino sequence encoded by any one of SEQ ID NOs: 24-47.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

A contemplated modified bacterium, for example, for use in a disclosed pharmaceutical composition or method, includes a bacterium of genus Bacteroides, Alistipes, Faecalibacterium, Parabacteroides, Prevotella, Roseburia, Ruminococcus, Clostridium, Oscillibacter, Gemmiger, Barnesiella, Dialister, Parasutterella, Phascolarctobacterium, Propionibacterium, Sutterella, Blautia, Paraprevotella, Coprococcus, Odoribacter, Spiroplasma, Anaerostipes, or Akkermansia. A contemplated bacterium, for example, for use in a disclosed pharmaceutical composition or method, may be of the Bacteroides genus, i.e., may be a Bacteroides species bacterium.

Exemplary Bacteroides species include B. acidifaciens, B. barnesiaes, B. caccae, B. caecicola, B. caecigallinarum, B. cellulosilyticus, B. cellulosolvens, B. clarus, B. coagulans, B. coprocola, B. coprophilus, B. coprosuis, B. distasonis, B. dorei, B. eggerthii, B. gracilis, B. faecichinchillae, B. faecis, B. finegoldii, B. fluxus, B. fragilis, B. galacturonicus, B. gallinaceum, B. gallinarum, B. goldsteinii, B. graminisolvens, B. helcogene, B. intestinalis, B. luti, B. massiliensis, B. melaninogenicus, B. nordii, B. oleiciplenus, B. oris, B. ovatus, B. paurosaccharolyticus, B. pectinophilus, B. plebeius, B. polypragmatus, B. propionicifaciens, B. putredinis, B. pyogenes, B. reticulotermitis, B. rodentium, B. salanitronis, B. salyersiae, B. sartorii, B. sediment B. stercoris, B. suis, B. tectus, B. thetaiotaomicron, B. uniformis, B. vulgatus, B. xylanisolvens, and B. xylanolyticusxylanolyticus.

As used herein, the term “species” refers to a taxonomic entity as conventionally defined by genomic sequence and phenotypic characteristics. A “strain” is a particular instance of a species that has been isolated and purified according to conventional microbiological techniques. The present disclosure encompasses derivatives of the disclosed bacterial strains. The term “derivative” includes daughter strains (progeny) or stains cultured (sub-cloned) from the original but modified in some way (including at the genetic level), without altering negatively a biological activity of the strain.

In certain embodiments, a contemplated modified bacterium is of a genus that makes up more than 0.1%, 0.5%, 1%, 5%, 10%, 20%, 30%, or 40% of the total culturable microbes in the feces of a subject to be treated, or in the feces of an average human. In certain embodiments, a contemplated modified bacterium is of a genus that is detected at a level greater than 10¹², 10¹¹, 10¹⁰, 10⁹, 10⁸, 10⁷ colony forming units per gram of feces of a subject to be treated, or per gram of feces of an average human. In certain embodiments, a contemplated modified bacterium is of a genus that makes up more than 0.1%, 0.5%, 1%, 5%, 10%, 20%, 30%, or 40% of the gut microbiome of a subject to be treated, or of the gut microbiome of an average human. Human gut or feces microbiome composition may be assayed by any technique known in the art, including 16S ribosomal sequencing.

rRNA, 16S rDNA, 16S rRNA, 16S, 18S, 18S rRNA, and 18S rDNA refer to nucleic acids that are components of, or encode for, components of the ribosome. There are two subunits in the ribosome termed the small subunit (SSU) and large subunit (LSU). rDNA genes and their complementary RNA sequences are widely used for determination of the evolutionary relationships amount organisms as they are variable, yet sufficiently conserved to allow cross-organism molecular comparisons.

16S rDNA sequence (approximately 1542 nucleotides in length) of the 30S SSU can be used, in certain embodiments, for molecular-based taxonomic assignments of prokaryotes and the 18S rDNA sequence (approximately 1869 nucleotides in length) of 40S SSU may be used for eukaryotes. For example, 16S sequences may be used for phylogenetic reconstruction as they are general highly conserved but contain specific hypervariable regions that harbor sufficient nucleotide diversity to differentiate genera and species of most bacteria. Although 16S rDNA sequence data has been used to provide taxonomic classification, closely related bacterial strains that are classified within the same genus and species, may exhibit distinct biological phenotypes.

The identity of contemplated bacterial species or strains may be characterized by 16S rRNA or full genome sequence analysis. For example, in certain embodiments, contemplated bacterial strains may comprise a 16S rRNA or genomic sequence having a certain % identity to a reference sequence.

In certain embodiments, a contemplated modified bacterium is capable of stably colonizing the human gut. A disclosed bacterium may, e.g., upon administration to a human subject, result in an abundance greater than 10¹², 10¹¹, 10¹⁰, 10⁹, 10⁸, or 10⁷ cfu per gram of fecal content. For example, administration of about 10³, about 10⁴, about 10⁵, about 10⁶, about 10⁷, about 10⁸, about 10⁹, about 10¹⁰, about 10¹¹, or about 10¹² cells of a disclosed bacterium to a human subject may result in an abundance greater than 10¹², 10¹¹, 10¹⁰, 10⁹, 10⁸, or 10⁷ cfu per gram of fecal content with 12 hours, 24 hours, 36 hours, 48 hours, 60 hours, or 72 hours of administration.

A disclosed bacterium may, e.g., have been modified to colonize the human gut with increased abundance, stability, predictability or ease of initial colonization relative to a similar or otherwise identical bacterium that has not been modified. For example, a contemplated bacterium may be modified to increase its ability to utilize a privileged nutrient as carbon source. A “privileged nutrient” is defined as a molecule or set of molecules that can be consumed to aid in the proliferation of a particular bacterial strain while providing proliferation assistance to no more than 1% of the other bacteria in the gut. Accordingly, in certain embodiments, a modified bacterium has the ability to consume the privileged nutrient to sustain its colonization and expand in the gut of a subject to a predictably high abundance, even in the absence of oxalate or other carbon or energy sources, while most other bacteria in the gut of the subject do not. Exemplary privileged nutrients include, e.g., a marine polysaccharide, e.g., a porphyran.

For example, a bacterium may comprise one or more transgenes that increase its ability to utilize a privileged nutrient, e.g., a marine polysaccharide, e.g., a porphyran, as carbon source. In certain embodiments, a bacterium may comprise all or a portion of a polysaccharide utilization locus (PUL), a mobile genetic element that confers the ability to consume a carbohydrate, e.g., a privileged nutrient, upon a bacterium. An exemplary porphyran consumption PUL is the PUL from the porphyran-consuming Bacteroides strain NB001 depicted in SEQ ID NO: 83. Accordingly, in certain embodiments, a modified bacterium comprises SEQ ID NO: 83, or a functional fragment or variant thereof. In certain embodiments, a modified bacterium comprises a nucleotide sequence having at least 80%, 85%, 86%, 87%, 88%, 89%, 90%, 91%, 92%, 93%, 94%, 95%, 96%, 97%, 98%, or 99% identity to SEQ ID NO: 83, or a functional fragment or variant thereof.

Additional exemplary bacterial modifications to increase abundance in the gut of a subject, privileged nutrients, transgenes that increase the ability of a bacteria to utilize a privileged nutrient, PULs, and other methods and compositions for modulating the growth of a modified bacterium are described in International (PCT) Patent Publication No. WO2018112194.

In certain embodiments, a disclosed transgene or nucleic acid comprising an exogenous nucleotide sequence is operably linked to at least one promoter, e.g., a constitutive promoter, e.g., a phage-derived promoter. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome. Exemplary phage-derived promoters include those comprising the nucleotide sequence of SEQ ID NO: 77, SEQ ID NO: 78 SEQ ID NO: 79, or SEQ ID NO: 80, or a nucleotide sequence having at least 80%, 85%, 86%, 87%, 88%, 89%, 90%, 91%, 92%, 93%, 94%, 95%, 96%, 97%, 98%, or 99% identity to SEQ ID NO: 77, SEQ ID NO: 78, SEQ ID NO: 79, or SEQ ID NO: 80. Additional exemplary phage-derived promoters are described in International (PCT) Patent Publication No. WO2017184565.

### II. Pharmaceutical Compositions/Units

A bacterium disclosed herein may be combined with pharmaceutically acceptable excipients to form a pharmaceutical composition, which can be administered to a patient by any means known in the art. As used herein, the term “pharmaceutically acceptable excipient” is understood to mean one or more of a buffer, carrier, or excipient suitable for administration to a subject, for example, a human subject, without excessive toxicity, irritation, allergic response, or other problem or complication, commensurate with a reasonable benefit/risk ratio. The excipient(s) should be “acceptable” in the sense of being compatible with the other ingredients of the formulations and not deleterious to the recipient.

Pharmaceutically acceptable excipients include buffers, solvents, dispersion media, coatings, isotonic and absorption delaying agents, and the like, that are compatible with pharmaceutical administration. Pharmaceutically acceptable excipients also include fillers, binders, disintegrants, glidants, lubricants, and any combination(s) thereof. For further examples of excipients, carriers, stabilizers and adjuvants, see, e.g., Handbook of Pharmaceutical Excipients, 8^{th} Ed., Edited by P. J. Sheskey, W. G. Cook, and C. G. Cable, Pharmaceutical Press, London, UK [2017]. The use of such media and agents for pharmaceutically active substances is known in the art.

Contemplated bacteria may be used in disclosed compositions in any form, e.g., a stable form, as known to those skilled in the art, including in a lyophilized state (with optionally one or more appropriate cryoprotectants), frozen (e.g., in a standard or super-cooled freezer), spray dried, and/or freeze dried. A “stable” formulation or composition is one in which the biologically active material therein essentially retains its physical stability, chemical stability, and/or biological activity upon storage. Stability can be measured at a selected temperature and humidity conditions for a selected time period. Trend analysis can be used to estimate an expected shelf life before a material has actually been in storage for that time period. For live bacteria, for example, stability may be defined as the time it takes to lose 1 log of CFU/g dry formulation under predefined conditions of temperature, humidity and time period.

A bacterium disclosed herein may be combined with one or more cryoprotectants. Exemplary cryoprotectants include fructoligosaccharides (e.g., Raftilose®), trehalose, maltodextrin, sodium alginate, proline, glutamic acid, glycine (e.g., glycine betaine), mono-, di-, or polysaccharides (such as glucose, sucrose, maltose, lactose), polyols (such as mannitol, sorbitol, or glycerol), dextran, DMSO, methylcellulose, propylene glycol, polyvinylpyrrolidone, non-ionic surfactants such as Tween 80, and/or any combinations thereof.

A pharmaceutical composition should be formulated to be compatible with its intended route of administration. Contemplated bacterial compositions disclosed herein can be prepared by any suitable method and can be formulated into a variety of forms and administered by a number of different means. Contemplated compositions can be administered orally, rectally, or enterally, in formulations containing conventionally acceptable carriers, adjuvants, and vehicles as desired. As used herein, “rectal administration” is understood to include administration by enema, suppository, or colonoscopy. A disclosed pharmaceutical composition may, e.g., be suitable for bolus administration or bolus release. In an exemplary embodiment, a disclosed bacterial composition is administered orally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit®”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as a taste-masking agent.

Tablets, pills, and the like can be compressed, multiply compressed, multiply layered, and/or coated. A contemplated coating can be single or multiple. In one embodiment, a contemplated coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, porphyrans, agar, alginates, chitosans, or gellan gum. In some embodiments a contemplated coating material comprises a protein. In some embodiments a contemplated coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments a contemplated coating material comprises at least one edible wax. A contemplated edible wax can be derived from animals, insects, or plants. Non-limiting examples include beeswax, lanolin, bayberry wax, carnauba wax, and rice bran wax. Tablets and pills can additionally be prepared with enteric or reverse-enteric coatings.

Alternatively, powders or granules embodying a bacterial composition disclosed herein can be incorporated into a food product. In some embodiments a contemplated food product is a drink for oral administration. Non-limiting examples of a suitable drink include water, fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

Pharmaceutical compositions containing a bacterium disclosed herein can be presented in a unit dosage form, i.e., a pharmaceutical unit. A composition, e.g., a pharmaceutical unit provided herein, may include any appropriate amount of bacterium, measured either by total mass or by colony forming units of the bacteria.

For example, a disclosed pharmaceutical composition or unit may include from about 10³ CFUs to about 10¹² CFUs, about 10⁶ CFUs to about 10¹² CFUs, about 10⁷ CFUs to about 10¹² CFUs, about 10⁸ CFUs to about 10¹² CFUs, about 10⁹ CFUs to about 10¹² CFUs, about 10¹⁰ CFUs to about 10¹² CFUs, about 10¹¹ CFUs to about 10¹² CFUs, about 10³ CFUs to about 10¹¹ CFUs, about 10⁶ CFUs to about 10¹¹ CFUs, about 10⁷ CFUs to about 10¹¹ CFUs, about 10⁸ CFUs to about 10¹¹ CFUs, about 10⁹ CFUs to about 10¹¹ CFUs, about 10¹⁰ CFUs to about 10¹¹ CFUs, about 10³ CFUs to about 10¹⁰ CFUs, about 10⁶ CFUs to about 10¹⁰ CFUs, about 10⁷ CFUs to about 10¹⁰ CFUs, about 10⁸ CFUs to about 10¹⁰ CFUs, about 10⁹ CFUs to about 10¹⁰ CFUs, about 10³ CFUs to about 10⁹ CFUs, about 10⁶ CFUs to about 10⁹ CFUs, about 10⁷ CFUs to about 10⁹ CFUs, about 10⁸ CFUs to about 10⁹ CFUs, about 10³ CFUs to about 10⁸ CFUs, about 10⁶ CFUs to about 10⁸ CFUs, about 10⁷ CFUs to about 10⁸ CFUs, about 10³ CFUs to about 10⁷ CFUs, about 10⁶ CFUs to about 10⁷ CFUs, or about 10³ CFUs to about 10⁶ CFUs of each bacterial strain, or may include about 10³ CFUs, about 10⁶ CFUs, about 10⁷ CFUs, about 10⁸ CFUs, about 10⁹ CFUs, about 10¹⁰ CFUs, about 10¹¹ CFUs, or about 10¹² CFUs of bacteria.

### III. Therapeutic Uses

Compositions and methods disclosed herein can be used to treat various bile acid disorders. As used herein, “bile acid disorder” refers a disorder or disease mediated by, or otherwise associated with, a bile acid or bile salt. In certain embodiments, a “bile acid disorder” is a disorder or disease mediated by, or otherwise associated with, an elevated amount of a bile acid or bile salt in a subject. In certain embodiments, a “bile acid disorder” is a disorder or disease mediated by, or otherwise associated with, a reduced amount of a bile acid or bile salt in a subject. As used herein, “elevated amount of a bile acid or bile salt in a subject” or “reduced amount of a bile acid or bile salt in a subject” may refer to an elevated or reduced amount of the bile acid or bile salt in a body fluid (e.g., blood, plasma, serum, or urine), tissue and/or cell in a subject, relative to a subject without the disease or disorder. The disclosure provides a method of treating a bile acid disorder in a subject. A contemplated method comprises administering to the subject an effective amount of a bacterium or a pharmaceutical composition disclosed herein, either alone or in a combination with another therapeutic agent, to treat the disease or disorder associated with an elevated amount of oxalate in the subject.

Exemplary diseases associated with bile acids include Irritable Bowel Syndrome (IBS), chronic diarrhea, bile acid diarrhea (e.g., type 1 or type 2 (idiopathic) bile acid diarrhea), a metabolic disorder (e.g., obesity, type 2 diabetes, hyperlipidemia, or atherosclerosis), cholelithiasis (e.g., intrahepatic cholestasis of pregnancy, or cholelithiasis associated with primary sclerosing cholangitis or primary biliary cholangitis), liver or gallbladder disease (e.g., Steatosis, Nonalcoholic Fatty Liver Disease (NAFLD), Steatosis, Non-alcoholic Steatohepatitis (NASH), cystic liver disease or non-alcoholic fatty liver disease), In-born Errors of BA Metabolism, Progressive Familial Intrahepatic Cholestasis (PFIC), or Primary Sclerosing Cholangitis (PSC)), cancer (e.g., colon cancer or gastrointestinal cancer), an autoimmune or inflammatory disorder (e.g., inflammatory bowel disease (IBD), or primary biliary cholangitis (PBC)), or a bacterial infection (e.g., a Clostridioides difficile infection).

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals, e.g., human, a companion animal (e.g., dog, cat, or rabbit), or a livestock animal (for example, cow, sheep, pig, goat, horse, donkey, and mule, buffalo, oxen, or camel)).

It will be appreciated that the exact dosage of a pharmaceutical composition, or bacterium is chosen by an individual physician in view of the patient to be treated, in general, dosage and administration are adjusted to provide an effective amount of the bacterial agent to the patient being treated. As used herein, the “effective amount” refers to the amount necessary to elicit a beneficial or desired biological response. An effective amount can be administered in one or more administrations, applications or dosages and is not intended to be limited to a particular formulation or administration route. As will be appreciated by those of ordinary skill in this art, the effective amount of a pharmaceutical unit, pharmaceutical composition, or bacterial strain may vary depending on such factors as the desired biological endpoint, the drug to be delivered, the target tissue, the route of administration, etc. Additional factors which may be taken into account include the severity of the disease state; age, weight and gender of the patient being treated; diet, time and frequency of administration; drug combinations; reaction sensitivities; and tolerance/response to therapy.

Contemplated methods may further comprise administrating a privileged nutrient to the subject to support colonization of the bacterium. Exemplary privileged nutrients include marine polysaccharides, e.g., a porphyran. For example, a disclosed privileged nutrient may be administered to the subject prior to, at the same time as, or after a disclosed bacterium.

Methods and compositions described herein may reduce a level of a bile acid in a subject, e.g., in a body fluid (e.g., blood, plasma, serum, or urine), tissue and/or cell in a subject, by at least about 10%, about 20%, about 30%, about 40%, about 50%, about 60%, about 70%, about 80%, about 90%, about 95%, or more, relative to the level of the bile acid in an untreated or control subject.

Contemplated methods may comprise administration of a disclosed bacterium or pharmaceutical composition to a subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In certain embodiments, the time between consecutive administrations of a disclosed bacterium or pharmaceutical composition to a subject is greater than 12 hours, 24 hours, 36 hours, 48 hours, 2 days, 3 days, 4 days, 5 days, 6 days, 7 days, 1 week, 2 weeks, 3 weeks, or 4 weeks.

In certain embodiments, a disclosed bacterium and a disclosed privileged nutrient, e.g., a marine polysaccharide, e.g., a porphyran are administered to a subject with the same frequency. For example, the bacterium and the privileged nutrient may both be administered to the subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In certain embodiments, a disclosed bacterium and a disclosed privileged nutrient, e.g., a marine polysaccharide, e.g., a porphyran, are administered to a subject with a different frequency. For example, the bacterium may be administered to the subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months, and the privileged nutrient may be administered to the subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. For example, in certain embodiments, the bacterium may be administered to the subject every week, 2 weeks, 3 weeks, 4 weeks, month, 2 months, 3 months, 4 months, 5 months, or 6 months, and the privileged nutrient may be administered to the subject every 12 hours, 24 hours, day, 2 days, 3 days, 4 days, 5 days, 6 days, or 7 days.

The use of the term, “complex-native microbiota” or “complex-native microbiome” should be understood to describe an aggregate of all microbiota of a subject that reside on or within tissues and biofluids along with corresponding anatomical sites in which they reside (e.g., gastrointestinal tract). In some embodiments, a complex-native microbiota comprises at least 10 bacterial species. In some embodiments, a complex-native microbiota comprises greater than 10 bacterial species.

In some embodiments, rate of metabolism of bile acids and bile salts for a bacterium provided by the present disclosure is measured. In some embodiments, rate of metabolism is measured in a subject's gastrointestinal tract by first calculating a linear rate of metabolism of a bacterium (e.g., a bacterium described herein) in a particular assay (e.g., an in vitro assay), and normalizing to number of bacterial cells in said assay (measured by CFUs) to calculate the rate of bile acid and/or bile salt metabolism on a per cell basis. This rate value then multiplied by the colonization levels of a bacterium (e.g., a bacterium described herein) and the colon volume to yield rate of metabolism in the gut in units mM/hour.

In some embodiments, percent conversion of one or more bile acids and bile salts to one or more different bile acid or bile salt products is measured. In some embodiments, percent conversion is measured by comparing the level of a bile acid and/or bile salt metabolite from a group of animals colonized with an engineered bile acid-metabolizing Bacteroides strain to a group of animals colonized with a control non-metabolizing Bacteroides strain. Percent conversion is calculated by first subtracting the level of a bile acid and/or bile salt in the engineered bile acid-metabolizing Bacteroides strain from the control non-metabolizing Bacteroides strain and the dividing this value by level of a bile acid and/or bile salt from the control non-metabolizing Bacteroides strain and multiplying by 100. The difference in bile acid and/or bile salt concentrations is calculated by simply subtracting the level of a bile acid and/or bile salt in the engineered bile acid-metabolizing Bacteroides strain from the control non-metabolizing Bacteroides strain.

Methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. In certain embodiments, a side effect of a first and/or second treatment is reduced because of combined administration.

In certain embodiments, a method or composition described herein is administered in combination with one or more additional therapies. In certain embodiments, a contemplated additional therapy may include a bile acid sequestrant.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

As used herein, singular forms “a,” “an,” and “the” include plural referents unless the context clearly indicates otherwise. Thus, for example, reference to “a bacterium” includes a plurality of bacteria and reference to “a bacterium” in some embodiments includes multiple bacteria (e.g., bacteria of the same strain, or multiple strains of bacteria, including those that carry different enzymes relative to each other), and so forth.

### EXAMPLES

The following Examples are merely illustrative and are not intended to limit the scope or content of the disclosure in any way.

### Example 1—Materials and Methods

### Chemicals

The bile acids CDCA, DCA, UDCA, CA-d4 were purchased from Sigma-Aldrich (St. Louis, MO). 7-Keto-lithocholic acid, DCA-d4, and UDCA-d4 was purchased from Cayman Chemical (Ann Arbor, MI). CDCA-d4 was purchased from Toronto Research Chemicals (Toronto, Canada). IsoDCA and lagoDCA were purchased from Steraloids (Newport, RI).

### Bacterial Strains and Culture Conditions

The described experiments were performed using Bacteroides strain NB144 (as described in International Patent Application No. PCT/US20/37571, herein incorporated by reference for all purposes), which can controllably colonize a host through use of porphyran. All Bacteroides strains were grown in BHIS media, comprised of Brain Heart Infusion media (Difco) supplemented with hemin (5 μg/mL), vitamin K (1 μg/mL), and cysteine (0.5 g/mL). Bacteroides strains were grown in an anaerobic chamber (Coy Laboratory Products Inc.) at 37° C. under an atmosphere of 20% CO₂, 5% H₂, and 85% N₂. When required, the following antibiotics were used for selection at the specified concentrations: erythromycin (25 μg/mL), tetracycline (2 μg/mL), and gentamicin (200 μg/mL). Bile acids were prepared in 100 mM stocks in dimethyl sulfoxide stocks (DMSO) and inoculated into BHIS media 1 in 1000 for in vitro assays. Routine molecular procedures were performed using E. coli. E. coli S1λ pir was used for conjugal transfer of genetic material from to Bacteroides. E. coli strains were routinely grown aerobically in Lysogeny Broth (LB) media with shaking at 250 rpm at 37° C. When appropriate, LB was supplemented with 100 μg/mL carbenicillin.

### Construction of Bile Acid Metabolizing Vectors

Microbial HSDH genes were codon optimized for expression in Bacteroides and synthesized into gBlocks by Integrated DNA Technologies (Newark, NJ). Using Golden Gate cloning, HSDH genes were assembled with the appropriate transcriptional and translational features into a vector suitable for heterologous expression, chromosomal integration, and conjugal transfer to Bacteroides. Vectors were based on the mobilizable Bacteroides element NBU2.

### Construction of Bile Acid Metabolizing Strains

Expression vectors were sequentially introduced into strain NB144 by conjugation using E. coli 517-1λ pir as the donor strain using a previously described protocol [Whitaker et al. Tunable Expression Tools Enable Single-Cell Strain Distinction in the Gut Microbiome. Cell 169, 538-546.e12 (2017), herein incorporated by reference for all purposes]. A control non-metabolizing Bacteroides strain capable of in vivo colonization was constructed that was identical to the metabolizing strains but only lacked the HSDH open reading frames and was named sPS064.

### Assay to Measure Bile Acid Metabolism In Vitro and Ex Vivo

A 5 mL starter culture of Bacteroides strains were grown anaerobically in BHIS media for approximately 16 hours at 37° C. Pre-equilibrated BHIS media supplemented with 100 μM CDCA (0.1% v/v final concentration of DMSO) in a total volume of 1 mL was inoculated 1 in 1000 with the starter culture. Assay plates were incubated anaerobically at 37° C. for 24 hours. Following incubation, assay cultures were centrifuged at 3,500×g for 2 min to pellet cells. A 30 μL sample of conditioned media was removed and mixed with 70 μL of 100% methanol and centrifuged at 3,500×g for 5 minutes. A 10 μL aliquot of the supernatant was further diluted with 90 μL of 100% methanol. 2 μL of this sample dilution was analyzed for bile acid metabolism using ultra-high-performance liquid chromatography-high-resolution mass spectrometry (UHPLC-HRMS). Colony-forming units (CFUs) were performed to enumerate the number of Bacteroides cells per assay. Rate determinations of bile acid metabolism were performed using mid-logarithmic growth phase Bacteroides strains. Final bile acid concentrations in these assays were 100 μM or 1 mM. Ex vivo assays were performed by resuspending cecal or fecal samples 1:15 (w/v) in pre-equilibrated BHIS media or PBS in an anaerobic chamber. The rate of metabolism in the human gastrointestinal tract was calculated by first calculating the linear rate of metabolism in a particular assay and normalizing to the number of bacterial cells in the assay (measured by CFUs) to calculate the rate of BA metabolism on a per cell basis. This rate value was then multiplied by the colonization levels of the engineered Bacteroides strains using porphyran (˜1×10⁹ CFU/L) and the colon volume (1 L) to yield rate of metabolism in the gut in units mM/hour.

### Mouse Experiments

All mouse experiments were approved by the Institutional Animal Care and Use Committee and conducted at and supported by Charles River Accelerator and Development Lab (CRADL) South San Francisco (South San Francisco, CA). Gnotobiotic experiments were performed with female Swiss Webster germ-free mice at 6-8 weeks of age, purchased from Charles River and housed using an Innovive Disposable IVC Rodent Caging System (San Diego, CA). Experiments with conventionally-raised mice were performed with female C57BL/6 mice 6-8 weeks of age, purchased from Charles River and were housed as described above. Prior to colonization, mice were fed a standard sterilized autoclaved diet ad libitum with free access to water. Housing conditions were at room temperature (24° C.) and 12 h/12 h light/dark cycle (7:00 am-7:00 pm). All handling and procedures of germ-free and gnotobiotic mice were performed within a Biological Class II A1 Biosafety Cabinet. Mice were acclimated for a minimum of 4 days prior to colonization. Before colonizing, mice were orally gavaged with 200 μL of a 5% (w/v) filter-sterilized solution of sodium bicarbonate in water. 15 minutes after sodium bicarbonate delivery, mice were gavaged with a mid-log growth phase Bacteroides culture (˜10¹¹ CFUs/mL) grown anaerobically in BHIS as described above. Colonization was verified by plating dilutions of fecal pellets on BHIS media supplemented with the appropriate antibiotics for selection to count CFUs. Following one week of colonization, mice were gavaged with bile acids (between 200-500 mg/kg) dissolved in corn oil or Captisol and singly caged. After 24 hours fecal pellets were collected, pooled, and stored at −80° C. Experiments with conventionally-raised mice were performed with female C57BL/6 mice 6-8 weeks of age, purchased from Charles River and were housed and colonized as described above. Following gavage, mice were transferred to a chow diet supplemented with porphyran for Bacteroides strain maintenance After one week of colonization, mice were sacrificed and cecal and fecal samples were harvested for ex vivo incubations to determine rates of metabolism.

Bile Acid Extractions from Murine Fecal Samples

Pooled 24-hour fecal samples were weighed and placed in a 15 mL conical tube with 3 Qiagen 5 mm stainless steel beads (Hilden, Germany). Fecal samples were diluted 1:15 (w/v) in sterile phosphate buffer solution pH 7.4. Fecal samples were disrupted by vortexing for 5 min followed by centrifugation at 3,000×rcf for 2 min. A 100 μL sample of the supernatant was transferred to a 2 mL 96-deep-well plate and mixed with 400 μL of 70% methanol solution. The 96-deep-well plate was vortexed for 5 seconds and stored at 4° C. for ˜16 hours. A 25 μL sample was then diluted with 75 μL of methanol supplemented with 0.25 μM d4-CAm which was used as an internal standard. 2 μL of this sample dilution was analyzed by UHPLC-HRMS.

### Measuring Bile Acids by UHPLC-HRMS

Bile acids were quantified using UHPLC-HRMS as previously described [Ding et al. High-throughput bioanalysis of bile acids and their conjugates using UHPLC coupled to HRMS. Bioanalysis 5, 2481-2494 (2013), herein incorporated by reference for all purposes]. Briefly, chromatography was performed using a Thermo Fisher Vanquish UHPLC system (Waltham, MA) with an Agilent Technologies Zorbax Eclipse XDB-C18 column, 1.8 μm, 50 or 100×2.1 mm internal diameter (Santa Clara, CA). Bile acids from in vitro conditioned media samples were extracted as described above and performed with a 50 mm column at 30° C. with the following mobile phases: (A) 0.01% formic acid in LC-MS grade water, and (B) acetonitrile (AcN). The flow rate was 0.7 mL/min and bile acids were separated using the following method: 75% A and 25% B for 1.5 min; 50% A and 50% B in 4 min; 100% B in 1.5; maintain 100% B for 1 min; immediate switch to 75% A and 25% B and maintain for 2 min to equilibrate the column. Bile acids from murine fecal samples were analyzed using a 100 mm column at 30° C. with the same mobile phases described above. The flow rate was 0.5 mL/min and bile acids were separated using the following method: 65% A and 35% B for 2 min; 50% A and 50% B in 15 min; 100% B in 3 min; maintain 100% B for 2 min; immediate switch to 65% A and 35% B and maintain for 2 min. Bile acid detection was performed using the Thermo Fisher QExactive. Detection of bile acids from in vitro conditioned media was performed in negative ion full-scan mode (mass range: 300-500 m/z) at 140,000 resolution with automatic gain control target of 1e6 and maximum ion injection time of 200 ms. The eluent from the column was introduced into the HESI ion source operating under the following parameters: spray voltage=3 kV; sheath, auxiliary, and sweep gases were 60, 15, and 1 arbitrary units, respectively; S-lens=50; capillary temperature=325° C., heater temperature=450° C.; and an in-source collision-induced dissociation=30 eV. bile acid concentrations in samples were determined by relating to a standard curve. Detection of bile acids from in vivo murine fecal samples was performed in negative ion mode using a scheduled targeted-selected ion monitoring method with the following parameters: 140,000 resolution with automatic gain control target of 5e4, maximum ion injection time of 200 ms, and an isolation window of 1.5 m/z. The eluent from the column was introduced into the HESI ion source operating under parameters described above. The schedule of the method was determined using commercially available authentic bile acid standards. Bile acid concentrations in samples were determined by relating to a standard curve. Bile acid concentrations from conditioned media samples were determined using a standard curve designed by spiking in known bile acid standards at various concentrations into BHIS media. Bile acid concentrations from fecal samples were determined using a standard curve designed by spiking in deuterated bile acid standards (d4-CDCA and d4-UDCA) at various concentrations into fecal samples.

### Example 2—Bacteroides Strains Engineered to Express 7α-HSDH and 7β-HSDH can Metabolize CDCA

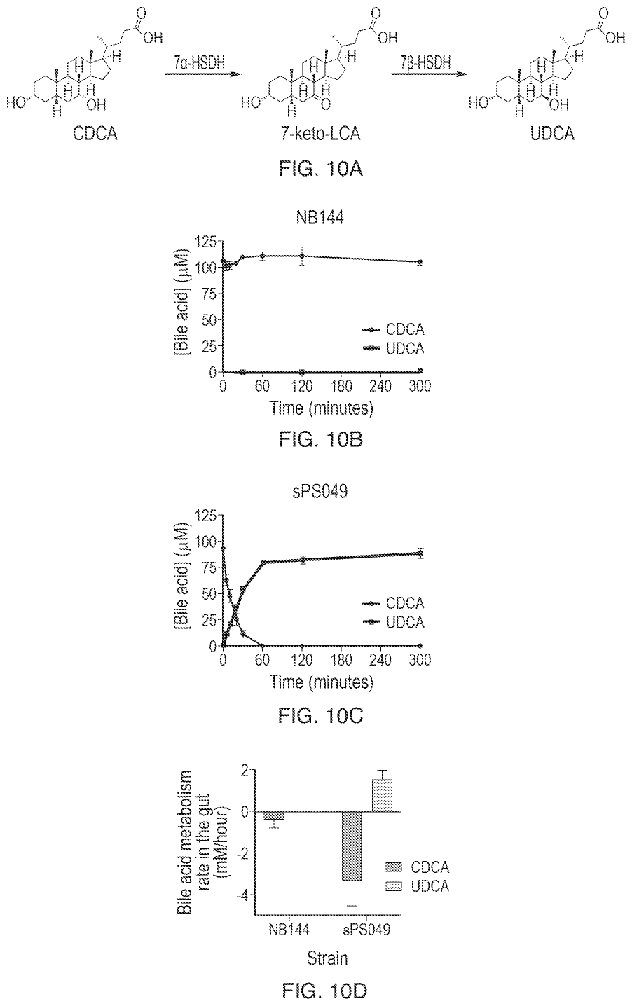

Bacteroides strains were engineered to metabolize CDCA, which is a dominant bile acid present in the human GIT and known to be elevated in fecal samples in a disease state (e.g., IBS-D). Bacteroides strains were designed to metabolize CDCA to UDCA, which is known to be non-secretory and is generally considered tolerable for humans, as it has been approved by the Food and Drug Administration (FDA) for use towards a number of bile acid diseases or disorders. CDCA is converted to UDCA by the sequential action of two enzymes: 7α-HSDH and 7β-HSDH (FIG. 2A). A panel of previously characterized 7α-HSDH genes (e.g., see 7α-HSDH genes having the nucleotide sequences of SEQ ID NOs. 1-6) and 7β-HSDH genes (e.g., see 7β-HSDH genes having the nucleotide sequences of SEQ ID NOs. 7-11) were selected from diverse human gut bacteria for heterologous expression in a Bacteroides platform. Each gene was codon-optimized for expression from select Bacteroides strains and paired with the appropriate transcriptional and translational features for maximally efficient heterologous expression. Engineered Bacteroides strains were screened for metabolism by culturing in BHIS media supplemented with 100 μM CDCA and sampling conditioned media over time and analyzing by UHLPC-HRMS. After screening strains of Bacteroides expressing 7α-HSDH and 7β-HSDH enzymes in pairs, strain sPS049, engineered to express codon optimized 7α-HSDH gene from E. coli Nissle 1917 (SEQ ID NO. 2) and codon optimized 7β-HSDH gene from Colinsella aerofaciens ATCC 25986 (SEQ ID NO. 8), was determined to demonstrate the greatest CDCA-metabolism. As shown in FIG. 10 Panels A-D, parental Bacteroides strain NB144 did not metabolize CDCA (FIG. 10A), whereas sPS049 was capable of completely metabolizing 100 μM of CDCA within 60 minutes (FIG. 10C). Extrapolating using predicted colonization levels within the human GIT, sPS049 was modeled to deplete approximately 3.5 mM of CDCA per hour (FIG. 10D). These data indicate that engineered Bacteroides strains engineered to express 7-hydroxysteroid dehydrogenases can efficiently metabolize physiologically relevant concentrations of the bile acid CDCA in a diseased state within a reasoned timeline in vitro.

### Example 3—Bacteroides Strains Engineered to Express 7α-HSDH and 7β-HSDH can Metabolize CDCA in Complex Microbial Communities

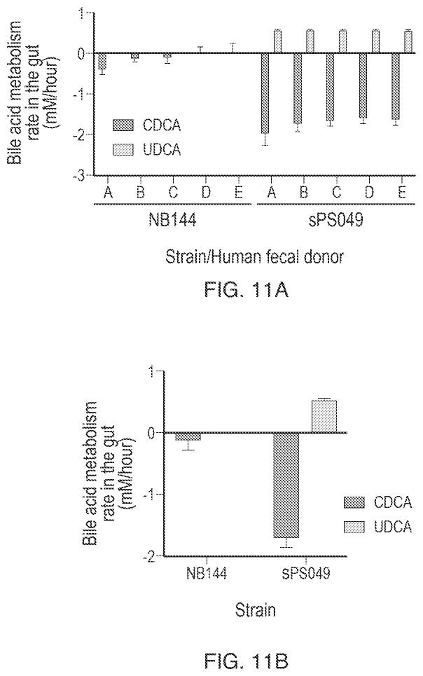

In addition to being colonized with the engineered strains, individuals are also colonized with a pool of other diverse microbes within their GITs, which generally outnumber the engineered Bacteroides strains 10 to 1. Accordingly, whether a complex microbial community impacts the efficiency of bile acid metabolism by the engineered Bacteroides strains was assessed through measuring the rate of CDCA metabolism of sPS049 (SEQ ID NO. 2 and 8) added to ex vivo cultures of human fecal bacteria. Cultures of actively growing human fecal bacteria from five healthy unrelated individuals (A-E) were spiked with sPS049 or a control non-metabolizing parental strain (NB144) at a ratio of 1:10 CFUs and rate of CDCA metabolism was measured. Very little metabolism of CDCA was observed from human fecal bacteria spiked with the non-metabolizing strain NB144 (FIG. 11A). In direct contrast, ex vivo cultures spiked with sPS049 showed significant CDCA metabolism with a concomitant production of UDCA (FIG. 11A). Interestingly, despite the differences in microbial compositions across human fecal samples, the rate of CDCA metabolism was highly consistent across combinations with human fecal bacteria from each of the five healthy unrelated individuals. In this complex human microbial community, sPS049 averaged a rate of CDCA metabolism of ˜2 mM per hour (FIG. 11B). This data demonstrates that the engineered Bacteroides strains can efficiently metabolize physiologically relevant concentrations of CDCA in the presence of complex human fecal microbial communities.

### Example 4—Bacteroides Strains Engineered to Express 7α-HSDH and 7β-HSDH can Metabolize CDCA in Mice Colonized with the Engineered Strains as Assessed Ex Vivo

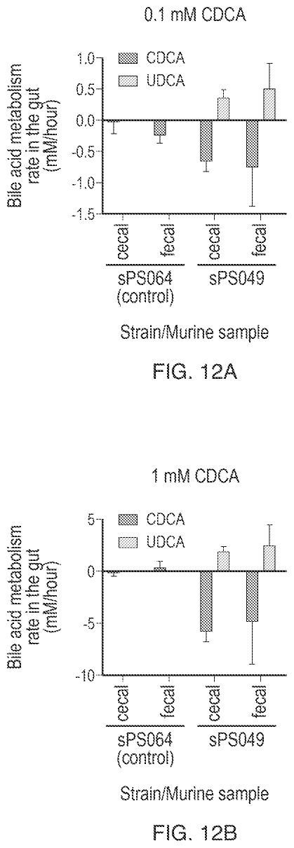

The ability of engineered Bacteroides strains to metabolize CDCA ex vivo following colonization of mice was assessed. Conventionally-raised mice were gavaged with sPS049 (SEQ ID NO. 2 and 8) or a non-metabolizing control strain (sPS064; identical to sPS049 but lacking the HSDH open reading frames) and then transferred animals to a chow diet supplemented with porphyran, which facilitates stable, high-level colonization in the GIT (see International Patent Application No. PCT/US20/37571, herein incorporated by reference for all purposes). After one week of colonization, mice were sacrificed and cecal and fecal samples were harvested for ex vivo incubations. Mouse microbial samples were resuspended and incubated anaerobically with CDCA to measure the rate of metabolism. Two concentrations of CDCA, 0.1 and 1 mM, were tested. Samples colonized with the control non-metabolizing strain sPS064 did not demonstrate substantial CDCA metabolism. In contrast, samples colonized with the engineered Bacteroidetes strain sPS049 showed a high-level of CDCA metabolism from both cecal and fecal samples, relative to the metabolism of the control strain (FIGS. 12A and 12B). Rates of metabolism were comparable across cecal and fecal samples but differed when assayed against various CDCA concentrations. CDCA metabolism rates were ˜0.7 mM per hour when assayed at 0.1 mM from both cecal and fecal samples (FIG. 12A) but were ˜5.5 mM per hour when assayed at 1 mM CDCA (FIG. 12B). Thus, the data demonstrates that the engineered CDCA-metabolizing Bacteroides strains colonized animals and metabolized physiologically-relevant concentrations of CDCA to UDCA ex vivo.

### Example 5—Bacteroides Strains Engineered to Express 7α-HSDH and 7β-HSDH can Metabolize CDCA in Mice Colonized with the Engineered Strains as Assessed In Vivo

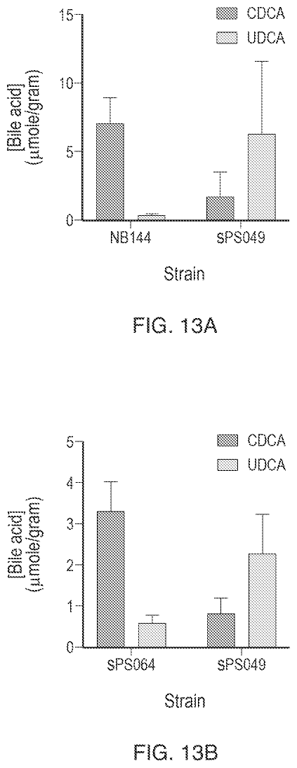

The ability of engineered Bacteroides strains to metabolize CDCA in vivo following colonization of mice was assessed. Germ-free or conventionally-raised mice were colonized with parental control non-metabolizing Bacteroides strain (NB144 or sPS064), or the engineered CDCA-metabolizing strain sPS049 (SEQ ID NO. 2 and 8). Following one week of colonization, mice were gavaged with a single dose of CDCA (500 mg/kg for gnotobiotic mice and 200 mg/kg for conventionally-raised mice) and singly housed. Fecal pellets were collected and pooled over 24 and analyzed for bile acid concentration. In mice colonized with the control parental non-metabolizing strain, high levels of CDCA were detected in the feces from both gnotobiotic and conventionally-raised mice (FIG. 13A and FIG. 13B, respectively). In direct contrast, animals colonized with the 7α-HSDH and 7β-HSDH encoding engineered strain sPS049 showed significantly lower levels of CDCA in the feces of both gnotobiotic and conventionally-raised mice, which was accompanied by an increase in UDCA levels. Thus, the data demonstrated that the engineered strain of Bacteroides successfully metabolizes physiological levels of CDCA to UDCA in vivo.

### Example 6—Bacteroides Strains Engineered to Express 3α-HSDH and 3β-HSDH can Epimerize DCA to isoDCA

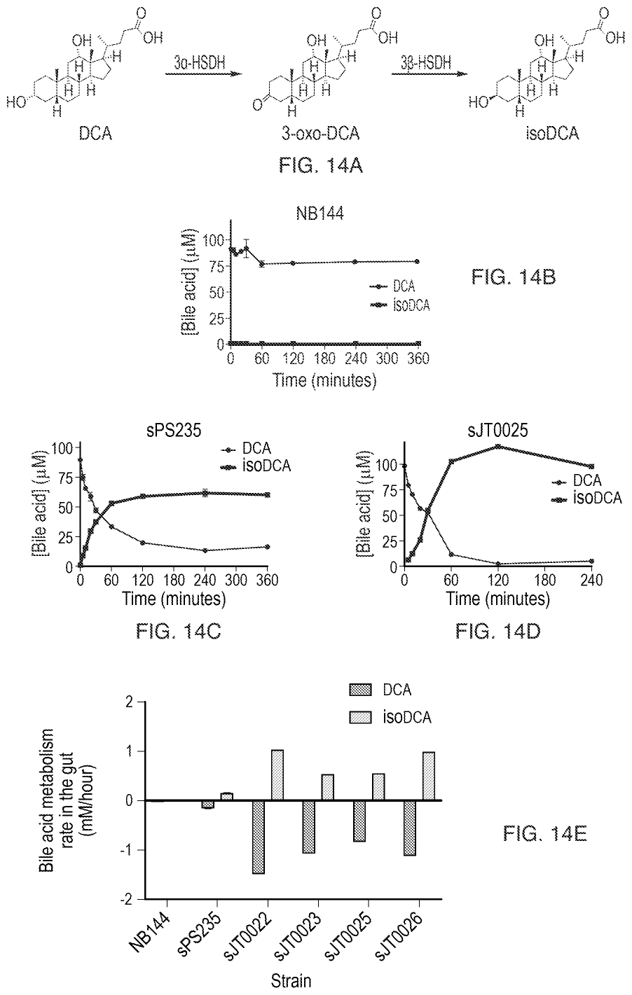

DCA is a major bile acid in the human GIT. Bacteroides strains were engineered to epimerize DCA. A panel of 3α-HSDH genes (e.g., see 3α-HSDH genes having the nucleotide sequences of SEQ ID NOs. 12-23) and 3β-HSDH genes (e.g., see 3β-HSDH genes having the nucleotide sequences of SEQ ID NOs. 24-47) from diverse human gut bacteria were tested for heterologous expression in a Bacteroides platform to metabolize DCA to isodeoxycholic acid (isoDCA) (FIG. 14A). Bacteroides strains engineered to express combinations of the various 3α-HSDH and 3β-HSDH to metabolize DCA were assessed and strain sPS235 engineered to express codon optimized 3α-HSDH gene from Eggerthella lenta DSM2243 (SEQ ID NO. 13) and codon optimized 3β-HSDH gene from Ruminococcus gnavus ATCC 29149 (SEQ ID NO. 24) was determined to demonstrate potent metabolism along with sJT0025 (SEQ ID NO. 18 and 32) (FIGS. 14C and 14D). Parental control strain, NB144, does not metabolize DCA (FIG. 14B). Extrapolation using predicted colonization levels within the human GIT, sPS235 was modeled to deplete ˜0.2 mM of DCA per hour (FIG. 14E). Superior DCA-metabolizing strains were also discovered: JT0022(SEQ ID NO. 18 and 28), sJT0023 (SEQ ID NO. 18 and 30), sJT0025 (SEQ ID NO. 18 and 32), and sJT0026 (SEQ ID NO. 18 and 36), which showed levels of DCA metabolism >0.5 mM/hour. Thus, these data indicate that the engineered Bacteroides strain efficiently metabolized physiologically relevant concentrations of DCA within a reasoned timeline in monoculture in vitro.

### Example 7—Bacteroides Strains Engineered to Express 3α-HSDH and 3β-HSDH can Metabolize DCA in Mice Colonized with the Engineered Strains as Assessed In Vivo

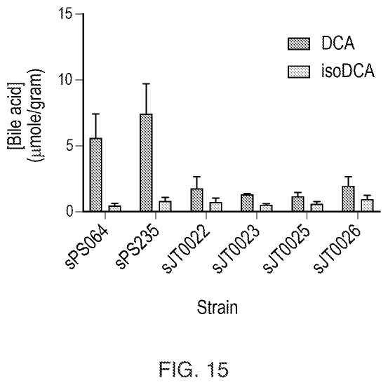

The ability of engineered Bacteroides strains to metabolize DCA in vivo following colonization of mice was assessed. Conventionally-raised mice were gavaged with isoDCA strains with varying rates of DCA metabolism (see Example 6 and FIG. 14E) or a non-metabolizing control strain (sPS064; identical to the isoDCA strains but lacking the 3α-HSDH and 3β-HSDH open reading frames) and then transferred animals to a chow diet supplemented with porphyran, which facilitates stable, high-level colonization in the GIT (see International Patent Application No. PCT/US20/37571, herein incorporated by reference for all purposes). The DCA-metabolizing strains that were used were as follows: sPS235 (SEQ ID NO. 13 and 24), sJT0022 (SEQ ID NO. 18 and 28), sJT0023 (SEQ ID NO. 18 and 30), sJT0025 (SEQ ID NO. 18 and 32), and sJT0026 (SEQ ID NO. 18 and 36). Following one week of colonization, mice were singly housed. Fecal pellets were collected and pooled over 24 and analyzed for bile acid concentration. In mice colonized with the control parental non-metabolizing strain, high levels of DCA were detected in the feces and low levels of isoDCA (FIG. 15). Interestingly, sPS235, which shows a rate of DCA metabolism ˜0.2 mM/hour (FIG. 14E) did not show DCA metabolism or isoDCA production. When compared to the control strain, sPS235 showed a 26% increase (1.63 moles/gram increase) in DCA levels. In direct contrast, animals colonized with the 3α-HSDH- and 3β-HSDH-encoding engineered strains sJT0022, sJT0023, sJT0025, and sJT0026, which all showed levels of DCA metabolism >0.5 mM/hour, showed significantly lower levels of DCA in the feces of mice, which was accompanied by an increase in isoDCA levels (FIG. 15). Compared to the control strain, sJT0022, sJT0023, JT0025, and sJT0026 showed an average of a 77% decrease (4.3 moles/gram decrease) in DCA levels Thus, the data demonstrated that the engineered strain of Bacteroides successfully metabolizes physiological levels of DCA to isoDCA in vivo. The data also showed that DCA-metabolizing strains withrates of metabolism of >0.5 mM/hour (FIG. 14E) achieve DCA metabolism in vivo in animals containing a complex-native microbiota.

### Example 8—Bacteroides Strains Engineered to Express 12α-HSDH and 12β-HSDH can Epimerize DCA to lagoDCA

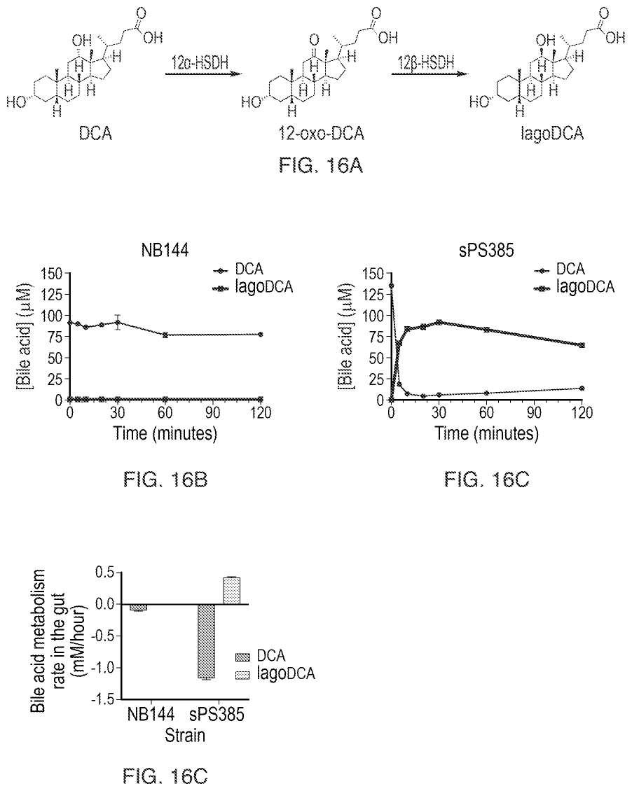

DCA is a major bile acid in the human GIT. Bacteroides strains were engineered to metabolize DCA into the epimer lagoDCA) A panel of 12α-HSDH genes (e.g., see 12α-HSDH genes having the nucleotide sequences of SEQ ID NOs. 48-54 and 12β-HSDH genes (e.g., see 12β-HSDH genes having the nucleotide sequences of SEQ ID NOs. 55-60) from diverse human gut bacteria were tested for heterologous expression in a Bacteroides platform to metabolize DCA to lagodeoxycholic acid (lagoDCA) (FIG. 16 Panels A-D). Bacteroides strains engineered to express combinations of the various 12α-HSDH and 12β-HSDH to metabolize DCA were assessed and strain sPS385 engineered to express codon optimized 12α-HSDH gene from Eggerthella lenta C592 (SEQ ID NO. 48) and codon optimized 12β-HSDH gene from Clostridium paraputrificum ATCC 25780 (SEQ ID NO. 55) was determined to demonstrate potent metabolism. Parental control strain, NB144, does not metabolize DCA (FIG. 16B). In contrast, engineered Bacteroides strains sPS385 rapidly metabolized DCA to lagoDCA (FIG. 16C). Thus, these data indicate that the engineered Bacteroides strain efficiently metabolized physiologically relevant concentrations of DCA to lagoDCA within a reasoned timeline in vitro. Extrapolation using predicted colonization levels within the human GIT, sPS385 was modeled to deplete ˜1 mM of DCA per hour (FIG. 16D). Thus, these data indicate that the engineered Bacteroides strain efficiently metabolized physiologically relevant concentrations of DCA within a reasoned timeline in monoculture in vitro.

### Example 9—Bacteroides Strains Engineered to Express Multiple 3-, 7-, and 12-HSDHs Capable of Generating a Diverse Collection of Bile Acid Products

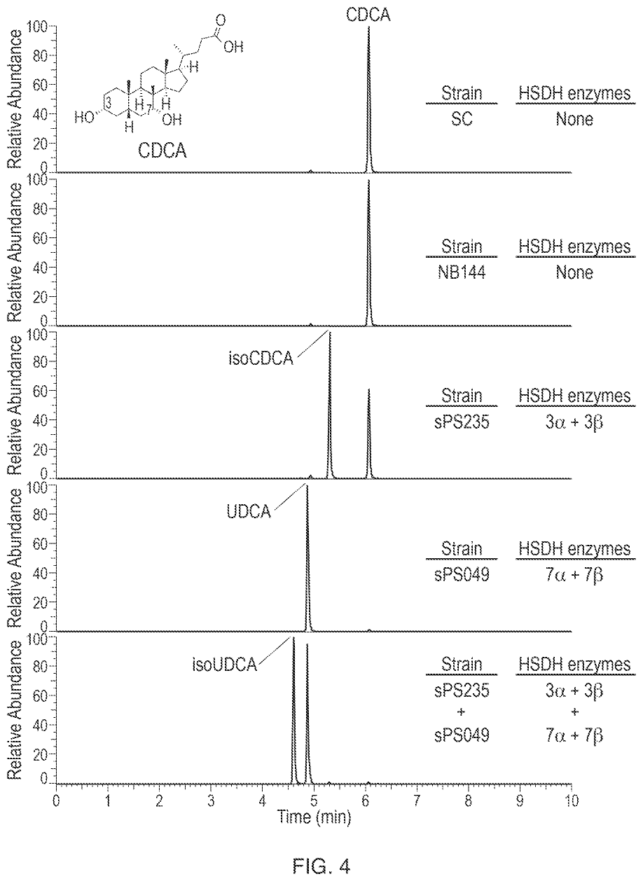

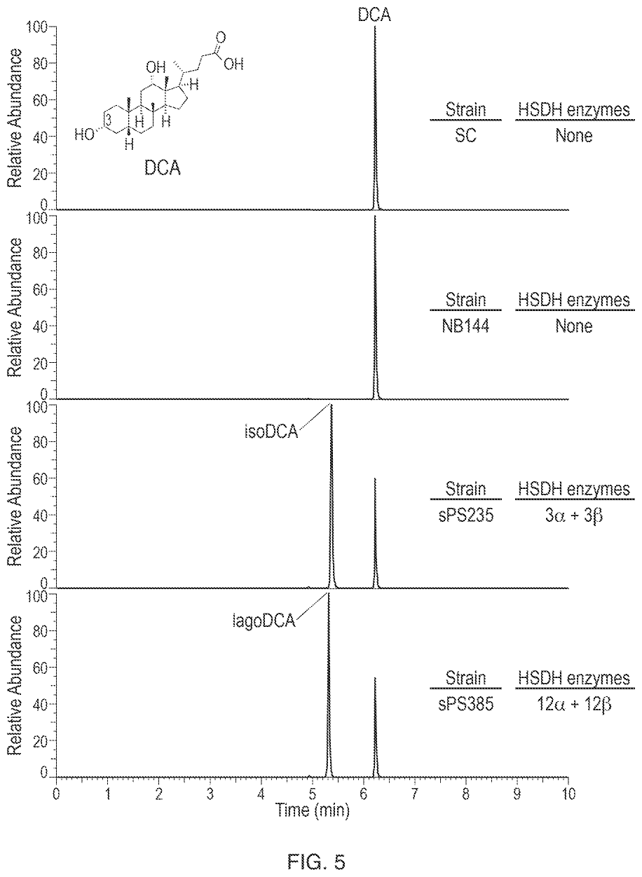

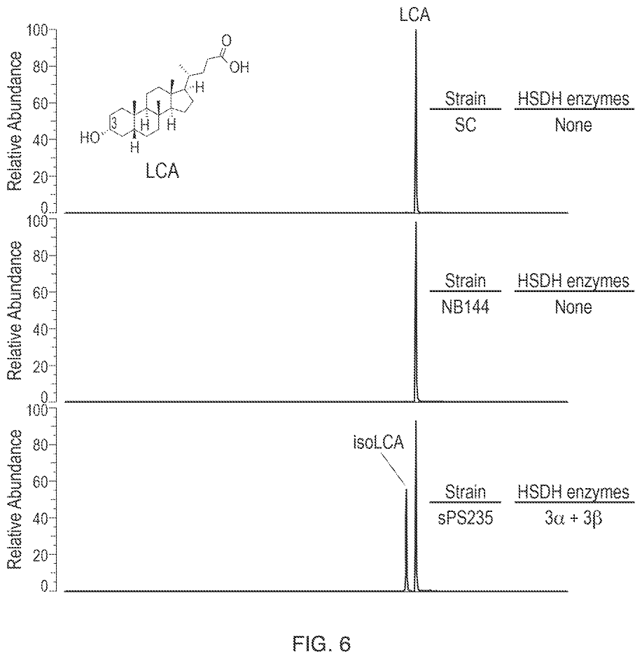

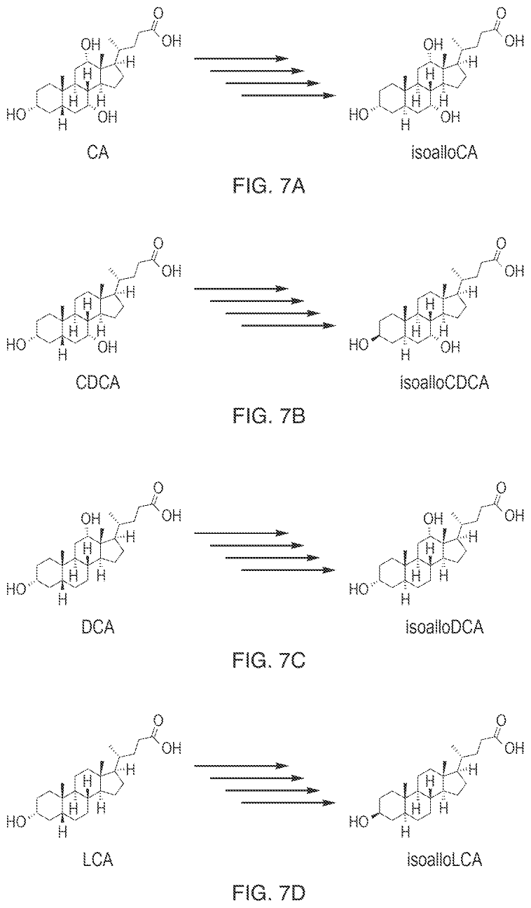

Most bile acids and bile salts possess multiple hydroxyl residues at either of the 3-, 7- and 12-positions of the steroid core. For example, the bile acid cholic acid (CA) contains hydroxyl residues at the 3-, 7-, and 12-position, which can all be positionally targeted for epimerization using site-specific HSDH enzymes (FIG. 2E). Using engineered Bacteroides strains expressing one or more site-specific HSDH enzymes, cholic acid was predictably and successfully converted to 7 different epimer products with various configurations of α-OH and β-OH residues (FIG. 3). Epimers of CDCA (FIG. 4), DCA (FIG. 5), and LCA (FIG. 6) with various configurations of α-OH and β-OH residues were also generated using engineered Bacteroides strains expressing HSDH enzymes. This data confirms that Bacteroides can be engineered to generate bile acid or bile salt epimers successfully and predictably using a combination of site-specific HSDHs. The enzymes used in this example are the 3-OH-targeting strain sPS235 (SEQ ID NO. 13 and 24), the 7-OH-targeting strain sPS049 (SEQ ID NO. 2 and 8), and the 12-OH-targeting strain sPS385 (SEQ ID NO. 48 and 55).

### Example 10—Bacteroides Strains Engineered to Express Enzymes to Generate Isoallo-Bile Acid Products

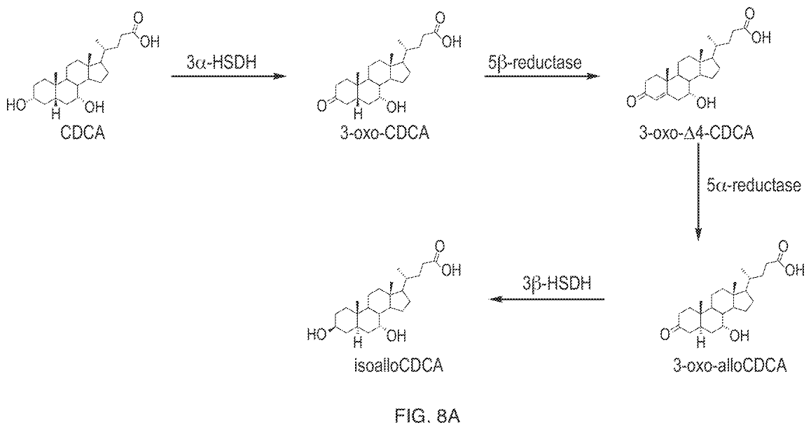

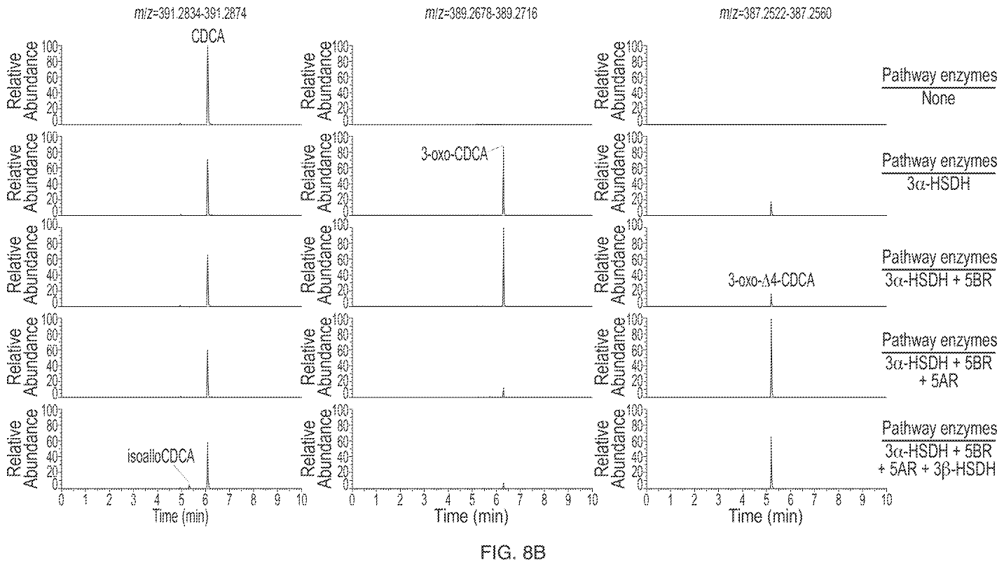

Isoallo-bile acids are generated by the action of four enzymes: 3α-hydroxysteroid dehydrogenase (3α-HSDH), 5β-reductase, (5BR), 5α-reductase (5AR), and 3β-hydroxysteroid dehydrogenase (3β-HSDH) (FIG. 8A). Engineered Bacteroides strains were designed to express each component of the isoallo-bile acid biosynthetic pathway. These strains were successful in converting the bile acid CDCA to isoalloCDCA (FIG. 8B), confirming that Bacteroides strains can be engineered to generated isoallo-bile acids from bile acid substrates.

### Example 11—Bacteroides Strains Engineered to Express Sulfotransferase Enzymes to Generate Sulfated-Bile Acid Products

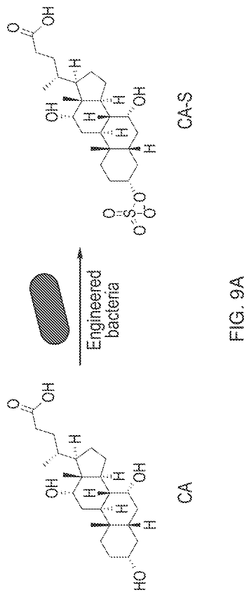

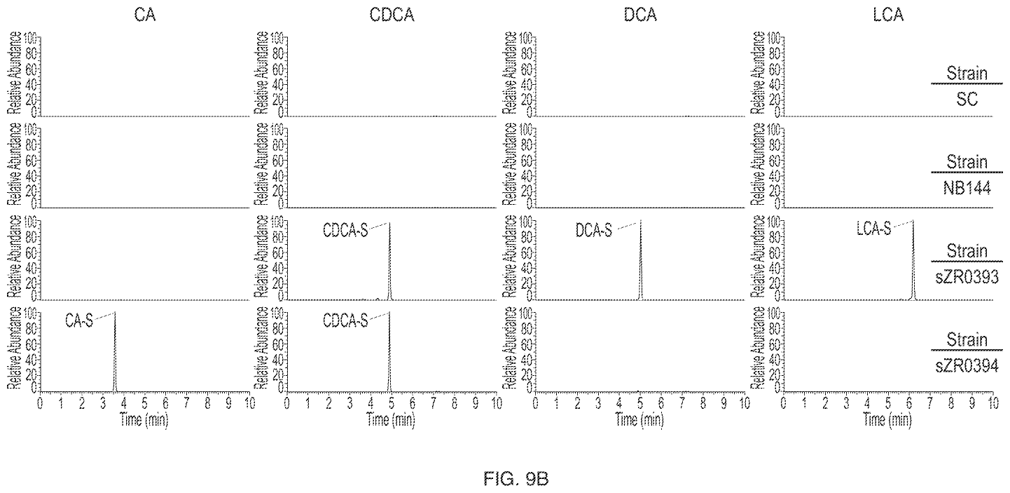

Sulfotransferases (SULTs) are enzymes that transfer sulfo groups from a donor molecule to an acceptor. Bacteroides strains were engineered to express sulfotransferases (FIG. 9A). These strains successfully sulfated multiple bile acid substrates including CA, CDCA, DCA, and LCA (FIG. 9B). These results confirm that Bacteroides can be engineered to sulfate bile acids.

### INCORPORATION BY REFERENCE

The entire disclosure of each of the patent and scientific documents referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

### Sequence Listing

SEQUENCE LISTING

| SEQ | | |
|---|---|---|
| ID | | |
| NO | Description | Sequence |
| 1 | 7α-hydroxysteroid | ATGAACAGATTTGAAAATAAGATAATCATTATCACGGGAGCTGCCGGTGG |
| | dehydrogenase gene | AATCGGCGCATCAACCACACGCCGCATTGTATCTGAAGGCGGCAAAGTAG |
| | from Bacteroides | TTATTGCTGACTATTCAAGAGAAAAAGCAGACCAATTTGCTGCCGAGCTT |
| | fragilis ATCC | AGTAATTCGGGAGCAGATGTACGTCCGGTTTATTTTTCTGCTACAGAATT |
| | 25285 | GAAAAGCTGCAAAGAACTAATCACCTTTACAATGAAGGAATACGGACAGA |
| | | TCGATGTACTGGTAAACAATGTAGGAGGTACAAATCCCAGACGGGACACA |
| | | AACATCGAAACTCTGGATATGGATTATTTTGACGAAGCCTTTCATCTGAA |
| | | TTTATCTTGTACCATGTATTTGTCCCAACTGGTTATCCCCATTATGAGCA |
| | | CACAAGGTGGTGGAAATATTGTAAACGTAGCCTCAATAAGTGGAATCACG |
| | | GCCGATTCGAATGGTACTCTTTATGGAGCCAGCAAAGCAGGAGTCATCAA |
| | | TCTGACCAAATACATTGCCACCCAAACGGGAAAGAAAAACATCCGTTGCA |
| | | ATGCAGTAGCACCAGGATTGATCCTGACCCCGGCCGCACTGAATAATCTT |
| | | AATGAAGAGGTACGCAAAATATTTCTCGGGCAATGTGCGACACCCTATTT |
| | | AGGTGAACCGCAAGACGTTGCCGCGACCATCGCTTTTTTAGCCTCCGAAG |
| | | ATGCACGTTACATTACCGGACAGACCATAGTAGTAGATGGCGGATTGACA |
| | | ATACACAATCCGACAATAAACTTAGTATAA |
| 2 | Codon optimized | ATGTTCAACAGCGACAATCTTCGTCTGGATGGTAAATGCGCAATTATAAC |
| | 7α-hydroxysteroid | AGGTGCAGGCGCCGGAATCGGTAAAGAAATTGCAATAACGTTTGCTACGG |
| | dehydrogenase gene | CTGGAGCTTCAGTGGTAGTGAGCGACATAAACGCCGATGCAGCGAATCAT |
| | from E. coli Nissle | GTGGTTGATGAAATCCAACAACTGGGTGGACAGGCCTTTGCATGTAGATG |
| | 1917 | CGACATCACTTCTGAACAAGAATTATCCGCCCTTGCAGATTTTGCAATTT |
| | | CGAAGCTTGGAAAGGTGGACATCCTTGTCAATAATGCTGGTGGAGGTGGA |
| | | CCTAAACCGTTTGACATGCCAATGGCGGATTTTCGGCGGGCGTATGAATT |
| | | GAATGTATTCTCATTCTTTCATTTAAGTCAGCTTGTAGCTCCCGAAATGG |
| | | AAAAAAATGGAGGAGGAGTAATACTGACAATTACGAGTATGGCTGCCGAG |
| | | AATAAAAACATCAACATGACGTCCTATGCTAGTTCCAAAGCAGCTGCATC |
| | | CCATCTTGTTCGTAATATGGCATTCGACCTGGGCGAAAAAAATATCCGCG |
| | | TAAATGGTATTGCACCGGGCGCTATATTAACTGATGCTCTTAAATCGGTC |
| | | ATTACGCCAGAAATCGAGCAAAAGATGCTGCAGCATACACCAATTCGTCG |
| | | TCTTGGCCAACCGCAAGACATTGCCAACGCTGCATTATTTTTATGTTCAC |
| | | CGGCCGCCAGTTGGGTATCAGGTCAAATCTTGACAGTATCTGGTGGTGGA |
| | | GTACAGGAACTGAACTAA |
| 3 | Codon optimized | ATGAATAAGTTGGAAAACAAAGTTGCTCTGGTAACATCTGCCACACGTGG |
| | 7α-hydroxysteroid | CATCGGATTAGCAAGTGCTATAAAATTAGCGCAGAATGGAGCCATCGTTT |
| | dehydrogenase gene | ATATGGGTGTACGCCGTCTTGAAGCAACGCAGGAAATATGTGACAAGTAT |
| | from | AAAGAAGAAGGCCTGATTCTTAAGCCCGTTTTCTTTGATGCATATAATAT |
| | Paeniclostridium | AGACATATATAAAGAAATGATTGATACAATTATAAAAAACGAAGGTAAGA |
| | sordellii | TCGACATCTTGGTCAATAACTTCGGTACAGGACGTCCGGAAAAGGATCTG |
| | | GATCTTGTAAATGGGGATGAAGACACATTCTTCGAATTATTCAACTATAA |
| | | TGTAGGTTCTGTTTATCGGCTGTCCAAATTAATAATTCCGCATATGATTG |
| | | AAAATAAAGGCGGCAGCATAGTAAATATCAGTTCTGTGGGTGGATCCATT |
| | | CCAGACATTAGTAGAATCGGTTATGGGGTATCTAAATCGGGGGTAAACAA |
| | | TATAACAAAACAGATAGCAATTCAATACGCTAAATATGGCATAAGATGTA |
| | | ATGCTGTGCTGCCAGGCCTGATCGCTACGGATGCAGCTATGAACTCCATG |
| | | CCTGATGAGTTTCGTAAAAGTTTCTTGAGTCATGTCCCGTTAAATCGCAT |
| | | TGGCAATCCTGAAGACATAGCCAATTCTGTGTTGTTTTTTGTACCATCAG |
| | | AAGATAGCTCGTATATCACCGGAAGTATACTGGAGGTGAGTGGTGGTTAT |
| | | AATTTGGGTACACCTCAATATGCTGAATTCGTGGGTTCAAAAGTGGTAGA |
| | | GTAA |
| 4 | Codon optimized | ATGAAACGTCTGGAAGGAAAAGTTGCCATCGTAACAAGTAGTACAAGAGG |
| | 7α-hydroxysteroid | TATTGGTCGTGCATCGGCTGAAGCATTAGCTAAGGAAGGTGCTTTGGTAT |
| | dehydrogenase gene | ATCTGGCAGCCCGTTCTGAAGAGTTGGCGAATGAAGTCATCGCCGATATA |
| | from Clostridium | AAAAAGCAAGGTGGCGTTGCCAAATTTGTGTATTTCAACGCAAGAGAGGA |
| | absonum | AGAAACGTATACTTCGATGGTAGAAAAGGTTGCTGAAGCAGAAGGAAGAA |
| | | TCGATATCCTTGTTAACAACTATGGTGGGACTAACGTGAACCTGGATAAG |
| | | AATTTGACAGCTGGTGATACGGACGAATTTTTTCGCATCCTGAAAGACAA |
| | | TGTACAGTCTGTTTATTTGCCAGCCAAAGCCGCTATACCGCATATGGAAA |
| | | AAGTGGGTGGAGGTTCAATCGTAAATATTTCGACTATTGGATCGGTGGTA |
| | | CCTGACATATCTCGCATCGCCTACTGTGTATCAAAGTCAGCAATTAATTC |
| | | CTTGACGCAAAATATTGCATTGCAGTATGCCAGAAAAAATATCCGTTGTA |
| | | ACGCCGTCTTGCCCGGTTTGATTGGAACCAGAGCAGCCCTTGAGAACATG |
| | | ACGGATGAATTCCGGGACAGCTTCTTGGGTCATGTTCCACTTAACCGTGT |
| | | AGGACGTCCTGAAGACATTGCGAATGCAGTATTATATTATGCAAGTGATG |
| | | ATTCCGGTTATGTGACGGGAATGATCCACGAAGTTGCAGGTGGATTTGCT |
| | | TTGGGAACACCACAGTATTCTGAATACTGTCCTCGTTAA |
| 5 | Codon optimized | ATGTCGTATGAATCGCCTTTCCACTTGAATGATGCGGTTGCCATAGTAAC |
| | 7α-hydroxysteroid | TGGTGCTGCAGCAGGTATTGGAAGAGCAATTGCCGGCACATTTGCAAAAG |
| | dehydrogenase gene | CCGGAGCATCAGTGGTCGTAACCGATTTAAAATCGGAAGGTGCTGAAGCT |
| | from Brucella | GTAGCTGCCGCAATTAGACAGGCTGGCGGAAAAGCCATTGGTCTGGAATG |
| | melitensis | CAATGTAACGGATGAACAGCACAGAGAAGCTGTCATTAAAGCAGCATTGG |
| | | ATCAGTTTGGGAAGATCACTGTCTTGGTAAATAATGCTGGTGGAGGAGGT |
| | | CCAAAGCCGTTCGATATGCCGATGTCTGATTTCGAATGGGCATTCAAGTT |
| | | GAACTTGTTTTCCCTTTTCCGTTTGAGTCAACTGGCGGCTCCACATATGC |
| | | AAAAAGCAGGCGGAGGTGCTATTCTGAACATAAGCTCAATGGCAGGTGAA |
| | | AACACGAATGTACGTATGGCAAGCTATGGTAGTTCAAAAGCAGCTGTAAA |
| | | CCACCTGACACGCAATATCGCATTTGATGTAGGTCCTATGGGTATCCGTG |
| | | TAAATGCAATCGCCCCTGGAGCCATAAAGACAGACGCTTTGGCTACCGTA |
| | | TTAACCCCCGAAATCGAACGTGCAATGTTGAAACATACGCCGCTTGGGAG |
| | | ATTGGGAGAAGCGCAGGATATTGCGAATGCTGCACTGTTTTTATGTTCCC |
| | | CTGCAGCGGCCTGGATTAGTGGTCAGGTATTGACAGTATCCGGTGGAGGG |
| | | GTCCAGGAATTAGATTAA |
| 6 | Codon optimized | ATGAAAAAACTGGAAGATAAGGTGGCAATAATTACCGCAGCAACTAAGGG |
| | 7α-hydroxysteroid | CATTGGACTGGCTTCGGCAGAAGTACTTGCAGAGAATGGCGCACTGGTAT |
| | dehydrogenase gene | ACATTGCAGCAAGATCTGAAGAGTTGGCAAAAGAAGTAATCTCGAATATT |
| | S1-a-1 from bear | GAATCCAATGGTGGTCGCGCGAAATTTGTGTACTTTAATGCTCGTGAACC |
| | fecal metagenomic | GCAGACATATACAACAATGGTGGAAACAGTTGCCCAAAATGAAGGACGGT |
| | sample | TGGATATCCTGGTGAACAATTACGGCGAAACAAACGTGAAGCTGGATCGT |
| | | GATTTAGTGAATGGCGACACGGAGGAATTTTTTCGTATAGTGCAGGATAA |
| | | CCTTCAGTCTGTATACCTGCCTTCAAAAGCCGCTATTCCGCGTATGGCGA |
| | | AAAATGGAGGAGGCTCCATTGTAAACATTTCAACAATTGGTTCCGTTGTC |
| | | CCGGATTTGGGACGTATTGCTTATTGTGTTTCGAAAGCCGCCATAAATTC |
| | | TTTAACACAGAATATTGCTCTGCAGTATGCTCGGCAAGGGGTGAGATGTA |
| | | ACGCGGTATTACCGGGATTGATAGGAACTAAAGCCGCGATGGAAAATATG |
| | | ACGGACGAATTCAGAGATTCCTTTTTGCGTCATGTCCCAATAAATCGCGT |
| | | GGGAAAACCTGAAGATATAGCCAAAGCCGTACTGTACTATGCATCGGATG |
| | | ATTCTGATTACGTAACAGGAATGATTCATGAAGTCGCAGGTGGATACGCT |
| | | CTTGGATCCCCTCAGTATGCTGAATTCAGTGCCATGATGGAACGTTCAAG |
| | | ATAA |
| 7 | Codon optimized 7β | ATGACTCTGCGCGAAAAGTATGGAGAGTGGGGAATTATACTGGGTGCAAC |
| | hydroxysteroid | GGAAGGTGTGGGAAAGGCATTTTGTGAACGTTTGGCTAAAGAAGGAATGA |
| | dehydrogenase gene | ACGTAGTTATGGTGGGAAGACGGGAAGAAAAACTGAAGGAATTAGGTGAA |
| | from Ruminococcus | GAACTTAAAAACACCTATGAGATAGACTATAAAGTGGTTAAAGCAGATTT |
| | gnavus ATCC | CTCTTTGCCAGACGCGACTGATAAAATATTTGCAGCCACAGAGAACTTGG |
| | 29149 | ACATGGGATTCATGGCATATGTAGCCTGCCTGCATTCATTTGGAAAGATC |
| | | CAAGACACACCTTGGGAAAAACATGAGGCTATGATAAACGTCAATGTGGT |
| | | AACCTTCATGAAGTGCTTCTATCATTATATGAAAATCTTTGCCGCCCAGG |
| | | ATCGTGGAGCAGTCATCAATGTAAGCAGTATGACTGGGATTAGTTCATCC |
| | | CCGTGGAATGGTCAATATGGAGCCGGAAAAGCCTTTATTCTGAAAATGAC |
| | | GGAAGCAGTGGCGTGCGAAACGGAAAAAACCAATGTCGATGTAGAAGTGA |
| | | TCACACTGGGAACAACACTGACACCGAGCTTACTGAGTAACTTGCCTGGT |
| | | GGTCCGCAAGGTGAAGCTGTAATGAAAACGGCTCAAACACCCGAGGAAGT |
| | | TGTAGATGAGGCATTCGAAAAGCTTGGAAAGGAACTTTCAGTAATCTCTG |
| | | GAGAGCGTAATAAAGCTTCAGTTCATGACTGGAAAGCGAATCACACCGAG |
| | | GATGATTATATCCGGTATATGGGTTCGTTTTACCAAGAATAA |
| 8 | Codon optimized | ATGAACCTGCGTGAAAAATACGGAGAATGGGGACTGATACTTGGTGCCAC |
| | 7β-hydroxysteroid | AGAAGGCGTAGGTAAGGCATTCTGTGAGAAAATTGCCGCAGGTGGTATGA |
| | dehydrogenase gene | ACGTTGTTATGGTCGGGCGCCGCGAAGAGAAGTTAAATGTACTTGCCGGG |
| | from Colinsella | GAAATCCGTGAAACTTATGGTGTGGAAACGAAAGTAGTAAGAGCTGATTT |
| | aerofaciens ATCC | TTCGCAACCTGGAGCCGCGGAAACAGTATTTGCAGCAACGGAGGGCCTTG |
| | 25986 | ACATGGGATTCATGAGTTATGTTGCATGCCTGCACTCCTTTGGAAAAATC |
| | | CAGGATACACCGTGGGAAAAGCATGAAGCAATGATCAACGTGAATGTGGT |
| | | AACCTTCTTGAAATGTTTCCATCACTACATGCGTATTTTCGCAGCCCAAG |
| | | ATCGTGGAGCCGTAATTAATGTGAGCAGCATGACCGGCATTTCAAGCAGT |
| | | CCTTGGAATGGACAGTATGGTGCAGGGAAAGCGTTTATCCTGAAAATGAC |
| | | TGAGGCAGTAGCATGCGAATGCGAAGGAACCGGAGTTGATGTGGAAGTAA |
| | | TTACATTGGGAACAACACTGACACCGTCACTTCTGAGCAATTTGCCCGGT |
| | | GGTCCGCAGGGTGAAGCTGTAATGAAAATCGCGTTGACACCCGAAGAATG |
| | | TGTGGATGAAGCGTTCGAAAAACTGGGGAAAGAACTGTCTGTAATCGCCG |
| | | GACAGCGTAATAAAGACTCTGTGCATGATTGGAAAGCTAATCACACCGAG |
| | | GATGAATATATCCGGTATATGGGGAGCTTCTATCGGGACTAA |
| 9 | Codon optimized | ATGAACTTCCGTGAAAAGTATGGGCAGTGGGGTATTGTATTAGGTGCTAC |
| | 7β-hydroxysteroid | AGAAGGGATTGGTAAGGCAAGTGCATTTGAACTTGCGAAGCGTGGAATGG |
| | dehydrogenase gene | ATGTGATTTTGGTTGGACGTCGTAAAGAGGCTTTGGAAGAATTGGCCAAG |
| | from Clostridium | GCAATCCACGAAGAAACGGGCAAAGAAATCCGTGTGTTGCCGCAGGATCT |
| | absonum | TTCAGAGTATGATGCCGCTGAACGTTTAATCGAAGCTACCAAAGACCTGG |
| | | ACATGGGCGTCATTGAATATGTTGCATGCCTTCATGCAATGGGTCAGTAT |
| | | AACAAGGTTGACTATGCCAAGTACGAACAAATGTACAGAGTGAACATTCG |
| | | GACGTTCAGCAAGCTGCTTCACCATTATATTGGGGAATTCAAGGAAAGAG |
| | | ATAGAGGTGCATTTATTACGATTGGATCCCTTTCTGGCTGGACTTCGCTT |
| | | CCTTTTTGTGCTGAATATGCAGCAGAAAAAGCCTACATGATGACCGTAAC |
| | | TGAGGGTGTCGCTTATGAATGCGCAAATACTAATGTGGACGTAATGTTGT |
| | | TGTCGGCAGGATCCACAATCACCCCAACATGGCTGAAAAATAAACCCTCT |
| | | GACCCTAAAGCAGTTGCGGCTGCCATGTATCCTGAAGATGTCATAAAAGA |
| | | TGGGTTTGAACAACTGGGGAAAAAGTTTACGTATCTTGCCGGAGAATTAA |
| | | ATCGCGAGAAAATGAAAGAAAATAACGCTATGGATCGGAATGATCTGATC |
| | | GCCAAACTGGGCAAAATGTTTGATCATATGGCATAA |
| 10 | Codon optimized | ATGAATCTTCGGGAAAAATACGGAGAGTGGGGAATAATCCTGGGTGCTAC |
| | 7β-hydroxysteroid | AGAAGGTGTGGGAAAAGCATTCGCCGAAAAAATCGCATCTGAAGGAATGA |
| | dehydrogenase gene | GCGTGGTTCTGGTTGGAAGACGTGAGGAGAAACTGCAAGAACTGGGCAAG |
| | from Ruminococcus | TCTATTTCCGAAACATACGGCGTTGACCATATGGTAATCCGTGCTGATTT |
| | torques L2-14 | TGCACAGAGTGATTGCACCGATAAAATATTTGAAGCTACGAAGGACTTGG |
| | | ATATGGGGTTTATGTCGTATGTTGCATGTTTCCATACTTTCGGTAAACTT |
| | | CAAGACACCCCTTGGGAAAAACATGAACAGATGATCAATGTTAATGTTAT |
| | | GACGTTCCTGAAATGCTTCTACCACTACATGGGCATTTTTGCAAAGCAAG |
| | | ATCGGGGAGCAGTAATAAATGTAAGTAGTCTGACAGCCATTTCTAGTAGT |
| | | CCTTATAATGCTCAATATGGTGCTGGTAAATCATATATTAAAAAATTGAC |
| | | AGAAGCTGTAGCAGCTGAATGCGAATCTACGAACGTGGATGTGGAAGTCA |
| | | TTACTTTGGGAACAACAATCACGCCGAGTTTGTTGTCCAATCTGCCGGGT |
| | | GGACCTGCAGGTGAAGCGGTAATGAAAACTGCCATGACGCCCGAAGCATG |
| | | TGTTGAAGAAGCTTTTGATAACCTGGGTAAAAGCTTGAGTGTAATCGCTG |
| | | GTGAACATAACAAAGCTAACGTGCACAATTGGCAAGCAAACAAGACGGAT |
| | | GACGAGTATATCCGTTATATGGGCTCATTCTATTCAAACAACTAA |
| 11 | Codon optimized | ATGAACATGAATCTTCGCGAGAAGTATGGTGAATGGGGTATTATACTGGG |
| | 7β-hydroxysteroid | GGCAACAGAAGGGGTTGGCAAAGCCTTCTGTGAAAAAATAGCTGCTGGTG |
| | dehydrogenase Y1- | GCATGAATGTTGTGATGGTTGGACGCCGTGAAGAAATGCTTAAAGATCTG |
| | b-1gene from bear | GGGGGGGAGATCAGCAACAAATATGGGGTGGAACATCTGGTAATCAAGGC |
| | fecal metagenomic | TGATTTTGCTGACCCATCGTCCGTAGATAAAATTTTTGAGCAGACCAAGG |
| | sample | AATTGGACATGGGTTTCATGTCATATGTGGCATGTTTTCATACCTTCGGA |
| | | AAGTTGCAAGATACGCCATGGGAGAAACATGAACAGATGATTAATGTGAA |
| | | CGTGATTACGTTCTTCAAATGCTTCTACCATTATATGGGAATTTTCGCTA |
| | | AACAAGATCGTGGGGCAATTATTAATGTATCTAGCTTGACAGGAATCTCT |
| | | TCATCTCCTTATAATGCACAATATGGCGCTGGCAAAAGTTACATCTTGAA |
| | | ATTAACAGAAGCTGTTGCGTGTGAAGCAGCCAAAACTAATGTGGACGTTG |
| | | AAGTGATAACACTGGGAACAACCATTACACCTAGCCTGTTGAAAAATTTG |
| | | CCTGGAGGACCGGCTGGAGAAGCCGTAATGAAAAGTGCATTAACACCTGA |
| | | AGCATGTGTTGATGAAGCGTTTGAAAATTTGGGTAAGACATTCAGTGTAA |
| | | TCGCTGGAGAACACAACAAAAAAAACGTTCATAACTGGAAGGCGAATCAT |
| | | ACAGCGGATGAATACATCACATACATGGGCAGCTTTTACGAGAAATAA |
| 12 | Codon optimized | ATGTTCATGATGCTTAAAAATAAAGTTGCCATAGTAACAGGAGGAACACG |
| | 3α-hydroxysteroid | TGGGATAGGCTTCGCAGTTGTGAAAAAATTCATAGAAAATGGAGCAGCCG |
| | dehydrogenase gene | TTTCTTTGTGGGGATCCAGACAGGAAACTGTTGACCAGGCATTAGAGCAA |
| | from Ruminococcus | CTGAAAGAATTGTATCCCGATGCAAAGATCAGTGGGAAATACCCTTCATT |
| | gnavus ATCC | AAAAGATACTGCGCAAGTGACAaCAATGATTAATCAAGTGAAAGAAGAGT |
| | 29149 | TTGGTGCAGTCGACATTTTGGTAAATAATGCAGGTATATCCCAATCCACA |
| | | TCATTCTATAATTATCAACCGGAAGAGTTTCAAAAAATTGTGGATTTGAA |
| | | TGTGACCGCTGTATTTAACTGTAGTCAAGCAGCCGCAAAAATAATGAAAG |
| | | AACAGGGAGGTGGTGTAATCTTGAACACCTCAAGCATGGTGAGCATTTAT |
| | | GGCCAACCGTCAGGATGTGGCTATCCTGCATCTAAATTCGCTGTGAATGG |
| | | ACTGACTAAGTCACTGGCCAGAGAATTGGGTTGTGACAATATAAGAGTTA |
| | | ATGCCGTGGCACCGGGCATAACAAGAACTGATATGGTTGCTGCCCTGCCC |
| | | GAAGCAGTAATAAAGCCCTTGATTGCAACCATTCCTCTTGGACGCGTGGG |
| | | CGAGCCTGAAGATATAGCAAACGCATTTTTGTTTTTGGCCTCTGATATGG |
| | | CATCTTATGTAACTGGAGAGATTCTTTCTGTAGATGGCGCCGCAAGAAGC |
| | | TAA |
| 13 | Codon optimized | ATGGGCATATATGTTATAACTGGAGCTACATCTGGGATCGGTGCTAAAAC |
| | 3α-hydroxysteroid | AGCCGAAATCCTTAGAGAACGTGGCCATGAGGTCGTAAATATTGATCTGA |
| | dehydrogenase gene | ATGGAGGAGATATTAATGCAAATCTTGCGACAAAGGAAGGAAGAGCTGGA |
| | from Eggerthella | GCAATTGCTGAATTGCATGAACGTTTCCCTGAGGGTATCGATGCTATGAT |
| | lenta DSM2243 | TTGTAATGCTGGGGTAAGCGGAGGAAAAGTGCCGATTTCGCTTATCATAT |
| | | CCCTGAACTACTTTGGAGCAACTGAAATGGCACGCGGCGTATTTGACCTG |
| | | CTGGAGAAAAAAGGGGGATCTTGTGTGGTAACAAGTTCGAATTCAATTGC |
| | | CCAAGGTGCCGCAAGAATGGATGTGGCTGGAATGTTGAATAACCACGCGG |
| | | ATGAAGACAGAATCCTGGAGCTGGTCAAAGATGTTGATCCAGCCATCGGG |
| | | CATGTATACTATGCCAGTACTAAATATGCCTTAGCTCGTTGGGTAAGACG |
| | | GATGTCGCCGGATTGGGGATCAAGAGGCGTCCGTCTGAATGCGATTGCTC |
| | | CTGGAAACGTGAGAACAGCTATGACCGCAAACATGCTGCCGGAACAACGT |
| | | GCTGCAATGGAAGCCATTCCTGTGCCCACACATTTCGGTGAAGAGCCGTT |
| | | GATGGATCCTGTGGAAATTGCTAATGCAATGGCGTTTATTGCTTCTCCTG |
| | | AAGCGTCCGGGATCAATGGAGTTGTGCTGTTTGTTGATGGTGGCACTGAC |
| | | GCACTGCTGAATAGCGAGAAAGTATACTAA |
| 14 | Codon optimized | ATGGGAAAGCTGGAAGGTAAAGTAGCAATAGTAACCGGCGGTACGCGTGG |
| | 3α-hydroxysteroid | AATCGGATTCGGAATTGTTGAAAAATTCTTGGCAGAAGGTGCAAAGGTCG |
| | dehydrogenase gene | CTTTATTTGGAAGTAGACAGGAGACAGTGGACGCGGCGCTTGAAAAGATT |
| | from Eggerthella sp. | AAACAGAATGATCCTGAGGCGCCAGTTATGGGATTGCATCCTGCTTTAAC |
| | CAG298 | TGACCCGGATGAAATAGCAGCCGCCTTTAAGTCCGTGGTAGATACTTTCG |
| | | GAAGTTTGGACATCTTAGCCAATAATGCTGGAACAGACTCAAGAACCAAA |
| | | CTTGTGGATTATACACTTGAGGAATTTCAGAAAGTAATGCGCCTTAATGT |
| | | GGAAGCTACATTCGTATGTAGTCAGGCAGCGGCTCGTATAATGATCGAGC |
| | | AAGGAACTGGCGGTGCCATAATCAACACATCCTCTATGGTTAGCATCTAT |
| | | GGACAACCTGCCGGATGTGCTTACCCCACGTCTAAATTTGCCGTCAATGG |
| | | ATTAACGAAAAGTCTTGCCCGTGAATTGGCTCCTCATAAAATTCGTGTGA |
| | | ACGCTGTTGCCCCAGGTGTTACGCATACAGATATGGTGGATGCCCTGCCT |
| | | CGTGAAGTCATCGAACCATTGATTAAAACCATCCCATTGGGACGCATGGG |
| | | AGAACCGGAAGACATTGCAAATGCTTTTGCATTCTTAGCTTCGGATGAAG |
| | | CCTCGTACATAAGTGGTGATGTGCTGTCTGTTGATGGGTTAAGCCGTAGC |
| | | TAA |
| 15 | Codon optimized | ATGGGAATATATGTTATTACTGGCGCATCCAGTGGAATTGGAGCAAAAAC |
| | 3α-hydroxysteroid | TGCCGAAATACTTCGGGAACGTGGCCATGAGGTGGTAAACATCGACCTTA |
| | dehydrogenase gene | AAGATGGAGACATCGAAGCGAATCTTGCAACCAAAGAAGGACGGGCTGGA |
| | from Gordonibacter | GCATTAGCGGAATTGCATGAACGGTATCCGGAAGGAATCGATGCCATGAT |
| | massiliensis | CTGCAACGCCGGCGTGTCTGGAGGTAAAGTGCCTATCTCCTTAATAATCT |
| | | CCCTGAATTATTTCGGTGCAACTGAAATGGCTCGTGGAGTCTTCGATCTG |
| | | TTGGAAAAAAAAGGTGGTAGTTGTGTTGTTACATCGAGTAATAGTATTGC |
| | | ACAAGGTGCAGCTCGGATGGACGTTGCCGGAATGTTAAATAACCAGGCCG |
| | | ATGAAGATCGCATTGTGGAACTGGTAAAGGATGTAGACCCGGCTGTGGGA |
| | | CATGTCTATTATGCCTCCACTAAATATGCCCTTGCCAGATGGGTAAGAAG |
| | | AATGAGCCCTGATTGGGGTAGCCGTGGAGTGCGGTTAAATGCAATCGCGC |
| | | CTGGTAATGTCCGCACCGCGATGACAAGCAATATGCTGCCGGAACAACGG |
| | | GCCGCGATGGAAGCAATACCTGTACCGACCCATTTTGGAGAAGAACCGCT |
| | | TATGGACCCGGAAGAAATTGCCAATGCGATGGCATTCGTTGCTTCACCTG |
| | | AAGCATCGGGTCTTAATGGTGTGGTTCTGTTCGTTGACGGTGGAACAGAC |
| | | GCCCTGTTAAACAGCGAAAAGGTGTATTAATAA |
| 16 | Codon optimized | ATGGGGATTTATGTGATTACGGGAGCCACATCTGGTATTGGCGCTAAAAC |
| | 3α-hydroxysteroid | TGCCTCGATTCTTAAAGAACAAGGGCATGAAGTTGTCAATATTGACTTAA |
| | dehydrogenase gene | AGGGAGGCGACATCAATGCTAATTTAGCAAGCAAGGAAGGAAGAGCTGCC |
| | from Raoultibacter | GCCATTGACGAATTACATAAACGCTACCCGGATGGGATCGATGCGATGAT |
| | timonensis | TTGTAATGCCGGTGTTAGTGCCGCGAATGGATCTATTCCTCTGATTATAT |
| | | CGCTTAATTATTTCGGTGCGACAGAAATGGCAATAGGTGTACGCGACTTG |
| | | TTGGAAAAGCGTGGGGGTAACTGTGTCGTAATATCATCCAATACCATTGC |
| | | TCAAGGAGCAGCGCGTATGGACGTGGTCGGTATGTTGAATAATCAAGCAG |
| | | ATGAAGACCGTATATTGGATTTGGTAAAGGATTACGACCCTGCGACAGCA |
| | | CATGCTTTTTATGCAGCCACTAAATATGCACTTGCGCGCTGGGCACGTAG |
| | | AATGTCTGCAGATTGGGGTGCACGTGGAGTGCGCGTGAATGCTGTGGCAC |
| | | CTGGAAATGTCCGGACCGCAATGACAGACCAGCTGACAGACGAAATGCTT |
| | | GTAGCTGTGCGTGCTCTTCCAGTTCCTACAAATTATGGAGGTGACCCTTT |
| | | GATGGACCCGACCGAAATCGCTAATGCAATTGCTTTTTTAAGCTCCCCTG |
| | | AAGCGCGTGGTATTAATGGAGTTGTCTTGTTTGTAGATGGAGGTACCGAC |
| | | GCTCTGCTGCACAGTGAAAAAGTTTATTAATAA |
| 17 | Codon optimized | ATGGGTGTGTATGCAATTACAGGAGCTTCTTCCGGAATTGGAGCCAAGAC |
| | 3α-hydroxysteroid | TAAAGAATTGCTGGAACTGGAAGGACATAAAGTAATCAACATCGATTTGA |
| | dehydrogenase gene | AAGGAGGTGACATTTGTGTGAATTTAGCTTCGGTGGAAGGCCGTGAAGAA |
| | from | GCAATCGCTAAACTGCATGAGATGTGTCCTGATGGATTGGACGGCATGAT |
| | Lachnospiraceae sp. | CTGTAATGCTGGAGTAAGTGGTGCTTGTGGCAACCTGGAACTTATAATCA |
| | | GCTTGAACTATTTCGGAACTATAGCAGTAGCGAAAGGGGTGTACGACTTA |
| | | CTGGAAAAGAAGCATGGATCATGTGTGGTAACCGCATCCAACACCATAAG |
| | | CCAAGGAGCTGGCCGTATGGATATTGCGGATTTGCTGAATAACATTGGTG |
| | | ACGAAAAACGGGTGTTGGAACTGGTAAGTAGCCTGGATTCTTCAAACTTA |
| | | TCGGTGGGCAATTCTATGTATGTAAGCACTAAATATGCCCTGGCAAGATG |
| | | GGTAAGAAGAGTATCCGCAACGTGGGCAGCCAATGGTGTCAGAATCAACG |
| | | CGATTGCACCTGGAAATGTAAACACAGCTATGACCGCTACTATGTCAACC |
| | | TCTGCAAAAATGGCACTTAATGCCCTTCCCATCCCAACTAAATTCGGACT |
| | | GGAAACTTTGATGGACCCAGAAGAGATTGCAGAAGTAATGATCTTCTTGG |
| | | TGTCGAAAAAAGCCTCTGGCATCAATGGAAATATCATGTTTGTCGACGGT |
| | | GGAACAGATGCTCTTCTTAACTCGGAGAAGGTATATTAATAA |
| 18 | Codon optimized | ATGCCTGTAACTGCTGTAACAGGTTCTGCGTCTGGAATCGGTGCAGCTGT |
| | 3α-hydroxysteroid | ATGTGATGTATTAAGAGCAGCAGGACACCAGATAATTGGTATCGACCGTG |
| | dehydrogenase | TGAATGCTGAGGTGATTGCAGATCTTTCCACGCCGGAAGGGAGACAAGCT |
| | HsdA 7 | GCTGTTGAAGAAGTTCTTGAAAGATGTGACGGAGTATTAGATGGGTTAGT |
| | gene from compost | ATGTTGTGCTGGAGTGGGTGTCACTGTACCGTCTTCCGGACTGATCGTAG |
| | metagenome | CTGTAAACCATTTTGGCGTTACTGCTTTGGTGGAAGGGCTGGCTTCGGCG |
| | | CTGGAACGTGGTGAACGCGGAGCAGCCCTGATCGTGGGATCGGTCGCCGC |
| | | GACTCATGCGGACGATTCTCAGCCCATGGTCGAAGCTATGCTTGCTGGTG |
| | | ATGAAGCACGGGCTATTGCACTGGCTAATGAATTAGATCAGGCACATATC |
| | | GCATATGCATCTTCGAAATATGCGGTAACCCGTTACGCCCGTCAACAAGC |
| | | TGTTGCATGGGGAGGAAGAGGATTACGTCTGAATGTGGTGGCCCCTGGTG |
| | | CCGTGGAAACCCCGTTACATCAGGCTTCCCTGGAGGATCCGCGCTTTGGA |
| | | CAAGCAGTACGTGATTTTGTTGCTCCGTTGGGACGGGCTGGACAACCGGC |
| | | CGAAATAGCAGCCCTTGTTGCATTCTTACAATCTCCGCAAGCTTCGTTTA |
| | | TCCATGGCTCTGTAATGTTCGTAGACGGTGGGATGGATGCAATGGTTCGC |
| | | CCTACAAAATTCTAATAA |
| 19 | Codon optimized | ATGGGAATCTATGTGATAACTGGAGCAACCTCCGGAATCGGTGCCAAAAC |
| | 3α-hydroxysteroid | AGCTGAAATTTTGCGTGGACGTGGCCATGAAGTAGTAAATATTGATCTGA |
| | dehydrogenase gene | ATGGGGGGACATCAATGCCAACCTTGCAACTAAAGATGGAAGAGCTCATG |
| | from | CTATTGCTGAATTGCATGAAAGATATCCGGAAGGAATTGATGCGATGATC |
| | Paraeggerthella | TGTAATGCTGGTATTAGCGGAGGAAAAGCTCCGATATCTCTGATCGTGTC |
| | hongkongensis | GTTAAATTATTTCGGGGCTACAGAGATGGCACGTGGCGTATTCGATCTTC |
| | | TTGAAAAGCGTGGTGGATCCTGCACTGTGACATCATCCAATTCAATTGCG |
| | | CAAGGGGCTGCTCGTATGGACGTTGCTGGTATGTTGAATAACCATGCAGA |
| | | CGAGGATCGCATTCTGGAACTGGTAAAAGATGTCGATCCGGCCATCGGAC |
| | | ACGTATATTACGCATCAACCAAATATGCTTTGGCAAGATGGGTGCGGAGA |
| | | ATGTCCCCTGAATGGGGAAGTCGTGGTGTACGGTTGAATGCCGTTGCGCC |
| | | AGGAAATGTACGTACAGCGATGACCGACAATATGCTTCCGGAACAACGTG |
| | | CAGCAATGGAAGCGATTCCCGTTCCAACACATTTTGGAGAAGAACCGCTT |
| | | ATGGAACCCATCGAAATCGCAAACGCTATGGCCTTCATTGCATCACCAGA |
| | | GGCCTGTGGAATTAATGGTGTCGTCCTTTTTGTGGATGGAGGTACCGATG |
| | | CTCTGCTGAACTCTGAAAAAGTCTATTAATAA |
| 20 | Codon optimized | ATGGGCATTTATGTGATCACTGGTGCCTCTTCTGGAATTGGTGCAAAAAC |
| | 3α-hydroxysteroid | TGCGGAAATATTAAGAGAGCGTGGGCACGAAGTGGTGAACATCGACCTTA |
| | dehydrogenase gene | ATGGAGGTGATATTAATGCGAATTTGGCAACAAAAGAAGGCCGTGCCGGC |
| | from Eggerthella | GCAATCGCGGAATTGCATGAGAGATACCCGGAAGGCATAGATGCAATGAT |
| | sinensis | TTGTAACGCCGGTGTAAGTGGAGGAAAAGTCCCGATCTCTCTGATAATTT |
| | | CCTTGAATTACTTTGGTGCTACTGAAATGGCCCGGGGTGTTTTCGACCTT |
| | | TTGGAAAAAAAAGGTGGTAGCTGTGTGGTAACTTCAAGTAATTCGATTGC |
| | | GCAAGGTGCCGCACGCATGGATGTAGCAGGCATGTTAAACAATCATGCAG |
| | | ATGAAGATCGCATCCTGGAACTTGTAAAGGATGTGGACCCAGCCATTGGT |
| | | CACGTTTATTATGCCTCCACTAAATATGCATTGGCTCGTTGGGTCAGACG |
| | | TATGTCACCAGATTGGGCTAGCCGTGGCGTAAGACTGAATGCGGTAGCAC |
| | | CTGGTAATGTCCGTACAGCAATGACAGCAAACATGCTTCCGGAACAGCGT |
| | | GCAGCGATGGAAGCCATTCCAGTACCTACGCATTTTGGTGAAGAACCGTT |
| | | AATGGATCCGGTTGAAATCGCTAACGTAATGGCATTTGTCGCAAGCCCGG |
| | | AAGCAAGTGGTATTAACGGAGTGGTTTTATTTGTGGATGGTGGAACTGAT |
| | | GCGCTTTTGAATAGCGAAAAAGTTTATTAATAA |
| 21 | Codon optimized | ATGGGTATCTATGTGATTACAGGAGCTAGTTCCGGAATTGGCGCCAAGAC |
| | 3α-hydroxysteroid | GGCAGAAATTCTGCGTGAACGTGGTCACGAAGTGGTCAACATTGACCTGA |
| | dehydrogenase gene | ATGGCGGAGACATCAATGCAAACCTGGCCACGAAAGAAGGAAGAGCGTCT |
| | from Eggerthella | GCTTTGGCGGAACTGCATGAACGTTTCCCGGAAGGAATAGATGCGATGAT |
| | guodeyinii | CTGCAACGCCGGTGTATCTGGTGGTAAAGTGCCGATATCACTGATCATAT |
| | | CCCTGAATTATTTTGGTGCTACAGAAATGGCCCGTGGAGTCTTCGATCTG |
| | | CTTGAAAAAAAAGGAGGTTCGTGCGTAGTAACATCTTCTAATAGTATTGC |
| | | ACAAGGAGCCGCTCGGATGGATGTTGCAGGAATGTTAAATAACCATGCGG |
| | | ATGAAGATCGTATACTGGAACTGGTAAAGGACGTTGATCCAGCTATTGGC |
| | | CATGTATACTACGCATCAACAAAATATGCTCTGGCACGTTGGGTAAGACG |
| | | GATGTCTCCGGACTGGGGAAGTCGTGGTGTCAGACTGAATGCAATAGCAC |
| | | CTGGAAATGTACGTACAGCCATGACATCAAACATGTTGCCCGAACAACGT |
| | | GCAGCAATGGAAGCAATTCCTGTTCCTACACACTTTGGAGAAGAGCCCTT |
| | | AATGGATCCGATCGAAATTGCAAATTCGATGGCATTTATTGCATCTCCGG |
| | | AAGCCAGTGGAATTAACGGCGTGGTTCTTTTTGTAGATGGTGGAACCGAC |
| | | GCTTTATTGAACTCAGAAAAGGTATACTAATAA |
| 22 | Codon optimized | ATGGGTATTTATGTCATCACTGGAGCCTCATCAGGAATCGGTGCCAAAAC |
| | 3α-hydroxysteroid | GGCCGAAATATTGCGTGAACGTGGACATGAGGTGGTTAATATTGACTTAA |
| | dehydrogenase gene | AAGATGGCGATATCGAGGCAAATTTGGCAACCAAAGAAGGGCGTGCTGGT |
| | from Gordonibacter | GCAATAGCGGAATTGCATGAGAGATATCCGGAAGGTATCGATGCAATGAT |
| | pamelaeae | ATGTAATGCTGGCGTTTCTGGGGGAAAGGTTCCGATTAGTTTGATAATAT |
| | | CTTTGAACTATTTCGGTGCTACAGAAATGGCGAAAGGAGCTCGTGACCTG |
| | | CTGGAAAAAAAGGGTGGAAGTTGCGTAATAACATCATCTAATTCGATCGC |
| | | ACAAGGAGCAGCCCGCATGGACGTCGCAGGAATGTTGAATAACCAAGCTG |
| | | ACGAGGATCGGATTTTGGAACTGGTGAAGGATGTTGATCCGGCTGTCGGA |
| | | CACGTGTATTACGCATCGACAAAATATGCCCTTGCAAGATGGGTCCGCCG |
| | | CATGTCTCCAGATTGGGGTTCCCGTGGTGTAAGACTGAATGCCATCGCAC |
| | | CGGGAAATGTTCGTACGGCAATGACAGCGAATATGTTACCAGAACAGCGG |
| | | GCTGCTATGGAAGCCATTCCTGTACCGACACACTTTGGAGAAGAACCATT |
| | | GATGGAACCGATTGAAATTGCTAATGCAATGGCTTTTATCGCATCACCAG |
| | | AGGCTTCTGGTATCAATGGTGTTGTTCTGTTCGTGGATGGAGGGACAGAC |
| | | GCATTGTTAAATAGCGAAAAAGTTTATTAATAA |
| 23 | Codon optimized | ATGGGAATTTATGTAATCACGGGAGCCAGCTCTGGAATAGGAGCGAAAAC |
| | 3α-hydroxysteroid | GGCATCGATATTGAAAGAACACGGTCATGAGGTGGTGAATATTGACTTAA |
| | dehydrogenase gene | AAGGGGGAGATATAGATGCCAACCTGGCATCTAAAGAAGGACGTGCTGCC |
| | from Raoultibacter | GCGATTGCTGAACTGCATGAAAGATACCCGGAAGGAATCGATGCAATTAT |
| | massiliensis | CTGTAATGCTGGAGTGTCCGCTGCCAATGGCAGTATCCCTCTGATAATCT |
| | | CATTGAACTACTTCGGTGCAACAGAAATGGCTATTGGAGTACGTGATCTG |
| | | CTGGAAAAAAAAGGAGGAAATTGCGTCGTAATCTCAAGTAATACAATTGC |
| | | CCAAGGAGCCGCTCGTATGGACGTTGTCGGAATGCTGAATAATCAGGCAG |
| | | ATGAAGATCGTATTTTGGAGCTGGTTAAGAACTATGATCCTGCAAGTGCC |
| | | CATGCGTTCTATGCCGCGACTAAATATGCTTTGGCGAGATGGGCCAGACG |
| | | CATGTCTGCCGATTGGGGTGCCAGAGGTGTTCGTGTAAATGCAGTGGCAC |
| | | CCGGTAATGTACGGACAGCAATGACGGACCAACTGACGGATGAAATGCTG |
| | | GTTGCTGTAAGAGCTTTGCCGGTTCCGACTAATTATGGTGGGGATCCCCT |
| | | GATGGATCCCGCCGAGATAGCTAATGCCATTGCCTTTTTATCTTCACCCG |
| | | AAGCTCGTGGAGTCAATGGCGTAGTGTTGTTCGTGGATGGAGGAACGGAT |
| | | GCCTTGCTGCATTCAGAAAAGGTCTACTAATAA |
| 24 | Codon optimized | ATGAATTTCGGCGGATTCATTATGGGGCGTTTTGACGAAAAAATCATGCT |
| | 3β-hydroxysteroid | GGTGACCGGAGCCACAAGCGGTATAGGACGCGCTGTGGCTATACGTGCAG |
| | dehydrogenase gene | CCAAGGAAGGTGCCACAGTAGTGGCAGTTGGCCGCAACGAAGAACGTGGA |
| | from Ruminococcus | GCTGCAGTAGTAGCTGCGATGGAGGAGGCTGGGGGTAAAGGTGAATTTAT |
| | gnavus ATCC | GAAATGTGACGTTTCCAACAAAGATGCTGTAAAAGCGTTGTTCGCGGAAA |
| | 29149 | TCCAGGAAAAGTACGGTAAACTTGATGTCGCTGTGAATAATGCCGGAATT |
| | | GTAGGCGCAAGTAAGACCGTGGAGGAATTGGAGGATGATGACTGGTTCCA |
| | | AGTTATTGATGCGAACCTGAATTCCTGTTTTTTCTGTTGTAGAGAAGAAG |
| | | TAAAGCTTATGCAGCCCTCTGGTGGTGCAATTGTAAATGTCAGCAGTGTA |
| | | GCTGGTATGCGGGGTTTCCCGTCTGCCGCTGCTTATGTTGCTAGTAAACA |
| | | CGCAGTATCTGGCTTGACAAAAGCCGTCGCTGTTGACTACGCCACAAAAG |
| | | GGATCACCTGTAATGCTATTTGTCCTGCTGGAACTGATACGCCGCTGACT |
| | | GAACGTTCCTCAGCAGACATCAAAACACGTATGGCTGAGATTGCAGCCCA |
| | | GGGTAAAGATCCCATGGAGTGGTTGAAGAACTCTATGCTTTCCGGAAAGA |
| | | CTGAAACACTGCAAAAAAAAAATGCAACACCCGAGGAGCAAGCGGCAACA |
| | | ATACTGTATTTTGCATCAGATGAAGCCCGTCATATCACAGGAAGCATAGT |
| | | AGCATCTGATGGAGGTTTCACGACCTATTAA |
| 25 | Codon optimized | ATGTATGACGACTTGAAAGGTAAAACCGTAGTGGTGACCGGATCATCCAA |
| | 3β-hydroxysteroid | AGGACTGGGTGCTGCTATGGCTCGTCGGTTTGGAGCTGAAGGGATGAACG |
| | dehydrogenase gene | TGGTTGCGAATTATCGTTCGGATGAGGAAGGTGCAAGAGAAACCGTCAGA |
| | from Eggerthella | GCAATAGAAGAGGCTGGAGGTGCTGCTGCTGCAGTACAGGCGGACGTGTC |
| | lenta DSM2243 | AAAGAACGAATGTGTTGATGCACTTTTTGATGCCGCAATGTTCTCGTTTG |
| | | GAGGTGTGGACATATGGGTGAATAATGCCGGTATTGAGGTGGCTTCTCCA |
| | | AGCGACCGTAAATCGATAGAAGAATGGCAACGTGTGATCGATGTGAACCT |
| | | GACAGGAGTATTTGCCGGTTGCCGCCGCGCAATAGACCACTTTTTAGATC |
| | | GTAAAATGCCCGGCGTAATAATCAATCTGTCTAGTGTGCATGAAATCATC |
| | | CCGTGGCCGCATTTTGCTGATTATGCCGCGTCAAAAGCCGGTGTAGGTAT |
| | | GTTAACCAAAACGCTTGCTTTGGAGTATGCAGATCGTGGTATACGTGTAA |
| | | ATGCAATTGCACCAGGAGCCATGAATACCCCAATCAATGCAGAAAAATTC |
| | | GCCGACCCGGAAGCCCGTGCCGCAACAGAAAGATTGATCCCTATGGGATA |
| | | CGTTGGAGCGCCTGAAGATGTTGCTGCTGCAGCTGCGTGGCTTGCATCGG |
| | | ATCAGGCCAGTTATGTAACTGGAACAACCCTTTTCGTAGACGGAGGAATG |
| | | ACTCTGTATCCAGGATTTCAATTTGGACAGGGATAA |
| 26 | Codon optimized | ATGAGCGAAGCACGTCACAATCCTGTTTTAGCTGGACAAACTGCAGTGAT |
| | 3β-hydroxysteroid | AACTGGAGGTGCCTCCGGAATCGGCAAAAGCATCGTACAAAGATTCTTGG |
| | dehydrogenase gene | AGGCTGGTGCTTCGTGTCTGGCAGCCGATTTGAATGAGGAAGCTCTGGCG |
| | from Eggerthella | GCATTGAAACAGGAACTGGCGGAATATGGCGATAAGTTAGACGTGGTCAA |
| | lenta DSM2243 | AGTGGATGTATCAAATCGTGATGATGTCGAAGGCATGGTAGACCGTGCAG |
| | | TTCAGACCTTTGGGCAAATGGACATCATAGTGAACAATGCAGGCATCATG |
| | | GACAACCTGTTACCTATCGCCGAAATGGATGATGACGTGTGGGAAAGATT |
| | | AATGAAAGTGAATCTGAATAGCGTAATGTATGGAACCCGTAAAGCAGTAC |
| | | GTTACTTTATGGAACGTGGAGAAGGCGGCGTGATAATAAATACAGCCTCT |
| | | CTTTCTGGTCTGTGTGCAGGAAGAGGTGGATGCGCCTATACAGCATCTAA |
| | | ATTTGCAGTAGTGGGACTGACTAAAAATGTCGCATTTATGTATGCAGACA |
| | | CTGGAATCCGTTGCAATGCCATATGCCCTGGAAACACCCAAACTAACATT |
| | | GGGGTGGGTATGCGTCAGCCTTCTGAAAGAGGAATGGCTAAAGCGACGAC |
| | | GGGATATGCTGGTGCAACAAGATCGGGAACGCCTGAGGAAATTAGTGCTG |
| | | CGGCAGCCTTCCTTGCCAGTGATCAAGCAGGTTTCATTAATGGCGAAACA |
| | | TTAACTATTGATGGGGGTTGGTCAGCTTATTAA |
| 27 | Codon optimized | ATGCAGGATGTATTCACCTTAAAAAACGGAGTAACCATGCCCAGAATCGG |
| | 3β-hydroxysteroid | ATTTGGAACTTACAATACCAGTGACGATGAAGCATGTCGTGTAGTCTGTG |
| | dehydrogenase gene | ATGCGGTGGAGGTGGGATATAGATTGATCGATACGGCTGCAATTTACGAG |
| | from Eggerthella sp. | AACGAAGCAGGTATTGGCCGTGCTTTGGCCACTTGTGGAGTTCCGCGTGA |
| | CAG298 | AGAACTGTTCATCACTAGTAAAGTATGGAATACTCACCGTGGCTACGACA |
| | | AAACGATGGAATCGTTTAATGCGAGCTGCGAACGTTTAGGTGTGGATTAT |
| | | CTGGACCTTTTTCTTATACATTGGCCGGCCAATGAAAAACAGTTTGGAGC |
| | | TGAAGCCGAGGCAATTAACGCTGACACATGGCGCGCATTAGAGGATCTGT |
| | | ACAAAAATGGCGCCGTCCGTGCTATTGGCTTGTCAAATTTCAAACCGCAT |
| | | CATATAGAAGCTTTGCTGAAACATGCCGAAATCGAGCCGATGGTGGATCA |
| | | AATCGAATTTTATCCTGGACGTATACAAGCTGAAACTCTGGAATATTGCC |
| | | TGGAACGGGATTTGGTAGTAGAGGCATGGTCACCGCTGGGTAGAGGTAAA |
| | | ACTTTGACTAATGAAGCTATCGCAGAAATAGGTGCACGGTATGGGAAGTC |
| | | CAATGCACAAGTATGCTTACGCTGGCTGATCCAGCTGGGAATGTTGCCAC |
| | | TTCCTAAGTCGGGAAACATTGAGCGCATGAAACAAAACTTGGAAGTTTTC |
| | | GATTTTGAACTGACACCCGAGGAGATGGCTGTAATATCTGCACAGGAGAA |
| | | TCCGACTGGACGGTTTTGGGACGCGGATGAAATCGACTTTTAA |
| 28 | Codon optimized | ATGAATTTTGGCGGTTTTATCATGGGCCGCTTCGATGAAAAAATTATGTT |
| | 3β-hydroxysteroid | GGTTACTGGAGCTACATCTGGAATAGGACGCGCAGTGGCGATTCGTGCTG |
| | dehydrogenase gene | CCAAAGAAGGTGCAACCGTGATCGCTGTAGGACGGAATGAAGAACGTGGT |
| | from Ruminococcus | GCAGCTGTAGTAGCAGCAATGGAGGAAGCGGGTGGCAAAGGTGAATTCAT |
| | gnavus | GAAATGCGACGTGTCGAATAAAGATGCGGTGAAAGCCCTTTTTGCCGAAA |
| | | TACAGGGCAAGTATGGTAAACTGGATGTAGCAGTAAACAATGCTGGAATC |
| | | GTTGGCGCCTCCAAAACAGTCGAGGAACTGGAGGATGATGATTGGTTCCA |
| | | GGTAATTGACGCAAACTTGAACTCCTGTTTTTTTTGCTGCCGTGAAGAAG |
| | | TAAAACTTATGCAGCCGTCCGGAGGAGCAATCGTTAATGTGTCATCAGTT |
| | | GCAGGAATGCGTGGTTTTCCGTCAGCGGCTGCGTATGTGGCCTCGAAGCA |
| | | TGCAGTTTCCGGATTGACCAAAGCCGTTGCAGTAGACTATGCCACCAAGG |
| | | GAATCACATGCAATGCTATTTGTCCTGCTGGAACTGATACGCCGCTTACG |
| | | GAAAGAAGTTCCGCTGATATAAAGACCCGTATGGCAGAAATTGCTGCACA |
| | | GGGAAAGGATCCGATGGAATGGCTGAAAAACTCCATGTTATCGGGGAAAA |
| | | CTGAAACACTGCAGAAAAAAAATGCCACACCGGAAGAACAAGCCGCGACC |
| | | ATCCTGTATTTTGCTTCTGATGAAGCACGCCACATAACTGGTAGCATTGT |
| | | GGCTTCAGATGGTGGTTTCACGACTTACTAATAA |
| 29 | Codon optimized | ATGGATTTTCTGGCGTTGCTGTGCTATAATACCATCAAAAGCAATAAAGA |
| | 3β-hydroxysteroid | AGTAATAAACCGTGGACGCTTCAGCGGTAAAATCATGTTGGTAACTGGTG |
| | dehydrogenase gene | CCACGAGTGGAATAGGCCGTGCAGTGGCTCTGCGTGGAGCGAAAGAAGGA |
| | from | GCTACCGTAATCGCGGTAGGCAGAAACGAAGAAAGAGGTAATGCTGTAGT |
| | Lachnospiraceae | GGAAGCCATTGAAAATAAGGAGGGAAAGGCAGTATTCAAAAAGTGCGATG |
| | bacterium | TATCGGATAAGGAGGCAGTTAAAAAACTGTTCGCGGAAATCAAGGAAGAA |
| | 2_1_46FAA | TTTGGCAAGTTAGATGTGGCAGTAAATAATGCTGGTATAGTGGGAGCATC |
| | | GAAAACTGTGGAAGAACTGGAGGATGATGATTGGTCGAAGGTTATTGATG |
| | | CAAACTTGAATTCATGTTTTTACTGCTGCAGAGAAGAAGTGAAACTGATG |
| | | AAAGAGAATGGAGGTGCAATTGTTAATGTTTCGTCGGTAGCGGGAATGCG |
| | | TGGATTTCCAAGTGCGGCAGCTTATGTCGCCAGCAAACATGCAGTTAGTG |
| | | GATTAACAAAAGCGGTAGCCGTAGACTATGCGACGAAAGGGATTACATGT |
| | | AACGCTGTATGTCCTGCTGGAACGGACACACCATTAACGGAACGTAGCTC |
| | | GGCTGATATAAAGACTCGGATGGCCGAAATTGCAGCACAAGGTAAGGACC |
| | | CTATGGAATGGCTGAAAAATTCTATGCTTTCAGGAAAAACAGAGACTTTG |
| | | CAGAAACGTAATGCCACTCCTGAAGAACAAGCTGCTACGATATTGTTTTT |
| | | TGCATCAGATGAGGCCAAACATATTACAGGATCGATAGTTGCTTCAGATG |
| | | GAGGATTCACCACCTACTAATAA |
| 30 | Codon optimized | ATGGACCGGTTCGAGAATAAGATAATGTTGGTGACAGGTGCAACCTCTGG |
| | 3β-hydroxysteroid | TATCGGAAAAGCTGTGGCTTTGCGTGCCGCATCTGAAGGTGCCACTGTAA |
| | dehydrogenase gene | TTGCAGTGGGAAGAAATGAAGAAAGAGGTCATGGTGTGGTTGAAGCGATT |
| | from Absiella sp. | ACTTCAGCAAACGGAAAAGCCGAATTCATGAAGTGCGATGTATCCGATAA |
| | AM29-15 | AGAACAGGTCAAAGAGCTTTTTGCAAAAATCAAGGAAAGTTATGGACGGT |
| | | TAGATGTAGCCATTAACAACGCTGGAATTGTCGGTGCAAGCAAAACGGTA |
| | | GAGGAACTGGAGGATGAAGATTGGTCGAACGTAATAGATGCCAATCTGAA |
| | | CAGCTGTTTTTACTGCTGCCGTGAAGAGGTAAAGCTGATGAAAGAAACAG |
| | | GGGGTGCTATTGTAAACGTATCCAGTGTAGCTGGAATGCGTGGATTCCCT |
| | | TCTGCAGCTGCCTATGTGGCATCCAAACATGGCGTATCTGGTTTGACTAA |
| | | AGCAGTTGCTGTTGACTATGCAACAAAGGGAATAACCTGTAATGCCGTAT |
| | | GTCCTGCCGGAACAAACACCCCTCTTACCGAAAGAAGTAGTGCAGACATT |
| | | CAGGAACATATGGCAGCTCTGGCTGCTCAGGGTAAAGATCCAATGGAATG |
| | | GCTTAAGAATTCCATGATGTCCGGAAAGACCGAAACTCTGCAAAAACGCA |
| | | ATGCCACACCGGAAGAACAGGCTGCAACCATATTGTATTTTGCCTCTGAT |
| | | GAAGCTAAGCACATCACTGGAAGCATCGTGGCTTCAGATGGGGGTTTCAC |
| | | AACATATTAATAA |
| 31 | Codon optimized | ATGTTCAAGGATCGTTTCAATGGCCAAACAATTATTGTTACTGGAGGTAC |
| | 3β-hydroxysteroid | GTCCGGTATCGGACGTGCTGTATGTATTCGTGCAGCACTGGAAGGAGCTA |
| | dehydrogenase gene | ACGTGGTAGTAAGTGGACGGAACAAAGAACGTGGCCAGGCAGTCGTTGAT |
| | from Clostridium | GAAATATTAAAGCAAGGTGGTGAGGCAATATTCGTACAGGGCGATATAAC |
| | cadaveris | TAAAAAAGAAGACGTCGTGCATCTGTATAAGGAGGCAGAGCAGAAATATG |
| | | GCGAAATTCATATCGCCATCAATAATGCTGGCATCGTCGGAGCATCAAAG |
| | | ATTCTGGATGAGGTAACGGACGAGGATTGGGGATCCGTAATTAATGCAAA |
| | | TCTTAACAGCATGTTTTATTGTTGCAGAGAAGCCGTTAAATACATGTTAA |
| | | AGCATGGAAAAGGTGGTGCCATTGTAAATACCAGTTCAGTAGCTGGCATG |
| | | CGTGGGTTTCCGTCTGCTGCAGCTTACGTGGCAAGCAAGCACGGCGTAAA |
| | | TGGCTTGACAAAAGCCGTGGCGGTAGATTATGCCACGAAAGGAATTCGTT |
| | | GCAACTCTGTAAATCCTGCCGGAACGGATACGCCTCTGACAGAAAATGCC |
| | | GCAGCTGGTATTAAGGCTAAAATTGCTGAACTGGTAAAACAGGGAATTGA |
| | | CCCACAGACTTTTCTGAAAGAAAGCATGACATCAGGAAAAACTCAGACTC |
| | | TTCAGAAGAGAAATGCATCACCGGAAGAACAAGCCAGTACCATTTTGTAT |
| | | TTCGCAAGTGATGAAGCAAAACATATCACTGGAAGCATCATAGCGAGTGA |
| | | TGGAGGATTCACTGTATATTAATAA |
| 32 | Codon optimized | ATGATTCAAGATCGTTTCGCTGGAAAAGTTATGGTAGTAACAGGCGGAAC |
| | 3β-hydroxysteroid | ATCTGGAATTGGTAAGGCAGTGTGCCTGCGTGCTGGAGCTGAAGGAGCAA |
| | dehydrogenase gene | AAGTGGTGATTGCTGGGCGTAATCAAGCACGTGGTCAAGCAATAGAAAAG |
| | from Holdemania | GAGATTCGCGAAGCAGGTGGAGAAGCTACATTTATCCAGTGTGATGTGAC |
| | filiformis | GCAGAAAGAGGACATCATAAATTTGTATGCAAAAACCATCGAGATTTACG |
| | | GTCAGTTGGACATCGCAATTAACAATGCCGGCATTGTTGGAGACTCTAAA |
| | | AAAATAGAAGATTTGACGGATGATGATTGGTTCTCTGTGGTTAACGCCAA |
| | | TCTGAACGCAATGTTTTACTGTATCCGGGAGGAAATTAAATACATGTTAA |
| | | AAAACGAGAATGGAGGAGCTATCGTAAACACAGCAAGTGTCGCTGGAATT |
| | | CGTGCCACGCCGGCCGGTCCTGCATACGTCGCATCGAAACATGGTGTGGT |
| | | AGGCCTGACAAAGTCCACAGCCATGGACTACGCGAAAAACAACATCATCT |
| | | GCAATGCAGTTTGTCCTGCTGGAACGGACACACCTTTGACAGAAGCAGCT |
| | | AAAGAAAAAATCTATGCGAAAATCGCCGAATTGAAAGCGCAAGGGATCGA |
| | | CCCTTCTGAATTTATGAAAAATTCCATGATCGCAGGAAAAACGCAGACCT |
| | | TACAGGGGAGAAATGCCACATCAGAGGAGCAAGCCTCGACAATTCTGTAT |
| | | TTTGCATCCGATGAGGCCCGCCATATCACCGGAAGCATAGTTGTTGCTGA |
| | | TGGAGGCTTTACCGTGTACTAATAA |
| 33 | Codon optimized | ATGCGTGATTATTTTGAGGGCAGATTCGAGGGAAAGAACATGTTAGTGAC |
| | 3β-hydroxysteroid | TGGTGGAACTTCCGGAATCGGCAAAGCAGTTTGTATAAGAGCCGCAAAGG |
| | dehydrogenase gene | AAGGAGCGTTTGTAATCATTGTTGGAAGAAATGAAGAACGGGCACAAGCT |
| | from Clostridium | GTTTTGTCCGAGATAGTGCAAAATGGTGGAAAGGCGCGCTTCATCAAAGC |
| | disporicum | GGATGTTAGCCGTGAAGATGAGGTAACATCGCTGTTTAACATAATCAACA |
| | | ATGAAGTTGGTGAATTGCACGTAGCCATAAATAACGCCGGAGTAGTTGGA |
| | | CATGGTGAACGTATCGATGAGCTTAGTACTGAAGAATGGAGCCGCGTGAT |
| | | CAATACAAACTTAAATTCCGCTTTTTATTGCTGTAGAGAGGAAGTGAAAA |
| | | ATATGTTAAATCATAAACAAGGTGGTTCGATCGTAAATGTATCCAGCGTA |
| | | GCCGGAACAACTGGGTTTTATCGCGCAAGTGCGTATGTAACGTCTAAACA |
| | | TGCACTGAATGGCTTAACAAAAGCTGTGGCAAATGATTTGGCAAAATTTA |
| | | ATATCCGGTGCAACTCTGTTTCCCCTGCTGTAACTGCTACGCCCCTTAAT |
| | | GATCGTAGTGCACAAGAGATAAAGGTTAAACTTGGAACAGCTATGGCGCA |
| | | GGGAAAATCACTTGAAGAAGCAAAATCAGAAACTATGATAGGAGGTAAAA |
| | | CCGAAACATTGCAGAAACGTAGCGCAACACCGGAAGAACAGGCAGCAACC |
| | | ATTTTGTACATTGCATCCGAGGAGGCTGCCCATATAACAGGCTCAATCAT |
| | | TATGTCAGACGGTGGATATACCGCTTACTAATAA |
| 34 | Codon optimized | ATGAAGGGATATTTTGAAGGAAGATTTGAGGGTAAGAATGTATTGGTGAC |
| | 3β-hydroxysteroid | GGGTGGTACTAGTGGAATTGGTAGAGCGGTATGTATTCGTGCAGGTAAAG |
| | dehydrogenase gene | AAGGTGCATATGTAATTGTGGTTGGTCGTAATGAAGCTCGTGGGCAGGCA |
| | from Clostridium | GTCGTTTCCGAGATCATTAACAATGGTGGAAACGCTATGTTTTTCCAAGC |
| | sp. CL-6 | AGACGTGAGCAAAGAAAATGATGTGATCAAGCTGTTCGAGGTTGTATCTG |
| | | ACAAGGTGGGAAAGATTCATGTAGTGATCAATAATGCGGGAATTGTTGGA |
| | | CACGGTGAACGCATCGATGAACTGAGCACAGACGAATGGTTAAATGTGGT |
| | | AAATACAAATCTGAATAGCGCATTTTATTGTTGCCGGGAAGCTGCGAAAA |
| | | ATATGATCAATCACAAAATCGGTGGAAGCATTGTGAATGTATCGTCCATC |
| | | GCTGGATCAACAGGTTTCTATCGCAGTTCCGCTTATGTGGCCAGTAAGCA |
| | | TGGCCTGAATGGTTTAACTAAAGCTGTGGCGAACGATCTGGCTATGTTTA |
| | | ACATTCGCTGTAACTCGGTATCCCCTGCTGGAACAGCTACACCTCTGAGT |
| | | GACAGAAGTTCCATGGAAGTGAAAACGAAATTGGGAGCAGCAATGGCAGC |
| | | TGGTAAAAGCCTGGAAGAAGCAAAATCGGAAACGATGATAGGAGGAAAGA |
| | | CGGAAACTCTTCAGAAAAGATCTGCTACATCTGAAGAACAGGCAGCCACC |
| | | ATTTTGTACGTTGCAAGTGATGAGGCCTCACACATCACGGGATCAATCAT |
| | | CATGTCCGATGGTGGTTATACCGCGTACTAATAA |
| 35 | Codon optimized | ATGCATATGAACCGGTTCGGAAATAAAGTTATGTTGATTACTGGAGCAAC |
| | 3β-hydroxysteroid | GTCTGGTATTGGAAAAGCTGTAGCGTTACGTGCTGCAATGGAAGGCGCTA |
| | dehydrogenase gene | CAGTGATAGCAGTTGGACGTAATGAAGAACGGGGAAACGCAGTTGTCGAT |
| | from | GAAATTGCAAAAGAAGAAGGTAAGGCTGTGTTCATGAAATGTGATGTAAG |
| | Erysipelotrichia sp. | CGATGTGGAACAGGTGAAACAGCTTTTCACAAATATCCAAGAGAAATATG |
| | | GGAAGATTGATGTCGCAATTAATAATGCCGGGGTAGTAGGTGCCTCAAAA |
| | | ACTGTTGAGGAATTGGCGGACGATGACTGGCTGAACGTAATTAATGCCAA |
| | | CCTGAATTCCTGTTTTTATTGCTGCCGTGAGGAAGTAAAATTGATGAAAG |
| | | AAAATGGTGGCGCGATCGTGAATGTTTCGTCTGTTGCTGGAATGCGTGGA |
| | | TTCCCTAGTGCTGCAGCCTATGTTGCGTCGAAACATGGCGTGTCTGGACT |
| | | GACAAAAGCGGTCGCAGTGGATTATGCCACGAAAGGCATCACATGTAATG |
| | | CAATTTGTCCAGCTGGAACTGATACACCCCTGAAAGAAAGATCGTCGGCG |
| | | GGAATCAAAGAACGTATGGCGGAATTAGCTGCGCAAGGCAAAGACCCCAT |
| | | GGAATGGCTGAAAAATTCCATGCTGAGCGGAAAAACAGAAACTCTTCAAA |
| | | AACGTAATGCGACACCGGAAGAACAGGCAGCAACTATCTTGTATTTTGCA |
| | | AGTGATGAAGCCCGTCACATTACCGGATCCATTGTTGCCTCCGATGGTGG |
| | | TTTTACCACATATTAATAA |
| 36 | Codon optimized | ATGATTCAAGACAGATTCGCGGGCAAGGTGATGGTAGTAACTGGTGGTAC |
| | 3β-hydroxysteroid | ATCAGGTATAGGCAAAGCTGTTTGCCTTCGTGCTGGCGCCGAAGGAGCAA |
| | dehydrogenase gene | AGGTAGTGATTGCAGGTCGCAATCAAGCACGTGGAGAAGCAATTGAGAAG |
| | from Holdemania | GAAATTCGTGAAGCAGGTGGAGAAGCGGTATTCATCCAATGTGATGTTAC |
| | sp | ACGCAAGGAAGATATCATAAATTTGTATGCCCGCACTGTCGAAATCTACG |
| | 1001302B_160321_ | GTCGTCTGGATATCGCAGTGAACAATGCCGGTATCGTGGGCGACTCCAAA |
| | E10 | AAAATCGAGGATTTGACTGACGATGACTGGTTTAGTGTCGTAAATGCAAA |
| | | CCTGAATGCCATGTTCTATTGTATTCGTGAAGAGATAAAATACATGCTGA |
| | | AGAATGAGAATGGTGGAGCAATCGTGAATACCGCATCTGTAGCTGGAATA |
| | | CGTGCAACACCTGCTGGTCCCGCTTATGTCGCATCAAAACATGGCGTGGT |
| | | GGGTCTGACTAAGAGTACTGCGATGGATTATGCCAAGAACAACATAATTT |
| | | GTAATGCAGTATGCCCTGCTGGAACGGACACTCCCCTGACAGAAGCAGCT |
| | | AAGGAAAAAATCTATGCCAAAATAGCGGAATTAAAGGCACAGGGAATTGA |
| | | TCCCTCGGAATTCATGAAAAATTCGATGATAGCTGGTAAAACGCAAACGC |
| | | TGCAGGGTAGAAATGCAACATCGGAAGAACAAGCCAGCACTATTTTGTAT |
| | | TTTGCTAGTGATGAAGCGAGACATATTACGGGAAGTATTGTCGTCGCTGA |
| | | TGGTGGATTCACAGTGTACTAATAA |
| 37 | Codon optimized | ATGTTCACAGAGCGCTTTAAAGACAAAGTGATGGTGGTAACGGGAGGAAC |
| | 3β-hydroxysteroid | ATCCGGAATAGGGAAAGCAGTATGTATCCGGGCTGGTGCCGAAGGTGCAA |
| | dehydrogenase gene | CCGTCGTTATCGCCGGTAGAAATGAAGAACGTGGAAAAGCGATTGAACAA |
| | from Clostridium | ACCATAACCGATAATGGCGGAAAGGCTCTGTTCGTACGTTGTGATGTAAC |
| | innocuum | CAAAAAAGAGGATATAATTGCTTTATACGCTAAGACAATGGAAGTCTACG |
| | | GACGTATCGATATCGCAATTAATAATGCAGGGATCGTGGGTGATAGTAAG |
| | | AAAATAGAGGACCTGACAGATGATGATTGGTTCAGTGTAGTAAATGCAAA |
| | | CCTGAACGCGATGTTTTATTGTATACGTGAAGAGGTCAAATATATGATGA |
| | | AAAATGAAAATGGAGGTAGCATTGTAAACACCGCGTCCGTGGCAGGAATT |
| | | CGTGCTACACCAGCTGGACCTGCTTATGTGGCCTCAAAACATGGCGTGGT |
| | | AGGACTGACAAAATCTACTGCAATGGACTACGCTGGGAAGAATATTACGT |
| | | GCAATGCCATTTGCCCAGCTGGGACGGATACACCTTTGACAGAAGCCGCT |
| | | AAGGAAAAGATCTATGCAATAATAGCTGATCTGAAAGCCCAGGGGAAAGA |
| | | TCCACAGGAATTCATGAAAAATTCTATGATAGCTGGTAAAACAGAAACTC |
| | | TGCAGCATCGTAATGCCACTTCTGAAGAGCAAGCAGCGACCATTCTGTAT |
| | | TTTGCCAGTGATGAAGCCAGACATATCACAGGTTCCATTGTTGCCTCAGA |
| | | CGGTGGATTTACAGTCTACTAATAA |
| 38 | Codon optimized | ATGTTTACACAACGCTTCAAAGATAAAGTTATGGTTGTTACCGGAGGGAC |
| | 3β-hydroxysteroid | CTCCGGAATTGGAAAAGCCGTATGCATTCGTGCTGGTGCTGAGGGAGCTG |
| | dehydrogenase gene | CAGTGGTTATTGCTGGAAGAAATGAAAATCGCGGAAAAGCGATTGAAAAA |
| | from | ACAATAACCGACAACGGTGGTACGGCCCTTTTCGTACGCTGTGACGTAAC |
| | Erysipelotrichaceae | CAAGAAAGAAGACATTCTTGCTCTGTACGCCAAAACAATGGAAGTGTACG |
| | sp. 66202529 | GAAGATTGGATATCGCGGTCAACAATGCCGGAATAGTGGGCGACTCAAAA |
| | | AAGATAGAGGACCTGACTGATGATGACTGGTTCAGTGTGGTAAACGCAAA |
| | | TTTGACGGCAATGTTTTACTGCATCCGTGAAGAAGTCAAATATATGATGC |
| | | AAAACGAAAATGGAGGATGTATCGTGAATACAGCATCTGTCGCTGGAATT |
| | | CGTGCTACACCTGCCGGTCCGGCGTATGTAGCGTCAAAACATGGAGTGGT |
| | | AGGATTGACAAAGTCTACCGCAATGGACTATGCTAATCGGAACATCACGT |
| | | GTAATGCAGTTTGCCCTGCTGGTACTGATACACCTTTGACTGAAGCCGCA |
| | | AAGGAAAAGATTTACGCAAAAATTGCAGAATTGAAAGCGCAGGGAAAGGA |
| | | TCCGCAAGAATTCATGAAAAATAGCATGATCGCAGGTAAAACTGAAACAT |
| | | TGCAGCACCGTAATGCTACAAGTGAAGAGCAAGCTGCAACTATTCTGTAT |
| | | TTTGCCTCTGATGAAGCCAGACACATTACAGGTAGCATTGTAGCTTCTGA |
| | | TGGCGGGTTTACCGTATATTAATAA |
| 39 | Codon optimized | ATGGATTTCCTGGCACTTCTGTGCTACAACACCATTAAATCGAACAAAGA |
| | 3β-hydroxysteroid | AGTTATAAACAGAGGTCGTTTTTCTGGCAAAATTATGCTGGTAACTGGTG |
| | dehydrogenase gene | CTACTTCAGGAATCGGAAGAGCAGTTGCGTTGCGTGGAGCAAAAGAAGGT |
| | from | GCAACAGTAATCGCTGTGGGCCGCAATGAAGAACGTGGAAACGCAGTGGT |
| | Lachnospiraceae sp. | CGAAGCCATTGAAAATAAAGAGGGTAAGGCCGTGTTCAAAAAATGCGACG |
| | 2_1_46FAA | TAAGTGATAAAGAGGCAGTCAAGAAATTGTTCGCCGAAATCAAGGAAGAG |
| | | TTCGGAAAATTGGATGTTGCAGTAAATAATGCGGGTATCGTGGGAGCATC |
| | | TAAGACTGTGGAGGAGTTGGAGGACGACGATTGGAGTAAAGTGATCGATG |
| | | CTAATCTGAATTCTTGTTTTTATTGCTGCAGAGAAGAAGTCAAGCTGATG |
| | | AAAGAAAACGGAGGAGCCATTGTCAATGTATCCAGCGTAGCCGGAATGAG |
| | | AGGATTTCCGAGTGCTGCTGCATACGTAGCTTCCAAACATGCAGTTAGTG |
| | | GTCTGACAAAAGCCGTGGCGGTGGATTATGCAACCAAGGGTATTACATGT |
| | | AATGCAGTATGTCCCGCTGGAACAGATACACCATTGACTGAACGTTCCTC |
| | | CGCTGACATTAAAACTCGTATGGCCGAAATTGCTGCTCAGGGAAAAGATC |
| | | CCATGGAATGGCTTAAGAACTCGATGTTGTCAGGAAAAACCGAAACATTG |
| | | CAGAAACGTAATGCAACGCCGGAAGAACAAGCAGCTACTATACTGTTCTT |
| | | TGCATCGGATGAAGCCAAACACATCACAGGATCTATTGTGGCATCCGATG |
| | | GGGGTTTCACTACTTATTAATAA |
| 40 | Codon optimized | ATGCGTGATTATTTTGAAGGCAGATTCGAGGGAAAAAACATGCTTGTGAC |
| | 3β-hydroxysteroid | AGGCGGAACTAGCGGTATCGGCAAATCAGTATGTATACGGGCCGCGAAAG |
| | dehydrogenase gene | AAGGAGCTTTTGTAATTGTAGTTGGACGTAATGAAGAACGTGGGCAAAGC |
| | from Clostridium | GTGGTTAATGAGATCGTCGAAAATGGGGGTAATGCCCGGTTTATCAAAGT |
| | sp. NSJ-6 | GGATGTATCTCGCGAAGATGAGGTCATAAATCTGTTTAATGTCATAAATA |
| | | ACGAAATTGGAGACATTCACGTAGCTATAAACAATGCAGGAATCGTCGGA |
| | | CATGGAGAACGCATTGATGAACTTAGCACAGAAGAATGGCTGAGAGTAAT |
| | | AAATACGAATTTGAACAGTGCTTTTTACTGCTGCCGTGAGGAAGCCAAAA |
| | | ATATGCTGAAACACAAACAAGGTGGAAGTATAGTAAATATTTCTTCTATC |
| | | GCAGGATCAACTGGGTTCTATCGCTCCAGTGCCTATGTATCATCGAAACA |
| | | TGCCCTTAATGGTTTGACTAAAGCTGTAGCAAATGACTTGGCAGCGTTCA |
| | | ACATCCGCTGTAACAGTGTAAGTCCTGCAGGAACTGCAACACCTTTGAAC |
| | | GATCGTTCAGCCGAGGAAATCAAAGGTAAGATCGGAGCAGCTATGGCGCA |
| | | GGGAAAATCGATCGAAGAGGCAAAAAGCGAAACAATGATCGGTGGGAAGA |
| | | CCGAAACTCTGCAAAAACGTAGCGCTACTGCAGAGGAACAAGCAGCAACC |
| | | ATCCTGTATGTGGCATCAGAAGAGGCTGCACACATTACCGGCTCAATAAT |
| | | CATGTCAGATGGCGGTTATACTGCGTATTAATAA |
| 41 | 3β-hydroxysteroid | ATGGAAAAAGGATTAGCCATCATAACCGGTGCCGACGGAGGCATGGGACA |
| | dehydrogenase gene | AGTAATCACGGCAGCCCTCGCAAAAGAAGGCTATCCGGTGATTATGGCTT |
| | from | GCCTCGATCCGGAAAAAGCCGTCCCCGTATGCACCCGGATCCAGCAAGAA |
| | Parabacteroides | ACCGGTAACACTCAGATCGAAGTGCGGGAAATCAATCTCGCTTCCCTCTC |
| | merdae ATCC | ATCCGTAAACAATTTTACCGGTCAATTATTAAAAGAAGGACGTCCCGTCA |
| | 43184 | GCCTCCTGATGAACAATGCCGGAATCCTGACGACTCCGGTACGCAAAACT |
| | | GAAGATGGGTTGGAAACAATCGTAAGCGTCAATTATGTGGCTCCCTACAT |
| | | GCTCACCCGCCAGTTATTACCATTGATGCAACCAGGATGCCGTATTGTAA |
| | | ATACAGTGTCCTGCACGTATGCCATCGGCCGGATCGAACCGGACTTCTTT |
| | | GAAAAAGGAAGGAACGGACGTTTTTTCCGCATTCCGGTCTATAGCAATAC |
| | | CAAACTGGCGCTGTTATTGTTCACCCAAGAGTTTGCCGAACGGCTGCAAG |
| | | ACAAAGACATCACCATAAATGCCTCGGACCCGGGAATCGTCAGTACGAAC |
| | | ATGATCACGATGCAAGCTTGGTTCGACCCGCTTACCGATATCTTGTTTCG |
| | | CCCCTTTATCAAAACACCGGCCCAAGGCGCGGCGACCGCCATCCATCTGG |
| | | CCCTTTCGGATGAGGCGAAAGATAGAAACGGTTGTTGCTATGCCAATTGC |
| | | AAAAAGAGGAATGTGTCAGAACGCATCCGGCATCATGCACAGCAAAAACA |
| | | ACTTTGGGACGATACGGAAATCTTGCTCCGGCAAAAAGGAATCCGGTTCT |
| | | GA |
| 42 | 3β-hydroxysteroid | ATGAGTAAATTAGCCATAATAACCGGTGCCGATGGAGGAATGGGTACTGA |
| | dehydrogenase gene | AATAACCCGTGCGGTAGCACAGGCCGGCTATCATGTAATAATGTTGTGTT |
| | from Bacteroides | ACACTCTTTTTAAAGGAGAGGAGCGTAAGAACCAGTTGATTTTGGAAACT |
| | dorei DSM 17855 | GGCAATAAAGAGATAGAAGTCAGACAAGTTGACCTTTCTTCCATGGCTTC |
| | | TGTGACTAATATCGCGGACGACTTGTTGGGGCGCGGAAAGCATATCGATT |
| | | TACTGATGAACAATGCGGGAACAATGAGTTCCGGCGGTTTGATTACAACG |
| | | GAGGATGGTTTGGAATATACGGTAGCTGTGAATTATGTGGCCCCTTTTTT |
| | | ACTGACTTTGAAATTATTGCCTCTGATGGGACAAGGAACCCGGATTGTGA |
| | | ACATGGTTTCTTGCACGTATTCCATAGGGAAGATTACTCCTGAATTTTTG |
| | | ATTCGTGGAAAAAGAGGCAGTTTTTGGCGTATCCCTGTTTACAGTAATAC |
| | | AAAATTGGCTTTGTGGCTTTTTACCCGTGAACTTTCTGAAAGGCTGAAAA |
| | | CAGAAGGAATTACTGTCAATGCTGCCGATCCTGGTATTGTTTCTACCAAT |
| | | ATCATCCGTATGGATATGTGGTTTGACCCGTTGACTGACATACTGTTCCG |
| | | TCCTTGTATCCGTACTCCGAAGCAAGGAGCCGCGACAGCTGTCAGTTTGC |
| | | TTTTGGATGATCGATGGAAAGAAGTTACAGGGCAAATGTTCGCTTCTTGC |
| | | AAGCCTAAAAAAGTAAAGGATAAGTTTATGAATCATCCACAGGCAAGACA |
| | | GCTTTGGGCGGATACGAAAGCATATTTGGAGAAACTGAAATTGGAGGAGC |
| | | CAATTGTCTGA |
| 43 | 3β-hydroxysteroid | ATGAGTAAATTAGCCATAATAACCGGTGCCGATGGAGGAATGGGAACCGA |
| | dehydrogenase gene | AATAACCCGTGCGGTAGCACAGGCCGGTTATCATGTAATTATGTTGTGCT |
| | from Bacteroides | ACACTCTTTTCAAAGGAGAGGAGCGTAAGAACCAGTTGATTTTGGAAACA |
| | vulgatus ATCC | GGCAATAAAGAGATAGAAGTCAGACAGGTTGACCTTTCTTCTATGGCTTC |
| | 8489 | TGTGACTAATATCGCAGAAGATTTGTTGGGGCGTGGAAAACATATCGACT |
| | | TACTGATGAACAATGCGGGAACCATGAGTTCCGGAGGTTTGATTACAACG |
| | | GAGGATGGTTTGGAATATACGGTAGCCGTGAACTATGTGGCCCCTTTTTT |
| | | ACTGACCTTGAAATTATTACCTCTGATGGGGCAGGGAACCCGGATTGTGA |
| | | ACATGGTTTCTTGTACGTATTCCATAGGGAAGATTACTCCTGAATTTTTT |
| | | GTTCGTGGAAAGAGAGGAAGTTTTTGGCGTATCCCTGTTTACAGCAATAC |
| | | AAAGTTGGCTTTGTGGCTTTTTACCCGTGAACTTTCTGAAAGGCTGAAAG |
| | | CAGAGGGAATCACCGTCAATGCTGCCGATCCCGGTATTGTTTCTACCAAT |
| | | ATCATCCGTATGGATATGTGGTTTGATCCGTTGACCGACATACTGTTCCG |
| | | TCCTTGTATCCGTACTCCGAAGCAAGGAGCTGCGACAGCTGTCAGTTTGC |
| | | TTTTGGATGAGCGATGGAAAGAAGTTACAGGACAGATGTTTGCTTCTTGC |
| | | AAGCCTAAAAAAGTAAAGGATAAGTTTATGAATCATCCGCAGGCAAGACA |
| | | GCTTTGGGCGGATACGAAAGCATATTTGGAGAAACTGAAATTGGAGGAGC |
| | | CAATTGGCTGA |
| 44 | 3β-hydroxysteroid | ATGAGTGAAGAGAAATGGGCAATCATCACCGGTGCCGACGGCGGCATGGG |
| | dehydrogenase gene | AACGGAAATAACCCGTGCCGTAGCCGAAGCCGGTTACCATATTATTATGG |
| | from Bacteroides | CTTGCTATCGTCCGTCCAAAGCGGAACCGATACGGCAGCGTCTAGTGAAC |
| | thetaiotaomicron | GAGACAGGAAACGCAAACATGGAAGTCATGGCAGTCGATCTGTCTTCTAT |
| | VPI 5482 | GGCATCGACAGCTTCTTTTGCCGATCGGATTGTGGAGCGTCATCTCCCCG |
| | | TTTCCCTGCTGATGAATAACGCCGGAACAATGGAAACCGGACTTCACATC |
| | | ACCGACGACGGCTTTGAACGAACGGTCAGTGTGAACTATCTGGGGCCGTA |
| | | CCTGCTTACCCGGAAACTCCTTCCGGCATTGACATGCGGAGCCCGTATTG |
| | | TAAACATGGTTTCTTGCACGTATGCGATCGGACACCTCGATTTTCCCGAT |
| | | TTCTTCCGGCAGGGAAGAAAGGGAAGTTTTTGGCGAATCCCTGTTTACAG |
| | | CAATACCAAACTGGCTTTGATGCTGTTTACGATCGAACTTTCGGAACGCC |
| | | TCCGTGAAAAAGGAATCACTGTCAATGCCGCCGATCCCGGCATTGTTTCT |
| | | ACCGACATCATCACTATGCACCAGTGGTTTGACCCTCTGACGGATATCTT |
| | | TTTCCGCCCCTTTATCCGCACGCCGAAGAAAGGGGCTTCCACTGCCGTCG |
| | | GCCTCTTGCTGGATGAGGCAGTGGCCGGAGTCAGCGGACAGCTTTATGCG |
| | | AGCAGCCACAGGAAGCAGCTGTCCGAAAAATACCTCTGCCATGTGCAGCA |
| | | AAAACAACTGTGGCAGGAAACGGAACAGGCTTTGGAACGCTGGTTGAAAT |
| | | AA |
| 45 | 3β-hydroxysteroid | ATGAATGAAGTGAAATGGGCGATCATTACCGGAGCTGACGGAGGCATGGG |
| | dehydrogenase gene | AACGGAGATAACCCGTGCCGTGGCCACAGCCGGCTATCATGTCATAATGG |
| | from Bacteroides | CTTGTTATAACCCGCAAAAAGCGGAAAACGTGTGCCAACGTTTAATGAAA |
| | caccae ATCC | GAAACCGGAAATCCGAATTTGGAAGTACTCGCTATTGATCTGTCTTCGAT |
| | 43185 | GCACTCCGTAGCCTCTTTTACTGATCGGATTTTGGAACGTAAACTTTCCA |
| | | TTTCCTTGTTGATGAATAATGCCGGGACAATGGAAACCGGATTTTCTATT |
| | | ACGAACGATGGATTTGAACGGACGGTCAGTGTAAATTATGTTGGTCCTTA |
| | | CCTGCTGACTCGTAAATTAGTTCCGACTATGGCATCCGGAGCACGTATTG |
| | | TAAATATGGTTTCGTGTACGTATGCAATCGGCCGTCTTGATTTTCCTGAT |
| | | TTCTTTCACAGGGGGAAAACGGGAAACTTTTGGAGAATACCTGTTTATAG |
| | | TAACACAAAATTGGCTTTGTTATTATTTACTTTCGAACTATCCGAGCAAC |
| | | TTCGGGAGAAAGGAATCACCGTCAATGCTGCCGATCCGGGAATTGTCTCT |
| | | ACTGATATCATCACGATGCATAAGTGGTTCGACCCTCTGACAGATATATT |
| | | CTTCCGTCCTTTTATTCGTAAGCCGAAGAAAGGAGCTTCTACGGCAATTG |
| | | GTCTGTTGCTGGACAAAAAAGAAGCCGGTGTGACAGGGCAACTCTATGTC |
| | | AATAATCACCGGAAAAGCTTATCCGATAAGTATGTGAACCATGTACAGAA |
| | | AGAGCAGTTGTGGGAAATAACGGAGCGTTTGCTGGCGCAATGGTTGGAGT |
| | | AG |
| 46 | 3β-hydroxysteroid | ATGAATGATATAAAATGGGCTATCATTACCGGTGCGGACGGAGGGATGGG |
| | dehydrogenase gene | AACGGAAATCACACGTGCCGTTGCCAAAGCTGGTTATCAGGTAATAATGG |
| | from Bacteroides | CTTGTTACAACCCCCAAAAGGCGGAGACTGTCCGCGCTTGTTTGATTGAA |
| | finegoldii DSM | GAAACCGGAAACCCGAATCTGGAAGTTATGGCTCTTGATTTGGCTTCCAT |
| | 17568 | GCAATCCGTAGCTTCTTTTGCCGACCGGATATTAGAACGTAACCTTCCTG |
| | | TTTCTCTGCTGATGAATAATGCGGGAACGATGGAAACGGGACTTCATATT |
| | | ACCGTAGATGGGTTTGAGCGAACGGTTAGCGTGAATTATGTAGGACCTTA |
| | | TCTGCTTACCCGGAAACTGATTCCTGCGATGGTGCGCGGTGCGCGAATTG |
| | | TAAACATGGTGTCTTGCACTTATGCGATCGGGCGTATTGAACTTCCCGAT |
| | | TTCTTTCACAGAGGCAAGGTCGGAGAATTTTGGAGAATTCCCGTTTACAG |
| | | CAATACGAAACTGGCTTTATTGTTGTTTACCATTGAACTGTCCAAGCTAC |
| | | TCCGTGATAAAGGAATTACCGTCAATGCTGCCGATCCGGGCATTGTCTCT |
| | | ACTAATATTATTACTATGCATAAGTGGTTTGACCCGCTGACGGACATTTT |
| | | TTTCCGGCCTTTTATTCGCAAGCCCGCACAAGGGGCTTCCACCGCTATCG |
| | | GTTTGTTGTTGGATGAAAAAGAAGCCGGAGTGACGGGGCAACTGTATGCT |
| | | AGTAATCGTCGGAAAGAATTATCGGATAAATACGTTCATCACGTGCAGAG |
| | | GGAGCTACTGTGGGAAGTCACGGAACGTTCGTTGGCACGATGGATTTCTT |
| | | CCTAA |
| 47 | 3β-hydroxysteroid | GTGAATTTAGCTGTTATAACTGGGGCAGACGGTGGCATGGGCATGGAAAT |
| | dehydrogenase gene | TACCCGCGCAGTGGCAACTGCCGGCTATCAGGTCATCATGGCATGCCGTG |
| | from Bacteroides | ACCCCCAAGCTGCCGAACCCAAGCGGCAACTACTGATGCGTGAAACCGGT |
| | uniformis ATCC | AATCCGCGTATTGAGACTGCTCCCATTGATTTGGCATCTCTGGCTTCAGT |
| | 8492 | GGCCGCATTTGCAGAGCATCTGTTGAAGCGGGGAGAGCCGTTGGCGTTGC |
| | | TGATGAACAATGCCGGAACCATGGAAACGGAACGCCGCATTACCGAAGAC |
| | | GGACTGGAACGGACGGTGAGTGTCAATTATGTAGGGCCTTACCTGCTGAC |
| | | CCGCAAGCTGCTACCATTGATGGGAGAGGGGAGCCGTATTGTGAATATGG |
| | | TATCTTGTACGTATGCCATCGGTCATCTTGACTTTCCGGATTTTTTCCTC |
| | | CGGGGAAGGAAGGGTGGCTTTTGGCGCATTCCTATATATAGCAACACGAA |
| | | GCTGGCATTGACTCTGTTCACCATCGACTTGGCCAGTCGCGTCAAACACA |
| | | AAGGTATTGTTGTGAATGCGGCCGACCCGGGGATTGTGTCTACCAATATC |
| | | ATCACCATGCATATGTGGTTTGACCCGCTGACAGATATACTTTTCAGGCC |
| | | TTTTATCCGTACTCCCCGTAAGGGAGCTGCAACAGCTGTCGGCTTATTGC |
| | | TGGATGAGGATGCCGGTAAACGTACGGGGACATTGAATGCCAGTTGCCGT |
| | | CCCAAGTCTCTTTCGGAGAAGTACACCCGGCATGTACAGATGGAAGAACT |
| | | GTGGGAGAGGACGGAAAGTATAGTGAAAAAATGGTTGTAA |
| 48 | Codon optimized | ATGGACATGGGATTGAAAGATAAGGTAGTCTTAATAACAGGTGGAGGAGG |
| | 12α-hydroxysteroid | CGGAATCGCACGTGGCATCGAACGTGCATTTGCAACAGAAGGAGCAAAGT |
| | dehydrogenase gene | TTATTCTGACGGACCTGTTCCCTGGAGGCTTGGAAGCCGCTAAGGAGGAA |
| | from Eggerthella | TTGGAACGCGATTTTGGATCCGAAGTCTTTACGATACTGGCAAATGGAAG |
| | lenta C592 | TGTAGAAGAAGAGGTGCGTGCTTCTGTCGAAGCAGGTGCCGAACATTTTG |
| | | GTGGCCGTATTGATGTTCTGATCAATAATGCTCAGGCTTCCGCATCCGGA |
| | | TTGACTTTGGTACAACATTCAGAGGAGGATTTCGATCTTGCAGTGCGCTC |
| | | TGGACTTTATGCTACGTTCTTTTATATGAAGCATGCCTATCCATATTTGA |
| | | AGGAAACTGCAGGGAGTGTCATTAATTTCGCAAGTGGTGCCGGGATCGGA |
| | | GGTAATCCCGGACAATCATCATATGCAGCTGCTAAGGAAGGTATTCGTGG |
| | | CATGAGTCGTGTTGCAGCTTCAGAATGGGGACCGGATAATATTAACGTAA |
| | | ACATCGTGTGCCCCATAGTAATGACCAAAGCACTGGAAGAATGGCGTGAA |
| | | AGAGAACCCGAAATGTACGAAAAAAACGTGAAAGCAATACCCCTGGGTCG |
| | | CTTTGGAGATGCGGAAAAGGATGTTGGACGCGTATGTGTATTTCTTGCAA |
| | | GCCCAGATGCCAGTTTTGTAACTGGAGATACAATTATGGTTCAGGGCGGT |
| | | TCCGGCATGAAACCATAA |
| 49 | Codon optimized | ATGGACATGGGATTAAAGGATAAAGTAGTTTTAATTACCGGAGGTGGAGG |
| | 12α-hydroxysteroid | AGGTATAGCACGTGGTATTGAACGTGCTTTCGCAACTGAAGGTGCCAAAT |
| | dehydrogenase gene | TCATTCTGACTGACTTATTTCCTGGAGGATTGGAAGCAGCTAAAGAAGAA |
| | from Eggerthella | CTGGAGAGAGACTTTGGTTCCGAAGTCTTCACAATCCTGGCAAATGGATC |
| | lenta DSM2243 | TGTAGAAGAAGAGGTCCGTGCAGCCGTCGAAGCTGGAGCCGAACATTTCG |
| | | GTGGCCGTATCGATGTGCTGATAAATAACGCGCAAGCATCCGCTTCCGGA |
| | | TTGACTTTGGTGCAGCATTCTGAGGAAGATTTTGATTTGGCAGTCCGGAG |
| | | TGGATTGTATGCAACGTTCTTCTATATGAAACACGCCTACCCGTATCTTA |
| | | AAGAGACAGCCGGATCAGTAATCAATTTTGCTTCAGGAGCTGGGATCGGT |
| | | GGTAATCCCGGTCAATCATCATATGCTGCCGCTAAAGAAGGCATTCGTGG |
| | | TATGTCACGTGTGGCAGCTTCTGAATGGGGACCGGATAATATCAATGTAA |
| | | ACATCGTGTGCCCTATAGTAATGACTAAAGCGTTAGAAGAATGGAGAGAA |
| | | CGTGAACCGGAGATGTATGAGAAAAATGTCAAAGCTATTCCGTTGGGTCG |
| | | TTTTGGTGACGCAGAAAAGGACGTTGGGAGAGTTTGTGTATTCTTGGCAT |
| | | CACCTGATGCATCCTTCGTAACAGGAGACACTATCATGGTACAAGGCGGA |
| | | TCAGGTATGAAACCGTAA |
| 50 | Codon optimized | ATGGGATTCTTAGAAGGAAAAACTGCCATAATTACCGGCGGTGGACGTGC |
| | 12α-hydroxysteroid | AGTCTTAAAAGATGGCTCTTGCGGTTCAATTGGATATGGTATAGCAACCG |
| | dehydrogenase gene | CGTACGCAAAAGAAGGCGCAAACTTAGTTATTACAGGACGTAATGTGCAA |
| | from Eggerthella sp. | AAATTGGAAGATGCCAAGGAAGAATTGGAGCGCTTATACGGAATCAAAGT |
| | CAG: 298 | ATTGCCGATTCAAGCCGATGTATCTGCAGGCAATGATAACGCCGCAACCG |
| | | TTCAAAATGTGATCGATAAAACTATTGAAGAATTCGGACGGATTGATGTG |
| | | TTGATCAATAATGCCCAAGCTAGTGCTTCTGGAGTATCCCTGGCGGAGCA |
| | | TACAACGGACCAATTCGATTTGGCAATCTATTCTGGTTTGTATGCTGCCT |
| | | TCTATTACATGCAAGCATGTTACCCTCACTTGAAAGAAACGAAAGGTACC |
| | | GTGATAAACTTTGCAAGTGGAGCAGGATTGTTTGGCAATGTGGGACAATG |
| | | TTCGTATGCAGCTGCGAAAGAAGGAATCAGAGGTTTGACACGTGTTGCCG |
| | | CAAATGAATGGGGTGCCGATGACATTAACGTGAATGTAATCTGCCCGTTG |
| | | GCTTGGACGGCACAATTGGAGAATTTTGCGGAGGCATATCCGGACGCATT |
| | | CGAAACGAATGTTCATATGCCGCCGATGGGACATTATGGTAATGTAGAGA |
| | | CTGAAATTGGACGTCCGTGTGTACAGTTGGCTTCACCTGATTTTCGTTTC |
| | | ATGTCCGGGGAAACAATCACGTTGGAAGGCGGTATGGGATTACGTCCATA |
| | | A |
| 51 | Codon optimized | ATGGATTTCATTGACTTCAAAGAAATGGGACGGATGGGAATTTTTGACGG |
| | 12α-hydroxysteroid | AAAAGTGGCAATAATCACTGGTGGAGGGAAGGCGAAAAGTATCGGATATG |
| | dehydrogenase gene | GGATAGCTGTAGCGTACGCAAAAGAAGGCGCAAACTTGGTCTTAACGGGA |
| | from Clostridium | CGTAACGAACAAAAACTGTTAGACGCTAAGGAAGAACTGGAACGCCTGTA |
| | sp. ATCC29733 | TGGAATCAAAGTCCTTCCTCTGGCTGTGGACGTTACGCCTTCGGATGAAT |
| | | CAGAAGATCGCGTAAAAGAAGCCGTACAGAAAGTCATTGCCGAATTCGGA |
| | | CGGATCGATGTTTTAATCAACAACGCACAGGCATCAGCCAGCGGTATTCC |
| | | GTTGTCGATGCAAACGAAGGATCATTTCGATTTGGGAATCTATAGTGGAT |
| | | TGTATGCTACGTTCTACTACATGAGAGAATGTTACCCGTATTTGAAAGAA |
| | | ACCCAGGGCTCCGTTATAAATTTTGCATCAGGCGCGGGTTTGTTCGGTAA |
| | | TGTCGGACAGTGTTCTTACGCAGCTGCCAAAGAAGGAATTCGCGGATTAT |
| | | CCCGTGTCGCTGCAACAGAATGGGGCAAGGACAATATTAATGTTAATGTC |
| | | GTGTGCCCTTTGGCTATGACGGCCCAGTTGGAAAATTTTAAATTATCGTA |
| | | CCCGGAAGCATATGAAAAAAATCTGAGAGGTGTCCCTATGGGACGGTTTG |
| | | GTGACCCTGAATTGGACATTGGCCGTGTATGTGTGCAGCTTGGATCTCCG |
| | | GATTTTAAATATATGTCTGGTGAGACACTGACCCTTGAAGGTGGAATGGG |
| | | ACAACGTCCGTAA |
| 52 | Codon optimized | ATGGGCATTTTTGACGGTAAGACGGCAATCATAACAGGTGGTGGGAAAGC |
| | 12α-hydroxysteroid | GCGTAGTATTGGTTATGGAATCGCCGTCGCATATGCGAAAGAAGGTGCTA |
| | dehydrogenase gene | ACCTGGCCTTGACAGGCAGAAACGAACAGAAGCTGTTGGATGCCAAGGAA |
| | from Clostridium | GAGCTGGAACGCCTGTACGGAATAAAGGTTTTGCCGTTACAAGCTGATGT |
| | hylemonae | GACACCTGATGAAAAAAGCGAGGAAGTCGTGAAAGAAACGGTGCAGAAAG |
| | DSM15053 | TGGTAGACACCTTTGGCCGGATTGACGTTTTGATCAATAATGCGCAGGCT |
| | | TCAGCCTCAGGTATTCCGCTGAGCATGCACATGAAAGACCATTTCGACCT |
| | | GGGTATTTATTCTGGCCTGTACGCGGTATTTTATTATATGCGTGCGTGCT |
| | | ACCCGTACTTAAAGGAAACCCAGGGATCCGTGATAAATTTTGCTTCTGGT |
| | | GCTGGATTGTTTGGAAATGCCGGTCAGAGCAGTTATGCCGCGGCAAAAGA |
| | | GGGAATACGTGGCATCTCTCGTGTGGCCGCTACAGAATGGGGTAAAGATA |
| | | ACATTAATGTGAACGTGGTCTGTCCGCTGGCCATGACTGCGCAGCTGGAA |
| | | AATTTTAAGGAAGCGTACCCGGAAGCGTACGAAAAAAACCTGAAAGCGGT |
| | | GCCGATGGGTCGGTTTGGTGATCCTGAGAAAGATATCGGAAGAGTCTGCG |
| | | TGCATTTGGGAAGTCCTGATTTGAAGTACATGTCTGGTGAAACTCTTACT |
| | | CTGGAAGGTGGTATGGGCCAACGGCCTTAA |
| 53 | Codon optimized | ATGGGTTTTCTGACAGGTAAGACAGCAATCATAACCGGTGGTGGACGTGC |
| | 12α-hydroxysteroid | TACCTTAAGTGATGGAAGCTGCGGAAGTATCGGATACGGCATTGCTACCG |
| | dehydrogenase gene | CATATGCCAAGGAAGGAGCAAATTTGACGCTGACAGGACGTAACGTGAAA |
| | from Clostridium | AAATTGGAAGACGCGAAAGAAGAACTGGAACGTCTGTATGGAATAAAAGT |
| | scindens | GTTGGCTGTCCAAGCTGATGTTTCAGCTGGAGCCGATAACAAAGCCGTAG |
| | ATCC35704 | TGGAACAGGTAATCAAGCAAACTGTCGAGGAATTCGGAAGAATCGATGTA |
| | | CTTATAAATAATGCACAGGCTTCCGCTAGTGGAGTATCAATAGCAGATCA |
| | | TACCACGGAACAATTCGATCTTGCGATATATTCCGGTTTATATGCAGCGT |
| | | ACTATTACATGCAGGCATGTTATCCATATTTGGCCGAAGCTAAAGGAAGT |
| | | GTTATTAACTTTGCAAGTGGTGCTGGACTTTTCGGCCATTATGGACAGTG |
| | | TTCGTATGCAGCTGCAAAAGAAGGTATTCGTGGTCTTACACGTGTTGCTG |
| | | CAACAGAGTGGGGCAAGGATGGAATCAACGTAAATGTCGTTTGTCCCTTG |
| | | GCATGGACTGTCCAGCTGGAAAATTTCGAAAAAGCCTATCCTGATGCATT |
| | | CAAGGCAAATGTAAAAATGCCTCCGGCAGGACACTATGGAGATGTAGAGA |
| | | AGGAAATTGGTAGAGTATGCGTTCAGCTGGCCAGCCCTGACTTCAAATTT |
| | | ATGTCTGGAGAAACGATCACTTTGGAAGGTGGCATGGGACTTCGTCCATA |
| | | A |
| 54 | Codon optimized | ATGGGCTTTCTGAACGGAAAAACAGTAATAGTTACTGGAGGTGGACGTTC |
| | 12α-hydroxysteroid | TGTACTTTCTGACGGCCGCTGTGGATCTATTGGTTATGGAATAGTAACTG |
| | dehydrogenase gene | CCTTCGCAAAAGAAGGAGCTAATATTGTAATCACGGGACGGAACGTAAAA |
| | from Clostridium | AAGCTTGAGGACGCAAAAGAGGAAATCGAACGCCTGTATGGAGTAAAAGT |
| | hiranonis | ACTGCCTGTAAGAGCCGATGTGTCGGCAGGTGGAGACAATAAGGCCGTGG |
| | DSM13275 | TCGACGAAGTGATAAAACAAACAATAGATACGTTTGGAAGAATAGACGTC |
| | | TTAGTAAATAATGCCCAGGCTTCGGCATCTGGTGTAACTCTGGAAGACCA |
| | | TACAACGGAACAGTTTGACTTGGCAATATACAGCGGTTTATATGCAACAT |
| | | TCTATTACATGCAAGCATGTCTTCCCTACTTAAAGGAAACAAAGGGTTCT |
| | | GTGATAAACTTTGCTTCAGGCGCGGGTCTTTTTGGTAACTATGGTCAATG |
| | | TGCCTATGCAGCAGCAAAGGAAGGAGTCCGTGGTTTAACACGCGTGGCTG |
| | | CAACTGAATGGGGCCAGTTCGGCATTAATGTTAATATAATCTGTCCCCTT |
| | | GCTTGGACAGCTCAACTGGAGAATTTTGAAAAGGCTTATCCAGAAGCATT |
| | | TAAAGAAAATGTAAAAATGCCACCAGCTGGCCATTATGGGGATGCGGAAA |
| | | AAGAAATTGGTAGAGTATGTGTCCAGTTGGCTTCACCGGACTTCAAATAC |
| | | ATGTCGGGAGAAACTATCACATTAGAAGGTGGTATGGGACTTCGTCCCTA |
| | | A |
| 55 | Codon optimized | ATGAAAGAACTGAATGAGAAAGTGGCCATTATCACAGGTGCTGGACAAGG |
| | 12β-hydroxysteroid | TATCGGCAAAGGGATAGCCTTACATCTGGGTAAACGTGGCGTGAAAGTTG |
| | dehydrogenase gene | TTTGCGTTGGACGCCGTTTGGATCCGATTGTCCAAACCGTGAAAGAAGTT |
| | from Clostridium | GAAGAAGCTGGTGGCCAAGGATTCGCTATAACTTGCGATGTGGGTAATCG |
| | paraputrificum | GGAGGATGTTAAAAAAGTGGTCAAAGCGACCGTAGAAAAATACGGAACGG |
| | ATCC 25780 | TCGATGTAGTTGTAAATAATGCACAGAGTTTGCCTGGGTCCGCCAAAGTG |
| | | GAAGATACAACGTACGAACAGATGCTTACTGCATGGCAAAGCGGAACAAT |
| | | CGGTTCACTGAACATGATGCAAGAATGTTTCCCATATATGAAAGACCAGA |
| | | ACGAAGGTAGAATCATCAACTTTGCTTCCGCAACAGGCATGTTTGGCTAT |
| | | GCAGGACAGCTTGCCTATGGCTGCAACAAAGAATCGATTCGTGGACTGAC |
| | | CAAAATTGCGGCAAAAGAATGGGCTCAATACAACATTATCGTAAATTGTG |
| | | TGCTTCCTGGGGCCGAAAGCCCAGCAGCAAAGGTATGGGCCGAGAAATTC |
| | | CCGGAAAAGTATAAGGAAATAATGGAAGCCCAACCAATGAAACGTTTTGG |
| | | GGATGGTGAAGATGACATAGGTCGTGTAATCGCTTTTCTTGCAGGTCCTG |
| | | ATTCTAAATATTACACTGGACAGTGCCTGTTGGTTGATGGGGGATATAGT |
| | | ATAGCCCCGTAA |
| 56 | Codon optimized | ATGAAGCAGCTGAATGAAAAAGTTGCTATTGTAACCGGTGCTGGTCAAGG |
| | 12β-hydroxysteroid | TATTGGACAAGGGATCGCTTTGTGTCTGGGCAAACGTGGGGTAAAGGTGG |
| | dehydrogenase gene | TATGCGTAGGAAGAAGACCTGAACCGATCGAAGCAACCGCCAAAGAAATC |
| | from Eisenbergiella | CGTGATTTGGGAGGTGAATCGTTTGCTATGACCTGTGACACAGCGGATCG |
| | sp. OF01-20 | TGACAGAGTAAAGGAAGTCGTGGCAAAAACTGTAGAGACATATAAAACAG |
| | | TTGATGTAATGATCAATAATGCCCAGAGTTTGCCTGGAAGCGCTCCTGTG |
| | | GAAGAGGTAACATATGAAATGATGTACACTGCCTGGTCTACTGGTACTCT |
| | | GGGAAGCTTGAATTTTATGCAAGAATGCTTCCCTTATATGAAAGAACAAG |
| | | GTGAAGGACGTGTTATAAATTTTGCAAGTGCCACGGGTATGTTCGGTTAT |
| | | GCTGGAAATCTTGCCTATGGCTGCAACAAGGAAGCGATTCGTGGATTAAC |
| | | CAAAATCGCGGCAAAGGAATGGGGAAAGTATGGCATTTGCGTCAACTGCG |
| | | TGTTGCCTGGAGCTGAAAGTCCAGCAGCTAAAATCTGGGCCGAAAAGTTT |
| | | CCCGAAAAATATGCAGAGATTCTGGAACAGCAGCCTATGAAAAGACTGGG |
| | | AGATGCCGAAAAAGACATCGCACCAGTCATTGCCTTCCTTTCCGGACCTG |
| | | ATTCTTGTTATTATTCTGGCCAGTGTCTTCTGGTTGATGGTGCCTATTCC |
| | | ATAATGCCCTAA |
| 57 | Codon optimized | ATGAAACAGCTGAATGAAAAGGTGGCCATCGTAACCGGCGCCGGACAGGG |
| | 12β-hydroxysteroid | AATTGGAAAGGGAATCGCATTGTGTCTTGCGAAGCGTGGCGTAAAAGTAG |
| | dehydrogenase gene | TATGTACCGGAAGACGGGAAGCTCCAATCCAACAGACTGTGGCTGAGATT |
| | from Olsenella sp. | GAAGAATTGGGTGGACAGGGACTGGCCATGACATGTGATTCGGCAGATCG |
| | GAM18 | TGCCCGCGTGGAAGAGGTAGTAAAAGCAGCCGTGGATACATTCGGCTCTA |
| | | TTGATGTTATCGTGAATAATGGACAGGCTATTGTGCCGTCCGCCCCTGTA |
| | | GAAGACACGACATACGAAAACATGTTAGCCGCATGGCAGTCTGGTACTAT |
| | | AGGATCATTAAATTACATGCAAGCTGCATTCCCGCATATGAAGGAACAAC |
| | | ATGAAGGCCGTATAATAAATTTCGCCTCTGCTACGGGAATGTTTGGAATC |
| | | GCTGGCCAGCTTGCTTATGGGTCCAATAAAGAAGCTTTGCGTGGGCTGAC |
| | | AAAAATAGCAGCCAAAGAATGGGGACAATATGGTATCTGCGTAAATGTTG |
| | | TATTACCTGGAGCGGAATCACCTGCAGCGAAGGCATGGGCTGAAAAATTT |
| | | CCGGAGGAATACCAGAAGCAAGTAATGCTGAACCCAATGCATAGATTTGG |
| | | TGACCCTGAGGATGATATCGCACCGGTAGTTGCATTTTTAGCAGGCCCTG |
| | | ATTCATGTTATTATTCCGGCCAGTCTGTAATCGTCGATGGCGCGAATTCC |
| | | ATTATGCCTTAA |
| 58 | Codon optimized | ATGAAACAATTGAATGAAAAAGTTGCAATTGTCACAGGAGCTGGACAAGG |
| | 12β-hydroxysteroid | AATCGGGAAAGGCATTGCCCTGTGTCTTGCTAAACGTGGAGTAAAGATTG |
| | dehydrogenase gene | TGGCCACTGGTCGTCGTTTGGAACCGATCGAAGCGACAATAGCAGAAATA |
| | from Collinsella | AAGGAACTTGGTGGTGATGGACTGGCGATGAGTTGCGATTCTGCAGACAG |
| | tanakaei | AGAACGCGTTTTCGAAGTAGTAAAAACCGCCATTGACACCTTTGGTAGTA |
| | | TTGATGTCATCGTAAACAATGGACAAGCCATTGTACCAAGCCAACCGGTG |
| | | GAGGATACTGAATACGAAAACATGTTAAAAGCATGGCAATCGGGAGTTAT |
| | | TGGAAGTTTGAATTACATGCAAGCAGCTTTTCCATATATGAAGGAACAGC |
| | | ATGAAGGTCGCATCATAAATTTTGCATCCGCGACTGGTATGTTTGGAATT |
| | | GCGGGTCAGTTAGCCTATGGTAGTAACAAGGAAGCTCTGCGCGGATTAAC |
| | | AAAAATCGCGGCTAAAGAATGGGGACAATACGGAATTTGTGTGAATATTG |
| | | TGCTGCCTGGAGCTGAATCTCCTGCCGCAAAGGCCTGGGCCGAAAAATTC |
| | | CCTGAAGAATATGCGAAACAAGTAAATTTAAACCCGATGAAACGGTTTGG |
| | | AGATCCGGAAGCTGACATCGCGCCTGTGGTAGCTTTTCTTGCAGGACCTG |
| | | ACAGCTGCTATTTTAGCGGACAATCCGTGATAGTAGACGGTGCAAATTCA |
| | | ATTATGCCGTAA |
| 59 | Codon optimized | ATGAAACAATTGAATGAAAAGGTGGCTATTGTGACTGGTGCTGGACAAGG |
| | 12β-hydroxysteroid | GATTGGAAAAGGAATTGCCTTATGCCTGGCGAAAAGAGGAGTCAAAATTA |
| | dehydrogenase gene | TTGCAACGGGACGTAGACTGGAACCCATTGAACAAACAATAGCGGAGATA |
| | from Ruminococcus | AAGGAGCTGGATTCTGATGGACTGGCAATTACATGTGACTCAGCGGATCG |
| | sp. AF14-10 | TGCCCGTGTTGAAGAAGTTGTGAAAACTGCTGCCGATACATTTGGAACAG |
| | | TGGATATCGTGGTTAATAATGCACAAGCTATCGTGCCGTCTGCGCCTGTG |
| | | GAGGAAACTAGCTATGACAACATGTTCAAAGCATGGCAGAGTGGAGTAAT |
| | | TGGCAGCCTGAACTATATGCAGTCCGTGTTTCCTTACATGAAGGAACAAC |
| | | ACGAAGGTCGGATCATAAATTTTGCAAGCGCTACCGGTATGTTTGGTATC |
| | | GCGGGACAGTTGGCCTATGGATCGAATAAAGAAGCTATCCGTGGAATGAC |
| | | CAAAATTGCAGCAAAGGAGTGGGGACAGTATGGTATCTGCGTCAATGTTG |
| | | TTTTGCCGGGTGCTGAATCCCCTGCTGCAAAGGCTTGGGCAGAGAAATTT |
| | | CCTGAGGAGTATGCGAAACAAGTGAATTTAAACCCAATGAAACGTTTTGG |
| | | TAGTCCCGAGAATGACATAGCTCCAGTGATTGCTTTTTTGGCCGGACCGG |
| | | ATTCTTGCTATTTTTCTGGACAATCAGTAGTGGTAGATGGAGCGAATAGC |
| | | ATTATGCCGTAA |
| 60 | Codon optimized | ATGAAACAACTGAATGAAAAAGTGGCTATAGTAACTGGGGCCGGACAAGG |
| | 12β-hydroxysteroid | TATCGGCAAGGGAATTGCATTATGCCTGGCTAAGCGCGGCGTAAAAATTG |
| | dehydrogenase gene | TTGCCACTGGACGTCGTTTGGAACCGATTGAACAAACGATCGCTGAAATT |
| | from Ruminococcus | AAGGAGCTGGGTGGCGATGGATTTGCTATGTCCTGTGATTCTGCTGATCG |
| | lactaris | TGCTAAAGTTGAAGAGGTGGTAAAAGCAACAGTGGATACCTACGGAATTG |
| | | TCGACGTCGTGGTAAATAATGCTCAAGCAATCGTTCCGAGTGCCCCTGTG |
| | | GAAGAAACGACGTATGAGAATATGTTGAAGGCTTGGGAATCAGGGGTAAT |
| | | CGGCAGCTTGAATTATATGCAGGCCGCTTTTCCATACATGAAAGAGCAGC |
| | | ATGAAGGTCGGATCATCAATTTTGCAAGCGCAACTGGAATGTTTGGCATT |
| | | GCTGGTCAGCTGGCCTATGGCAGTAACAAGGAAGCCTTACGTGGTTTAAC |
| | | TAAAATTGCTGCCAAAGAATGGGGACAGTACGGAATATGCGTAAATATAG |
| | | TCCTTCCGGGTGCGGAAAGTCCTGCAGCCAAAGCATGGGCAGCCAAATTC |
| | | CCGGAAGAGTATGCGAAACAAGTAAATTTAAATCCGATGAAAAGATTCGG |
| | | TGATCCGGAAAATGACATTGCACCTGTCATCGCGTTTTTAGCTGGCCCGG |
| | | ACTCATGCTATTACAGTGGACAAAGTGTTATTGTGGATGGAGCTAATTCA |
| | | ATTATGCCGTAA |
| 61 | 5α-reductase gene | ATGACAACTGAACATTTCACCTTATTTCTAATTGTTATGGCAGCTATCGC |
| | from | CGCCATAGTCTTCATAGCCCTTTATTTCGTCGAAGCCGGTTATGGAATGT |
| | Parabacteroides | TGTTCGATAAAAAATGGGGACTTCCGATACCGAACAAGATTGCTTGGATT |
| | merdae ATCC | TGCATGGAAGCGCCGGTTTTTATCGTCATGTTTTTGTTATGGAACGGATC |
| | 43184 | GGAACGACAGTTCGAGACAGTACCGTTCCTGATATTCTTATTCTTCGAAC |
| | | TGCATTATTTCCAACGATCTTTTATTTTTCCTCTGTTGATAAAAGGCAAA |
| | | AGTAAAATGCCGGCAGGCATCATGCTTATGGGAATCACCTTTAACCTCCT |
| | | GAACGGTTATATGCAGGGAGAATGGATTTTCTACTTAGCACCGCAGGATA |
| | | TGTATACGAAAAGCTGGCTGCACAGCCCTCAATTTATAGTCGGGACAATC |
| | | TTGTTCTTCACCGGCATGGCAATCAATATCCAGTCAGACCATATTGTCCG |
| | | CCACCTCAGAAAGCCTGGCGACACGAACCATTATCTGCCTAAAAAAGGCC |
| | | TGTTCAAATATGTGACATCAGCCAACTACTTTGGCGAAATCGTGGAATGG |
| | | TGCGGATTTGCAATCCTGACCTGGAGTGCAAGCGGAGCTGTTTTCGCTTG |
| | | GTGGACATTTGCAAACCTTGTACCTCGCGCAAACACCATCTACCATAAAT |
| | | ACAAAGCGATGTTTGGTAACGAACTGGAAAACCGTAAACGGGTTATTCCT |
| | | TTTATATATTGA |
| 62 | 5α-reductase gene | ATGGGACAACAGACTTTTGAATTTTTGCTATTGGCAATGTCCGCACTTGC |
| | from Bacteroides | GGTGATTGTATTTGTAGCCCTCTATTATGTACGTGCCGGTTATGGTATAT |
| | dorei DSM 17855 | TCCACACCCCGAAATGGGGACTTTCAGTGAACAATAAATTAGGTTGGGTG |
| | | CTGATGGAAGCGCCTGTATTCCTTGTAATGCTTTATCTGTGGTGGAACAG |
| | | CAGCGTGCGTTTTGATGCCGCTCCTTTCCTCTTTTTTCTTCTTTTTGAAT |
| | | TACATTATTTCCAGCGCTCTTTTATCTTCCCTTTCCTGATGAAAGGAAAG |
| | | AGCCGGATGCCCCTTGCCATTATGTTGATGGGAGTGGTCTTTAATGTCCT |
| | | GAACGGACTGATGCAGGGCGAATGGTTGTTCTATCTGGCTCCGGAAGGAC |
| | | TCTATACAGATGCCTGGCTCAGTACTCCTTCTTTTTGGTTTGGGATCATT |
| | | TTGTTCTTTATAGGGATGGGCATTAATCTACATTCCGACAGTGTGATCCG |
| | | CCATTTACGTAAACCGGGCGATACACGTCATTATTTGCCGCAGAAGGGAA |
| | | TGTACCGATATGTCACTTCGGGCAACTATTTTGGCGAGTTGGTGGAATGG |
| | | ATAGGGTTTGCCGTACTCACTTGTTCGCCTGCTGCATGGGTGTTTGTACT |
| | | GTGGACGTTTGCTAATCTGGCTCCACGTGCTAATTCCATCCGTAACCGTT |
| | | ACCGGGAAGAGTTTGGTAAGGATGCGGTAGGAAAAAAGAAAAGAATGATT |
| | | CCTTTTATTTATTGA |
| 63 | 5α-reductase gene | ATGGGACAACAGACTTTTGAATTTTTGCTATTGGCAATGTCCGCACTTGC |
| | from Bacteroides | GGTGATTGTATTTGTAGCCCTCTATTATGTACGTGCCGGTTATGGTATGT |
| | vulgatus ATCC | TCCACACCCCGAAATGGGGACTTTCAGTGAACAATAAATTAGGTTGGGTA |
| | 8489 | CTGATGGAAGCGCCTGTATTCCTTGTAATGCTTTATCTGTGGTGGAACAG |
| | | CAGCGTGCGTTTTGATGCCGCTCCTTTCCTCTTTTTTCTTCTTTTTGAAT |
| | | TACATTATTTCCAGCGCTCTTTTATCTTCCCTTTCCTGATGAAAGGAAAG |
| | | AGCCGGATGCCCCTTGCCATTATGTTGATGGGAGTGGTCTTTAATGTCCT |
| | | GAACGGACTGATGCAGGGCGAATGGTTGTTCTATCTGGCTCCGGAAGGAC |
| | | TCTATACAGATGCCTGGCTCAGTACTCCTTCTTTTTGGCTTGGGGTTATT |
| | | CTGTTCTTTATAGGGATGGGCATTAATCTACATTCCGACAGTGTGATCCG |
| | | CCATTTACGTAAACCGGGCGATACACGCCATTATTTGCCGCAGAAGGGAA |
| | | TGTACCGATATGTCACTTCGGGCAACTATTTTGGCGAGTTGGTGGAATGG |
| | | ATAGGGTTTGCCGTACTCACTTGTTCGCCCGCTGCATGGGTGTTTGTGCT |
| | | GTGGACGTTTGCTAATCTGGCTCCACGTGCCAATTCCATCCGTAACCGTT |
| | | ATCGGGAAGAGTTTGGTAAGGATGCGGTAGGAAAAAAGAAAAGAATGATT |
| | | CCTTTTATTTATTGA |
| 64 | 5α-reductase gene | ATGAGTATAGCTGCCTTTAATCTATTTTTGGGCGTCATGAGTCTGACCGC |
| | from Bacteroides | TCTGATTGTTTTCATCGCCCTCTACTTTGTGAAAGCCGGTTACGGGATAT |
| | thetaiotaomicron | TTCGCACCGCCTCCTGGGGAGTTGCCATTTCCAACAAGCTGGCGTGGATA |
| | VPI 5482 | CTGATGGAAGCCCCCGTATTTCTGGTCATGTGCTGGATGTGGATACACTC |
| | | GGAACGTCGTTTTGATCCGGTCATACTGACATTCTTTGTCTTCTTTCAGA |
| | | TTCATTATTTTCAGCGCGCCTTCGTCTTTCCCCTGCTACTGACCGGAAAG |
| | | AGTAAAATGCCGCTGGCAATCATGTCGATGGGAATCCTGTTCAATCTATT |
| | | GAACGGCTATATGCAGGGTGAATGGATATTTTATCTCTCACCCGAGGGAA |
| | | TGTATCATTCCGGCTGGTTCACTTCCGCATGGTTTATTGCGGGCAGTCTG |
| | | CTTTTCTTTGCGGGCATGTTGATGAACTGGCATTCGGACTATATCATCCG |
| | | CCATTTGCGCAAACCGGGGGATACCCGTCATTATCTGCCACAAAAAGGGA |
| | | TGTACCGCTATGTCACTTCCGCCAATTATCTGGGCGAAATCATTGAATGG |
| | | GCAGGCTGGGCAATACTGACTTGTTCACTATCCGGACTTGTATTCTTCTG |
| | | GTGGACAGTGGCCAATCTCGTCCCCCGTGCCAATGCAATCTGGCATCGCT |
| | | ACCGTGAAGAATTTGGCTCGGAAGTAGGCGAACGCAAACGTGTATTTCCT |
| | | TTTATCTATTGA |
| 65 | 5α-reductase gene | ATGACTATGAATGCATTTAATCTGTTTTTGGGCATAATGAGCCTGATCGC |
| | from Bacteroides | TCTGATTGTTTTTATTGCCCTTTACTTTGTGAAAGCCGGATATGGTATTT |
| | caccae ATCC | TTCGTACTGCTTCGTGGGGTGTGGCTATTTCCAATAAGTTAGCTTGGATA |
| | 43185 | TTAATGGAGGCCCCTGTATTTTTAGTTATGTGTTGGATGTGGGTGCATTC |
| | | GGAACGCCGTTTTGATCCCGTCATACTGATGTTCTTCATATTCTTCCAGA |
| | | TTCATTATTTCCAGCGTGCATTCGTTTTTCCTCTATTGCTGACCGGAAAG |
| | | AGTAAAATGCCGTTAGCTATTATGTCAATGGGCATTCTTTTTAATTTGTT |
| | | GAACGGATATATGCAAGGACAATGGATATTTCATCTTGCGCCTGAAGGAA |
| | | TGTACGGCATTGATTGGTTTATGTCACCATGGTTTATTCTCGGAACTCTG |
| | | CTTTTTTTTACTGGTATGCTGGTGAACTGGCACTCGGATTATATCATCCG |
| | | GCATTTGCGAAAGCCGGGAGATACCCGCCACTATCTGCCTCAAAAAGGGA |
| | | TGTACCGCTACGTTACTTCCGCCAATTACTTCGGCGAAATAGTAGAGTGG |
| | | GCAGGCTGGGCGATACTCACTTGTTCACTTTCCGGACTTGTGTTTCTTTG |
| | | GTGGACGATCGCTAACCTTGTCCCGCGTGCCAACGCAATCTGGCACCGTT |
| | | ACCGCGAGGAATTCGGTGATGAGGTGGGAAATAGGAAACGTGTATTCCCT |
| | | TTTCTGTATTAA |
| 66 | 5α-reductase gene | ATGAATTTAGCAGCTTTTAATCTGTTTTTGGGTGTAATGAGCTTGATTGC |
| | from Bacteroides | CCTGATTGTTTTTGTCGCTCTCTACTTCGTGAAAGCAGGATACGGAATCT |
| | finegoldii DSM | TCCGCACGTCTTCGTGGGGAGCGGCTATTTCAAACAAGCTGGCTTGGATA |
| | 17568 | CTGATGGAAGCTCCGGTCTTCCTCGTGATGTGCGTGATGTGGATGTATTC |
| | | GGAACGCCGCTTTGAGCCGGTGATATTGACCTTCTTTTTATTCTTCCAAC |
| | | TGCATTATTTTCAACGGGCTTTCATTTTCCCTTTGTTATTGAAAGGAAAA |
| | | AGTAAAATGCCGTTGGCCATCATGTCAATGGGAATCCTTTTCAATCTGTT |
| | | GAATGGATATATGCAAGGAGAATGGATTTTCTACCTTGCCCCCGCAACGA |
| | | TGTACCAGTCCGATTGGTTCACCTCCCCGTACTTTATAATAGGTACTTTG |
| | | CTGTTTTTTACGGGTATGCTGGTGAACTGGTCGTCCGATTATATCATCCG |
| | | CCATTTGCGTAAACCGGGAGACACACGGCATTATCTGCCACAGAAAGGTA |
| | | TGTACCGCTATGTGACTTCCGCCAATTATTTCGGTGAAATCGTAGAATGG |
| | | GCAGGCTGGGCAATTCTTACCTGTTCGCTTTCCGGACTTGTTTTCCTTTG |
| | | GTGGACGATTGCAAACCTCGTTCCGCGAGCCGACGCCATCTGGAAACGTT |
| | | ATCGCGAGGAATTCGGCGACGCGGTAGGCACACGGAAGCGGGTGTTTCCT |
| | | TTTCTCTACTAG |
| 67 | 5α-reductase gene | ATGAATCAGGAAACTTTTCAGATATTTCTGTGGGTAATGAGTGCTGTGGC |
| | from Bacteroides | ATTGGTTGTCTTTATTGCACTCTATTTTGTCAAAGCGGGTTATGGCATGT |
| | uniformis ATCC | TCCGTACTGCCTCGTGGGGAATCTCCATCAATAATAAACTGGCGTGGGTG |
| | 8492 | CTTATGGAAGCGCCGGTATTCATCGTCATGTTTGGGTTGTGGGGGAAGAG |
| | | TGGAGCGGGATTTGCCGTGCCGGTATATTTCTTCTTCCTGCTGTTTCAGT |
| | | TGCACTATCTTCAGCGGGCCTTTATTTTTCCGTTCCTGCTGAAAGGTAAA |
| | | AGCCGGATGCCGGTAGCTATTATGGCGATGGGTATCGTCTTCAACCTTTT |
| | | GAACGGGATGATGCAGGCGGGCGGTTTGTTCTATTTCGCTCCCGAAGGCT |
| | | TGTATGCCGATGGCTGGGCCTATTTGCTGAAACCTCATGCCTTGTTGGGA |
| | | ATCATTCTGTTTTTTGCAGGTATGTTCGTCAATTTGCATTCCGACTATGT |
| | | GATACGTCATCTGCGCAGGCCAGGTGATACGAAGCATTATCTTCCCGGAA |
| | | AAGGGCTTTACCGATACGTCACTTCTGCCAATTACTTCGGTGAACTGGTG |
| | | GAATGGACGGGGTTTGCCATACTCACAGCTTCTCCCGCCGCCTGGGTGTT |
| | | CGTCTGGTGGACGTTTGCCAACCTTGTTCCCCGTGCCGATGCCATTCACC |
| | | GCCGTTATCGGGAGGAGTTTGGTGATGAGGCGGTAGGAAAGCGCAAACGC |
| | | ATCATTCCATTTCTTTATTAA |
| 68 | 5β-reductase gene | ATGGAAAAAGAATCTATACTGTTCACTCCCGGTAAAATCGGGCCGTTGAC |
| | from | CCTGAGAAACCGGACGATACGGGCGGCTGCATTTGAAAGCATGTGCCCAG |
| | Parabacteroides | GAAACGCGCCTTCCGACATGTTGTATGACTACCATAAATCGGTTGCCTCC |
| | merdae ATCC | GGCGGGATCGGTATGACGACTTTGGCCTATGCGGCTGTTACGCAAAGCGG |
| | 43184 | ACTTTCTTTCGAACGTCAGCTCTGGATGCGCCCAGATATCATCCCCGGAT |
| | | TAAAACGCATCACCGATGCCATCCACAAAGAAGGAGCGGCCGCCTCCGTA |
| | | CAACTCGGACATTGCGGAAACATGTCTCACAAAAACATCTGCGGGTGCAG |
| | | GCCTATCTCCGCATCCAGCGGTTTCAATATCTATTCCCCTACCCTTGTCC |
| | | GTGGAATGAAACCTTCCGAAATCACAGCTATGGCAAAAGCGTTCGGACAA |
| | | GCAGTTCATCTGGCGCGCGAAGCCGGAATGGATGCAGTGGAAATACATGC |
| | | CGGTCACGGCTATCTGATCAGCCAGTTTCTTTCTCCCTATACCAATCATC |
| | | GGAAAGACGAATATGGCGGTAGCTTGCAAAACCGGATGCGCTTTATGAAA |
| | | ATGTGCATGGACGAAGTGATGAAAGCTGCCGGTCAGGATATGGCAGTGTT |
| | | GGTAAAGATGAATATGCGCGATGGCTTCAAAGGAGGAATGGAGCTTGATG |
| | | AGACACTTGAAGTGGCTCGTACCCTGCAGAACGAATGCGGAGCACACGCT |
| | | TTGATCCTTAGCGGTGGCTTCGTCAGCCGTGCCCCGATGTATGTGATGCG |
| | | GGGTTCCATGCCGATTCATACGATGACGCATTATATGCCTTTCGGCTGGC |
| | | TACCGCTCGGAGTCAAAATGGCCGGACGGTTCATGATCCCGTCTGAGCCG |
| | | TTCAAAGAGGCTTACTTCCTGGAAGATGCCCTAAAATTCAGGGCGGCATT |
| | | GAAAATGCCACTTGTCTATGTAGGCGGTCTGATCTCACGCGAGAAGATAG |
| | | ACGAGGTCTTGAACGACGGTTTCGAATTCGTGAGTATGGCACGTGCCTTG |
| | | CTGAACGATCCGTCATTCGTAAACAAAATGAAGGAAGACGAACATGCCCG |
| | | TTGTGACTGCGGACATAGCAACTATTGCATCGCCCGCATGTATTCCATCG |
| | | AAATGGCATGCCACAAACATATTCAGAACTTGCCCAAAAGCATTGTCAAA |
| | | GAAATAGAGAAATTAGAATATAAGTAA |
| 69 | 5β-reductase gene | ATGATGAACTCTAAATTATTTACTCCCGCCTCTATCGGGCCGCTGACTTT |
| | from Bacteroides | GCGTAACCGTACGATTCGTTCGGCTGCTTTTGAGAGCATGTGTCCGGGCA |
| | dorei DSM 17855 | ATGCGCCGTCCCGGCAATTGAAGGATTATCACTGTTCGGTGGCAGCAGGT |
| | | GGAGTGGGAATGACTACTATTGCTTATGCAGCTGTTACACAGAGTGGCCT |
| | | TTCTTTCGACAGGCAATTGTGGATGCGCCCTGAAATTATACCGGGATTAA |
| | | GGGAAATAACCGATGCGGTTCATAAAGAAGGAGCTGCTGCAAGTATTCAG |
| | | TTGGGACATTGTGGAAATATGTCGCACAAAAGTATTTGTGGGGTAACACC |
| | | CGTAGGAGCTTCTTCCGGTTTTAATCTTTATTCGCCTACTTTCGTGCGTG |
| | | GCTTGCGCAAGGAGGAACTGCCGCAGATGGCAAAGGCATACGGTCAGTCG |
| | | GTCAACTGGGCACGTGAGGCCGGATTTGACGCGGTGGAGATACATGCGGG |
| | | GCATGGCTATCTTATCAGTCAGTTTCTTTCACCTTACACCAATCATCGTA |
| | | AGGACGAGTTCGGTGGCTCGTTGGAGAACCGTATGCGCTTTATGGATATG |
| | | GTAATGGAGGAAGTGATGCGTGCCGCAGGTAATGACATGGCCGTTCTGGT |
| | | AAAAACCAATATGCGTGACGGTTTTAAAGGCGGCATGGAAATAGATGAAG |
| | | CTGTGCAGGTAGCGAAACGGTTGGTACAAGATGGGGCTCATGCGTTGGTG |
| | | CTGAGCGGAGGCTTTGTAAGCAAAGCGCCTATGTATGTCATGCGGGGAGC |
| | | GATGCCTATAAAAAGTATGACACATTATATGAGCTGCTGGTGGCTGAAAT |
| | | ATGGGGTACGTATGGTAGGTAAATGGATGATTCCGACAGTACCTTTTAAA |
| | | GAGGCTTATTTCTTGGAAGATGCGTTAAGATTCAGAACAGAAATAAAGGA |
| | | AATTCCGTTAGTATATGTGGGAGGGCTGGTATCTCGTGAAAAGATAGATG |
| | | AGGTATTGGATGATGGTTTTGAATTTGTACAGATGGGAAGGGCGTTGCTG |
| | | AATGAACCTGGTTTTGTGAATCGGTTGCGGACTGAAGAAAAGGCTCGTTG |
| | | CAATTGCGGTCATAGTAATTATTGTATTGCGAGAATGTACACTATTGATA |
| | | TGGCATGTCACAAACATCTGGAAGAGAAATTACCCCTTTGCTTGGAACGT |
| | | GAAATAGAAAAATTAGAGAACCAATGA |
| 70 | 5β-reductase gene | ATGAACTCTAAATTATTTACTCCCGCTTCTATCGGACCGCTGACTTTGCG |
| | from Bacteroides | TAACCGTACGATTCGTTCGGCTGCTTTTGAGAGTATGTGTCCGGGCAATG |
| | vulgatus ATCC | CTCCGTCCCGGCAATTGAAGGATTATCACTGCTCGGTGGCGGCAGGTGGA |
| | 8489 | GTGGGAATGACTACTATTGCTTATGCAGCAGTTACACAGAGTGGCCTTTC |
| | | TTTCGACAGGCAATTGTGGATGCGCCCTGAAATTATACCGGGATTAAGGG |
| | | AAATAACCGATGCGGTTCATAAAGAAGGGGCTGCCGTAAGCATTCAGTTG |
| | | GGACATTGTGGAAATATGTCGCACAAAAGTATTTGTGGGGTAACACCCAT |
| | | AGGAGCTTCTTCCGGTTTTAATCTTTATTCGCCTACTTTCGTGCGTGGCT |
| | | TGCGCAAGGAGGAACTGCCGCAGATGGCAAAGGCATACGGTCAGTCGGTC |
| | | AACTGGGCGCGTGAGGCCGGATTTGACGCGGTGGAGATACATGCGGGGCA |
| | | TGGCTATCTTATCAGTCAGTTTCTTTCACCTTACACCAATCATCGTAAAG |
| | | ACGAGTTCGGCGGCTCGTTGGAGAATCGTATGCGCTTTATGGATATGGTG |
| | | ATGGAGGAAGTGATGCGTGCCGCAGGTAATGACATGGCCGTTCTGGTAAA |
| | | AACCAATATGCGTGACGGTTTTAAAGGAGGAATGGAAATAGATGAAGCTG |
| | | TGCAGGTAGCGAAACGGTTGGTACAGGACGGGGCTCATGCGTTGGTGCTG |
| | | AGTGGAGGTTTTGTAAGCAAAGCGCCTATGTATGTCATGCGGGGAGCGAT |
| | | GCCTATAAAAAGTATGACACATTATATGAACTGTTGGTGGCTGAAATATG |
| | | GGGTGCGTATGGTAGGCAAATGGATGATTCCGACAGTACCTTTCAAAGAG |
| | | GCTTATTTTTTGGAAGATGCATTGAGATTCAGAACAGAAATAAAGGAAAT |
| | | TCCGTTAGTATATGTGGGGGGGCTGGTATCTCGTGAAAAGATAGATGAGG |
| | | TATTGGATGATGGTTTTGAATTTGTACAGATGGGAAGGGCGTTGCTGAAT |
| | | GAACCTGGTTTTGTGAATCGGTTGCGCACTGAAGAAAAGGCGCGTTGCAA |
| | | TTGCGGTCATAGTAATTATTGCATTGCGAGAATGTACACTATTGATATGG |
| | | CATGCCACAAACATTTGGAAGAGAAATTGCCCCTTTGCTTGGAACGTGAA |
| | | ATAGAAAAATTAGAGAACCAATGA |
| 71 | 5β-reductase gene | ATGGAATCTAAACTTTTCACCCCTGTTACTTTCGGTCCATTGACGCTGCG |
| | from Bacteroides | GAATCGTACGATCCGCTCTGCCGCTTTTGAAAGCATGTGTCCCGGTAACG |
| | thetaiotaomicron | CCCCTTCACAAATGCTGCTCGATTACCACCGCTCGGTGGCTGCGGGCGGA |
| | VPI 5482 | GTCGGGATGACGACTGTAGCCTATGCAGCCGTGACACAAAGCGGACTTTC |
| | | CTTCGACCGTCAGTTGTGGCTGCGTCCGGAAATCATTTCCGGTTTGCGTG |
| | | AAGTGACCGGAGCTATACACACGGAAGGTGCGGCAGCAGGCATCCAGATA |
| | | GGGCACTGCGGAAATATGTCCCATAAAAAGATTTGCGGAACCACTCCCAT |
| | | TTCAGCTTCTACCGGTTTCAATCTCTATTCTCCTACATTCGTGCGTGGCA |
| | | TGAAGAGAGAAGAGTTGCCGGAGATGGCCAGAGCCTACGGACGGGCTGTC |
| | | CACTTGGCACGGGAAGCCGGCTTCGACGCCGTCGAGGTACACGCCGGACA |
| | | CGGATATCTGATCAGCCAGTTCCTGTCTCCCTACACCAATCACCGGAAAG |
| | | ACGACTACGGCGGCTTGCTCCAAAACCGGATGCGCTTTATGGAAATGGTG |
| | | ATGAACGAGGTGATGACAGCGGCGGGAAGCGACATGGCAGTCATTGTAAA |
| | | AATGAATATGCGCGATGGCTTCAAAGGCGGCATGGAAACCGATGAATCTC |
| | | TGCAAGTGGCCAAACGCCTGCTGGCATTGGGCGCGCACGCATTGGTACTG |
| | | AGCGGAGGATTCGTCAGCAAGGCCCCGATGTACGTCATGCGGGGAGCGAT |
| | | GCCGATTCGTTCGATGGCTTACTACATGGACTGCTGGTGGCTGAAATACG |
| | | GAGTCCGGATGTTTGGAAAGTGGATGATTCCGACCGTTCCTTTCCGGGAA |
| | | GCCTATTTCCTGGAGGATGCACTGAAATTTCGGGCGGCACTTCCGGAAGC |
| | | CCCGTTGATTTATGTGGGCGGACTGGTGTCCCGCGAAAAGATAGATGAAG |
| | | TATTGGATGCCGGCTTCGATGCCGTCCAGATGGCACGTGCGTTGCTCAAC |
| | | GAACCGGAGTTTGTCAACCGGATGAGGCGGGAAGAGCAGGCGCGCTGTAA |
| | | CTGCGGACACAGCAACTACTGCATCGGGCGGATGTACACAATCGAAATGG |
| | | CCTGCCACCAACATCTGAAAGAAAAACTGCCTCCCTGCCTGCAACGGGAG |
| | | ATTGAAAAACTGGAAAAGCAATGA |
| 72 | 5β-reductase gene | ATGAAATCAAAGTTATTTACCCCGGTCACTTTTGGACCTTTGACACTTCG |
| | from Bacteroides | CAACCGGACCATTCGTTCGGCAGCTTTTGAAAGTATGTGTCCGGGAAACG |
| | caccae ATCC | CCCCTTCACAAATGTTGTTGGATTATCACCGTTCGGTAGCCGCAGGCGGG |
| | 43185 | GTTGGCATGACGACGGTTGCTTATGCTGCCGTGACACAAAGCGGGCTTTC |
| | | TTTTGACCGCCAATTATGGTTACGATCTTCCATAATTCCCGGTTTACGCG |
| | | AAGTGACCGACGCCATACACGATGAGGGTGCCGCAGCAGGTATACAAATC |
| | | GGTCATTGCGGTAATATGTCCCACAAGAACATTTGTGGAGTAACTCCTAT |
| | | CTCTGCTTCTTCCGGTTTCAATCTTTACTCTCCGACCTTTGTGCGCGGAA |
| | | TGAGGAAAGAAGAACTTCCTGAAATGGCGCGTGCTTATGGAAAGGCTGTC |
| | | AATCTGGCTAGAGAAGCTGGTTTCGATGCTGTTGAGGTTCACGCCGGACA |
| | | TGGATATCTGATTAGCCAGTTCCTTTCCCCTTACACGAATCACCGGAAGG |
| | | ATGAATACGGAGGTTCGTTGGAAAACCGGATGCGTTTTATGGATATAGTG |
| | | ATGAAAGAAGTGATGAAAGCTGCCGGTAGCGATATGGCGGTTCTTGTGAA |
| | | AATGAATATGCGCGACGGTTTCAAAGGTGGAATGGAGTCGGAGGAAACGA |
| | | TACAAGTAGCCCGACGCCTGCTCGAACTGGGTGCTCACGGGCTGGTATTG |
| | | AGCGGTGGTTTCGTCAGTCGTGCGCCGATGTATGTGATGCGAGGGGCGAT |
| | | GCCTATCCGTTCTATGGCTTATTATATGAATTGCTGGTGGCTGAAATATG |
| | | GCGTCCGGATGTTCGGAAAATGGATGATTCCGACTGTTCCTTTCAAAGAG |
| | | GCATATTTTCTCGAAGATGCGCTGAAGTTCCGTGAGGCCCTTCCGGGTGC |
| | | TCCGTTAATTTATGTAGGCGGCCTTGTTTCTCGTGAGAAAATTGATGAAG |
| | | TGCTGGATGCCGGCTTTGATGCTGTTCAAATGGCACGTGCCCTGCTGAAT |
| | | GAACCGGGATTTGTGAACCGTATGGCACGTGAAGACCGTGCACGTTGTAA |
| | | TTGCGGGCATAGCAATTATTGCATTGGCCGTATGTACACGAACGAAATGG |
| | | CTTGTCATAAACATTTGAATGAAGAACTGCCTCCTTGTCTGCAAAAGGAG |
| | | ATTGAAAAATTGGAAAAACAATGA |
| 73 | 5β-reductase gene | ATGAAATCTAAACTTTTCACTCCGGTCACTTTCGGCCCTTTGACGTTGCG |
| | from Bacteroides | GAACAGGACGATACGTTCGGCAGCGTTTGAAAGTATGTGTCCGGGCAATA |
| | finegoldii DSM | CTCCCTCGCAGATGCTATTGGATTACCACCGCTCGGTTGCTGCGGGAGGA |
| | 17568 | GTGGGGATGACGACTGTAGCTTATGCCGCCGTGACGCAAAGCGGGCTCTC |
| | | TTTCGATCGTCAGCTATGGATGCGTCCGTCCATAATTTCCCGTTTGAATG |
| | | AATTGACAAAAGCCGTGCACGACGAGGGTGCTGCGGTAGGTATCCAGATA |
| | | GGGCATTGCGGAAATATGTCCCACAAAAAGATTTGCGGTGTCACGCCCAT |
| | | ATCCGCTTCTTCAGGTTTTAACCTCTATTCGCCTACGTTTGTGCGTGGAA |
| | | TGGAACGGGAAGAACTTCCGGAAATGGCACAGGCTTACGGAAACGCTGTC |
| | | AACTTGGCACGGGAAGCGGGATTTGATGCGGTGGAAGTGCACGCTGGACA |
| | | CGGTTATTTAATCAGCCAATTCCTTTCACCTTACACCAACCATCGCAAAG |
| | | ACGAGTTCGGAGGCTCGCTGGAGAACCGGATGCGTTTTATGGATCTGGTG |
| | | ATGGAAGAGGTGATGAAAGCGGCGGGCAATGACATGGCGGTGATTGTCAA |
| | | AATGAATATGCGCGACGGTTTCAAAGGCGGAATGGAAATAGACGAATCCA |
| | | TTCAGGTGGCGAAACGGCTTCTGGAGCTGGGTGCTCACGGACTGGTGCTG |
| | | AGTGGAGGCTTTGTCAGCAAAGCCCCGATGTATGTGATGCGGGGAGCGAT |
| | | GCCGATTCGCTCGATGGCGCACTATATGGACTGTTGGTGGCTGAAATACG |
| | | GTGTCCGGATGTTCGGAAAATGGATGATTCCGTCCGTGCCTTTTGAGGAG |
| | | GCTTATTTCCTGAAAGACGCCTTAAAGTTCCGGGAAGCTCTTCCGGAAGC |
| | | TCCCTTGATTTATGTTGGCGGACTCGTTGCCCGTCAGAAGATAGATGAAG |
| | | TGCTCGATGCGGGCTTTGAAGCTGTACAGATGGGACGTGCCTTACTCAAC |
| | | GAACCTGGATTTGTGAACCGGATGAAGCAGGAGGAGCAGGCCCGTTGCAA |
| | | CTGTGGACACAGTAATTACTGCATAGGCCGTATGTATTCGATAGAAATGG |
| | | CCTGCCATCAACATTTAAAAGAAACGTTACCTCCTTGTCTGCAAAAGGAG |
| | | ATTGAAAAATTGGAAAAGAAATGA |
| 74 | 5β-reductase gene | ATGGAATCCAAGTTATTCACCCCCGTCACTTTCGGACCGTTGACTTTGCG |
| | from Bacteroides | TAACCGTACCATCCGTTCGGCTGCTTTCGAGAGCATGTGTCCCGGAAACG |
| | uniformis ATCC | CTCCGTCGGACATGTTGCTCGACTATCATCGGTCGGTAGCGGCGGGCGGT |
| | 8492 | ATAGGTATGACTACTGTTGCTTATGCGGCAGTTACTCAGAGCGGCTTGTC |
| | | TTTTGACCGCCAGTTGTGGATGCGTCCTGAGATTATCCCCGGACTCCGTA |
| | | GGCTGACTGATGCCATACATGCGGAGGGGGCTGCGGCAGGCATCCAGTTG |
| | | GGACACTGTGGCAATATGTCCCATAAGAGTATTTGCGGTTGTATGCCTGT |
| | | CGGCGCATCTTCCGGTTTCAATCTTTATTCTCCTACTTTTGTGCGCGGAC |
| | | TTCGTGCCGATGAGCTGCCGGAGATGGCGCGCGCTTATGGACGTTCGGTG |
| | | AATCTGGCACGGGAGGCCGGATTTGATTCTGTAGAGATTCATGCAGGGCA |
| | | TGGCTATCTCATCAGCCAGTTCTTGTCCCCGTCCACCAACCATCGGAAAG |
| | | ACGAGTTCGGAGGCTCGTTGCAGAACCGTATGCGTTTTATGGATATGGTG |
| | | ATGGAAGAAGTGATGAAGGCTGCCGGCAGTGATATGGCTGTGCTGGTGAA |
| | | GATGAACATGCGTGACGGTTTCCGTGGAGGGATGGAACTGGATGAGACGA |
| | | TGCAGGTGGCCCGGAGGCTGGAGCAGTCGGGAGCACATGCGCTGGTCTTG |
| | | AGCGGTGGGTTCGTCAGTAAGGCGCCGATGTATGTCATGCGGGGCGAAAT |
| | | GCCTATCCGCAGCATGACGCATTACATGACCTGCTGGTGGTTGAAATACG |
| | | GTGTACGCATGGTAGGCAAATGGATGATACCGAGCGTACCGTTTAAGGAA |
| | | GCCTATTTCCTGGAAGATGCCTTGAAATTCCGTGCCGCCTTGAAGATACC |
| | | GTTGGTTTATGTGGGTGGCCTGGTTTCCAGGGACAAGATAGATGAGGTGC |
| | | TGGATGACGGTTTCGAGGCTGTGCAAATGGCACGTGCTCTGCTCAATGAG |
| | | CCGGGGTTCGTCAACCGCATGCGTGCCGAAGAGAATGCACGTTGCAACTG |
| | | CCGGCATAGTAATTATTGCATTGCCCGGATGTATTCCATCGAGATGGTAT |
| | | GCCATCAGCATTTGAAGGAGGAGCTTCCACCTTGTCTGAAAAAGGAGATA |
| | | GAGAAAATCGAAGCGAAAGGCTGA |
| 75 | SULT2A1 from | TGTACCGGTATTTCATTCCTCGCTGAAGATGGCGGATATGTGCAGGCACG |
| | Homo sapiens | TACTATAGAGTGGGGGAACAGTTATCTTCCGAGTGAATATGTTATTGTTC |
| | | CCAGAGGACAGGATTTGGTATCTTATACTCCAACGGGTGTAAATGGCTTG |
| | | AGATTTCGGGCTAAATATGGTCTGGTAGGACTGGCTATCATTCAGAAAGA |
| | | GTTTGTGGCTGAAGGACTGAATGAAGTAGGGCTTTCGGCTGGATTGTTTT |
| | | ATTTTCCCCATTATGGGAAGTATGAAGAATATGATGAGGCTCAAAATGCA |
| | | ATTACTTTGTCGGATTTGCAGGTGGTGAACTGGATGCTTTCCCAATTTGC |
| | | TACTATAGACGAAGTGAGAGAAGCTATAGAAGGGGTGAAGGTGGTGTCTC |
| | | TTGATAAACCTGGTAAAAGTTCTACGGTACATTGGCGCATTGGCGATGCT |
| | | AAAGGAAATCAAATGGTGTTGGAATTTGTAGGTGGTGTTCCTTATTTTTA |
| | | TGAAAATAAAGTAGGAGTACTCACCAATTCTCCCGATTTTCCATGGCAGG |
| | | TGATTAACTTGAATAATTATGTAAATCTATATCCGGGAGCTGTCACTCCA |
| | | CAGCAATGGGGTGGGGTGACTATTTTCCCTTTTGGCGCAGGTGCCGGATT |
| | | TCATGGTATTCCGGGGGATGTAACTCCTCCATCCCGTTTTGTTCGTGTAG |
| | | CGTTTTATAAGGCAACAGCTCCGGTGTGTCCTACAGCGTATGACGCTATA |
| | | TTACAAAGCTTTCATATCCTGAATAATTTTGATATTCCTATTGGTATAGA |
| | | ATATGCGTTAGGGAAAGCACCTGATATTCCTAGTGCCACACAATGGACTT |
| | | CGGCTATTGATTTGACAAACAGGAAAGTGTATTATAAAACAGCATACAAT |
| | | AACAATATTCGTTGTATTAGTATGAAGAAGATTGATTTTGATAAAGTGAA |
| | | GTATCAGTCGTATCCATTGGATAAGGAGTTGAAACAGCCTGTAGAAGAGA |
| | | TTATTGTGAAATAA |
| 76 | SULT2A8 from | ACGGACGAGTTCCTGTGGATTGAAGGCATACCCTTTCCCACTGTATATTA |
| | Mus musculus | CTCTCAGGAAATTATACGTGAGGTGCGTGATCGTTTTGTTGTAAGAGATG |
| | | AAGACACCATCATTGTCACATACCCCAAGTCGGGAACTCATTGGCTGAAT |
| | | GAGATCGTGTGCTTGATATTAACAAAAGGCGATCCGACCTGGGTACAGAG |
| | | CACAATTGCAAATGAACGTACTCCTTGGATTGAATTCGAAAACAACTATC |
| | | GCATCCTGAATTCAAAGGAAGGTCCTCGTTTGATGGCAAGCTTGTTGCCT |
| | | ATACAGCTGTTTCCTAAAAGCTTTTTTAGTAGCAAAGCCAAAGTCATATA |
| | | CTTAATCCGTAATCCACGCGACGTACTGGTATCCGGTTATCACTATTTCA |
| | | ATGCCCTTAAGCAGGGCAAGGAGCAAGTTCCATGGAAAATCTACTTCGAA |
| | | AATTTTCTGCAAGGAAAGAGTTACTTTGGGTCATGGTTCGAGCATGCGTG |
| | | CGGCTGGATCTCGCTTCGTAAGCGTGAAAATATACTGGTGTTGTCGTACG |
| | | AACAATTGAAAAAGGATACACGTAACACTATAAAGAAAATTTGTGAATTT |
| | | CTTGGGGAGAACTTAGAATCAGGAGAACTGGAGCTGGTATTAAAGAATAT |
| | | AAGCTTCCAAATCATGAAAGAACGGATGATCTCACAGTCATGCTTAAGTA |
| | | ATATTGAGAAGCATGAATTCATTATGCGTAAAGGCATTACAGGGGATTGG |
| | | AAAAATCATTTTACCGTAGCTCAGGCCGAAGCCTTTGATAAAGCTTTCCA |
| | | AGAAAAAGCAGCAGACTTCCCACAGGAGTTATTTTCTTGGGAATAA |
| 77 | P_BfP1E6 | GATAAAACGAAAGGCTCAGTCGAAAGACTGGGCCTTTCGTTTTACAATTG |
| | | GGCTACCTTTTTTTTGTTTTGTTTGCAATGGTTAATCTATTGTTAAAATT |
| | | TAAAGTTTCACTTGAACTTTCAAATAATGTTCTTATATTTGCAGTGTCGA |
| | | AAGAAACAAAGTAG |
| 78 | P_BfP4E5 | GATAAAACGAAAGGCTCAGTCGAAAGACTGGGCCTTTCGTTTTACAATTG |
| | | GGCTACCTTTTTTTTGTTTTGTTTGCAATGGTTAATCTATTGTTGAAATT |
| | | TAAAGTTTCACTTGAACTTTCAAATAATGTTCTTATATTTGCAGTGTCGA |
| | | AAGAAACAAAGTAG |
| 79 | Phage promoter | GTTAA(n)₄₋₇GTTAA(n)₃₄₋₃₈TA(n)₂TTTG |
| | consensus | |
| 80 | Phage promoter | GTTAAnnnnnnnGTTAAnnnnnnnnnnnnnnnnnnnnnnnnnnnnnnnnn |
| | | nnnnTAnnTTTG |
| 81 | consensus | TCCGTCTCAGACTGCTATGACTTGATACCGGCTATTACGAGCGCTTAAAC |
| | Bacteroides NBU | GGCGCGCCTGATAGGTGGGCTGCCCTTCCTGGTTGGCTTGGTTTCATCAG |
| | integration vector | CCATCCGCTTGCCCTCATCTGTTACGCCGGCGGTAGCCGGCCAGCCTCGC |
| | | AGAGCAGGATTCCCGTTGAGCACCGCCAGGTGCGAATAAGGGACAGTGAA |
| | | GAAGGAACACCCGCTCGCGGGTGGGCCTACTTCACCTATCCTGCCCGGCT |
| | | GACGCCGTTGGATACACCAAGGAAAGTCTACACGAACCCTTTGGCAAAAT |
| | | CCTGTATATCGTGCGAAAAAGGATGGATATACCGAAAAAATCGCTATAAT |
| | | GACCCCGAAGCAGGGTTATGCAGCGGAAAAGCGGGATTAAAAGTCGGGGA |
| | | TTGGTGAACAAAAAGGTGTTTCTCTCTTTAAGAGAAATATCGTTTTGCTA |
| | | AACAGTTGATATTGAGGTATCATTTTATCGTAAAAGACATTTTTGCTCAA |
| | | CAATTGCTTGACGGAAATCAACAAATTTTAGCATTTTGTAAAAAAGTCGC |
| | | TATATAATTTGGTGAATTGGAGTTATTTTCATATTTTTGCATCCCGAAGA |
| | | GTTTCTCTTAAAGAGAGAAACATCTTTTGCATACCTTTTCCGACCGAATT |
| | | TTTATGTCGTAAAGAGGGGCTTTGCAGGGGGTGGACTCAGAAAGATGAGA |
| | | ATAGATGACTATTGTAGTTGAAACACATAGAAAGTTGCTGATATACAGAC |
| | | CGATACGCATATCGGGATGAACCATGAGTACGTTCTTTTCTCAAAAAACA |
| | | TAAATATTCGAAAAGAGATGCAATAAATTAAGGAGAGGTTATAATGAACA |
| | | AAGTAAATATAAAAGATAGTCAAAATTTTATTACTTCAAAATATCACATA |
| | | GAAAAAATAATGAATTGCATAAGTTTAGATGAAAAAGATAACATCTTTGA |
| | | AATAGGTGCAGGGAAAGGTCATTTTACTGCTGGATTGGTAAAGAGATGTA |
| | | ATTTTGTAACGGCGATAGAAATTGATTCTAAATTATGTGAGGTAACTCGT |
| | | AATAAGCTCTTAAATTATCCTAACTATCAAATAGTAAATGATGATATACT |
| | | GAAATTTACATTTCCTAGCCACAATCCATATAAAATATTTGGCAGCATAC |
| | | CTTACAACATAAGCACAAATATAATTCGAAAAATTGTTTTTGAAAGTTCA |
| | | GCCACAATAAGTTATTTAATAGTGGAATATGGTTTTGCTAAAATGTTATT |
| | | AGATACAAACAGATCACTAGCATTGCTGTTAATGGCAGAGGTAGATATTT |
| | | CTATATTAGCAAAAATTCCTAGGTATTATTTCCATCCAAAACCTAAAGTG |
| | | GATAGCACATTAATTGTATTAAAAAGAAAGCCAGCAAAAATGGCATTTAA |
| | | AGAGAGAAAAAAATATGAAACTTTTGTAATGAAATGGGTTAACAAAGAGT |
| | | ACGAAAAACTGTTTACAAAAAATCAATTTAATAAAGCTTTAAAACATGCG |
| | | AGAATATATGATATAAACAATATTAGTTTCGAACAATTTGTATCGCTATT |
| | | TAATAGTTATAAAATATTTAACGGCTAAAAACAATAGGCCACATGCAACT |
| | | GTAAATGTTTACGCGGGTACCGACACCGCGGTGGAGGGGAATTGTGTTAC |
| | | AACCAATTAACCAATTCTGATTAGAAAAACTCATCGAGCATCAAATGAAA |
| | | CTGCAATTTATTCATATCAGGATTATCAATACCATATTTTTGAAAAAGCC |
| | | GTTTCTGTAATGAAGGAGAAAACTCACCGAGGCAGTTCCATAGGATGGCA |
| | | AGATCCTGGTATCGGTCTGCGATTCCGACTCGTCCAACATCAATACAACC |
| | | TATTAATTTCCCCTCGTCAAAAATAAGGTTATCAAGTGAGAAATCACCAT |
| | | GAGTGACGACTGAATCCGGTGAGAATGGCAAAAGCTTATGCATTTCTTTC |
| | | CAGACTTGTTCAACAGGCCAGCCATTACGCTCGTCATCAAAATCACTCGC |
| | | ATCAACCAAACCGTTATTCATTCGTGATTGCGCCTGAGCGAGGCGAAATA |
| | | CGCGATCGCTGTTAAAAGGACAATTACAAACAGGAATCGAATGCAACCGG |
| | | CGCAGGAACACTGCCATGAGACGTCGATTATCAAAAAGGATCTTCACCTA |
| | | GATCCTTTTAAATTAAAAATGAAGTTTTAAATCAATCTAAAGTATATATG |
| | | AGTAAACTTGGTCTGACAGTTACCAATGCTTAATCAGTGAGGCACCTATC |
| | | TCAGCGATCTGTCTATTTCGTTCATCCATAGTTGCCTGACTCCCCGTCGT |
| | | GTAGATAACTACGATACGGGAGGGCTTACCATCTGGCCCCAGTGCTGCAA |
| | | TGATACCGCGGGACCCACGCTCACCGGCTCCAGATTTATCAGCAATAAAC |
| | | CAGCCAGCCGGAAGGGCCGAGCGCAGAAGTGGTCCTGCAACTTTATCCGC |
| | | CTCCATCCAGTCTATTAATTGTTGCCGGGAAGCTAGAGTAAGTAGTTCGC |
| | | CAGTTAATAGTTTGCGCAACGTTGTTGCCATTGCTACAGGCATCGTGGTG |
| | | TCACGCTCGTCGTTTGGTATGGCTTCATTCAGCTCCGGTTCCCAACGATC |
| | | AAGGCGAGTTACATGATCCCCCATGTTGTGCAAAAAAGCGGTTAGCTCCT |
| | | TCGGTCCTCCGATCGTTGTCAGAAGTAAGTTGGCCGCAGTGTTATCACTC |
| | | ATGGTTATGGCAGCACTGCATAATTCTCTTACTGTCATGCCATCCGTAAG |
| | | ATGCTTTTCTGTGACTGGTGAGTACTCAACCAAGTCATTCTGAGAATAGT |
| | | GTATGCGGCGACCGAGTTGCTCTTGCCCGGCGTCAATACGGGATAATACC |
| | | GCGCCACATAGCAGAACTTTAAAAGTGCTCATCATTGGAAAACGTTCTTC |
| | | GGGGCGAAAACTCTCAAGGATCTTACCGCTGTTGAGATCCAGTTCGATGT |
| | | AACCCACTCGTGCACCCAACTGATCTTCAGCATCTTTTACTTTCACCAGC |
| | | GTTTCTGGGTGAGCAAAAACAGGAAGGCAAAATGCCGCAAAAAAGGGAAT |
| | | AAGGGCGACACGGAAATGTTGAATACTCATACTCTTCCTTTTTCAATATT |
| | | ATTGAAGCATTTATCAGGGTTATTGTCTCATGAGCGGATACATATTTGAA |
| | | TGTATTTAGAAAAATAAACAAATAGGGGTTCCGCGCACATTTCCCCGAAA |
| | | AGTGCCACCTGTCATGACCAAAATCCCTTAACGTGAGTTTTCGTTCCACT |
| | | GAGCGTCAGACCCCGTAGAAAAGATCAAAGGATCTTCTTGAGATCCTTTT |
| | | TTTCTGCGCGTAATCTGCTGCTTGCAAACAAAAAAACCACCGCTACCAGC |
| | | GGTGGTTTGTTTGCCGGATCAAGAGCTACCAACTCTTTTTCCGAAGGTAA |
| | | CTGGCTTCAGCAGAGCGCAGATACCAAATACTGTTCTTCTAGTGTAGCCG |
| | | TAGTTAGGCCACCACTTCAAGAACTCTGTAGCACCGCCTACATACCTCGC |
| | | TCTGCTAATCCTGTTACCAGTGGCTGCTGCCAGTGGCGATAAGTCGTGTC |
| | | TTACCGGGTTGGACTCAAGACGATAGTTACCGGATAAGGCGCAGCGGTCG |
| | | GGCTGAACGGGGGGTTCGTGCACACAGCCCAGCTTGGAGCGAACGACCTA |
| | | CACCGAACTGAGATACCTACAGCGTGAGCTATGAGAAAGCGCCACGCTTC |
| | | CCGAAGGGAGAAAGGCGGACAGGTATCCGGTAAGCGGCAGGGTCGGAACA |
| | | GGAGAGCGCACGAGGGAGCTTCCAGGGGGAAACGCCTGGTATCTTTATAG |
| | | TCCTGTCGGGTTTCGCCACCTCTGACTTGAGCGTCGATTTTTGTGATGCT |
| | | CGTCAGGGGGGCGGAGCCTATGGAAAAACGCCAGCAACGCGGCCTTTTTA |
| | | CGGTTCCTGGCCTTTTGCTGGCCTTTTGCTCACATGTTCTTTCCTGCGTT |
| | | ATCCCCTGATTCTGTGGATAACCGTAGTCCGTCTCAGCCAGCGCATCAAC |
| | | AATATTTTCACCTGAATCAGGATATTCTTCTAATACCTGGAATGCTGTTT |
| | | TCCCGGGGATCGCAGTGGTGAGTAACCATGCATCATCAGGAGTACGGATA |
| | | AAATGCTTGATGGTCGGAAGAGGCATAAATTCCGTCAGCCAGTTTAGTCT |
| | | GACCATCTCATCTGTAACATCATTGGCAACGCTACCTTTGCCATGTTTCA |
| | | GAAACAACTCTGGCGCATCGGGCTTCCCATACAATCGATAGATTGTCGCA |
| | | CCTGATTGCCCGACATTATCGCGAGCCCATTTATACCCATATAAATCAGC |
| | | ATCCATGTTGGAATTTAATCGCGGCCTGGAGCAAGACGTTTCCCGTTGAA |
| | | TATGGCTCATAACACCCCTTGTATTACTGTTTATGTAAGCAGACAGTTTT |
| | | ATTGTTCATGATGATATATTTTTATCTTGTGCAATGTAACATCAGAGATT |
| | | TTGAGACACAACGTGGCTTTGTTGAATAAATCGAACTTTTGCTGAGTTGA |
| | | AGGATCAGGTCATTGGTAACTATCTATGAAACTGTTTGATACTTTTATAG |
| | | TTGATTAAACTTGTTCATGGCATTTGCCTTAATATCATOCGCTATGTCAA |
| | | TGTAGGGTTTCATAGCTTTGTAGTCGCTGTGTCCCGTCCATTTCATGACC |
| | | ACCTGTGCCGGGATTCCGAGAGCCAGCGCATTGCAGATGAATGTCCTTTT |
| | | TCCTGCATGGGTACTGAGCAAAGCGTATTTGGGTGTGACTTCATCAATAC |
| | | GTTCATTTCCCTTGTAGTAGGTTTCCCGTACAGGCTCGTTGATTTCTGCC |
| | | AGTTCGCCCAGCTCTTTCAGGTAATCGTTCATCTTCTGGTTGCTGATGAC |
| | | GGGCAGAGCCATGTAATTCTCGAAATGGATGTCCTTGTATTTGTCCAGTA |
| | | TGGCTTTGCTGTATTTGTTCAGTTCAATCGTCAGGCTGTCGGCAGTCTTG |
| | | ACTGTGGTTATTTCGATGTGGTCGGACTTCACATCGCTTCTTTTCAGATT |
| | | GCGAACATCCGAATACCGCAAACTCGTAAAGCAGCAGAACAGGAAAACAT |
| | | CACGCACACGTTCCAGGTATTGCTTATCCTTGGGTATCTGGTAGTCTTTC |
| | | AGCTTGTTCAGTTCATCCCAAGTCAGGAAGATTACTTTTTTCGAGGTGGT |
| | | TTTCAGTTTCGGTTTGAACGTATCGTATGCAATGTTCTGATGATGTCCTT |
| | | TCTTGAAGCTCCAGCGCAGGAACCATTTGAGGAATCCCATTTGCTTGCCG |
| | | ATGGTGCTGTTTCTCATATCCTTGGTGTCACGCAGGAAGTTGACGTATTC |
| | | GTTCAATCCAAACTCGTTGAAATAGTTGAACGTTGCATCCTCCTTGAACT |
| | | CTTTGAGGTGGTTCCTCACTGCTGCAAATTTTTCATAGGTGGATGCCGTC |
| | | CAGTTATTCTGGTTACCGCACTCTTTTACAAACTCATCGAACACCTCCCA |
| | | AAAGCTGACAGGGGCTTCTTCCGGCTGTTCTTCACTGGTATCTTTCATTC |
| | | TCATGTTGAAAGCTTCCTTCAACTGTTGGGTCGTTGGCATGACCTCCTGC |
| | | ACCTCAAATTCCTTGAAAATATTCTGGATTTCGGCATAGTATTTCAGCAA |
| | | GTCCGTATTGATTTCGGCTGCACTTTGCTTTAGCTTGTTGGTACATCCGT |
| | | TCTTTACCCGCTGCTTATCTGCATCCCATTTGGCTACGTCAATCCGGTAG |
| | | CCCGTTGTAAACTCGATACGTTGGCTGGCAAAGATGACACGCATACGGAT |
| | | GGGTACGTTCTCTACGATTGGCACACCGTTCTTTTTCCGGCTCTCCAATG |
| | | CAAAAATGATGTTGCGCTTGATATTCATAATTGGGTGCGTTTGAAATTCT |
| | | ACACCCAAATATACACCCAATTATTGAGATAGCAAAAGACATTTAGAAAC |
| | | ATTTACTTTTACTCTATATTGTAATTTACACTTGATTATCAGTCGTTTGC |
| | | AGTCTTATGATATTCTGTGAAAGTATAAGTTCGAGAGCCTGTCTCTCCGC |
| | | AAAAAACGCTGAAAATCAGCAGATTGCAAAACAAACACCCTGTTTTACAC |
| | | CCAAGAATGTAAAGTCGGGTGTTTTTGTTTTATTTAAGATAATACAACCA |
| | | CTACATAATAAAAGAGTAGCGATATTAAAAGAATCCGATGAGAAAAGACT |
| | | AATATTTATCTATCCATTCAGTTTGATTTCTCAGGACTTTACATCGTCCT |
| | | GAAAGTATTTGTTGCAGTTCAACCTGTTGATAGTACGTACTAAGCTCTCA |
| | | TGTTTCACGTACTAAGCTCTCATGTTTAACGTACTAAGCTCTCATGTTTA |
| | | ACGAACTAAACCCTCATGGCTAACGTACTAAGCTCTCATGGCTAACGTAC |
| | | TAAGCTCTCATGTTTCACGTACTAAGCTCTCATGTTTGAACAATAAAATT |
| | | AATATAAATCAGCAACTTAAATAGCCTCTAAGGTTTTAAGTTTTATAAGA |
| | | AAAAAAAGAATATATAAGGCTTTTAAAGCTTTTAAGGTTTAACGGTTGTG |
| | | GACAACAAGCCAGGGATGTAACGCACTGAGAAGCCCTTAGAGCCTCTCAA |
| | | AGCAATTTTGAGTGACACAGGAACACTTAACGGCTGACATGGGGCGGCCG |
| | | CACGACGTACCGGACTCAGTAGGGAGAGCTGTATGTGGGTAGTGAGACGT |
| | | CGATTATCAAAAAGGATCTTCACCTAGATCCTTTTAAATTAAAAATGAAG |
| | | TTTTAAATCAATCTAAAGTATATATGAGTAAACTTGGTCTGACAGTTACC |
| | | AATGCTTAATCAGTGAGGCACCTATCTCAGCGATCTGTCTATTTCGTTCA |
| | | TCCATAGTTGCCTGACTCCCCGTCGTGTAGATAACTACGATACGGGAGGG |
| | | CTTACCATCTGGCCCCAGTGCTGCAATGATACCGCGGGACCCACGCTCAC |
| | | CGGCTCCAGATTTATCAGCAATAAACCAGCCAGCCGGAAGGGCCGAGCGC |
| | | AGAAGTGGTCCTGCAACTTTATCCGCCTCCATCCAGTCTATTAATTGTTG |
| | | CCGGGAAGCTAGAGTAAGTAGTTCGCCAGTTAATAGTTTGCGCAACGTTG |
| | | TTGCCATTGCTACAGGCATCGTGGTGTCACGCTCGTCGTTTGGTATGGCT |
| | | TCATTCAGCTCCGGTTCCCAACGATCAAGGCGAGTTACATGATCCCCCAT |
| | | GTTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAAT |
| | | TCTCTTACTGTCATGCCATCCGTAAGATGCTTTTCTGTGACTGGTGAGTA |
| | | CTCAACCAAGTCATTCTGAGAATAGTGTATGCGGCGACCGAGTTGCTCTT |
| | | GCCCGGCGTCAATACGGGATAATACCGCGCCACATAGCAGAACTTTAAAA |
| | | GTGCTCATCATTGGAAAACGTTCTTCGGGGCGAAAACTCTCAAGGATCTT |
| | | ACCGCTGTTGAGATCCAGTTCGATGTAACCCACTCGTGCACCCAACTGAT |
| | | CTTCAGCATCTTTTACTTTCACCAGCGTTTCTGGGTGAGCAAAAACAGGA |
| | | AGGCAAAATGCCGCAAAAAAGGGAATAAGGGCGACACGGAAATGTTGAAT |
| | | ACTCATACTCTTCCTTTTTCAATATTATTGAAGCATTTATCAGGGTTATT |
| | | GTCTCATGAGCGGATACATATTTGAATGTATTTAGAAAAATAAACAAATA |
| | | GGGGTTCCGCGCACATTTCCCCGAAAAGTGCCACCTGTCATGACCAAAAT |
| | | CCCTTAACGTGAGTTTTCGTTCCACTGAGCGTCAGACCCCGTAGAAAAGA |
| | | TCAAAGGATCTTCTTGAGATCCTTTTTTTCTGCGCGTAATCTGCTGCTTG |
| | | CAAACAAAAAAACCACCGCTACCAGCGGTGGTTTGTTTGCCGGATCAAGA |
| | | GCTACCAACTCTTTTTCCGAAGGTAACTGGCTTCAGCAGAGCGCAGATAC |
| | | CAAATACTGTTCTTCTAGTGTAGCCGTAGTTAGGCCACCACTTCAAGAAC |
| | | TCTGTAGCACCGCCTACATACCTCGCTCTGCTAATCCTGTTACCAGTGGC |
| | | TGCTGCCAGTGGCGATAAGTCGTGTCTTACCGGGTTGGACTCAAGACGAT |
| | | AGTTACCGGATAAGGCGCAGCGGTCGGGCTGAACGGGGGGTTCGTGCACA |
| | | CAGCCCAGCTTGGAGCGAACGACCTACACCGAACTGAGATACCTACAGCG |
| | | TGAGCTATGAGAAAGCGCCACGCTTCCCGAAGGGAGAAAGGCGGACAGGT |
| | | ATCCGGTAAGCGGCAGGGTCGGAACAGGAGAGCGCACGAGGGAGCTTCCA |
| | | GGGGGAAACGCCTGGTATCTTTATAGTCCTGTCGGGTTTCGCCACCTCTG |
| | | ACTTGAGCGTCGATTTTTGTGATGCTCGTCAGGGGGGCGGAGCCTATGGA |
| | | AAAACGCCAGCAACGCGGCCTTTTTACGGTTCCTGGCCTTTTGCTGGCCT |
| | | TTTGCTCACATGTTCTTTCCTGCGTTATCCCCTGATTCTGTGGATAACCG |
| | | TAG |
| 82 | Bacteroides site- | TCCGTCTCAAAACGGCGCGCCTGATAGGTGGGCTGCCCTTCCTGGTTGGC |
| | specific integration | TTGGTTTCATCAGCCATCCGCTTGCCCTCATCTGTTACGCCGGCGGTAGC |
| | vector | CGGCCAGCCTCGCAGAGCAGGATTCCCGTTGAGCACCGCCAGGTGCGAAT |
| | | AAGGGACAGTGAAGAAGGAACACCCGCTCGCGGGTGGGCCTACTTCACCT |
| | | ATCCTGCCCGGCTGACGCCGTTGGATACACCAAGGAAAGTCTACACGAAC |
| | | CCTTTGGCAAAATCCTGTATATCGTGCGAAAAAGGATGGATATACCGAAA |
| | | AAATCGCTATAATGACCCCGAAGCAGGGTTATGCAGCGGAAAAGATAAAA |
| | | CGAAAGGCTCAGTCGAAAGACTGGGCCTTTCGTTTTACAATTGGGCTACC |
| | | TTTTTTTTGTTTTGTTTGCAATGGTTAATCTATTGTTAAAATTTAAAGTT |
| | | TCACTTGAACTTTCAAATAATGTTCTTATATTTGCAGTGTCGAAAGAAAC |
| | | AAAGTAGCCTGGATCACACAACATTTAAAAAATAACATTATGAAAGCACT |
| | | CGAAAAGAGATTCAAAGGTAATATTAACAATAATTTATTTTCAATGGAGA |
| | | AAAAAATCACTGGATATACCACCGTTGATATATCCCAATGGCATCGTAAA |
| | | GAACATTTTGAGGCATTTCAGTCAGTTGCTCAATGTACCTATAACCAGAC |
| | | CGTTCAGCTGGATATTACGGCCTTTTTAAAGACCGTAAAGAAAAATAAGC |
| | | ACAAGTTTTATCCGGCCTTTATTCACATTCTTGCCCGCCTGATGAATGCT |
| | | CATCCGGAATTTCGTATGGCAATGAAAGACGGTGAGCTGGTGATATGGGA |
| | | TAGTGTTCACCCTTGTTACACCGTTTTCCATGAGCAAACTGAAACGTTTT |
| | | CATCGCTCTGGAGTGAATACCACGACGATTTCCGGCAGTTTCTACACATA |
| | | TATTCGCAAGATGTGGCGTGTTACGGTGAAAACCTGGCCTATTTCCCTAA |
| | | AGGGTTTATTGAGAATATGTTTTTCGTTTCAGCCAATCCCTGGGTGAGTT |
| | | TCACCAGTTTTGATTTAAACGTGGCCAATATGGACAACTTCTTCGCCCCC |
| | | GTTTTCACCATGGGCAAATATTATACGCAAGGCGACAAGGTGCTGATGCC |
| | | GCTGGCGATTCAGGTTCATCATGCCGTTTGTGATGGCTTCCATGTCGGCA |
| | | GAATGCTTAATGAATTACAACAGTACTGCGATGAGTGGCAGGGCGGGGCG |
| | | TAAGGTTCCTAGCTGATTAGAAGGCCATCCTGACGGATGGCCTTTTTTTT |
| | | GTGTTACAACCAATTAACCAATTCTGATTAGAAAAACTCATCGAGCATCA |
| | | AATGAAACTGCAATTTATTCATATCAGGATTATCAATACCATATTTTTGA |
| | | AAAAGCCGTTTCTGTAATGAAGGAGAAAACTCACCGAGGCAGTTCCATAG |
| | | GATGGCAAGATCCTGGTATCGGTCTGCGATTCCGACTCGTCCAACATCAA |
| | | TACAACCTATTAATTTCCCCTCGTCAAAAATAAGGTTATCAAGTGAGAAA |
| | | TCACCATGAGTGACGACTGAATCCGGTGAGAATGGCAAAAGCTTATGCAT |
| | | TTCTTTCCAGACTTGTTCAACAGGCCAGCCATTACGCTCGTCATCAAAAT |
| | | CACTCGCATCAACCAAACCGTTATTCATTCGTGATTGCGCCTGAGCGAGG |
| | | CGAAATACGCGATCGCTGTTAAAAGGACAATTACAAACAGGAATCGAATG |
| | | CAACCGGCGCAGGAACACTGCCATGAGACGTCGATTATCAAAAAGGATCT |
| | | TCACCTAGATCCTTTTAAATTAAAAATGAAGTTTTAAATCAATCTAAAGT |
| | | ATATATGAGTAAACTTGGTCTGACAGTTACCAATGCTTAATCAGTGAGGC |
| | | ACCTATCTCAGCGATCTGTCTATTTCGTTCATCCATAGTTGCCTGACTCC |
| | | CCGTCGTGTAGATAACTACGATACGGGAGGGCTTACCATCTGGCCCCAGT |
| | | GCTGCAATGATACCGCGGGACCCACGCTCACCGGCTCCAGATTTATCAGC |
| | | AATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGT |
| | | AGTTCGCCAGTTAATAGTTTGCGCAACGTTGTTGCCATTGCTACAGGCAT |
| | | CGTGGTGTCACGCTCGTCGTTTGGTATGGCTTCATTCAGCTCCGGTTCCC |
| | | AACGATCAAGGCGAGTTACATGATCCCCCATGTTGTGCAAAAAAGCGGTT |
| | | AGCTCCTTCGGTCCTCCGATCGTTGTCAGAAGTAAGTTGGCCGCAGTGTT |
| | | ATCACTCATGGTTATGGCAGCACTGCATAATTCTCTTACTGTCATGCCAT |
| | | CCGTAAGATGCTTTTCTGTGACTGGTGAGTACTCAACCAAGTCATTCTGA |
| | | GAATAGTGTATGCGGCGACCGAGTTGCTCTTGCCCGGCGTCAATACGGGA |
| | | TAATACCGCGCCACATAGCAGAACTTTAAAAGTGCTCATCATTGGAAAAC |
| | | GTTCTTCGGGGCGAAAACTCTCAAGGATCTTACCGCTGTTGAGATCCAGT |
| | | TCGATGTAACCCACTCGTGCACCCAACTGATCTTCAGCATCTTTTACTTT |
| | | CACCAGCGTTTCTGGGTGAGCAAAAACAGGAAGGCAAAATGCCGCAAAAA |
| | | AGGGAATAAGGGCGACACGGAAATGTTGAATACTCATACTCTTCCTTTTT |
| | | CAATATTATTGAAGCATTTATCAGGGTTATTGTCTCATGAGCGGATACAT |
| | | ATTTGAATGTATTTAGAAAAATAAACAAATAGGGGTTCCGCGCACATTTC |
| | | CCCGAAAAGTGCCACCTGTCATGACCAAAATCCCTTAACGTGAGTTTTCG |
| | | TTCCACTGAGCGTCAGACCCCGTAGAAAAGATCAAAGGATCTTCTTGAGA |
| | | TCCTTTTTTTCTGCGCGTAATCTGCTGCTTGCAAACAAAAAAACCACCGC |
| | | TACCAGCGGTGGTTTGTTTGCCGGATCAAGAGCTACCAACTCTTTTTCCG |
| | | AAGGTAACTGGCTTCAGCAGAGCGCAGATACCAAATACTGTTCTTCTAGT |
| | | GTAGCCGTAGTTAGGCCACCACTTCAAGAACTCTGTAGCACCGCCTACAT |
| | | ACCTCGCTCTGCTAATCCTGTTACCAGTGGCTGCTGCCAGTGGCGATAAG |
| | | TCGTGTCTTACCGGGTTGGACTCAAGACGATAGTTACCGGATAAGGCGCA |
| | | GCGGTCGGGCTGAACGGGGGGTTCGTGCACACAGCCCAGCTTGGAGCGAA |
| | | CGACCTACACCGAACTGAGATACCTACAGCGTGAGCTATGAGAAAGCGCC |
| | | ACGCTTCCCGAAGGGAGAAAGGCGGACAGGTATCCGGTAAGCGGCAGGGT |
| | | CGGAACAGGAGAGCGCACGAGGGAGCTTCCAGGGGGAAACGCCTGGTATC |
| | | TTTATAGTCCTGTCGGGTTTCGCCACCTCTGACTTGAGCGTCGATTTTTG |
| | | TGATGCTCGTCAGGGGGGCGGAGCCTATGGAAAAACGCCAGCAACGCGGC |
| | | CTTTTTACGGTTCCTGGCCTTTTGCTGGCCTTTTGCTCACATGTTCTTTC |
| | | CTGCGTTATCCCCTGATTCTGTGGATAACCGTAGTCGGCGTCTCAGCCAG |
| | | CGCATCAACAATATTTTCACCTGAATCAGGATATTCTTCTAATACCTGGA |
| | | ATGCTGTTTTCCCGGGGATCGCAGTGGTGAGTAACCATGCATCATCAGGA |
| | | GTACGGATAAAATGCTTGATGGTCGGAAGAGGCATAAATTCCGTCAGCCA |
| | | GTTTAGTCTGACCATCTCATCTGTAACATCATTGGCAACGCTACCTTTGC |
| | | CATGTTTCAGAAACAACTCTGGCGCATCGGGCTTCCCATACAATCGATAG |
| | | ATTGTCGCACCTGATTGCCCGACATTATCGCGAGCCCATTTATACCCATA |
| | | TAAATCAGCATCCATGTTGGAATTTAATCGCGGCCTGGAGCAAGACGTTT |
| | | CCCGTTGAATATGGCTCATAACACCCCTTGTATTACTGTTTATGTAAGCA |
| | | GACAGTTTTATTGTTCATGATGATATATTTTTATCTTGTGCAATGTAACA |
| | | TCAGAGATTTTGAGACACAACGTGGCTTTGTTGAATAAATCGAACTTTTG |
| | | CTGAGTTGAAGGATCAGCAAAAAAACACCCGTTAGGGTGTTTTTTCGAAA |
| | | AAAAAGGGGGAAACTCCCCCTTTCGCATTAATATGCCGCTTCGAATTCTT |
| | | TTAGGAAGCGTGTATCGTTTTCAGAGAACATACGGAGGTCTTTCACCTGA |
| | | TATTTCAGGTTTGTGATACGCTCGATACCCATACCGAGTCCATAACCGCT |
| | | GTATATTTTGCTGTCTATACCATTTGATTCAAGTACGTTCGGGTCTACCA |
| | | TACCGCAACCGAGGATTTCTACCCAGCCGGTGTGTTTACAGAACGGACAT |
| | | CCTTTACCGCCGCAGATATTACAGCTGATATCCATTTCCGCACTTGGTTC |
| | | AGCAAACGGGAAGTAAGACGGACGCAGACGGATCTTTGTATCAGCACCGA |
| | | ACATTTCTTTGGCAAAGAGCAGCAATACCTGCTTCAAGTCGGTGAATGAT |
| | | ACGTTTTTATCTACATACAGCGCTTCTACCTGATGGAAGAAACAGTGTGC |
| | | GCGATAGCTGATAGCTTCGTTACGATATACACGTCCCGGACAGATGATGC |
| | | GGATAGGAGGCTGTGAAGTTTCCATCACACGAGTCTGTACAGAAGAAGTA |
| | | TGTGTACGCAATACTACGTCCGGGTGAGCTTCGATAAAGAAAGTGTCCTG |
| | | CATATCGCGTGCCGGATGATCTTCGGCAAAGTTCAGTGCCGAGAACACGT |
| | | GCCAGTCATCTTCAATTTCCGGACCTTCGGCAATGCTGAATCCCAGACGG |
| | | GCAAAGATATCAATGATTTCGTTCTTTACAATGGTGAGCGGGTGGCGTGT |
| | | ACCGAGTTCTACAGGATAAGCCGAACGCGTCAAATCCAGTCCGTCACAAT |
| | | CGTTGTCCTGACTTTCAAACATTTCTTTCAGCGCGTTGATTTTGTCCTGC |
| | | GCTTTTGTTTTCAGTTCATTCAGTCTCATGCCGACTTCTTTTTTCTGTTC |
| | | GGCAGCTACATTACGGAAATCTGCCATTAAGTCGTTAATGGCTCCCTTCT |
| | | TACTTAGGTATTTGATGCGGAGAGCTTCGAGTTCTTCGGCATTGGAGGCG |
| | | TGTAAGGCTTCCACCTCTTTCAGAAGTTGTTCAATCTTAGCTATCATTTT |
| | | CTTATATTTTTTTGGTTGGTGATGCCAGGCTACTTTGTTTCTTTCGACAC |
| | | TGCAAATATAAGAACATTATTTGAAAGTTCAAGTGAAACTTTAAATTTTA |
| | | ACAATAGATTAACCATTGCAAACAAAACAAAAAAAAGGTAGCCCAATTGT |
| | | AAAACGAAAGGCCCAGTCTTTCGACTGAGCCTTTCGTTTTATCCTAGGAT |
| | | CAGCTGTACGTACTCGCAGTTCAACCTGTTGATAGTACGTACTAAGCTCT |
| | | CATGTTTCACGTACTAAGCTCTCATGTTTAACGTACTAAGCTCTCATGTT |
| | | TAACGAACTAAACCCTCATGGCTAACGTACTAAGCTCTCATGGCTAACGT |
| | | ACTAAGCTCTCATGTTTCACGTACTAAGCTCTCATGTTTGAACAATAAAA |
| | | TTAATATAAATCAGCAACTTAAATAGCCTCTAAGGTTTTAAGTTTTATAA |
| | | GAAAAAAAAGAATATATAAGGCTTTTAAAGCTTTTAAGGTTTAACGGTTG |
| | | TGGACAACAAGCCAGGGATGTAACGCACTGAGAAGCCCTTAGAGCCTCTC |
| | | AAAGCAATTTTGAGTGACACAGGAACACTTAACGGCTGACATGGGGCGGC |
| | | CGCACGATGAGACGGACCTCGATTATCAAAAAGGATCTTCACCTAGATCC |
| | | TTTTAAATTAAAAATGAAGTTTTAAATCAATCTAAAGTATATATGAGTAA |
| | | ACTTGGTCTGACAGTTACCAATGCTTAATCAGTGAGGCACCTATCTCAGC |
| | | GATCTGTCTATTTCGTTCATCCATAGTTGCCTGACTCCCCGTCGTGTAGA |
| | | TAACTACGATACGGGAGGGCTTACCATCTGGCCCCAGTGCTGCAATGATA |
| | | CCGCGGGACCCACGCTCACCGGCTCCAGATTTATCAGCAATAAACCAGCC |
| | | AGCCGGAAGGGCCGAGCGCAGAAGTGGTCCTGCAACTTTATCCGCCTCCA |
| | | TCCAGTCTATTAATTGTTGCCGGGAAGCTAGAGTAAGTAGTTCGCCAGTT |
| | | AATAGTTTGCGCAACGTTGTTGCCATTGCTACAGGCATCGTGGTGTCACG |
| | | CTCGTCGTTTGGTATGGCTTCATTCAGCTCCGGTTCCCAACGATCAAGGC |
| | | GAGTTACATGATCCCCCATGTTGTGCAAAAAAGCGGTTAGCTCCTTCGGT |
| | | CCTCCGATCGTTGTCAGAAGTAAGTTGGCCGCAGTGTTATCACTCATGGT |
| | | TATGGCAGCACTGCATAATTCTCTTACTGTCATGCCATCCGTAAGATGCT |
| | | TTTCTGTGACTGGTGAGTACTCAACCAAGTCATTCTGAGAATAGTGTATG |
| | | CGGCGACCGAGTTGCTCTTGCCCGGCGTCAATACGGGATAATACCGCGCC |
| | | ACATAGCAGAACTTTAAAAGTGCTCATCATTGGAAAACGTTCTTCGGGGC |
| | | GAAAACTCTCAAGGATCTTACCGCTGTTGAGATCCAGTTCGATGTAACCC |
| | | ACTCGTGCACCCAACTGATCTTCAGCATCTTTTACTTTCACCAGCGTTTC |
| | | TGGGTGAGCAAAAACAGGAAGGCAAAATGCCGCAAAAAAGGGAATAAGGG |
| | | CGACACGGAAATGTTGAATACTCATACTCTTCCTTTTTCAATATTATTGA |
| | | AGCATTTATCAGGGTTATTGTCTCATGAGCGGATACATATTTGAATGTAT |
| | | TTAGAAAAATAAACAAATAGGGGTTCCGCGCACATTTCCCCGAAAAGTGC |
| | | CACCTGTCATGACCAAAATCCCTTAACGTGAGTTTTCGTTCCACTGAGCG |
| | | TCAGACCCCGTAGAAAAGATCAAAGGATCTTCTTGAGATCCTTTTTTTCT |
| | | GCGCGTAATCTGCTGCTTGCAAACAAAAAAACCACCGCTACCAGCGGTGG |
| | | TTTGTTTGCCGGATCAAGAGCTACCAACTCTTTTTCCGAAGGTAACTGGC |
| | | TTCAGCAGAGCGCAGATACCAAATACTGTTCTTCTAGTGTAGCCGTAGTT |
| | | AGGCCACCACTTCAAGAACTCTGTAGCACCGCCTACATACCTCGCTCTGC |
| | | TAATCCTGTTACCAGTGGCTGCTGCCAGTGGCGATAAGTCGTGTCTTACC |
| | | GGGTTGGACTCAAGACGATAGTTACCGGATAAGGCGCAGCGGTCGGGCTG |
| | | AACGGGGGGTTCGTGCACACAGCCCAGCTTGGAGCGAACGACCTACACCG |
| | | AACTGAGATACCTACAGCGTGAGCTATGAGAAAGCGCCACGCTTCCCGAA |
| | | GGGAGAAAGGCGGACAGGTATCCGGTAAGCGGCAGGGTCGGAACAGGAGA |
| | | GCGCACGAGGGAGCTTCCAGGGGGAAACGCCTGGTATCTTTATAGTCCTG |
| | | TCGGGTTTCGCCACCTCTGACTTGAGCGTCGATTTTTGTGATGCTCGTCA |
| | | GGGGGGCGGAGCCTATGGAAAAACGCCAGCAACGCGGCCTTTTTACGGTT |
| | | CCTGGCCTTTTGCTGGCCTTTTGCTCACATGTTCTTTCCTGCGTTATCCC |
| | | CTGATTCTGTGGATAACCGTAG |
| 83 | NB001 porphyran | TAAGGATTGATTCGCTAGCTCAGCAGGTAGAGCACAACACTTTTAATGTT |
| | PUL | GGGGTCCTGGGTTCGAGCCCCAGGCGGATCACTGAAACAAAAAGCAAAAC |
| | | AATGAAAACCGCTGATAATCAATCATTATCAGCGGTTTTTCTTTTTATCC |
| | | ATACTGCAAATTGAAGCAGAATACCGCATTTTACTGGAGGTGAAATAGGT |
| | | GGACTTAATTTCCACATAAAAACAAGTCCACCTGATTGGATTATATTTCA |
| | | CTGATTCTCTGCGTTTTGCATAAAACAAACTCTTTTCAAAACATGTATTT |
| | | TTACACCATCAAAAAAAGAAGAGTATGGCAATGCAAAGAAACTATTTTAC |
| | | GGTATTGTTTTTCCTGAAGAAATCAAAGCTGCTTAAAAATGGAGAAGCAC |
| | | CAATCTGTATGCGTATCACAATAAACGGAAAACGTGCAGAGGTACAAATC |
| | | AAGCGAAGTATAGATGTTACAAAATGGAATACGCAAAAAGAATGCGCGAT |
| | | TGGCAGGGAAAAGAAGTATCAAGAAATAAACCACTATCTTGATACGATAA |
| | | GAACTAAAATCCTTCAAATTCACCGTGAACTTGAGCAGGACGGTAAACCT |
| | | ATTACAGCAGATATTATAAAAAATATCTATTATGGAGAACACTCTACTCC |
| | | CAAAATGCTGCTTGAAGTATTCCAGGAACACAATTCGGAATATCGGGAAT |
| | | TAATGAACAAGGAATATGCCGAAGGTACTGTACTTCGATACGAACGTACA |
| | | GCAAGATATTTGAAGGAGTTTATCAGTGAACAATATAAACTGGCTGATAT |
| | | TCCATTAAAATCAATCAACTATGAATTTATAACCAAATTCGAACATTTCA |
| | | TTAAAATACAGAAAAACTGTGCGCAAAATGCGACAGTGAAATATCTGAAA |
| | | AATTTAAAGAAAATCATCAAAACTGCATTGATAAAGAAGTGGATAACTGA |
| | | TGATCCGTTTGCAGAAATACACTTCAAACAGACCAAGTGTAACCGTGAAT |
| | | TCTTAAACGAAATGGAACTTCGCAAAATCATCAATAAAGATTTTGATATT |
| | | CAACGATTACAAACCGTAAGGGACATATTCATCTTCTGTTGTTTCACCGG |
| | | TTTGGCTTTCACAGACGTAAAGAATCTGAAAAAGGAACACCTTGTACAGG |
| | | CTGATAATGGTGAATGGTGGATAAGAAAAGCAAGGGAAAAGACCGATAAT |
| | | ATGTGCGACATTCCATTGTTGGATATACCAAGACTTATTTTAGAGAAATA |
| | | TCAGTCAAATCCAATCTGCAATGAAAAAGGATTATTACTTCCTGTTCCCA |
| | | GCAACCAACGAATGAACAGTTATTTGAAAGAAATAGCTGATGTATGTGGT |
| | | ATTCAGAAGAATCTTTCCACACATATTGCAAGACATACATTTGCATCACT |
| | | GGCTATTGCAAATAAGGTTTCCTTGGAATCCATTGCCAAAATGTTAGGAC |
| | | ACACGGACATTCGTACAACTCGTATTTATGCCAAAATAATGAATTCTACC |
| | | ATTGCCAATGAAATGAAAGTACTGCAAAACAAGTTCGCAATATAATTTTC |
| | | AACCATTATTTCATTTCTTACAGCAAATATCGCACTTTGCCACTGACTGT |
| | | GCAAGGCGGCCCTGTCGGGCTGGTTGGCGGAAAAAAATCATCCTCGCTTC |
| | | GCTCCGGTATTTTTTTCCGCCAAGCCTTGCACCGGTCATTGGCAAAGAAC |
| | | AGCCGGGCCAGTAAGAAATTGAAATACTGGCTCCACGGAGCCGGTCATGT |
| | | CTAATTTAAATAAAAGAATATGACTGAAGAAGTTGGAAAGAAGGTATGTG |
| | | AAGGTACAGTAGCAGACCTCATGAAGGACAAGACCGGAAAACAGACGGTT |
| | | GTCACGTTGACAAGAAAGAATGCTTACCGAGTGAAGAAAATCAGAGAACA |
| | | AGGGACGGATGACGAAGCTGTCCTTTTTCATTTCCGTGAACGCTGTACGG |
| | | GAATGGGCTCCTATGTACACACAATCGAAGCGGCAGACGGAGAAACAGAA |
| | | CTTCATCCGTCTGAATTTGAAAAATGGGAAGCTGTGGAATTCCTGTATCC |
| | | CGGCTATCTGGAAGACCTGCTTGATGCTGCATACAACGCATACAGATGGA |
| | | GTTCCTTCGAACCTGAAGCAAGGGCGGAAACAGACATCATGCAATATGAA |
| | | AAACAACTTGTAGAGGATCTGAAACAGATTCCGGAAGAAAAACAGAACGA |
| | | GTATACCAGTGCATACCATAGCAAGTTCTCTGCCTTGCTGGGCTGTCTCT |
| | | CACGATGTGCCAGTCCGATGGTGACAGGGCCTGCCAAATTCAACTGCCAG |
| | | CGCAACAACAAAGCCTTGGATGCATACCAGAACAGATTTGATGAATTTCA |
| | | TGATTGGCGTAACCGCTTCAAGGCTGCCATGGAAAGGATGAAAGAGGCTG |
| | | CCAAACCGGAAGAACAGAAGCAAGAGGAGGCATGGAACCGCCTGAAGCGT |
| | | GACATTGCAAGCAGCGCACAGACCATTCATGATATTGATACCGGTAAAGC |
| | | AAGAGGATACAGCCGTGCCTTGTTTGTCAGCAGTATCCTTAATAAAGTAA |
| | | GCACCTATGCAGGAAAAGGAGAAGTGGAAATCGTACAGAAAGCGGTGGAC |
| | | TTCATTACAGACTTCAATGCACAATGCAAAAAACCGGTTATCACTCCGCG |
| | | GAACCGTTTCTTCCAACTGCCGGAAATGGCACGCCAGGCCAGACTGAAAC |
| | | TTCAGGAAATCAGAGAACGGGAAAACCGTGAACTGAAATTTGAAGGCGGA |
| | | ACGCTGGTATGGAACTATGAGGCAGACCGCCTGCAAATCCAGTTTGACAA |
| | | TATTCCGGATGACCAGAGGCGCAAGGAACTGAAATCATACGGTTTCAAAT |
| | | GGTCGCCGAGATACCAGGCATGGCAACGGCAACTTACACAGAATGCCGTA |
| | | TATGCAGTCAAAAGAGTGTTGAACCTTCAAAACCTATAAGACATGAAAGA |
| | | CCGATTGAAATATGTAATCGATTCCCGCTACTTCGACGGAACATGCCTGA |
| | | CAAGTATGAGTGACGGATTCCATAATGACTATGGTGGGGAAACAATCGAA |
| | | GAACTGCGCATACGGGAAAACAATCCCTATCTGAAAGCAGTAACACCTTC |
| | | TGATATAGACAAGAAGCTGCGGCTATACAATCAGTCCCTGTCCGAACCGT |
| | | TCAAGGAAATCACTGAAGAAGAATACTATGACCTGCTGGATGTACTGCCA |
| | | CCCTTGCGCATGAGACAAAACTCGTTCTTTGTAGGAGAACCGTATTACGG |
| | | AAATATGTACTCTTTCTGCTTTACTCGTCAAGGAAGATATTTCAAGGGCC |
| | | TACGCTCCGTACTTACTCCGCAATCCGAACTGGACAGTCAGATAGACCGT |
| | | CACATGGAAATCATCAACCGGAAAGCCGTGATCTCAAAAGAGGAAACAAG |
| | | TAAAACGGTCACAACCGGAACCAGACTCATTCCCTATTATTTTTCACTGG |
| | | ACGGAAAACAGCCCGTATTCATCTGCAACCTTGTCATCCAATCAGATTCC |
| | | AGTCAAGCAAGGACGGACATGGCGAATACCCTGAAAAGTCTTCGCCGGAA |
| | | CCATTATCAGTTCTATAAAGGAAAAGGGCATTACGAAACTCCGGACGAAC |
| | | TGATAGACCATGTATCAGGAAAGAAGCTCACCCTTGTTTCCGACGGACAT |
| | | TTCTTTCAATATCCTCCCGGCAGGGAATCCGCAACTTTCATCGGACACAT |
| | | CAAGGAGACATCAGAGGAATTTCTTTTCCGGATCTATGACCGTGAATATT |
| | | TCCTGTATCTTCTTAAAAGACTGAGGACCGTGAAAAAGGAATCGGCACAG |
| | | GAACAAATAAATATCAAATCATAACATTCGGGGGAATGCGGTAAAATGAC |
| | | TGCCGTATTCCCTCATAAAAACAATACAAGTATGAACAAATCAAACACTC |
| | | TATACTGGAAAACAGCCACAGATCCGGCTGAACGCATTGAGGTCAGACTC |
| | | GTCCTGAACAGTTATATCGACAATGACAATCTGTATGTAGGACTTGAATC |
| | | CCGGTCTAAGGAGAATCCGGAATGCTGGGAATCCTACACGGACATCACCG |
| | | TCAACCTCAATTCTCTTCCCCCGTTCCATGCCTATGTGGACAACCGGGAC |
| | | TGCAACAGACATGTGCATGATTTTCTGACCAGTAACAGAATAGCAGAACC |
| | | TGCCGGATTTGAATATCAGGGATTCAGAATGTTCCGCTTCAATCCTGACA |
| | | GGTTGAAGGAACTCGCACCCGAACAGTTCAAGACAATCAGCGCCAAACTG |
| | | CCACCACAGGATGACATGATAAAGGACATCATCTATCAGGAAAGACGTTT |
| | | CCCTTTGAGAACTGTTCAAGACATTCACGGAATATATCTTGTTTCAAGCA |
| | | AGGAACTGGAAGAATCTCTGATCGAAGGAGTACGGAACCTGGATGCTGCG |
| | | GCATATGAACTGCTGGATGGCATCTGCCTGTTCTGCTCCACACAGGAACT |
| | | GCGCTATCTTACGGATGCAGAACTGATAGAAACAATCTACGCACAATAAA |
| | | AAGGAGGAACAAATATGAAAACCGGAGACATTGTATTTCTGAGACGTCCC |
| | | TATAAGGGATACCGTGCCGTCGAACTGATGGAAAGACTGGAATGCCGCTG |
| | | GCTGGTCAGGATTGTCGAGAGCGGTCTTGAACTGGAGGTATATGAAGATG |
| | | AACTTATATCAGAATTTTAATACAGACAAAGTGTTATGGAAAAATATCAG |
| | | TTTGCATTCCATTCGGAAATAATCGGCTATACCTCTCCTCATATCGGTGA |
| | | GGTCAGAAAAGCCATACACAGAAAAGTGGAAAAGGAAAAGTCTGCCGCCA |
| | | TAAAGAATGATATTGAGCTGCACATGTACAAAGTGCATGACGGCATACCG |
| | | GTTCTCCTTAACACCTGCTACCTGTACGATGAAAAAGGATGTATGGTACA |
| | | CGGAAGTATCAAGGGAACCAAGGATTATCTGCTTGAGACATGGAGATACC |
| | | ATACAAACAGACATTCTAAAGGCATCAGTTCCACAAGAATCAGGCCTTGC |
| | | ACGACAAGCAGGGCTTTTTCATTTGTATAACTCTTAAAATCAGAAATCAT |
| | | GAACCAGACATTACAACTTACAGACTATATTCCACAGAATGTAAGCCTCT |
| | | ACTACGTGGACTACCGGGATGATCTTGATGAGCATGAAGACATCCAGGAG |
| | | GAATGCATCCGTTCCAACAAAATGGAAAAACTCTATGAAAAGGCATACGA |
| | | ATGGTATGAGGAACAGGAAAGTTCAAACATGCACGACTATCTGGAGGAGA |
| | | CAAGAAAGAATATGGAAACGGACAATTTAGCCGGAGAGTTTGAAGAGCAT |
| | | GAAGATGAAATCAGGGAACTTATCTACGACCGGAACGATTCCGACCCGGT |
| | | AAAGGATATGATACGCAACTCGTCCGTCACTAATTTCTTCTATTCGCTCG |
| | | GAGTGGAAATCAGCGGATATCTGACCGGTTGTTCACTGCGGGGAGAATCA |
| | | GTCGCCATGGCCTGCCATAAGGTACGTCGCGCACTGCATCTGAAAAAGGG |
| | | GCAGTTTGACGAGAAGATTGAAGAACTGGTAGAGAATGCCACATACGGTG |
| | | GAGAACTGCGCATCTACTTCAACGCCATGTTTGACAGGCTCATCAGCAAA |
| | | GGCCCTGAGAACGATTTCAAGAGCATCCGTTTCCACGGGAATGTAGTGGT |
| | | GGTCATTGCCGACAGCCGGAACGGTTCCGGACATCATGTACGGATTCCGC |
| | | TGGACATCACTTTCCCTTTCCGAAGGGAGAACCTGTTTGTCGATTCACAG |
| | | GTACACTATTCCTATGCCAATGAAGTCTGCGGCATGACCAATGACTGGTG |
| | | TGATTCCACAAAATGGGAAACAGGCATGATACCTTTTACCGGATCTGTCC |
| | | GAAAAAGCCGGATGGCTGAATACAAGAAACAGGAAGCCGCTTATGAGCAG |
| | | ACATTCCGAGACGGGAAATGCACCTTCGGTGACATGAACTACAAACGCCA |
| | | CCGTGACGTGCGGTATTCGAATGAATATCCTGCCGGATGCAGGTGCCCTC |
| | | ATTGCGGTACATTCTGGATTGACTGAAAAAACATTTACCAACCAATAAAT |
| | | TCAAACGATATGAAAATCTGCTGTTCACAAGAGCATTACGACAAGGTCGT |
| | | ACAGTATGCAAAATCAATCAATGACAAGACACTGGAAAACTGTCTTGAAC |
| | | GTCTAAAACAATGGGAGAAGAACGAGAACCGTCCATGCGAAATCGAACTC |
| | | TATTACGATCATGCGCCGTATTCGTTCGGATTCTGCGAACGTTATCCGGA |
| | | CGGAAATACAGGCATTGTCGGAGGACTGCTGTATCATGGAAATCCGGACG |
| | | AATCCTTTGCCGTCACCATGGAACGTTTCCACGGATGGAGCATACATACC |
| | | TGACATATATGCGACAGTCTGTATTGGGGAGCCTCATGCAATATGGGGTT |
| | | CCCTTTTTTTATGCCGCAGACATGATGACAGCATCCTCATTTCTTGCTGC |
| | | AAAAATAGCTGTTTGCCGCGCAACTCCCGCAAGGCGGCCCTGCCGGGCTG |
| | | GTTGTCTGGAAAAAAATCATCCTCGCTTCGCTCCGGTATTTTTTTCCGCC |
| | | AAGCCTTGCAGGGATGCGGGCAAACAGACAACAGGGACAACAAGAAATAA |
| | | GAATGCCTGTACCTTACAGGCAGACAATGTATAACAATAAATATCAGAAG |
| | | TCATGATTACAGACCAGAAGACACAGAACAGGCTTCACGCGGATACCGGA |
| | | ACGGAACTGTTCTCCATCAGACAAAGGAAGGAAGCCGTCACAAGGATGCT |
| | | GGACATTCTGAAAGAGACTCCGGAATACCTGCAGGTTATGAACCATATAC |
| | | CGGCTTATGCCATGGATGACGATACGTCAGAATGGTGGAAATCGGAAGAA |
| | | TCGGAAAATTTCATGAACTCACTCCTGGAAGTGATGGAAAGCTATACTCC |
| | | GGACGGATACAGGTTCGGACCGAAATCCGGCACGACTGACCTTTACGGCT |
| | | ACTGGGAAAGCAAGACCGGGCGGACAACCCTCTTCCATCTGCTTTTCAGT |
| | | CTGGAAAGCGGATATGAATGGGGAAAAGGTCTTTCCCATGAGAAAACGGA |
| | | CGCATTCTACAAGGAAATAAAAGAGAAATTTCATGGAGAAGGATTCGACA |
| | | CGGACAGAACCGGCTGTACATCACAGGCCATGTATCTTGTAAAAGGAAAA |
| | | ACACGCCTGTACGTGCATCCGATGGAAATAAGCGGCTACTGTGAAACACT |
| | | GCATATTCCACAGATTACAGCCATACTGAAAAAAGGAGGCCGTACATTCC |
| | | GTCTTGTAAAGGATACGATAGCGGAAGAGGTGTATTCCTTCACCGATGAA |
| | | GAAGAACTGGAATATTACCGTGCCAGATACGGAACGTGCATCCACCGGAA |
| | | TATACTGGATGCCTTCAGCAACCGCCACGCAGGGAAAGAGGACATACTTT |
| | | CCATGATGGCATCACGGATAAATGTGGCTACGACATCACATCTTTACGGT |
| | | ATCGGATATGATTCGCCTGCATACAGGTTTGTGCATGAGGCATACGACAG |
| | | ACTGGTAAACAATGGAAAGCTGAAGGAGAATGTCCGGGAAATCGGTTGCT |
| | | GCAACATCATAATGGCCATTTCAAATACCAACGCAATATGAGACTGAATT |
| | | ACAATGACATGCTGCTTCTGGCAATATGGGAATACAACAGGAGACAGGAC |
| | | GAGGATCTGACCCTGGAACTGTTTCAGGAAACATTCGGACAGGTTCCCGG |
| | | CGCACATTTCCATGACAAATGGGTGCATTATTACAACAAGAACCTGCTGA |
| | | TGATGGCCGCCTATTTCAGGGGTGAGGAAGAAAACGGCCAGAAATTCTGT |
| | | GATATGATCACCCGACAGGTTGAACGCTATACACAAAACAGGAGGAGAAC |
| | | AGGATGAATACAAAGATACGATATGACCTTGACAGTCTTGAACTGGCAAA |
| | | CGGTGACTTCGGGTATCCCATTACAGAAAAGGAAGTACGGAAAGTGAACC |
| | | GTATGCTGGAACTGATGGAGAATGTCCGAAGCAGGCAGATGTGCCCGACA |
| | | GAAGGAGACTGCGTGGAATTTGTCTCACGTTCTGGTGACTATTTCGGAAA |
| | | AGCTCATATAGAACGGATAACAGGAAAATATGCGGATATATGCCTGATAC |
| | | CGGAAACGGTATTCTGTTTTGATGACATGGGAAAAGCCGCCTATGATACC |
| | | ACCGGAAGTCCCTGGACGCAGGTCAATATCCGGAACATGAAACCCGCAGG |
| | | TTCTGAAATCCGCATATTCAGAACATGGGGATTCGGGAAGCGCAGCAATA |
| | | CGGGCAGTCTCAGGTTCGATGCTCCGGTCAGGAAATGGGAATACAGAGAA |
| | | CCGAATCCGTTATATGACGGTTACACCACCCGTAACTGGTTCCGCTATCA |
| | | TATCATGAAACACCGGGACAGGGAAAGGACAGGCGAATACACCTTCCGCA |
| | | GCGATTCATTCACGCTGTACAGCCGGAGCGAGCTGGACGAGCTGGCCGCA |
| | | ATCCTGAAAGGCAGACTCTACAAGGGAATCCTGCCTGACTCTCTTGTACT |
| | | TTGGGGATACCGCATGGATATTAAGGAAATATCACGTGAACAGTGGAACG |
| | | GTATGGGACAGCACGGACAAATCCGCATGAAATTCATGGGATACGGTCCG |
| | | GTCAGAATCCACACGGACAATGAAAACCATACCGTAACAGTATACAGAAT |
| | | CAACGACATATTGTCTTCAACTATCAGAATTTTCATATTTTTTCAGTTCT |
| | | TTTTTTGTTTCTTCTATTAATATTTTAAGCCACTCCATGATTTGTATTGC |
| | | ATGTTCATGAACAGTTTCATTTTGGCTATCACTGTCGTGTAGTAGCCTTT |
| | | GAAAATCACGTAAAATATTGTCTTTCCCAAGCATCTCCCATACAGGCATC |
| | | ATCCGGTGGATTATTTTTCTCATGGTCTCACGGTCGGTTATCCTGTCAGC |
| | | AGATTCCATCTCCTCCAGTTCTTTTTCAGATTCCATAACAACGAGAGAAA |
| | | GCATATGACTATAATCATCCGTATTCTCCAGTAAACTGGAAAAATCGAAT |
| | | TCTCCGGAAACTGAAACTTGTGTACGAGATATAATGGTGGATAAAAAAGC |
| | | AAGCAGTCCGTGGATATTGAACGGTTTATGAATACAGCCTACAAATCCTT |
| | | CTTTTTCATAAATTCCGGAATTTCCGTCACCACGGGCAGTCATGACTGCT |
| | | ACTGGAACAGTTCTAGAATTGCCGATGTCCGAATTGCGAAGCAATCTTAA |
| | | CAAACCGAATCCGTCAGTATCAGGCATTTGTACATCTGTCAAGATCAAAT |
| | | CATATTCAGAATTTTCAAGAGCGGCCACTACTTCACGTGCATTCTTACAG |
| | | GTTTTACAGGATATACCTTTGCGCCCGAGCATATCTTCCGCTATTTTCAG |
| | | TTGTATAGGATCATCGTCCACTACAAGAACATTCTTAGGCAATATAGTTA |
| | | TTGTATTATGGTCCGATTTGTCTTCCTCAACTAACTCATCCGTTTCAGGC |
| | | AAAGAAAGTTCCAGTCTGAACATGCTTCCTTTACCGAGTACACTTTCTAC |
| | | ATCCATTTTTCCTTCCAAAACCTTAATTAATCCTTTGGTAAGGAAAAGTC |
| | | CCAAACCAAACCCTTCAGAATTGACATTCTGTGCGGCACGCTCAAATGGA |
| | | GCAAATATTCTTTTCAGTGTTTCCTCATCCATACCGATACCAGTATCCCT |
| | | TATTTCAATACGAAGTTTTCCTTCTGAATATTCTGAATGGAAATTGACGT |
| | | TACCCCTGGAAGTAAACTTAATAGCGTTTGTAAGTAGATTGGCTAAAACC |
| | | TGTTCAAGTTTGTCCGCATCACCTTTTACTATTACATTTGATCCTTTATG |
| | | TTCAGAATATAAAATCAGACCTTTTGAAGTCGCTTTACGAGAAAACTCAT |
| | | CTGAAATTCGTTGCAAGAAACGGTCAAGATAAAATGGTGTGTCGTTACGC |
| | | AAATTACCGGCTTCATTGATTCGGTAAGCATCCATCAAATCATTAACCAG |
| | | ATGTAAAACGTGTCGACAAGAATGACGGATGTCATCTAAATATTTTTCGC |
| | | GCTTCCTCTTTTCACGCGTTTCAGATACCAAATCTGCACAGTTATGGATA |
| | | TTACCAAGTGGACCTCTAATATCATGAGAAACTGTCAGGATGATTTTCTT |
| | | ACGCATATCAAGCAAATTCTCGTTTTCTTGAATAGCTTGTTGTAATTTAA |
| | | ATTTAATTATTTCTTCCTTACGTAAATCTGATTGTATAATTAAAAATGAA |
| | | ATTAATATTATAAAAACCGCAATACTCATCATTACGATAAATAATCGAAA |
| | | GGATTCTTGTTTGACTTCCGTTACCTCTAAGTTTCGTTCTATAAATGACA |
| | | GCTGTACCTGATTATCTAAAAAAGATACAAAATCATATAATTTTTGATTT |
| | | AACAGCCTATTCTGCAAACGCAAGCTATCCACATAAGTTTCTATCTGATT |
| | | GTTTCGCATATCTATGACAGAAACCAATCTATTATTAAAATTCTGTATTT |
| | | CATTAGTTATATAGGGGACTTGTATCGTCTCCTTCTTTCCGAATAATCCG |
| | | GCAATTCCTTTCTTTTTCTGAGTTATTGTCTTCACTTTTACTGTTTGAGT |
| | | AGCTATTACAGGCAATTCATTAGTAAGAATACTATCAGATTTATTCGCAA |
| | | ATTGGACTGCTTTCATTATTTGAAACAAGTGCATTTCTTTCGTTTTAAGC |
| | | AATTCCCGTAAAGAATCAATTTGAACTGGACATAAAAAATCACAACTCCT |
| | | TAATTTTATTTCAAGTAGAACACTATCTGTTTTAAAACGTTGATTATGAA |
| | | ATATGTTATAATCAGACTCATCCCATACTATAACTGATTCGCCTAAAGTT |
| | | GCCAACTTAGTAATATACAAATGAACTTTATTAGTATTCTCATAAGCTTC |
| | | ATTAATTTGAATTATCAGATTCTCAAGTTCTTTCAACCGGCAACGTTCAT |
| | | TTATCATTACAGTAACCATACTTAAGACTATAAATCCTGTAATAAAATAT |
| | | CCAATAAATAGTCTTTTGCGTAATAATGAAGTCATCAGGAACATTCTATT |
| | | GATTTATTTGACATCATAATTCTATATATTTAACTAGTCATAGTATATAT |
| | | CATTCTCAAATATTTATTTCAAATTCAAGCAATAAAATAAAAAAACACTT |
| | | CATATTACAACTGAACTCTTTTATGAAAAAGTTGAATATATGAAGTGTTT |
| | | TTTTATTACGATATAAACTATAAAATCCTATTCTTCGGGAACTGGTGTAT |
| | | AAACCCTTATCCAGTCCACCAGGAAGGTGTGGTCTTCCACATTTTTCAGT |
| | | TCCTCATCCGTAGGGCTTAAACCTTTAACGGCTCTCCAGCTTTGGTCTTC |
| | | CATATTTATTATGATGTCCATGTCTTTTACCAGACCTGTACCACCAGTGT |
| | | AGTTGTTGGGGTCGATAATATCCTTGCCGCTTACGGTTCTGACAAGTTCT |
| | | CCATCTACATAATATTCAAGTGTGAAAGGGTCTTTCCAGAACACTCCTAC |
| | | ACGATGAAAATCGTCGCGCCACAATGTTCCCTTGTCATCCTTATACCATG |
| | | AGCCAAGATCTTTCGGCTGATAATCCTTGAATGGCTGGCGGATGAATATG |
| | | TGATGGCTCAGGTGAAGTCTGTCGGCACCGTAACCTCCGCCGTCTCTGTC |
| | | GCCGCCGTATGCTTCTATGATGTCGATTTCCTGAGTATCGTCAGGGCTGA |
| | | GCATCCATACATCGGATGCCATGGTTGAATTTGAAAGTTTTGCGTATGCC |
| | | TCTACATAAACCGGATACTTTACACGTGTCTTCGATGTGATACATCCCGT |
| | | ATAGGTTCCCGGCAGTTCCTTTGTGTTGGGTCCGCTTACAACTTTCTTCA |
| | | TGGGGACATCTTCAGGACGGCTGGCTCTTATTTTAAGGTATCCGTCGGAA |
| | | ACGGAAACATGGTCTCTCTGCCATATTGTAGGAGCAGGTCCTGTCCAATG |
| | | ATTATGATAGAAATCGGTCCATTTGGCATAGAACTCTTTTCCTTTATCCT |
| | | TTTCGTCGGCAACATAATTAAAGTCGTCCGACTGTGGATGGAGTTTCCAC |
| | | ACCATACCGTCGCCGGCATCAGCGGGTACAGGATAGATATCCCACTCGTA |
| | | CGATTTATTATTGAAATCTTCTGCTGCACAGGCTATTTGCAGCGATGCTA |
| | | AACAAATGGTAAACAGTTTTCTCATCGTGGTATCTTAGTTTAAGTTATAA |
| | | TAATTATTTTCGTTCTTTTGATTCACCTTTAGCGGTATGTGTCTGCAATG |
| | | TCCAGGTAGAAAATCTCATTATGCTCTGATAGTCTGAACTGTTGTATATA |
| | | TGAGTAAGACCCCATCTCAATATTTCGGTAGGTTCTTTTTCGGCATCTGC |
| | | ACTGCGGTTCAGGCCAATGGCGTGTGGCGCGCCTTTTACTACTGACATTA |
| | | TTTCAAAGTTTATTCCGTCAGGCGACCACTGGAGTGTGTTCTTTTCAGGA |
| | | CCGTCGGTGGTGATAAGTGAAGCTATACCTCCTTTGTAAGGCCATACGCA |
| | | AACTTCATGCCCGCTGTTTGAAATAGGATTATATTCCGATTTCACATACG |
| | | GACCCATAGGATTTTCCGCAATAGCCACTCCGTGTTTGATTTCACGGCCG |
| | | CCCCATGTTATTTCTTCTCCCATACGTTCGCCTTTGTAGTACATATAGAA |
| | | CTTACCTTTATAAGGTATTATACACGGGTCGTGTACCTTATGACTGTCGA |
| | | AATCACCTTTCGACACTACCTTGAATCTGTTATCCTCATCGCCTTCCCAT |
| | | TCGCCGGTATTAGAAGGTTCCAGTACAGGCTTGTCTGTCTTGATCCACGG |
| | | TCCTTCAGGGGAATCAGCACATGCCATACCGATAGTATTCTTTACACGGA |
| | | CTGTGTAAGGGGATTTTACCGCCTGATAGCAAAGATAATACTTTCCTTTC |
| | | CATTCCATCACCTCAGGAGTGAAGACTGAACGGTCGTCGTAAGCACCTTT |
| | | TTCACCACGTTTCACTGCAATTCCCTGTTCCTTCCATGTCCATCCGTCTT |
| | | TTGATGTGGCATACCATATATCACATCTGTCCCATGGGAAAACCTTATCT |
| | | TTCTCTATATCTCCAGCAAATCCTTGGGTAGGTCCATAGCTCTTTGAATA |
| | | CCATACATAATATGTATTACCTATTTTCAGCATTGCACTCGGGTCTCTTC |
| | | TTACTACGCCCTCTTCATAAGCAAGATCACCTTTAAGTGGTTCCATCTTA |
| | | TACTCAAAGAACCATTTATTGTCGTGATTTTCCCATTTCATGGCACGTTT |
| | | CATAGCTGCACTTAACTTATTTCCCTTAGGTATTCCCAATGAATCGGCCT |
| | | TACGCTCATCATAATTCTGAGTGTCGTCAACGGCAATAGTCTGTGTATTG |
| | | CCTGTATTTCCGCATGCTGCCAATAGCGACATCATGCCGGCTGCAAGAAT |
| | | AATTTTTCTCATACTAGACTTTATTTTATATTAATTGTTAGTTTATTCGA |
| | | GTGTAATTCACTTGTTTCTGCACTGATATTCAGTACCGATGATTTTTCTG |
| | | TCGACTGAAGCATCAGCATACATCTTCCCTGATATGTCATAATATCCTTA |
| | | CTTTGATATGGAGAAACGTTCTTCACGTTTCCATTGTCTATACCAAGCAG |
| | | ACGGTACTCTCCATCAATGTTGAACTTAAGCATCTGTTCTGTTGTCTTTA |
| | | CAGGATTACCTTTTTTGTCTGTTAGCTGAGCTGTGACATGCAGAACATCC |
| | | TTTCCATTTGCTGCGATACTTTGTTTGTCAACCGTCAGCAATATCGAATG |
| | | TTCTTTGCCTGAAGTCCTTATAGCTGTAGTGGTATTACCTAACTTATTTT |
| | | TTCCTTTTGCGGTAATAGTGCCAGGCTTGTACTGAACTGCCCATTTATAG |
| | | ATATGATCCTCAAAATCGTCTATATACTTCTTTCCCATCGACTTACCGTT |
| | | AACGAAAAGTTCCACTTCATCACAATTGGAATATATCTCTACTATTACCG |
| | | AGTCACCTTTCTGATAATTCCAGTGAGAGTTTACATCATCCCAAACCCAT |
| | | AATTTTCTATCCCATTCATGTCCTTTCTTATCAGTAAATCCATCTTTTAC |
| | | ATGGAGATACGAAGATTTGTCTGTAGTCTGTGAATATATAGCAATAAAAG |
| | | GCTTGTCTGTCCACAATGATTTCATCATGTCGTACGAAGGCTTCACATAG |
| | | CCGCACATATCCAGGAGACCACATCCTATCGACTTTTGAGGCCATTTTGA |
| | | AAGACGGCTTTCACTTTCTCCCAGATAATCGACTCCTGTCCATATAAACA |
| | | TACCCGGAACGAAATCCCTTTCAATCACCGCCTTCCATTCGTGCCACTGA |
| | | CCGAGATTTTCTGTACCCATTATAGGCTTGTCAGGATAATTCTTCTTAGC |
| | | ATAATCATACATCACGCGACGGTAGCTGAAGCCTGCCACATCGAGCGCGT |
| | | CGATATATCCTGACTCAAAGCTTATGGAAGGCAGGATGCAGTTGGCGGTA |
| | | ACTACACGTGTGGTGTCCATCTGGCGTGTCCATGCAGCTAATTTTTGCGC |
| | | TGTACGGCCAATGTCGTATGCATGTTTAGGCTGGATTTTCCACATTTCTC |
| | | TGATTTTTTCTTTAGAGTATGGAGGCTGATTCCAGAAATAATTACCGTTG |
| | | GAATCGGCACCGAAGAAACCTGTCGCCTCGCGGCATCCGGTATAAGTCCA |
| | | TTCTATTTCATTACCTATACTCCACTGGAAGATACAGGCATGATTACGGC |
| | | TTCTCCTCATTACGTTTTTCAAATCTCTTTCTGCCCATTCCTGGAAATGC |
| | | TCGCAATAGCCATGCGTAGGATAGTCTTCTACAGTTTCCTTCATATTGAG |
| | | TCTTTTATCTTTGGGATAATCCCACTCATCGAAGAATTCTTCCTGAACCA |
| | | GAAGACCTATCTCATCGCACAAAGACAGAAACTCTTCCGCTCCCGGATTG |
| | | TGCGAGAGGCGGATGGCATTGCATCCTCCTTCCTTTAGGGTTTTCAGACG |
| | | CCGGTACCACACATCGCGTATCATTGCCGCGCCAACCATTCCGGCATCAT |
| | | GGTGCAGGCATACTCCTTTTATCTTCATGTTTTTCCCGTTAAGGAAGAAA |
| | | CCTTTGTCTGCATCAAAACGGAATGTCCGTATGCCGAACCTGACAGTGTT |
| | | TTCAGAAATTACTTCATCGCCATTCTTGATGCGTGTCTCGGCTGTATAGA |
| | | GGACAGGTGTATCGACGCTCCACAAATCAGGCTGTTTAATCTCAGATACG |
| | | ATGTCGATAATTTTCTCCTCACCAGCATTCAGTTTTATACTGAAGACCTC |
| | | AAAGGCTGCGATATTGCCTTTATTATCCTTATATACTACCTCAACAACTG |
| | | CAGCTCTGGGTTCGGAGTAGCTGTTGCACACGGTAACCTGGTTGTTTACT |
| | | TTAGCATATTTATCAGTAACCACGGGAGTAGTGACAAATGTTCCCCAAAC |
| | | CGGAATATGCAGTCTGTCGGTTACAATCATTTTCACATCCCTGTATATAC |
| | | CTGAACCGGTGTACCATCTGCTGTCGGCATAATGGCTGTGGTCGACCCTT |
| | | ACAGTCATACGGTTATCCTCATTGGGATTGAGATAGTCTGTGACATCAAA |
| | | ATAAAAAGGAGCATATCCCGAAGGATGATATCCAAGCTTTTTGCCATTTA |
| | | TCCAATACTCAGAATTATTATATACTCCATCGAACACTATATAGCATTTC |
| | | TGATTTGCACTGATTGTTGTGGGAAATGATTTGCTATACCATCCTATTCC |
| | | TCCCTGAAGGAAAGCTACACATCCTTCACCCGAAATGGAATCGTAAGGTA |
| | | AACCAACACTCCAGTCATGTGGCAGGTTCACTTTCTTCCATTCATCACCA |
| | | GGGACATAAGAAGTATATGAATAATGAGCAGAATCTTTCAGTACGAATTT |
| | | CCAATCTTTATTGAAATCAACATTTGAATCAGATGCTGAAACCTTTAGGG |
| | | TTGATAATAGGATTATTAAAGCTAAAAGATTTTTATTTCTCATAATCTTA |
| | | GGTTTTACATGTTTTTTGATGTCACAAAACTATATCTTTCACTTATAATA |
| | | TATGAGGGGGATATTAATGTGATATAGGGTGGGAAATCAGAATTTTACAT |
| | | CTGCCCTGTATTCCACCGTCACCTACAACCTTGACAAAGGATGTTCCTTT |
| | | CTTCCCTCTTATGGTTCTCAGGACAAACAGACACTTTCCGTTATATGTCC |
| | | TTACACTATTGTTTATGACGTTGATGTTCAAATCTTCTATCGAAGGCGAT |
| | | CCATTGTCGAGTCCGGCAAGTTCAAGCTTGTCGTCGAGGATTATCCTCAC |
| | | ATCCGAAGGTATATCGACTACTGTGTTTCCTTCTTTATCTTCAATGGATA |
| | | CTTCTACATGGATAAGGTCATAACCGTTGTCGGTAGCTGTTTTGCGGTCG |
| | | CAGTTCAGTGCCAGACGGCACGGCTTGCCGCTTGTGGACAAAGTGTCTTT |
| | | CGACAATATTCTGTCGCCGTCCTTGCCTACCGCAAGGAGTGTTCCTTCCT |
| | | TGTATGCCACCTTCCACATCAGTATATTATGCTCCATGAAATCGCTGCGT |
| | | TTCTTTGTTCCCAACGATTTGCCGTTCAGAAACAGTTCCACTTCTGGGGC |
| | | GTTGGTATATACCTGCACCAGTATGTCCTCGTCCCTGCGGTACTTCCATT |
| | | TATCGCGTGTGTCGTACCACTCCCAGCGTCTGATCCATCCCGGGCGTGGA |
| | | GTGTAGGTGAAACTTCCGTCAGTATCCATCTTGAACTCGCTTTCCTTTTC |
| | | AGGTATTGTTACAATATGGGTTTTCGGTGTGTCTTTCCACAGACATTCAA |
| | | AGAAATGGCCACGCGCTGTCTTGTTGCCCACGAAATCGAAGAAAGAACAG |
| | | TCTCCACCCCTTGCAGGCCATGGGCCGTTCTCGCCAAGATAGTCGAATCC |
| | | TGTCCACACGAAGATGCCCGCTATGTACTTCTTGTCGGCCACGGCTGTCC |
| | | ATTCAAAGAGCTGACCAACATTCTCCGAACCGATAATAGGCTGATATGGA |
| | | TATAGCTTATGGTCGATTTCATAATATTTGTCTTTATAGTTATATCCCAC |
| | | TACATCAAGAACGTCTGTATATCCGGAGAGACGCGAAACTGACGGAACAA |
| | | CGACTCCTGAAGAGACGGGACGGGTAGTGTCCACATCCTTAACCCAACCG |
| | | GCAAGGACAGCGGCTGTTTCAGCCAAATCGTCTTTTCCTCCTGACAGACG |
| | | GTTGAACTCTTTCAGTATAGACTTGTTGTCTGTTTCCGGGTCGCCCGTAT |
| | | GGATAAGACCCTTGAACCCTTTATTGTCTTTGCTCGATGCCCAGTAATAT |
| | | GGATAGGTCCATTCTATTTCATTGCCTATACTCCAGAGTATCACGCAAGG |
| | | ATGATTTCTGTCTCGCCTGATGAACGACTTGAGGTCGTGCTCGGCATGCG |
| | | TATCGAAGTATCTGGTATATCCTATTGATATGCTGTCGGGCGCATCTTCC |
| | | TTAGCTCGCTCAGTAATCCACTTTTTCTTTGCCACCTTCCATTCGTCGAT |
| | | AAATTCATTCATTACAAGAAGTCCCAGACTGTCGCACATTTCCAGCAGAC |
| | | TTTCCGAATGCGGATTATGGGCTGTACGTATGGCATTGCAGCCTATGGAA |
| | | CGAAGTTTCAGAAGGCGTCGCAACAGGGCATCATCGTATGCGGCAACACC |
| | | CATACATCCCAAGTCGTGGTGTATGTTCACTCCTTTTATTTTTACTGATT |
| | | TTCCGTTTAGAAGGAAGCCTTCATCCGCATCGAATTTAATGTCGCGGATA |
| | | CCAAATTTTGTTGTTTTCTTATCCATCACATATCCGTCAGAAGCAATCAG |
| | | AGTAGTATGAAGCTCATACATCGAAGGCGTTTCAAGACTCCAGAGATGAC |
| | | AATTCTCCAGTTCAACAGATGCAGTGAACTCATTGAAATCGCCTTTCAGG |
| | | GCAACAAAATCATCGGAAACAGAAGCTATTGTCTTGCCGTCGTACACTAC |
| | | TTCGTGCTTCACGGTGACTCCTTTTACACCTGTTCCAGCATTCTTCACCT |
| | | CGCATACCACATTCACCATCGAACGGTTGCCTACCTGTGGTGTGGTAACG |
| | | AATATTCCGTCTGAAGGAATATAGAGCTCGTTTCTTAGAATAAGACTCAC |
| | | ATTCCTGTATATACCGGCACCGACATACCATCTGCTATCGGCATACGCTC |
| | | TTCTGTCAACGCAGACAGTTATTGTATTCATCGAACCTTTTGGTTTCAGA |
| | | TATTGAGTAAGTTCATATTCAAATCCCACATATCCGTTAGGACGGAATCC |
| | | CAACATATGCCCGTTTATCCAAACCTTTGAGTTATTATATACACCTTCGA |
| | | AATGAATGAACACTTTTTTCCCATTCATATCATCCGAGGTGAGAAAATTC |
| | | TTCATGTAAATCCCCACACCGCCAGACAGAAAACCATTGCTTCCGGCTGT |
| | | CTGAGTCTTGGTATATCCTTCGCTGATACTCCAGTCATGAGGCAGACACA |
| | | CATCCTCCCACTTTATATCTGGACTCAGGAACAAAGTGTCCTGAGGCACG |
| | | AAACCTGCTGGTTTGCTGAATTTCCAATCGAAGTTGAAATCCACTTTAGT |
| | | GGAGGTTCCGGCATAACAGAATCCGGACAGAAAGATAGTTAAGACTGTGA |
| | | TAATGTTTTTTATGGTCATATCGATTTTCAGATTAATATTAATGACAAAA |
| | | ATAATTTCAAAAGTGTAAAAACAAAAAAACTCTCCATTTATATTTCAGAT |
| | | ATCAACGGAGAGTTTCATCATTAAAAAAAATAAAACATTTTATAAAGTTA |
| | | CTCCTTGCTTAAGGATAGCTATTTCCCGGTATCCCTTCTTTTCGTTCAGT |
| | | GCCTGCTTTCCGCTTGCCACTTCCACCACAAAGTCTATAAAACGTCTGCT |
| | | TAAAGATTCCATGCTTTCTCCCTCTACCAGAGTTCCGGCATTGAAATCAA |
| | | TCCACGTATGTTTCTGTTCATAAAGCGGAGTGTTGGTCGAAACCTTCACG |
| | | GTTGGAACGAATGTTCCGAACGGTGTTCCGCGGCCTGTTGTGAACAGCAC |
| | | GATATGGCATCCGGCAGAAGCAAGAGCCGTACTTGCCACTAGGTCGTTGC |
| | | CTGGTGCGCTCAACAGGTTAAGTCCGTGTGTTGTGACACGGTCGCCATAT |
| | | TTCAGAACATCCTCCACCATCGAGCTTCCCGACTTCTGTGTACATCCCAA |
| | | TGATTTCTCCTCAAGCGTGGAAATACCTCCCGCCTTGTTTCCCGGTGAAG |
| | | GATTTTCATATATTGGCTGGTCGTTGCGGATGAAGTAGTTCTTGAAGTCG |
| | | TTTATCATGGCCACTGTGTCGTCGAATATCTCCTTCGTGCGGCAACGGTT |
| | | CATGAGCAGTGTCTCGGCTCCGAACATTTCAGGTACCTCCGTGAGGACTG |
| | | TTGTCCCACCCTGGGCAACAAGATAGTCAGAGAACACCCCAAGCATCGGA |
| | | TTGGCCGTGATACCGGACAGTCCATCAGACCCGCCGCACTTGAGTCCTAT |
| | | ACGCAGTTTTGACAGGGGGACATCAGTCCGCTTGTCTTCCCTGGCTATGG |
| | | CATACATCTCACGGAGAAGTTTCATACCCTCTTCTATCTCATCATCTACT |
| | | TTCTGAGAAACAAGGAAACGGATCCTTTGGGTATCATAGTCACCTATAAA |
| | | CTCACGAAAGGCATCAGGCTGGTTGTTCTCACAGCCAAGACCTACGACAA |
| | | GGACAGCTCCGGCATTGGGATGAAGGACCATGTCACGCAATATCTTACGG |
| | | GTGTTCTCATGGTCGTCACCCAACTGCGAGCATCCGTAGTTATGAGGGAA |
| | | AGATATAATGGAGTCAACCCCCTCGCAACCTGTTTCCTTGCGAAGCTGCT |
| | | CGGCCAACTGGTTTACTATTCCGTTCACGCAACCCACCGTAGGGATAATC |
| | | CATATCTCATTACGTATGCCGGCTTCTCCGTTAGCACGCAAATACCCTTT |
| | | GAATGTATGGTTCTCGTTCGTGAATGTCTGTTTCTCGAACTTCGGAGTGT |
| | | AAGTGTATGTACTCAGACCGGAAAGGTTCGTCTTGACGGTTTTCTCGTTC |
| | | AGCAGATGTCCTTTCCTGACTTCCTTTACAGCGTGCGATATGGGGAAACC |
| | | GTATTTTATCACCATATCACCTTCTGCAAAATCCTTCAGGGCAATCTTAT |
| | | GACCGGCAGGTATATCCTCCATTAATTCTATGGAATTGCCGTTCACCTCT |
| | | ATTACAGTCCCTTTGGACAATGGGTGCAGTGCCACAGCCACATTGTCCGC |
| | | AGGGTTTATCTGGATATATTCAGTCATAACAAACTAACATTTATAAATTG |
| | | AAGAATACAGGTAGAAGTATCAACCTACAAGGTCTTTTACTGTCTGAAGC |
| | | ATTCCTTCGCTCTGGATTTTGTTGATATAGTAAATTACACGGTCTGCCAG |
| | | TCCCGAGATAGTATTAAGGTCTTCACCCCAAATGGAAGTATCGGCGAGAA |
| | | CTGTCTTCACAAGATTTTCTACCGAGCCATCGTTCCACAAACTTGTAAGC |
| | | ATCGCCATGATTTCCTGTGCATCGTTAGGAACTATCTCTACACCATCGGC |
| | | ACGCTTTCCACCTTTGTAGTATACTATGATGGCTGCAAGACCGAGTACAA |
| | | GTCCTTCAGGAAGCACACCCTTACGTTTCAGATATTCCTTCACTCCTGGA |
| | | AGGTCGCGTGTGGCATACTTAGGGAATGAGTTAAGCATGATTGATGTTAC |
| | | CTGATGGTCTACGAAAGGATTATTGAAACGTTCCAGGACATCATCGGCAA |
| | | ACTTCTTGAGTTCCTCTTTCGGCAGGTTGAGGGTCTCCATCAGCTCGTCG |
| | | AACATCACACGTTTGATGAACTTGCCTATCACCTCATGTTGGCATGCGTC |
| | | TCTCACGATATTGACGCCCGAAAGGAATGCCACCGGCGACAATACAGTGT |
| | | GAGGACCGTTCAGCAGAGTAACCTTGCGTTCATGATAAGGCTCCTCCGAC |
| | | GGGACGAACAGAACGTTCAGTCCCGCCTTGTTTGCAGGAAATTCTTCGGC |
| | | AACCGATTCCGGTGCTTCGATAACCCACAGATGAAAAGCCTCGCCCTGTA |
| | | CAACTAAATTGTCATCAAAGTATAGTTTAGTTTTTATGTTGTCTATGTCT |
| | | TTACGAGGGAAACCCGGTACGATACGGTCCACCAGTGTGGCATATACACC |
| | | ACATGCAGTTTCAAACCATGACTTGAACTCTTCGCCAAGGTTCCACAATT |
| | | CAATATACTGATAGATTGTTTCCTTCAGTTTGTGACCGTTGAGGAAGATA |
| | | AGCTCGCATGGGAAGATGATGAGTCCTTTCGACTTGTCACCGTTGAAATG |
| | | TTTGAATCTGTGATAAAGCAACTGTGTCAGCTTGCCCGGATAAGAGCTTG |
| | | CAGGAGCATCCTCAAGCTTGCACGACGGATCGAAGTTGATACCGGCCTCA |
| | | GTAGTGTTCGAGATTACGAATCTCATATCAGGCTGTTCCGCCAGTGCCAT |
| | | GAAGTCATTATACTGGCTGTATGGATTCAGCGCGCGGCTGATGACATCAA |
| | | TCATTCTGAATGAGTTCACCACCTCGCCATTGTTCAGTCCCTGAAGATTG |
| | | ACATGATACAGACAGTCCTGGGCATTGAGGGCATCAACCATACCTTTTTC |
| | | TATAGGCTGCACCACAACAACACTGCTGTTGAAATCTGTCTTTTCATTCA |
| | | TATTCGAGATAATCCAGTCGACAAACGCACGAAGGAAATTACCTTCGCCA |
| | | AACTGTATGATACGTTCCGGACGTACTGCCTTTACTGCAGTCTTACTATT |
| | | TAAAGCTTTCATTGTAATGCCAAAAAATTAAAATTGATAAGATTAAAATT |
| | | CAACCAACATTCTGAATACCTTACCTGGATTTTCCGACCATTTCTGCAGA |
| | | GCCTCGCCTGCCTCTTCAGGTTTCACTACGGCAGAGATAAGTTCGTTCAT |
| | | CGGGCAGTTGCCATTCTGAAGATAATGTATCACGGCACGGAAATCCTCAG |
| | | GCATTGCATTGCGCGAACCGCGTATGTCGAGTTCCTTCTGGACAAAATAT |
| | | TTTGTCTGGAAAGCCACTTCACTCTTGGCATAGCCGATACATGCCACACG |
| | | GCCTGTGAAACCTACAATGTCGATGGCAGTAACATATGTGATAGGACTAC |
| | | CCACAGCCTCTATCACCACATCAGCCATATAGCCGTCAGTAAGTTCCCTT |
| | | ACTCTTTCCACCACATTTTCAGTCTTCGAATTGATAACCATCGAAGCACC |
| | | CAGGCGTTTTGCCAGTTCAAGCTTCTCATCGTCAATATCCAATGCTATTA |
| | | CCCTTGCGCCACGAAGCGATGCTCTTACTATGGCGCCAAGTCCAATCATT |
| | | CCGCAACCAATCACGGCCACAGTATCAATGTCAGTTACCTGAGCTCTCGA |
| | | CACGGCATGGAAACCTACGCTCATAGGCTCAATCAGCGCACATTCCTTAT |
| | | CCGAAAGACCGGCAGCCGGAATAACCTTTGTCCAAGGGAGGACAAGGAAC |
| | | TCCTGCATAGAACCGTTACGCTGAACACCCAAAGTCTCGTTGTGTTCGCA |
| | | GGCATTCACACGTCCGTTGCGGCATGAAGCACACTTTCCGCAGTTGGTAT |
| | | ATGGATTTACTGTCACGTTCATTCCCTTCTCGAAACCGACAGGAACGCCT |
| | | TCGCCTATTTCCTCTATCACAGCACCCACTTCATGTCCCGGGATGACAGG |
| | | CATCTTCACCATAGGATTTCTTCCCAGGTAAGTATTAAGGTCGGAACCAC |
| | | AGAATCCGACATATTTGATACGAAGTAAAATTTCTCCGGCTCCAAGTGTT |
| | | GGTTTAACTATATCAGCTACTTGAACCTTTCCGGCTTCAGTAATTTGTAC |
| | | AGCTTTCATAATCTATGTATTTATTTAAATTTGTTATTGTATTATTTTGA |
| | | TGTTGCATTAATTCAATGTTGTTTTTTCTCTATCTTATATCCTCTCCAGC |
| | | CATAATATGCCGTAAAGAAGAAACATATCAGAGGTATTACATATGCCACC |
| | | TGATAGAAGTCCGCGTTATGATTCATCACAAATGCGGTGAACTGAGGGAT |
| | | GCACGCATTACCTATAATAGCCATCACAAGGAATGCCGAACCACTCTTTG |
| | | TGTCCTCGCCAAGGTCGCGTAGTGCAAGTGAGAACTGGGTTGGATACATT |
| | | ATCGACATGAAGAACGACACTGCAAGCATGGCATAAAGTCCTGTCATACC |
| | | ACCGAACATGATAATTACTCCACACAGTATGATATTTACTATAGCGTATG |
| | | TAAGCAGCATATCCTGAGGTCTGAATTTCGACATTAGCATAGTACCTATC |
| | | CATCTGCCGCCAAGGAAAGCCAGCATATACAGTCCGAAGAATGTGGTCGC |
| | | CTCATCCTCCGACAGACCTGCATACATGCAGCAGTAAACTAGGAACAGGC |
| | | TGTTGATGGCTGTCTGCCCTCCGTTATAGAAGAACTGTGCGATAACTCCC |
| | | CATCTCAGGTGTTTGCGTTTCAACACTGCAAAATTGATAAGCTTGCCCTT |
| | | CTCGCCGTGCGATTCCTCCTTGTCAATATCAGGCAACTTATACAGTGCAA |
| | | ACACCACAGCAAGAATAATCAGCAGGACTGCAAGAACCAGATAAGGCATC |
| | | TTCATGGAGTCTGTCTCCATCTGAATAAATCCGTCCCAACCTCCGGGAAA |
| | | GTCGGCAGGCAGAGTCTCGCGAGTATAGTTCTGTCCGGTAAGTATAAGCT |
| | | TACTCAGAAACATTGCGGATATGAAAGCACCAAGACCGTTGAACGACTGT |
| | | GCAAGATTCAGTCTTCTTGAAGCCGTATCGTGTGTACCCAGAGCTGTCAC |
| | | ATACGGATTGGCAGCAGTTTCGAGGAAGCACATTCCCGTTGCCATGATGA |
| | | AGAAGATTACAAGATATGCCCAGTATTCCTTTATCTCGGCTGCAGGGAAG |
| | | AAAAGCAGACCACCGATGGCTGCAAGAATGAGACCGACAATTATACCCGA |
| | | CTTATAGCTGAAACGTTTCATGAACATTGCTATCGGTATGGGAAACAGGA |
| | | AGTAGGCCAGCCAATAGGCAGCTTCAGTGAACGAGGCCTCAAAAGCATTC |
| | | AGTTCACAGGTTTTCATCAACTGCCTGATCATTGTAGGCAATAGATTACT |
| | | GCTGATAGCCCACATGAAGAACAAGCTGAATATCAGTAAAAGCGGTATAA |
| | | AATATTTGTTTTTCATTCTGACATGTTTTTAATATAAGGTAACTCAGGCA |
| | | GATTCTTGAAACCGTAAAAGGCTTTCGCGTTCTCGCCCAAGAAAAGTTTT |
| | | TTGCTTCTCTCTTCCAATTCTTTTGATTTAATCACAAAGTCGTACGACAT |
| | | CTTGTAGGTAATGGCTGTGATTGTGCGTGGATAGTCGGAACCCCACATCA |
| | | GTTTCTCGAAGCCAACAAGGTCGGCAGCTTCGTTGATGGCTCTGACAGCG |
| | | CTGCGGAACGGATAGAACTCGTCATTGAACAGCCAAGTGATACCGCCCGA |
| | | CTCAATCATCACATTCTTATGACGGGCAAGCATTATCTGCTTCTTCCAAT |
| | | CCGGTTTAGTCACCATACCGAAATGCCCGATGGCAATCTTCAAGTACGGA |
| | | CATTCTGAAATGATTTCTTCCATCTCGCCCACCTGGAGGTCTCCCTCTGC |
| | | CATATCTATGGAAAGAATCACCCCCTTGTCTTCCATTAGATGAAACATCC |
| | | TCATCATCTCGTCCGAGTTGAGCATCACCCTACCGTCCTTCAGTTGCAGG |
| | | CGGTGTCCCGGAATCTTTATGGCCTTGAACCCTTTGTCTATAAGTTCAAC |
| | | CGCCTGGTTATAGAAACCCGGTTTTCTGAATTCACACATACCACACACGA |
| | | AGAACCTGTCCGGATATTTCGTCATCACCTCCATCAGATAGTCATTCTGA |
| | | ATGCCGTCGATATACTCCTGTGTGACAACAGCCGCGCCAATCAGGGCATA |
| | | ATTCATATTAGCCAGGAAAACCTCAGCCGTGTTTCTTCCGTCAATCATAA |
| | | AGGGGGGGGGAGCATTTGTCTCACCTCCCCCATAAACAATGATTGACCGT |
| | | TCTCTGTAGTCTTGATTTTCAGGCCATCTACTTCAGTGTCCTGATAAAGC |
| | | CACAGATGCGAATGGGCGTCAATTATTGTATAATCCATAGAAACAGTATT |
| | | TATGAATTTGCCCAACTTACTCTTTGCTGATCGCCTATTATCTCCTTAAC |
| | | CTTTTCCACAAGGCTCCAGTCTATCGGTTCCTCAATGTATTTTATGTTCT |
| | | GAAGCACAGACTCTGTTCTTGCCGAGCTGAACAATGTTGTAGGTATTCTC |
| | | GGATTGCTTACAGAGAACTGCACCGCAAGTTTCTCGATAGGGTATCCCTG |
| | | TTCAGCACAATACTTGGCAGCCTTTGCACACACCTCAATCAATGGTTTTG |
| | | GAGCCGGATGCCATTCAGGAACACCTCTATGTGTGAGAAGTCCCATACCG |
| | | AACGGCGAAGCGTTTATCACTCCCACACCATTTTCGTCAAAATAGTCGAG |
| | | GAAGTCCACCAGCTTGTCGTCGTTCAATGAATAGTGACAGAAGTTAAGCA |
| | | CCGCCTCTACTGTACCCGGAGCGGCATGGTCGATAATCCATTTCAGGTTT |
| | | TCGAGCTGCAGGTCGGTGATACCCACGTGGCCCACCACGCCTTTCTTCTT |
| | | CAGTTCCACCAGAGCAGGCAATGTCTCGTTCACCACCTGGTTCATATCCG |
| | | AGAACTCAACGTCGTGAACGTTGATAAGGTCGATATAGTCGATGTTCAGA |
| | | CGTTCCATACTTTCGTAAACACTCTCCTGAGCGCGTTTGTCCGAGTAGTC |
| | | CCACGTATTCACACCGTCCTTGCCATAGCGTCCCACCTTTGTAGAAAGGA |
| | | TGAACGATTCTCTTGGCAATTCCTTCAGAGCCTTACCCAATACGGTTTCG |
| | | GCTTTATAATGTCCGTAATATGGAGAAACATCAATAAAGTTCAGTCCGCG |
| | | TTCCACTGCTGTAAAAACAGACTGTATAGCGTCACTTTCTTTGATAGAAT |
| | | GAAAAACTCCGCCCAATGAAGATGCGCCATAACTCAATACAGGAACCTTA |
| | | AGTCCTGTCTTTCCCAATTCACGATATTCCATTTTTGATAAATAATTTAA |
| | | AGGTTAATATTTTTTACTCTGTTTATTCTTATTCATACAGATAGAACATA |
| | | CGTTCCATCATCTTCCATTTCTCGTCCGATGTGGCCCCCTCGGCACACTG |
| | | CTGGAATTTGGCTACGTATTCTTCCCATTCGGCCTGACGCGGCAGAGTGG |
| | | CAAGCTTTGCCATAGCTGTATCCCAGTCAAAATCCAGAGGTGTTTCCACT |
| | | ATCATAAAGAGTTTTGACCCCAATATGTATATTTCCATTTCCAGGATTCC |
| | | CACCTCGCGTATTCCGGCGCGTATCTCAGGCCATGCCTCTTCCTTACTGT |
| | | GAGCCTTTCTGTAGGCTTCAATCAATTCCGGATTCTCACGCAGACTCAAT |
| | | GTCTGACAGTATCTCTTCACAGGCAGGGAATAACTTTTCACTTTATATCC |
| | | TTCTGTCTTCATGATATTATTGATATTAATATGTTAGTATTACATGTCAC |
| | | TGTCTTTATCTTTTCGACGATGCTAAAGTATGAAGTATCCATCAAAACAA |
| | | TAGAGGAGATTTTCAAAAAAGAAAGAGGGGATATTATACCCCCTCTTTTT |
| | | CGACATTTTTACCCCTCATAAAGGAGATAAAAAGTCACCCCAAACTCTAT |
| | | AAAAAATCAAAACAGATTGAACTGCATTCCTGTGTAGAAAAATCCCTGGT |
| | | TGGATTTCGGATTCCAATACGTCATCACCGTCAACGGGATTTCATATTCC |
| | | ATAATCCGAAGTTTATAAATCACATTCAGGGACACCTGAGTAATTCCTGC |
| | | CGATTCGGCATACATGGTTCTGTTCACCATTTCCCCGCTTTCATTTCTTG |
| | | AATTTCTCAATGCGAAAGCTGTTCCAATACCAGGACCGACCCTTAGCTTT |
| | | TCGTTCTGATAGATGGTATAGCCCACATATACGAAACTGGAGTAGATGTT |
| | | CTTGCTGTTGTCCAGATCCCTGTCGCGACCGTAAACAAGTGTAGAGAAGC |
| | | TCAACTCCAGCGGAAATTTCCTGTCGCCCGTATAATTGACCATGAGATCA |
| | | ACGAAACGTCCAGTTTCATCAGGCTTATAGTTGAAGAACTCCTTATTATT |
| | | ATATGTAGCCCCGGGCGAGAAATTATATGTATCTATAGCCTTTATCTGAA |
| | | ACCTGCCATGAGTATATGCTATATACTGGCTCAGCTCCTTATAACTCCCC |
| | | CTGGTGTTCGATCCGCCAAGGAAACCGGCGGTAAACCTCCCCGATGGGTC |
| | | GGAAACCGACAAATCGGACGAGAGAATCAGTCCGTCGGCCACTTCAATGC |
| | | CACGCCATAGAATCATGTTCTGTAGAGTAGTACTGAAATGAAGCTGAGCC |
| | | TGAACATTTGCTGACAAAAATATAAATACAGGAATTAACAGTCGCTTTTT |
| | | ATACTTACAGGTATCCAATGATAATATATGTATCATACTCAGAGCAGTAG |
| | | AAAATCGGTTTTAAATTATTATTATGGATTTATTTGTCGAAATACTCTAT |
| | | AAGATTATAAACATTCCAGTTAATATCCGACATGTATTTGGTCAATGATG |
| | | TATAAGGTTTATAGTTATAATCGAGCATACCTTTATTGCAATCCTCATCA |
| | | TCCAGATACTTGAAGAAAACCCATCCTACACAATTCTTGGCTTCGAGCAG |
| | | TCCCAAGGTAAAATGCTGGTAAGCGAATCCACGGTTTTGCTGGTCGCGTA |
| | | CCACGAAACCAGCTCCACTTGAATTGTCAAGCTTAGTATCCTCACCCTTG |
| | | GTATAGAATTCCGTTACCATGAAAGGAGTACCGCCCGCCTGGTTCTTCCA |
| | | GCCATCCATGTAGCCTTTTTCAGGCGACCATTTACTATAATAATTTATGG |
| | | AAATGACATCACAATATTTTCCCGCTGCCTTAATTATATAACTGTTGTAT |
| | | TTAGGAAGGCTGTGCAGGCGTGAACCCAGATAAAGCAATTCAGGATCCTT |
| | | CGATGCCTTAACCGCATTCTTTATGGCAGAATAATATTTTTCCGCACAAA |
| | | TACCGGCAAACTCATTGTTCAGTTCATCCGTTACATCAGAAACATTTGCA |
| | | CTCTTGTCCTTATCCGTCATAAACTTGGCGGCTGCAATATAAGCAGGATC |
| | | CTGCTTGTTTGAAATTTTCAGGAATCTGTCGAGCAGCCTGTTTCCCCATG |
| | | TAGAGAAGTCTATCTCATTATCCGAGAAGAATCCCAACACATCCGGGTTG |
| | | TTTCTGAACATGCCGAAAGCATCCGAATTGAGATACTCCTTGCACCATTC |
| | | ATCCCATCCATCATAAAACACAAGACCTATCTTAAGATTCACGTTCTGCC |
| | | CCGGATAGCTAATTCCCTTGCTATTCTTGAACTCTGCAAGGAATGAAAAG |
| | | GAAGGAGCCTGTGTCAGAGGACTTGAAGCCGATTTATTATAATCATTTAC |
| | | AGCCTTGTCGCCTTCTTCCTTACCGAAAGCGCAGACACTATGAAATCCTA |
| | | TTTCAGAGAATTGTTTCTGCGACTTTGCCACCCAGTCATCTACTGAACTG |
| | | TAAAGCTTGCCGAAAGCTGAGCTGTTGCCATCCATTCTGAATGAGGCGAT |
| | | ACCCCTTACATAATATGGATAACCTTCGGGGTCGACTATCCAACTTCTTC |
| | | CATTTGAGTTTTTCTCAACCCTGAACCGTCCAGTAGCCTTGGATTTTTGC |
| | | CCTTTTGCGTATGAGCCATATTTATTCACGCTTTGCAAATACTCATCCTG |
| | | TGTTTTTGTCTGCTGTTCATAACCAACCAGGTATGGCAATATCCTTGTCT |
| | | TTGCCTCTATAAAAGCCTTGTCAGGTTTTTCCGCATACTCGACAATTATC |
| | | GGTTGATACTGCTTGGTGCTATTAGGATAGGTTTCAGCAGGACCGGGAAC |
| | | AGGCAGTTGCAGTTCTACATCATCATCGTCATTATCGCCTGCATTGTCGC |
| | | CGGGAGTATTATAGTCCTCCACATTCCCCGGTTGTGAGTAAATAACCTCA |
| | | GGCGGAATATATGAGAACTCCTCCTGAGGGTCTTCACATGACAAAGCGAA |
| | | GAACGGAACACTCAAGCAAATGGTTTTAGTAATAATAGTAGAATATTTCA |
| | | TTGTTGCAAATATTTAGTAAATTAATATAAATCCCATGTCCTGATTGTAT |
| | | CCCCCCATCGGTGGTCTATCGGGAACTCCATTTCTCCCCATGCCTTAACA |
| | | GAAGTCCAAGGTTGGTCGGCATCAGTCCAGAATGGGTCAGAGGCAGGCAA |
| | | TCCCAACGGAAGGAATGCAAGTGTAGTCATATACAGGCTGCCATTGTTTG |
| | | TATAATGATTCGAAATGCCAGTCTGATGTCCGCAGAATCCTATGGTGAGG |
| | | AATCCGCCCTCATTGAAGTTATTGCCCGACTTGAACATACGTTTCATACA |
| | | CGCTGTCAGCGCACATCTCACCTGTGCTTTCGATACTCCCGCCGGCAACT |
| | | CATTATACCATGCTATAAGAGCCAGTGGCTGCATTGTTGCCATACGGTAA |
| | | GGTATAGAGCGTCCGAAAACAGGGAATGTTCCTTCAGGAGATATGAAACG |
| | | CTCCAGAATCATGGCGAACCTCTGTGCCCTCATCAATGCCCTGTCATAGT |
| | | ACTTGCGATAGTCGAAACGTGTCCTCACGCCCGATTCCATTATTGCATGT |
| | | ATAGATTCGAGATACATAGGATGGAACACATAACTGCTATAATAATCGAA |
| | | TGCAAAGTGCTGTCCGTCTGCGTACCATCCGTCGCCTACATACCATTCCT |
| | | CCACCTTGCGGAAAGTAGAATTTATACGATATGTATCCTGTCCGGCATCA |
| | | ATTTTGGCAAGGAAGCTTTCAATGGTGGCCGAGAACAGCAGCCAGTTAGT |
| | | GTAAGGAGGGTCAATGCGTCGGAGACCTTTGAACTCTTTTATGTAGCGTT |
| | | CCTTTGTTGTCTGGTCCAGCGGTTTCCACAGCTGGTCGAACGCGCGCAGG |
| | | AAACTTTCCGCAATATAGGCAGCATCAACCAGTGCCTGACCATGACCGTT |
| | | CCACAACAGATAATCCGGACTATTAGGGTCCACCGCATTTGCATAACTCT |
| | | TCAATGCCCATTCTTTCAGTTGCTTGCGCTGCTGTCCTTCTGCTGTATCA |
| | | TCGTCAGGCAGGCTCAACCATGGAGCTATACCGGCCATGAGACGTCCGAA |
| | | AGTTTCCATATATGCAACCTTCTTGTTACGGTTATCCCAGTTTGGACTTA |
| | | CCTCAAGAATCATATTTTTCTGCAGTTCCCCTTTCGCCATATTGCTCAAC |
| | | ACAGGAGCAGCCATCCTGTAAGCCATATCCGTCCAGTATTTTCTTGTCTC |
| | | GTTGTTGTTTGCCTCGAGATAACGCACATACTCGCAAGCGGCAAGAAGGA |
| | | ATGCGCCTACCCCAAAGTTGGCAGTCGACTTGGCGTCAACCACCTGTCCC |
| | | GGAATAGCCTTTTCACCGATTGGCTGGACATAACCCACCGACCAGTCTTT |
| | | CTGCAGTGCAGTCTTGGTAAGATATTTCCATGCTTTCCCCACTACAGGCA |
| | | TAAATTCATCCTTGTCAAGATAACCGTTGTTTATCCCCCAAAGCATACCG |
| | | TAAGTGAAGAAAGCGGTACCGCTTGTTTCCGGTCCCGGAGCATGTTCCGG |
| | | ATCCATCATACTTCTTGTCCAGTAGCCCTCCGGCTGCTGCAGACATGCAA |
| | | CCGCCTTTGCCATACGCACAAACTTATCCTCGAAAAAAGACAGATGCTCA |
| | | TAACCCTCCGGCAGGTCCTTCAGCACCTTTGCCAGAGCGGCAAGCACCCA |
| | | TCCGTCGCCTCTTGCCCAGAAATCCTTCTTTCCGTTCAGACTCTTATGCT |
| | | TGGGATAAACATATTTTGCGTCGCGATAATAGAGTCCTTCCTCCTCATCA |
| | | TACATTATTGAGTCCGACGTACAAAGATATTCATACAGTTTCTTAAGATA |
| | | CCGGTGATTATGCGTAATCTTATACATCTTCGTCATTACCGGCATCACCA |
| | | TATAAAGTCCGTCGCTCCACCACCAGTAATCCTTACGCGGTGTGCTCATC |
| | | TGGTACTCCATGACTTCGCGTGCACGCTTGATTTTATAATTCTCCGGCAT |
| | | GACGTTATACAAGTCCGCATAAGTCTGGAAGCACACCTGATAATCGCCGA |
| | | ACAGCACATAATCATCCTTTACCCCGTATTTATACTTCCATTCAGATTTG |
| | | TTGTTGCTTTTCGCACCCATCCACTGGTTATACTCAGCCCATGCCTCCGA |
| | | ATACTTTCTGTATTCTTCTTTCCCAGTAAGGAAATAGGCTTCCATATTAC |
| | | CGGTGTGATATGCCGCATAATCCCAGAAAGACCTTGCTTCGGGGGCATGA |
| | | TTTTTCTGCCAGGCATCGTTCACTTTTTCAATCATCTCCCTAACTTGCTG |
| | | AGCCTCAGTTTTTTTTTGCGAAGGAAAATGAAGGTAAAACAGCTATAAGG |
| | | ATGTATAACATCCAGTAGTATCTATAACAGTTCATCTTTGTGATATTGTT |
| | | TACATTTTCTAAAACGAAATGGGGAAGAATATATATTCCTCCCTCATTTC |
| | | ACGAATAATTGTATTATTATATTTATTTGTTAGGAGTCCATTCTGCTCCG |
| | | TTGTTGAAACCTTCTGTTGTAGAGTCAAAACTTGCATCTGCTCCTGTACT |
| | | TGGTCTTTCTGTAATTTCTTCAATCTTAAAAGAAGTGATTTTAGCGGTTC |
| | | CAGTAGCATCAGTACCACCAGGGACATTAGTCTGTACAGTTAAAATAACG |
| | | TTCTCAAGAACCGGCCACACAAGTGAACCATCTGCTCTTGAAGCTGGAGT |
| | | TTCAGCAGAAGTAGAACTACTGATTGTGAATGTATTTGTATAGGTTCCAC |
| | | TTCCGGTATTTCTTCCAATCCAGAATTTATATTTATCAGATGCTCCCAAT |
| | | CTGAATGTTGTTGCACAGTCGTTAGATGCGTATGTATAAGTAAATTTGTA |
| | | AGTACAACCATCACGGAATGACATTGATTTAGTAACTGGGAATTGATTAT |
| | | CTGCTGGAACAATTTCCAATTCTCCACTTGCATTAATTTTTTCGGCAACT |
| | | CCTTCTGCAAGATATTCCTTAACTGCATCTATGTTAGCGAAGTTAAAATC |
| | | AAAAGCATCTGCATGAGTCAAAGCAACATTGGCAGATTCAATCTTGATAT |
| | | TAGCATCTTCGTTGTTTTCGTTTTTAGCAGTCAAAGCACTAACAGCATAA |
| | | TCAGTGTTATAACTTACGCTAATATTTGCGTCATTACTATAAATCTTATC |
| | | ACCAAGAATAAGAGTCATAGTAGTTCCATTCACAGAACCGGAAGCAACAG |
| | | GAATTGTTTTTCCTGCTACTGTTATGGTAAATGCTTTGTTAACAGCATCA |
| | | GTGAATGTTCCAGAAACTTCCTTATCGAGTGTAAGTTCAATTCGGTCATT |
| | | ACCTGTTGTCTGATCAGGAACAATTTCTTTAGCTGAAGAAACGGCAACAG |
| | | TAGTTTGTTTTTCCAAATCCACAGGAGGTTCACCGCCTCCTTGATCATCA |
| | | TTCAATACTATCGTTACAATCTGTCCTTTAGTAACTATAAGGTTTTCACC |
| | | ACTGAAGTTATAAGTTTTAGTACCAGAATTTCTTGTAAGTTCTAAAGTAA |
| | | ATCCATCGGTAAATGTCACCGGAGCTACAACCATTGAGTATTCCTTGGCA |
| | | TTTTTATTTTGTTCATTAGGACCAACAAATGTTCCCTCTTTAGCGGTTAG |
| | | AGTTATAACATTAGAACCGGATTCCACTGTCAGGTTTGCTGAAGCATCAA |
| | | TTTTTACGTTCCCTGCAATCTTTACATCACCACCAGCAGTAAGTTTAATA |
| | | CCTGTAAGGTCAGTAAGATTATTTTTAAACTTAACCAATCCACAAGTATT |
| | | CTGGAAAGTTAAAGATTTGTTATTATCTGTTGCAGTAGCATAAGATATAT |
| | | TTGCATTTGCATCGAATCCCCAAGCCGGAGCTGTCTGTTCAGATGGCAGT |
| | | GTAGTAGTTACGACACCTTCAAGACACACAGCTTCGGCATTATAAGGATA |
| | | AAGAGCTGTATATGAATTGTTAGGTGTAGCCTTACCTGTAAACGTTGTAA |
| | | CTGTGCTACCACCTGTAGCGGTAGTAAACTTGTTATTTTCTTGGCCTGAA |
| | | AAGATATTGATTGCATCTCCTGTTGTCCACCACACCGTTGTTCCATTCTG |
| | | CAACGAACTACGGCTTGAAGGCGTACCGGCAACAAAAGTCATATCCTGAG |
| | | GACCACTGACTGCATTTACATTCGACAGTTCGTCTTTTGTACAAGACTGG |
| | | AGCATTGCAATACTCATCAAAGCCGCTCCACAAAATAGCATCGTATTTTT |
| | | CATGACATAAATTATTTGTTAAACAGTTTCAATAATAAAAAATCACATCA |
| | | CTTGTTATTCATATTCTTATTCTTTAGGATCAGGTTTCCATTCAGTACCG |
| | | TCATCTTCAAAATCATCATGACCGCCATCTACAATTCCGGGAGGTATTGA |
| | | TATTCGGCATACCGCACTTTTTATTCCATTACCCGTATCTACAGAAGCAC |
| | | CGATATTAGAATCTCTGCCCCCGTCGATTGCCACGACCGTACATCTCATT |
| | | TTATCGTCCGATGGTGTAATCATCAACACATCAGGGAAAGAAGTTCCCCA |
| | | AACTATTGACTTGTAACCGGTATAAGGGAGATTATCCTTGGTTATATTAA |
| | | TACCCAACTCCACAGTGCCACTATATGGTAATTCTATATAACTGACAGGT |
| | | TTGTTGTCAGTCTGCCCATCCTTGAACACTACATATTCAATTTTTATCTC |
| | | CTCAGCTGGTGTTCCATCACCTCCACCTACGCCATCATCATCCTTATCAC |
| | | ACGAGATTGCCGTAAACTGTATAAAAAGAAGTATGAAAAGGTTGTATACT |
| | | GACAGAATCCGTGGTTTTATATCAACCATAATAAAATGTTATTTAAGCGC |
| | | CAAACAAAATTTTCAATATTCAAAAGGCATAAGAGGAAACCCTGAATATG |
| | | CCTTATTACCATGAAAACAAATCAATCTACCTTTTTCAATCCGGAATCAG |
| | | AAAAATATGTTATTTATTTAGAACATATTTTTCCGATTTGCCAGATTACA |
| | | ATCACAATAAATAAATCAACAACTAAATCTAATTACCTAATCTTATAACT |
| | | AAACCCTCAAACAATGTTATTTAACCTTTTCTATCTTGACATCATCAAGC |
| | | AGGAAGCATCCACCATTACCTGAACCCGGAACAGCTGTGAAACGATATAC |
| | | AAAACCATTTTCCTGCAATTTGAATTTAACTGTTGTAAGATTGTAATTCT |
| | | TACGGTCTTTCTTGACCTCAGCAGTGGCAATTTCTTCCAGTTTCTTTGAA |
| | | TCCGGATTATAGTACTCAATCCTGAAGTTAGGTTTGTCACCCCAACTGTA |
| | | TTTGGTATAAGCTGAAATCTGATATTCTGCTCCAGTTTCATAGCTGATGT |
| | | TTACAGCCTGCCACATACCAACCTTCACCTCAACAGCATAGTTGCCTGAA |
| | | TGTGCCTTTTTCGCATCAACTATTTTGTTATCTTTCTTTTCCCAGACATT |
| | | CCATGATGTCAAGTCACCTGACTCAAAATCACCGTTCTTAATTTCCTGAG |
| | | CGTATGCAGAAGTCATCATCATTCCGCAAGCCATCATTGCTAAAATTTCT |
| | | TTTTTCATTTTTTCTAAGGTTTTTAATTTAAGTATTATGTTGTATCTATT |
| | | AAAATCACTCTTCTATTGGAACCAACTTATAAGCCCTGACCCAGTCATAA |
| | | TAAGTAGTACTTTTGTCCTTATCCTTCAAGTCCTCAGCTGTAGGTACTTG |
| | | TTTTTCCCAATCGTATGTTTCAGTAACTATATGTATGAACATAGGTCGGT |
| | | CAAACGGAGTATCTGTATATTTTGTTGTAGGCTTGATAGTGTACATATAC |
| | | TTTCCGTCATAATAGAATTTCACGGTATTTGCATCCACCCACCAACAACC |
| | | GTAAGTATGGAAATCTTCTGCCGATGGGTCCGTCATATACGAAACCACAT |
| | | CCGAACGTTTCGCCGTATTGTCAGTACGTTTGCCTCCTTGTTCCTGATAC |
| | | CAATAGTGAGTATTACTGTTCATCTGCATATTCCATGTCTTGTTCCACGG |
| | | ATTATCAGGGTTGACACTTCTTATTATACCCATTGTTTCTATAATATCAA |
| | | GTTCCTGACTGCTCCATGTCTTTATCTTCTTGCCGCCTTTCATTATTTCC |
| | | TTCATTACCGGGCGGTTGGAAAGCCAAAAAGTAGACGACATGGTAGTGAG |
| | | CGAAGCCTTCATCCTTGTTTCATAATACCCATAATGTGCCTGGTTCTTTG |
| | | CAGAAGCAACCGCTCCACCGGCAAGACGATATTTATCGCCCGGCTTTCCA |
| | | TCAAGTCCTTCTGTTGGCGACAAAACGGTATTGATTATACGAAGACAACC |
| | | TTTCTTGACACTAACATTCTCTGCCTTGAAAGTTGCAGGCGGCCGACCGT |
| | | TAGTCCAATAAGGACTTTTAGCATGCCATTTAGCGGCATTAAGACGTTTA |
| | | CCATTGAATTCATCAGTATAATCTTCGTTAACTACCCATTTATAACCCTC |
| | | AGGAGCCTCAGGCAAATTTTTTATATGCTCTTCAGCCAAAGAATATTCCT |
| | | TATCATTTTTTAATGTATAAGATGACAGGAATAAAGATGCAGCAGATAAA |
| | | TACAATACTGTTTTTCTCATAAACTTTGTCGTTTTAGATTTTTTGTTACA |
| | | CGACAAAAGTATATAAGTTTCATGAAAGCATTAAGGGGGATTTACATCGT |
| | | AAAAGGTGGGGTAAAATTCTACCACTCCCTGAAACACAATTATTTCACTC |
| | | ATGAAACCATGTGTTTTTACGATATATAAAACCCGACAGAAGAATAATAC |
| | | CGTATTACCGGCTAATTTACATAAGAATAACTTTTCAAACCGCCATATAC |
| | | CCCACTTTACGTCCGTACCCTCAGTCCTCGACTCCGGCAATATGTTTTCC |
| | | ATATCGAGATCTATGGTTTTCTGCCTCGGATTCAACCACTAACTGTCGAG |
| | | CATGTGGATTGCGTATCTGTCATAGAATCTCTTTCCGAACCATATTATCT |
| | | CGTCTGTGCTAAGTATGTTGTTCAGACGGATAATCTTTCCGGTATTTTAC |
| | | CACCTACTTCTCTTGCAAATCCTGATCTGATATAACCGGATACTCTCAAT |
| | | TCATTGATTTCCGACTTGTATACAGTCTGCGAAGAGGCATTGAAACTACT |
| | | GCACAGACTGAACAGCAGCAGGGGAATAATTTAACTGATTTTAATAGTAG |
| | | ACATTCTGTGTTCATAATATTTCATTTTAATGATTACGTTTCTGACTTTC |
| | | GTCTGATGCAAAATTATGAGGTATCGGACGGGGTTGTATCTTTCAGTAAA |
| | | AATCAGTAAAGTCTTGGCAAGGGGTAAAAAACTTAACATCTTGTATATAA |
| | | ATATATTACAAACAAGGTGCAAAGATTTTCAGTAAACGATGGCGAATACA |
| | | GAACCTATATATTTACACGCCATAAAATGAAGAAAAAGCAGTAGGAAAAA |
| | | AATGCGGGCAAGTTCCGGATAAAATGTGGGCAAGTTTAAGGTAAAACTTG |
| | | CCCGCATTTTAGATAGAATGCGATCGCATTTAAAACAAGTAAAAAACGAA |
| | | GAAAAAAAATATGTGTTCTTCACAGAACACATATTTCAAAAATAGGTATA |
| | | AACACGCTAAACAATGTTAACAAAATCTATTTATAAAAAAAGCTCACATC |
| | | AATAATATCTGCAACATTTTTACAATACTCCATAAATGAAGAGACCTTGG |
| | | GATGATTTATACACAGAGCTATCTGTGATGTAGGCGAAAAACGTCCTGTC |
| | | CCGTCAAGAAACGCTGTAAGCTCAGATGGGAGGAGTATACTGCCAATACC |
| | | TGGATTTACGTCAGTCAGAACGACTGTATTTACAGCTTCCACCGCTGACA |
| | | CATCAAGATAATCGAGTGCCGGAAGATCTGCGAAGTGCAATTTTCCTATC |
| | | ATATTGCCGCCTTTGCTGCCCTGAAGAGAGACACTCTCCAATGAAGAACA |
| | | ACCGGATATATGGATTTCACTGTCGAATATTGAAGTTTCGGAAACATCAT |
| | | CATTAAGTATAACAGAAGGAACAACTACCAATTGAAGCGAACTGTTATTC |
| | | TCCACCCTAAGTACTTTCAATGATGATGCGGAACTTAAATCCATTCCCAA |
| | | AGGTGTATCAATATTAGAGATTGAAAATACTGAAACTCCCGAAGACGGCT |
| | | TGACATACGACATGGAATAATGCTTGGACTTCACTCCCGAAATGTCTACT |
| | | TTTCCTCTGAAACCGGGATTTGACAATATATACTCCACACCCTCAAGGTT |
| | | AGCTGTCTGCGACAGGAAAATGAGGTCGTTCCCTTCGGTTATCCTCTTCG |
| | | TGACATCAATCTCCAACGATGAGACAAACACCGACGGGAAGTTTCTGTAA |
| | | AGATATGAACGGAGCAAAGGATCCGGTACTCTTCGGTTTACTGTATATTC |
| | | AGTGTAATTTCCATCCTCGTCCGACATCACGACAAGACATTTGTCCGTCA |
| | | TGGCTTTATAGAATGCAGGTATGACATCTGTATTCCATTTTGCAAAGTAA |
| | | GGAAGTTTCAGATTTGTAAGACTTTTACAAAGCACCGTAGTGCCATCGGT |
| | | TGAAATAAGATTGAGATACGAAGTTATGCCGTCATTGCCGCGTAAAGCTA |
| | | CCGATTTTATTCCTTCGGGCAGGTCAGCAAAGTCGAATATAGAAAAACTG |
| | | TTACACTCAAGATTGACATCTGCGAGCGAAGGAAAACTCCTCAAACCGCT |
| | | AATAGATGTAAGTTCGCATCTACTCAAGTCCAAAGAAGTGGTATTGAGAA |
| | | CTTGATTGTCACAAATCAGCTCTCCGTTTTCGCTGAAATTAAATCCTTTC |
| | | CGGGTCAAGACATCGCGTAACTTTGTATCAAAAGTCACTTCAGACACTTC |
| | | AAAGTCGGAAATTTCTGTTTCATCCTTACACGAGATTATTGTGAAACAGA |
| | | GAACTATCAGTACATAAAAGCTAATAAAATTCCTCATAACAATCAGTTTT |
| | | GTGGTAATAAGACTATATTATCAATCCAAGCCGCGTCGTTCTGTCTTTCG |
| | | CACACAATGGCACACACTACTTTTTTCACTGTAGAATTAAAATCGAAAGA |
| | | TACGGCTTTATAATTGCCGGGAGAAGAAAATTCCTCTGTATATACCGTTC |
| | | CTGTAGACATATCCTGTAGCATGACTTTCAACTTACATGCTCCTTCGGTC |
| | | TTTACATCAGCAGAGAAGCGATAAGTCCTGCCACTCTCCATGTCAACCCT |
| | | CTGCATGAGTCCTGCATGACCAGATATACAGGCTACATTATTGCCTGCAT |
| | | TGTCAGTCTGTACGCAAACCGTACCATAGTTACCCAATGGCTGCCATGCT |
| | | GAAAGTCCTTCGCTGAAGGTTCCATTCTGCAAGGTAGAGACAGTATATTT |
| | | CTCAACCTGCAGTATCATGGACGATACGTGACCTCCTCCGTCGGAAAAGG |
| | | TGATGTCGACATTATTATCGCCATTCTTCAGCAGCTGTATGTCGAACGGT |
| | | ACTTCTATCATACCGAAAAATATATTGCGGTTGCTCTGGCCGTAGCCTTT |
| | | CCAGTTGTCGGGAACACTCACAGCGGTACCATTAATCTTTACCACCGGTT |
| | | TCTTGGAAGCAGAGACAGGACGGCCTATCGACATACGCAAGCTTGCTCTG |
| | | CCCGAACCGGACTCGATTCCTGTGAAGGGGAACGAAAGGGATGATCCGGC |
| | | GGAAATCGGTTTCAGATACTCACTGCTGTAATATTTATTGCGGATTATGG |
| | | AGTTCGTGAATGCTGACGAAGACACATCTGCTACAAGGACTATGGTCTGA |
| | | TTTGGGACAATTGAGATGCTTTCAGGCATGGACGGGACATTCTGTTCCGT |
| | | ATATTCTATACCTGCGTTATAATTGACATATAGAGAACGCTTTGTGACAT |
| | | TCGATACATCCTTCCAGCTATTCTTATTGTTCAGATATACAGTCTGCGGG |
| | | TTATCATCAAGATTATCAAGGGCGATATAGAGTCTGCCTCCATCCTTGAA |
| | | TGCCTGTACCTGAATATCAGGATTACTGCTGGTTATATCAACACGTTCGC |
| | | CTTTTACATTCTTCCAGAGTTCGAAGAAATATTTTTTGTCATTAAGCCTC |
| | | CATGTGGTATTCTTCAGATTCTGAGGATTGTCGGGAATAAACAGTGCCGC |
| | | ACTATATGAAGTATAATTGTTTGCAGCGGTGATATGCCACTCAGCCTTAT |
| | | CTGAGACAAAAGGTATTGAGATAAACAAATTGTCCTGACGTTCCATCAGA |
| | | TTAAACAGAAAATGATTAAACGACGAAACACTCCGCACACTGCTTATGTC |
| | | ATCATAGCTGTCGTCGGGCTTGCTGTTGTCAATACCTCCAAACTCGGAAA |
| | | TGGCAAGAGGCTTGACATGTCCGAACTTAATATAGGAATACGCCTCAACC |
| | | ATATCAAGAACTGCTTCGGAGTTACTTCCTGAACGTTTCGTATCGGTGCC |
| | | GGTTACATTTATTCCATCATAAAGATGTACAGAGAATCCATCCATATATG |
| | | CACCTGCCCGATCGATGAACATTTTCATGCGGGTGTTCCAGTAATTGAAG |
| | | TTCCCATCCTCCCAGGCGGGGTAGGCTGCGGCATAGCCTATCACCTTCAT |
| | | CTTTCCGTTAAGACGCGGATTATTGTGTATATGTTTACCTATTGAAGCAT |
| | | AAAAATCGACCATCAGTTCGCGCATAGCCTGTCCCTGAACGGTAAAACCG |
| | | GCATCATTTGCATGAACGAACGGTTCATTGAGGGGTTCAAAAAACTCAGG |
| | | TACCAGCTCGCTGTTGGAATAATACTCAGCCGACCATGCACCTGCAGCCT |
| | | GAACGTCTATGCCGCCCTGTATGTGCTGTACATAGGGATGCTCTGTGGCA |
| | | ATATATCTTTTTACGGAAATATTTCCGCTGTATGGTTTCATCTGAGGATA |
| | | TTTGCCTACCTCATGCGTCTTGTTATACGCATACGAGTATGGTCCCCAGA |
| | | ACTTTCTTCCAAGACCGACCTGATAGTCGGCAAGAAACTTGCCTACATCC |
| | | TTATCATCATCGGAGGTGGAATGAATATTGAAATATTTAGAACGGTCGAG |
| | | TTCTGAAACACCGCTCAAAAAGCGACGGGTATTATAGTCGACAACCACCT |
| | | CGTTCCTTTCCTGACAATAAATACCGGGAGGAACACCTAGGGTAAATGCC |
| | | GATAACAGAAAAATATATTTATAGCTCATAATTTCTTTCCTTTTAGACAC |
| | | AGAAACTTGTCAGTCCTGATGTGGATACATTATTTTCTCACTTTCTTATC |
| | | GTAGCGTTCAGTCTGAAGAATCATAGTAGCCACACGGCCTCCATTATCCG |
| | | GGAATGTTACTGACACCGAATTTTTTCCTTTTCTGATTAACCGGTAGTCG |
| | | AAAGGTATTTCTATCATACCGAAGAAATCGTCTCTGCCGGTCTGGTCATA |
| | | TCCTCTCCAATTGTCGGGCATGTCGACTTTCTTGCCATTAACCATTATTT |
| | | CAGGTTTCTTCGACATCTCGTGCTTCCTGCCTATTGACATACGCAGAACA |
| | | GCTCTTCCTGTACCCGGTTTCAGACCATCGAAATCAAACACAATTGGTTT |
| | | TCCGGCTTCCACCGGCTGAAGATAAGTGTTGCTATAATATTTAGTACGAA |
| | | CTATTCTGTTTGAATACTTTTTACGGATGATGTCGGCACACAATATTATT |
| | | GTCTCATCTTTTATAATGTCAATACTTTGAGGCATCGAGTTCAGCGTCTT |
| | | TTCATCATAAACTATACCTTTATCGAAAATCATCTTCAAAGAGCGCACAG |
| | | AAACATTATCTACACCCTTCCAATTCAGTACGTTTTTCAAGTTTACCTTA |
| | | TGTGTATAGTCATCAAGATTGTCGACAGCTATGTAAAGCCTGTCATCGTC |
| | | CTTAAAAGCTGCCACCTGTATGTCCGGATTGTCGGAAACAATATCTACAC |
| | | GTTCGCCTTTCACATCCTTCCATAACTTGAAGAAATATTTCTTGTCGTTC |
| | | AGTTTCCATGCGGTATTCTTCAAGTCGTGAGGATTGTTGGCAACAAATAA |
| | | AGCAGCTCCGTATGGTTCGAAATTATATTGTTTCGTTATATGCCATTCGG |
| | | CCTTGTCAGAAACAAAGGGTATTGAGATGAGCATCTTGTCTTCGCGTTCA |
| | | AGAAGATTGAACAGTATATGATTGAACGAAGCGACAGTTCGTACAGAGGC |
| | | TATCGGATTATATCCTTTGGAAGTGTTGTCTATTCCTCCATATTCGGTTA |
| | | CGGCAAGAGGAAGAACTTTCCCCAAGCGGATGAACGAGTAGTTTTCCATA |
| | | AGGTCGAGAATAGCTTCGGAATTACTTCCCGAACGGCGGGAACTCTTGCC |
| | | TACTATGTTTATTCCATCGTAAAGATGTACCGACAAGCCATCCATGTACT |
| | | CCCCGGCACGGTCAATGAACATCTTCATAGTATTATTCCAATGGTCGAAA |
| | | TCGCGCAACTCCATAGCCGGATATGCCGCGGCATATCCAATGATTTTCAT |
| | | TTTTTTCAGACTTGGCTCAGCGTGAATATGCTTTCCTGTCTGTGCATAAA |
| | | AATCTGCCATGAGCATCCTCATTTCCTGACCATGCATATTGAAACATTTG |
| | | TCGCGTGCATGGACAAAGGGTTCGTTAATGGGTTCGAAAAATTCAGGAAC |
| | | TGCCCCTTTCACATGCTTGGAATAGTATTCGGCAGCCCATGCACCCGCCT |
| | | TCACTGGGTCTATGCCCCATTGTATGGTACGCGCGTTGGCATGTTCCGTA |
| | | GCGACATATCGTTTTGTTTCCTTCAAATCAGTGTAGTTCAAAGGCTTTTC |
| | | TGAAAAAGGATATTCGCCAACCTTTTTTGTCTTGCCATATGAATAAGAGA |
| | | ACGGTCCCCAGAAAGAGCGGCCGATTCCTACACCGTAATCTGCAAGAAAT |
| | | TTCCTGACATCTGGATCAGAATCTTTAGATGTGTGTATATTGAAATATTT |
| | | ACCTCTGTCAAGTGCCGATACATCATTCAGGTATCTCTGAGTGGCATAAT |
| | | CCACTGTGACAGTAGTGTTATAAGTCTTATTCTCGGAAGATGATAAAGGA |
| | | AAAACCGAGAAAGACAAACACACAGACAAAGCTGTAAGAATTATGTTATT |
| | | CATTGTATTATCAAAATTTAAAAGGCAGAGAACACTCCGATAGTTCAATT |
| | | AAAGTATTCCCTGCCATTAAGATTATCACTTCTGTTTAAACACTAATATC |
| | | AGAAATCGGCCGGTTTGAGTACATCGTTCAGCACCACTTCATATTCAACT |
| | | TCTGTTCCGTCGTTTTCAGTAACAGTAAGATGGCCGTAACCGCCACTTGA |
| | | GTTATTTTCTTTCTTACCTTCAAACATGAACATTCTCTTCTTCGTCACTT |
| | | CCTGTTCTTCTTTATCGCCTGTTTCAGGATTGATAACTTCTTCCTTTTCA |
| | | GTATAGACTTCATTGAAAGAGAATGAGAGATGTTTTTCTGTATCCGAATT |
| | | GATTTTCAGCCACTCGGGCAATTCCGAAGGAGCCTCAGACGACTCGGCAA |
| | | AGAACTCGATCTTATTCATTCTCAAAGTCTGATAGTCATTCTTCCAGGCA |
| | | ATGAGGTCGAACAATTCGCGATAAACAGAAAACTTGGAAACCTCGCCTGT |
| | | CTTTCCTGTTTCCACATTCTTGAGATAGGTAAGTTCATACACGGGAGTAG |
| | | AATCAAGTTCGACCTCAGCCCATTTGTCATTATCACATGCTCCGAACAAA |
| | | ACCAAAGCACATAAGAATGTAATTGTCTTATAAATTTTATCTATTAGCTT |
| | | CATTGTTACTATAATTTATTATGGTCTTACTTCAATATATCCGAAAAATA |
| | | TATCGTCAAAATAAATATTATCCTTAAAGGCATTAAAGCGCATACTGAGC |
| | | AATATATTGTCCATTTCAGCCTTTGAAGTCACAGTGGTTGTGGCCGACAT |
| | | CCATTTGCTGTCGGAGCCATTCACAATGCCGCACCATGGTCTATCGCTCT |
| | | GCCATGTCATATCTTCAGCTCCTTCTTTACCTGCCGGAACGAAATACGGA |
| | | CTCATACCCTTACCCTGTTTATACCCCGGTGTATAATATTTGTAGCTGAA |
| | | AGTATATGTACCTTTACCACCAGTAAATGTCTTGGAGAGTAATGCCCTGC |
| | | ATCGGTCAAATGCTTCGACAAACATACATTTTGCACTGTTGTTTATTCCA |
| | | TCCTTCAGAGGATTGTCCACAACCTGTGAAGGAACTACAGGATGTGTTTT |
| | | GGTATCGGCATCAATAACTTTCCAGTCGGCATATGTGTCAGAATTTTCAA |
| | | AATCTTCATCCAGGAACGCACCAAAAGTAGTCGCTACGTTTGGAGCTGTA |
| | | GCCTTTATCTCAAGGTTCTGATATCCAACCAACGCTTCAGTTAAAGTTCC |
| | | TGTAAGGGTCAGTTCATCTGTGTTATAGATTTTCTCAACCAAAGTAAGAA |
| | | TCAGTTCATATCTGCTTTGCTTGTTTACTTCTGCTGCTGTGATGTTTACG |
| | | CTACCCCTGACAGCTGACGGTCTGTTATACGAGTTGGAGTAAGTAAGCTT |
| | | TAGAGATGATGGATTTATCTCTTTATATCCAAACTCAGAATTATCCAAAT |
| | | CTATAGCAATGTGTGTTTGGTCAATCTGACGGATGTTATAAGTAATAGGA |
| | | TCATCACTAGGTACTACTGTAATAGCCAAAGGCACAACAAGAGTTTTTGG |
| | | CGAAGCTTTTGGAGTGTACTTACCTTTACCCTCACTGGCAGAAGTTCTTT |
| | | CTATTGTCATGGAAAGAAGCAATGGCTTATCGCTGAATTTCTTTGCAGTG |
| | | AACTGGTATGGAGTGTCAAAACTGGTTAATTCGTCATTTACGCCAGTATC |
| | | CGCACATTTGAAAGTCCATTTGTTAGGCAATCCGTATGAATCGTCCTTAA |
| | | TATAGACAGACTTACCATATTCAAGTTCGTATTTTTCGTATTCGGGAGCT |
| | | TCCTCAGTTCCACCGACTATTCCGGTCTTTATTTCCTGTGTACACTCCGG |
| | | ATCACTGTATACCTTTACGGCCGGTACGAGGTTAGGATCATACACGCGGA |
| | | TATGGAAAGTTGTATCCATCACATATACATCACCCTCCTGCTTAGCATAA |
| | | CAATATTTTTTGATATATCCTCCGGTATTGTCGTCATACACCGAATATGG |
| | | ATATACAACCTGTCTGCGGAAAGTATTGCACAAACGTACCGTATGGTCAC |
| | | CGGGTTTAGTGAAATACACATGTATGGTTTTCAAATCGTTGGTATGAGGG |
| | | ATGGATTCATCAATCAGGTTTGTATAGTCTGTCTGTCCCCACTCCATCTT |
| | | ACCATTAAGGAACTTTGTACCATCATCCGACACAACCCACTGATGCGACA |
| | | ACATGCCTTGGGATAAGTCCATTATACTTATATAGTTATTAAGATTCAGC |
| | | TGAATAGGTGAAACGTTTTCCTGATCTGTACTCACATGCCAGGTACATTC |
| | | AGCCACGTTATTCAACGGTTCAAACTCATCATCCTTACAAGATGTCAGAA |
| | | CCGAGATTAATGAAAGAGCAATATATAAAAATCTATTTTTCATCGTATTT |
| | | ATTTATTAATATCAGGATTTGATGTAATTTCTATATTTGGAATAGGCCAG |
| | | TATGCCACTTGCGGACCGTAGTTCAATGATGCTTGGAAATAATCCACAAA |
| | | AGCGTTTCCTCTCTTTTCTGGCGGCAGCTCATAAAATCTGTACTGCTTTC |
| | | CAAAGTTGAATGCTGATACCAAAGCATTAGGGTCATCAGGATTAGGCTTA |
| | | AGATATTTGGTCTGAATCATACAGTACTTATATTCGTCGGATGCCAACTG |
| | | ATCAAACCTTTCCTTAGTTATATTCCAGCGTCTCAAATCAATGACACGTA |
| | | TGGCATGTCCTTCCATACACAGTTCAAGAGGACGTTCCACATACATCAGA |
| | | TGATTCATTACATCACTTGCAGCATATTCCTTCTCATCGTATGTATATCT |
| | | CTTGAATTCTCCCTGTTCCGATTTTCCGATAAGCACAACTCCAGCACGGT |
| | | GACGTACCTTGTTGATGGCATTGATAGCTGACTGAACATTTCCATCGCTT |
| | | GCACCGCCTTTAATCAGACATTCTGCATACATCAGATATATATCTGCCAA |
| | | ACGGATAAGACGATAGTTTATTCCTGAGGCCATAGCAGGCTTAAATTCAG |
| | | TTTCACTCTTACGTGTATCCCAATTTGATAATTTTCTGAAATACGCTGAA |
| | | GAGCCACGGTTGAATTTTGATACCTGTTGTGGGAGAGACTGATAATATAT |
| | | CAGACTTTCATCGCCGTTTATTGCAAGAGAGGCAGATGCACGCATGGAAT |
| | | AGCTTCTGAGGCGATATGCCTGACCGTCTTCCCATTTAAATTCCGGAACT |
| | | ATGTCATCGTAGCCGGTAATCTTATTGTATAAAACTTTATTATCTCCGAC |
| | | AGTTGAGACGAGTCGTTCGCGTACTCCAACATATTTTCCTGCTGTTGCAT |
| | | CCCACGTATAAACGTACGTTCTGTTATATACGACACCCTGACGGTCCACC |
| | | TGCGAGCTGAAAGTTGTTCCCAACTGGTCGTATATAATATCCCTATGTTC |
| | | AGGATCACCATAATTGTCGGACTGCATTTTTATCCAGTTACGTTCATCAA |
| | | GTCTGTCCACCGGCTCTGTTTCGAATGCTTCAACAAGCCAAAAAGCAGGA |
| | | ACAGTGTTAAGCCAGGCATCGCCCAAGCCATTTACATTCATTCCCCATAT |
| | | ATTATATAAGGTAGACTCCGACCATGTACCGAATTCTGTATTATACTGTG |
| | | TAGAATAGGAAACCTCGAGAATAGATTCCGAATTGAATTCATTGGCAGCA |
| | | GTAAAATTATCGACTATGTCATCAACCAAAGCAAAACCTCCATTATCAAT |
| | | AATATCCTTAAAATATTCGGCAGCTTTATTATACTCTTTATCATAAAGGT |
| | | AGCTTTTGCCTAATATTGCCTTTACAGCCCAAGAGGTGATACGTCCCAAA |
| | | TCGGTTTTCTCCCATTTGTCATTCAAGCCAAGGTCAAGAGCTTTCTGTAA |
| | | ATCTTCTCTGTAATATTTCTTGATTTCATCACTTGGTGTAACCTTTTTAT |
| | | AGTAATCTTCTTCTACCTCTGCAATTTCATTAATATAAGGAACATTACCA |
| | | TTATTGAATGAATTATTGAGATAAAAATAAAACAAGCCACGCAAAGAATA |
| | | TGCCTGTGCCTCAATCTGAGCAAGCTTGGTTATTTGAGGTTCATCTGTAA |
| | | CATTTGGACGGATTTTCTCTATACTGGCCAGAACCTGATTCGCACGGAAC |
| | | ACACCAGTATACAGTGCAGACCATTTACCACGGACTGTTCCGTATGAATC |
| | | ATTAAAGGTTTGCTTATAGGCTTCGTTATCAAACTGCTTTCTGTCCTTAT |
| | | TACCTTCAACTGCTATATCACTTCTACGGTTCTCATCGAGCGGATGATAA |
| | | ATATTGGTATTTTTCAAAGCATTATATACAGCAGCCAGTCCTTTCTCGCA |
| | | GTCGCCTATTGTTTTATAAAAATTCTGTGTTGTCAGCTGATGTATGTTTT |
| | | CCTGCGTAAGGAAATCGTCGCATGAAACCAATGTCATGCCCGACATCAAC |
| | | AGACTGAATACTATTGTTTTATATCTGAAGTTCATATATTTATATTATTA |
| | | AAAGTTAGAAATTAATCTGGAATCCGCCACGCATCTGGATACTTATAGGA |
| | | TATGTTCCATAGTCCAAACCACGACGTGACAATCCATTACTACCGACCTC |
| | | AGGGTCGTATCCGTCGTATTTTGTCAGTGTAAGAAGATTATCGGCTGCAA |
| | | CGTATAAACGGAACTTGCCCAATCCAAGCTTTGATACCCAACTCTTGGGG |
| | | AATGAATATCCTAACATAATATTTTTAAGTCTGACAAATGAACCGTCCTC |
| | | AATCCACATATCAGTATGAGCACGATAGTTGTTATGCCCCTCTGTACGAT |
| | | AAGAAGGAATGGTAGAGGTATAGTTGGTAGGGGTCCACATGTATATCAGT |
| | | TCCTTATTGGTTCTTCTTTGATATGTATATATCTTCGTACCGTTTATTAT |
| | | TTCATTTCCAACTGAAGCATACCAGTTCATAGAGAAATCGAAGCCTCTAT |
| | | AGTCGGCCGAGAAGTTCAAACCAAGTTCATAATCCGGCATACCACTACCG |
| | | GCATAAACACGGTCGTCATCATTAAGAACACCATCATTATTGGTATCGAT |
| | | ATACATAAGGTCACCCATACGGGCACTTGACTGTAATTTCTGATATTCTG |
| | | CAAGCTTCTGTTCAGTATTGATTACCCCTGCGGTTGGCATAACAAAGAAA |
| | | GCACCGGCTTCATATCCTTTCTTGATTGCAGTTACATAATCACTTCCTGA |
| | | TGAAACAGGTTTACCGTCGGGGAAGAAATATAACTCATTTTTTCCTGCCA |
| | | TAGACACAATCTCATTCACGTTTTTGGTAAATGTACCAGTCAAGCTGTAA |
| | | TTAACACCACGTATTTTGTTGCGGTGAGTAAGTGAAAACTCAACACCACG |
| | | GTTTTCCATATCTCCGGCATTCAATGTAACAGTTGAACTCTGGCCCCCTC |
| | | CATTTGACGGTGGCACGACCATCGGGAAAAGCATATTCTTCTTGTTACTC |
| | | TTGTACAAATCAAGACCTAAGATAAGCTTGTTATTATATAAAGCCATGTC |
| | | GATACCGGCATTAAGCTGCTGGGTTGTTTCCCATTTCACATTCGGATTGG |
| | | CAAATCCCAATTGGGTAAAACCATTTGCAAGAATTTCGGAAGTTCCGGTA |
| | | CCAAAAGTATAGTCGTAGTTTTTGTATATAGCTGGTGCGTATGAATAATC |
| | | AGGGAAGTTCTGATTACCGGTAGTACCATAGCTGAATCTTAATTTTAACG |
| | | AATTTACTAGCCACCTGAATCTGTCGAAGAATGATTCCTCAGAAATATTC |
| | | CATCCTACAGACAATGACGGGAACAATCCCCAACGATTTTCTTCGGAGAA |
| | | CTTAGATGAACCGTCGCGCCTGATACTGGCACTTGCCATGTATTTGTCTG |
| | | CATAGCTATATTGTAGACGACCCAACATACCAACCATTGTACTGATACGG |
| | | TCCTGTCCCCACTGGCCACTGCCTGTACCCACAGTCATATCGGATGTTCC |
| | | CGCATTTAGGTTCGGAATCTCGTTAGTAACCAAATCCATTATACTGGCAT |
| | | AGAACATCTCGTATGTATATTTCTCCATACTGAAAACTCCGGTAAATTTA |
| | | ATATCATGCTTTTTTATCTTCTTATTATAATTTACCATTGTTTCCCAAGT |
| | | GAGACTGGTATTCTTTGAATGAGTATCTTTTAATTGCGAACGGTAATTAG |
| | | AGCTGGTTACCTTTTCGCCTTTCTGATTATATACCTCAAACTCAGGTCGA |
| | | ATTGAGACAGCTTTCTGATTGTTATATCCAAAGCCCAAACGTGTGGAAAC |
| | | ATTCAGTCCGGGAATTACATTATAAGCAAGATAAAAATTACCGTTAAATG |
| | | ATTCTGTGTCCTTATGATTTTCCTCTTTCAATCTTCCCAATGTATAACTT |
| | | ACGCCCTGTAAATCTGCAGGATCGCCAGCTGCATTTACTATACTTGCCTG |
| | | TGGATAAATCTGAGAACGAGTAGGCGAGTAGTCATAACATTCGTTCAATA |
| | | ACCCCCAAGCCGGAGATAACTGGTTTTCTATCTTCATAGCGATGTTAGTG |
| | | TTGATAGTCCATTTTCCGCGCTGAAAATGTGTATTCGAACGAATATTATA |
| | | TCTTTTGTAATCGGAATTTATCAACACACCTTTCTGGTCGAAATAGTTCG |
| | | CGGTAAGGTTATATGTCAAATCTTTCTTGCCGCCATTCGCAGTAACAGAA |
| | | TAATTCTGTATTGGTGCGTTATTATTGACTACATATTCATATAAACTAGA |
| | | GTTGTTGAAGAAATTCACAGGATATGTTTTCAGATTAGACCAGGCCAGGT |
| | | CGTCTGTATTCTGGTTTCCTTCCATCATTCTGTTAGACATCACTTTTACA |
| | | AATATACTCTCGTTGGCATCAAGCAAATGAATATTCGAAGTAATGTGCTG |
| | | TACACCATAATATCCGTCGACAGCTATCTTCATTTCTCCTTCCTTACCCT |
| | | TCTTTGTGGTAATAAGGATAACACCGGAAGCACCGCGAGTACCATAAATG |
| | | GCAGCCGAAGCAGCATCCTTAAGAATATCTATACTTGCTATTTCGCTACT |
| | | ACTCAATCCCGGGTCGCCCTCGAACGGGACACCATCGACAACATATAAAG |
| | | GAGAACTGTCGCCTGAGATAGAACTTAAACCACGAATCTGGATGTTGGAT |
| | | TTGGCTCCAGGCTCACCAGAACTTGCCTGAACGTTAACTCCGGCAACCAT |
| | | ACCCTGAAGAGCTGTACCCAAGTCGGAAGTACTGATCTTAGTAATCTCAT |
| | | CTGAGTTTACACGTGCCACTGCACCTGTCACCTCTTTTTTACGCATTGAG |
| | | CCATAACCTACAACAACCACTTCATCCAACACTTTTGTGTCTTCCTGAAG |
| | | CTTGATATTATAAATCTGACCATTCTTGATTGCAGCTTTTACAGTTTTAT |
| | | ACCCAACAAAACTGAACACTAAGTTACCTTTAGTCGGTACCCCTTGAAGA |
| | | ACGAAATTACCATCCATATCAGTAATAGTTCCAAGAGAAGTACCTTCAAC |
| | | TTGAACAGCTGCGCCTATAACTTCAAGGTTATTGGCAGCATCAATCACCT |
| | | TTCCTTTAACTGTTATCTTCTGTGAATACATAGACAATGTATAGAAGATA |
| | | AGCATCACGAACAACATGTACCTGCCATGGTACCATTTTTTCTGATTTCT |
| | | CATTTGTAAAAATTTTAATTTAGCAATAGGTTATGAAATTCCTTTTATAA |
| | | CTGACGCTAAATTATTTATTTATAATGGTACAAAAGGGGAGAATTATATA |
| | | TTTAAAAAGGGGGTAAAATTTTACCCCCACTTATATTAAGAATCCAAATC |
| | | GGTCTGTATACTCTGTTCTTTGTACTGTTGCGGCAATACACCGAATTCTT |
| | | TCTTGAAACATTCTCTGAAATACTTCAAATCATTGAACCCTACATCGTAT |
| | | GTCACCTCTGATACAGAATACCGTCCTGTCTTCAACAGTTCTGCCGCTCT |
| | | CTTCATTCTTATTGAACGTACAAAAGCATTGGCTGTTACTCCCATAAGTG |
| | | CTTTCAGCTTCTTGTTCAGAACCAAGGCCGTCACGCCAAGACCTTTACAT |
| | | ATATCCTCTATCTGGAACGAAGAGTCTGTAATGTTGTCCTCTATTATCTT |
| | | TACAAGTTTCTCAAGGAACTTATCGTCGGTAGATGTAGTGCTTACCTCGG |
| | | AAATCTTTATTGCCGGAACTTTCTTGTGTTGAAGAATCCGCTTCCTGTTG |
| | | GTTATAATGGAATTAAGCAGCTCTTTCATTATCTTGTTGTCGAAAGGTTT |
| | | AGGGCAATAAGCATCTGCATGGAATTTATATCCGATGAAATAATCCTGCA |
| | | ATGTAGTCTTGGCTGAAAGCAATACTACAGGAATATGAGATGTCCTTACA |
| | | TCCTGCTTGATTCTCTCACACAGTTCCAGACCATTCATGCCCGGCATCAT |
| | | TATATCGGATAAAACAAGATCCGGTTGCAAATCTGGAATCATGTTCCATG |
| | | CCATCTCCCCATCATGGGCTATCATTATCTTATACTTATCCGACAACAGT |
| | | AATGACAACATATTACATATATCCTTATTGTCATCAACAATCAATATAGC |
| | | CGGAGATTCTCCGTCCACTTCTATGTCTATCATCTCTTCATGCTCGCACG |
| | | ATTCACTTCTTAACACATCAGCAAACTTTTCATCCTCCCCACTGTTGGCA |
| | | GAGATATTCTCCGTAACCATGTCCCCCTCAGTTATCATAGGAATTACAAC |
| | | ATGGAAAACAGTGCCTTTACCTTCCTCTGATACAAACGTAATATTTCCAT |
| | | TATGTATCTCTACAAGCCGCTTGGTCAGAAACAGACCTATACCGGTACCT |
| | | CCTTCAGCAGAGTTTTTATTCTGACTGTAGAAACGCTCGAAGAGGTGTGT |
| | | TTTCAGGTTGTCGGATATTCCGTTTCCCGAGTCTGCCACAGAGATGTTTA |
| | | TTTTGTTATCCTGTTCATTGACAGTAAACGATACAAATCCTCCGGCAGGA |
| | | GTATGCTTAATGGCATTCGATACGAGATTATAGATTATCTGTTCCATAAG |
| | | ATGAGGGTCGAACAGAAAGCTTATATCACTGCGTGAGACAGAATATTCCA |
| | | GCCCTACACCTTTCTGTTTTGCCCAATACGTGAACTGCTGAAATACTTCT |
| | | TTTGAGAAAGACGAGAAGTTGCCATATTTGAGATTCAGACTAAGCATTCC |
| | | TTTCTCGCTCTTTGAGAAGTTCATCAGCTGGTTGACAAGACTTAACAGGA |
| | | ACTTACTGTTATGCTCCATTGTCTGCAGCATGCCGGCAAGATACTTGTCG |
| | | GACGAATACTTGCCCGATTCAATAATCATACTAAGTGGAGAATGAATAAG |
| | | TGTGAGTGGTGTCCTCAATTCATGCGATATGTTGGTAAAAAATGTAGTCT |
| | | CCTTTTCAAGAAGTTCTTCAGTCTTGCGTTTTTCCATGTTTGCTATATAT |
| | | AGAGCATTTCTGCGCTGCACCCGTGAGGTATAATACACCTTGAACCGGTA |
| | | TAAAGACAAGACAAGCAATATAAAATAGAGTGTATAGGCATACCATGTAC |
| | | GCCAGAAAGGAGGGTTAATAATGACAGGTATGGAAAGTTCATTCAAACTG |
| | | TAGACTCCATCGCTATTCCTGACCCTCAGTCTGAACATATATTCGCCTGA |
| | | AGGAAGCTTTGTGTAGAAAGCCTCACGATGAAAAGCGGAGGTGGAAATCC |
| | | ATGAATCATCTACGCCTTCGAGCATATATTCGTAACCAACCTTATAAGGA |
| | | CTTCTGTAATCCAGGGAGCTGAACTGGAATGAGAAAGTGTTTAAATTATA |
| | | AGGCAATTCAATGTGCTCTGTAAAACTTACACTTTTGTCGAAATAAGCTG |
| | | AATATGTGGAATCTGCCTCAACGCTGTGATTGAAGATTTTAAAATCAACG |
| | | AGTGTAGGACTACCGTTGAAATCTATCACATCAAAGTCATTAGGTCTAAA |
| | | GACGTTAATTCCGTTTACGCCACCGAATATCATTGTTCCATCCGTCATTA |
| | | CTCCAGCAGAAAGTTCCATAAATTCATAATCCTGAAGACCATCGAAAATA |
| | | TCATAAGATCTTATTCTCTGTGTGTTGATATTCAACGAATTAATTCCTTT |
| | | ATTGGTAGAAATCCATAATGTTCCATCCGTGCCATTAACAATTGATTTTA |
| | | TTGTATTGCTGCTCAACCCGTCTGCAGAGCTAAAATTTTCAACGCAGGCA |
| | | TTATGGTTTTCATCCAAATCCACGATTTTCCTTAACCCACGTCCAAGTGT |
| | | TCCATACCAGATATTATGATTCAAGTCTTCACATACAGGCACTATATAGT |
| | | CGAGTTCATCAAGTCCCTTGACTGAGTTCAAAACAGGATTATCTATATAC |
| | | AAATCTGCAGATTCCAATACTTTAAGACCGAAGCTGGAAGCTACCCATAT |
| | | ATTACCCTTATGATCTTTAATGATGTTTCTTACTATCTTAAGTTCTTTAT |
| | | TGTCAGATGTTTTGATTTCCTTCATCACACCTGTGGACAAATCATATCTG |
| | | AAAAGACCTTTATTATATGTGCCAATCCACAAATATTTTCCATCGGCAAG |
| | | CATTGCGCGCACATTTCTCAAACCTGAGATCTTTTTATAATCATTATCAG |
| | | AAGTGAAACTGTAAATACCATCGTACATCAGAGACACATACATGCAGTCG |
| | | GTGTAGTTTGAGTATGCTGTTGAGTATACTATCCTGTTTGCCGTGAAAGG |
| | | AATAAGTCTGGCATTACCGGTAATGGAATTAAAATGATATAGCCCTGAGC |
| | | CTTCTGTGCCTAAATATATATCAGATTTGGCAAATGTATAAACGGACGAT |
| | | ATATGATCATTTCCTATTCCTCTGAATAAATCTATAGGTTTATTATTTTC |
| | | GCGTATACTCATAAAGCCACTCTTGAAAAATCCTATCCAAAGAATATCGT |
| | | TTTTATCAAGAACTACAGTTTGCGGATAGCTGTAAGAATATGTAGCAATA |
| | | ACCTGTGGTTTTGACTCGATGGCATGCAATACATCAAAAGTCAACACATT |
| | | CACAGTGCTTGTAGTGGCATAAAATAATCTTTTGTTTTTATATACCATTT |
| | | TTCGTATATCACAGTTTTCCAACAGGGTACTTACCTTGCAGGTATGCTTG |
| | | TCGTATAAACATAATTGATGATTTTCCAGATTTGAGTACAATATTTGAGA |
| | | AGATGAGATGACTATGGCTGAAGCTATAGGGCATCCCAATAGTTTGTTAA |
| | | GCAGTAATTCATCTCCATCGACGTTACATTCGTACAGGCCGTCTTCGGAG |
| | | GAGAGCATTATCGTATTATCTATTTCTATGATGTCGGAAATGTATGGTAA |
| | | TTTTAATGTTGATCTTAAGACAGTATTTATTTTGCCATTTTGAAAATCAT |
| | | AATTTACAAGGTATATACTTTCATCAGAGGAATGAAACCAGACTCTGTCT |
| | | TTAGAGTCGACAAGAATCTTATCGCAAGTGAAATTTTTATCAATACCGCT |
| | | GTGACCAAGATTTAATGAAACGAATTCGTTCTTTACAGAATTGAACAGGA |
| | | ACACTCCTCTATCGGCTGTACCTATCCACAGATTTCCATGTGAATCTTCG |
| | | TCAATACATACTATCAGATTACTGTTAAGACCGTTTGACTGATATCCGTA |
| | | AACCTTAAATTCATATCCGTCAAACCTGTTCAGTCCGTCGTTCGTGGCCA |
| | | ACCATATAAAGCCTTTTGAGTCTTGATAAATACATTGCACATCATTTTGG |
| | | GAAAGTCCATCAAGAGTAGTGTACTTTCTTGTGACAAACTCATTGGATGC |
| | | AAAGGATTTGCAAACTATAATCAGAACTGATATTAAACTTAAGATTAATC |
| | | TAAACATATAACTATTATTCTTTATATTTCATCAAGATTACAAAGTTATT |
| | | GATTTTATCTAAAACATCAAGTATTTACAGTAGTTAATAGATAATTATAG |
| | | ATATTTTCCACTTTAGAATGCGTATCAAAATCAATCAAGAAAAAAATAAA |
| | | TCTTTAACTTCATTTCATAGTATAAAACAAAAAAAGCATCGTACCATTAC |
| | | ACTCAATAATAGATACGATGCCCGAAAGAAATTACAGTAACAGACTGTAT |
| | | TGGGATTGTTCTTAAAAAGACTTATCTGTATGACTTTATATATATGTCGA |
| | | GTATTTCGGTATCCGACAGTTCATGAGGGTCCAGACTGAACAATGCACCC |
| | | ATGGCAGTTCGCGCATTATCAATCATCTTAGGGAAATCTTCCTTTACTAT |
| | | TCCCCAGTCGCTAAGCTTCAAATCGCGGACATTGCATTCCTTCTGCATTC |
| | | TCACCAAAGCATCTATAAAATGTTCGGGATTAAGGTTCTTGCATCCGGTC |
| | | ATAACATCTGCCATGCGCATATATCTCTTTGTCCTGTCATAAATAAAAGT |
| | | AGAGAAATAGGCCTCGCTTATAGCTATCAGGCCAACACCATGAGGAAGAG |
| | | CGGGATAGTATGCGCTGAGAGCGTGCTCGAGAGAATGTTCGGAAGTACAA |
| | | CTGGATGTGGATTCAACCATTCCCGCCAGCGTACTTGCCCAAGCCACCTT |
| | | TGCCCTCGCTTTCAGGTTATTTCCATCCTTCACCGCAACAGGTAAATATT |
| | | TATACAGCAGTCTGATGGCCTCAAGAGCGAAAATATCACTTATTGGGGTT |
| | | GCACAATTGGCAATATAGCCTTCGGCTGCATGAAAGAATGCGTCGAATCC |
| | | CTGATAGGCAGTCAGATGTGGCGGAACTGAAACCATCAGTTCCGGGTCGA |
| | | TTATCGACAGACATGGGAAAGTTAAAGTGGAGCCGATACCTATCTTTTCG |
| | | TTTGTTTCCAGATTGGTTATGACAGTCCATGGGTCAGCCTCGGTTCCGGT |
| | | TCCGGCTGTTGTAGGAATGGCTATGATGGGCAATGCTTTGCTGTAAGGAA |
| | | GCCCCTTGCCGGTACCTCCTTCAACATATTCCCAATAATCGCCATCATTA |
| | | CATGCCATGATTGCAATGGATTTGGCCGTATCTATCGAACTTCCGCCTCC |
| | | CAAACCTATAATCATATCGCAATTTTCCTCACGACAGATTGCCGTACCTT |
| | | CCATTACATGGTCTTTTATTGGGTTAGGCAATATCTTGTCGTACACCACG |
| | | GCATCAACATTATTTTCTTTCAGCAGACCAATCACCTTATCCAGATAACC |
| | | ATATTTACGCATTGATGTTCCGGATGAAATGACTATCAAAGCCTTTTTGC |
| | | CGGGCAATGTCTCTGTTGAAAGACGTTTAAGTTCGCCACATCCGAAGAGA |
| | | ATCTTCGTCGGAATATTATAACCAAAAACAAAATTATTGTCCATAAATAT |
| | | TATCAGTCAGTCAACTTACTATCTTAAAGCCTCATCAATCACTTTCTTGA |
| | | GTTCAGGATAAGCCTCATCTGTATCGCCCACCTGTTTTCTCAACTCACGC |
| | | AGTTTCTTTTTCATGTCCTTAAGAACTTTGGCGTATTTAGGATTATCAGC |
| | | CAGGTTTACCATTTCGTAAGGGTCGTTCTTCACATCGTAGAGTTCGAAAG |
| | | AAACCGGAGTAGGAACAATCTTGTGGCTGTTCTTCAACCATGACATTGAT |
| | | TTCTGTCCGTAACGTTTGTCGTCGTAATGACGGCCATAGAAAAGTATCAG |
| | | CTTATAGTTTTCCGTGCGGATACCTATGTGTGCCGGAACGTCGTGATGAA |
| | | TCATGTGCATCCAGTATCTGTAGTAAACAGCATCCTTCCAGTTTTCTGGC |
| | | TTTTTGCCTTCGAACACAGAGGCAAAGCTCTTTCCATCCATGTATGAAGG |
| | | TTCTTTGCCACCGACCATCTCTATAAGAGTTGGAGCAAAATCAATGTTGT |
| | | TAATCATCAGGTCCGACTTGGCTCCCTTGTAAGGACATCTCGGGTCGCGG |
| | | ACTATGAAAGGCATTCTTTGAGATTCTTCATACATCCATCTCTTATCCTG |
| | | CAGATCGTGTTCGCCAAGCATCATACCCTGGTCGCCTGTATATACGATAA |
| | | TGGTATTTTCCCAGAGTCCTTCCTTCTTGAGATAGTCGAAAAGACGTTTC |
| | | AGGTTGTCATCCACACCCTTTACGCAACGCAGATACGATTTCAGGTAATG |
| | | CTGGTAGGCAAGGTATGTATTCTCCATTTCATCACCTGTATTGCACTTAT |
| | | ATTCCATTACATAATTGCGGATTTCATGACGGCTTGAGACAGAAGTTCCG |
| | | ATGAAGTGACGAAGTGAATCGTTCTTGCCTCTTGTGCCTTCGGAGCCCCA |
| | | TTTGTCTGTATCGAACAATGACAATGGAACAGGCACTTCCACATCGTCAA |
| | | GATAATATTCATAGCGCGGTGCGTACTCGAACATATCGTGCGGTGCCTTG |
| | | TAATGATGCATCATGAAGAAAGGTTTGGACTTGTCGCGTCTGTTCTTCAA |
| | | CCAGTCAATAGCAAGGTTGGTCACGATATCCGAGGAGTAACCCATTTTCT |
| | | TTATCTGGTTATTAGGCCATTTCTTGTCAGTTACGTCACTTGTAAGGAAA |
| | | ATAGGGTCGAAGTATTCGCCCTGTCCGCCATGACCGTTGAATACAGAATA |
| | | ATAGTCGAAGTGCGACGGTTCGCATCCCAAATGCCATTTACCGATCATGG |
| | | CAGTCTGATATCCCATATTATGGAACTCATCAACCAGATATTCCTGGTCC |
| | | GGCTGAAGCACTTCATCCAAAGTGAGCACCTTGTTACGATGGGAATACTG |
| | | TCCGGTCATGATACATGCACGGCTTGGGGTACTGATGGAGTTTGTACAGA |
| | | AACAGTTCTCGAAGAGCATACCGTCCCTTGCCAGTTCATCAATTGTAGGA |
| | | GTAGGGTTCAGTACTGCAAGACGACTTCCGTATGCGCCGATAGCCTGCGA |
| | | AGTATGGTCGTCCGACATGATGTAGATGACATTCATCTGTTTCTGCTGTG |
| | | CTGCGACACCAACACATACAGACAGGAATGGCATAACAGCCATTCCCTTC |
| | | ATTATATTATTTTTTAAATTCGTTTTCATAAGTCAGATTATCATTGAAAT |
| | | AGAACTTGCAAGACATATCATCGAATGATTTTACGTCCTTATTCTGCATT |
| | | TTAACCCATTGTTCTGATTTAGCCTTGACAGCGACCTGAGTTGAAACCTC |
| | | ATTACCGTCGACTACACTTTTAAGAGTGACATTTGCATCCTCTGCATTAT |
| | | GGTTTGCCACACGTACAGTGATAAGGCATCCGTTATCAACCTTATCGTAT |
| | | AGCGGTTTGGAAACCACCGCCCCTTTAAGCTTAATCTTGAACACATGTGC |
| | | ATATTCAGTAGGTTTGTTCTTAGGGAAGTTTACTACAAGACCCTCGTCAG |
| | | TCATCTTATAGTCAATCTTCTCTGAGCTTCCAAGCATTTCAACCGACTCA |
| | | ATTTCCACGTTCTGGCAATACTTAGGAGCAAATGACTTGATAGTAACACT |
| | | ACCATCTGTCCAAGCCAGAGACACGGCATAGAGGTTATTGTCGCGTGTAG |
| | | TAAAGCGAATGTCGTCCGCTGTATATTCAGTTTTTGTATTGTCTGTCATA |
| | | TAACCTGCGGTGCCTGCGTTATGTCCTTCGAAAGCAATCACCCATGGTCG |
| | | TGAGCCATAAATAGCCTCACCGTTAGTCTTCAACCATTTACCTATCTCGG |
| | | CAAGTACGTTCTTCTGTTCGTCTGTAATAGTACCGTCGGCCTTAGGACCT |
| | | ATATTCAGCAATAAGTTACCGTTCTTGCTGACAATATCAACAAAGTCGTC |
| | | GATGATATGGTCAGGACTCTTGTTTTCCTCGCCCACACAATAGCTCCACG |
| | | ATTTCTTGCCTACAGAAGTATCAGTCTGCCATGGATATTCACGGATTCTG |
| | | TCGCTCTTACCTCTTTCTATATCGAACACCTGGATATTGTCGCCATATCC |
| | | GAATTTAGTGTTAACCACAACTTCTTTATTCCAATCAAGAGCCGAATTGT |
| | | AATAATAAGCCATGAATTTATAGAAAGTAGGCTGGAACGGATATTTTCCC |
| | | ACAGTCCAGTCGAACCATATCAATTCAGGCTGATATTTGTCGATAAGCTC |
| | | GTATGTATGCATAAGGAACTGACGGCGTGAACGTTCGTTCGAGCCTTCAT |
| | | ACTTACCACAATAAGGTGTCATACCCTGACCTTCGGGCTCATGCAGTCTT |
| | | TCGCCATACAGAGTGATTGTAGTGTCCTGAACATCAGAAGGAGTTTCCAT |
| | | TCCATATTCATAGAACCATGCATTCTCGCATCTGTGAGAAGAAAGTCCGA |
| | | AACGCAGACCGGCTTTCTTGGTAGCTTCCTTCAATTCGCCGATTATATCC |
| | | CTTTTCGGTCCCATATCCACAGCATTCCACTTATTGAAAGTACTGCTGTA |
| | | CATGGCAAATCCGTCGTGATGCTCGGCCACCGGAACAATGTATTGTGCTC |
| | | CAGATGATTTTACCACTGCCAGCCACTCGTCGGCATTGAAATTTTCGGCT |
| | | TTGAACATAGGGATGAAATCCTTATATCCGAATTTGGTCAAAGGACCGTA |
| | | AGTCTGTACGTGATACTTATTAATAGGATGACCTTCCTTGTACATCCAGC |
| | | GGGAATACCATTCACTGCCGTATGCAGGAACGGAATAAACTCCCCAGTGG |
| | | ATAAAGATACCGAACTTGGCATCCTTAAACCATTCAGGAATAGTGTAATT |
| | | TTGAGCAATCGATGCCGAATCGGCCTTGAACACATCAGTACCTTTTAAAG |
| | | ATACAGTAGAATCTACATTAGGAGCGTATGTAGAATTGCACGACGCCAAC |
| | | AGGCTTAATGCCGCAACTCCTAAAACCGTTTTCATGGATTTCTTATTCAT |
| | | AATAATCTTATTACATTAAATAATGACATTAATTTTTTCTGTAAGCAAAG |
| | | ATACACTTGAGTTCCATTTACAATAAATAATTTAATTACTATAGTAAGGG |
| | | GTAAAATATTTACCACCTATTATTGAACAAATTTACCCCCTCTCATATAT |
| | | GATAATAAACTGCCAATATCGAATTACAAGTAAATATATATTTCAACAAA |
| | | AAAGGTTTAGCCTATTATTACACAACAATTTCACCCTAAGAATAAAATAT |
| | | ATATAGAGTAAATTTGCCAATATAACAAACTGTAAAAACAAATTTATGAA |
| | | AAACTATTTGATTTACTTACTCGCAGCAGTATCGTGTACAACTGTAGCAG |
| | | ACCTAAATGCTCAAGTCAGTACAAAAACAGGTAATGAAACCACAGAACTT |
| | | ACAATTCCGAAAAAGTTCTACAAGGACAGCATTGATTTCAGCAATGCTCC |
| | | GAAAAGACTTAACAACAAGTACCCTCTTTCCGACCAGAAGAACGAAGGCG |
| | | GATGGGTTCTAAACAAAAAGGCCTCTGACGAGTTCAAAGGAAAGAAGCTG |
| | | AATGAGGAAAGATGGTTCCCGAACAACCCTAAATGGAAAGGAAGACAACC |
| | | TACTTTCTTTGCAAAGGAGAATACTACATTTGAAGACGGCTGTTGCGTGA |
| | | TGAGAACTTACAAGCCAGCAGGATCACTGCCCGAAGGATATACTCACACT |
| | | GCCGGTTTCCTGGTAAGCAAAGAACTTTTCCTTTACGGATATTTCGAAGC |
| | | AAGACTGAGACCAAACGACTCGCCATGGGTTTTCGGTTTCTGGATGTCGA |
| | | ACAATGAAAGAAACTGGTGGACTGAAATAGACATTTGCGAGAACTGCCCC |
| | | GGCAATCCTGCCAACAGACATGACCTGAACTCGAACGTGCATGTATTTAA |
| | | AGCTCCAGCAGATAAGGGTGATATAAAGAAACATATCAACTTCCCTGCCA |
| | | AATACTATATACCATTCGAATTGCAGAAAGACTTTCACGTATGGGGACTT |
| | | GACTGGAGCAAGGAATATATCCGACTATATATAGACGGAGTACTGTACAG |
| | | AGAAATAGAGAACAAGTACTGGCACCAGCCATTACGCATCAATCTTAACA |
| | | ACGAATCGAACAAATGGTTCGGAGCCTTGCCGGACGACAACAATATGGAT |
| | | TCTGAATATCTGATAGATTATGTAAGGGTGTGGTACAAGAAATAAGAAAT |
| | | AACATAATCTGAAATTATAAAAGGCAGTCTTCATTATCAGTATGCTGATG |
| | | ATAAAGTCTGCCTTTTTAACAAGAAGATAAAGATTTTAATCTGCCCTATC |
| | | ACTCATTTACTTCATCCGGATACTCTGTAAGCGAGTTTCCCGAATTGCTT |
| | | ATTTCAATAGAGCCGATAGGAAGATAATTGAACTTCTTGCTCCATGCAGA |
| | | GATACCATAATCTCTTCTAAGAATAGGCATCATGACCTCCTCGGCACGTC |
| | | CTGAGCGGACGAGGTCAAACCATCTGTCACCCTCGCATGCCAGTTCACAA |
| | | CGACGCTCATACCATAGAACATCAATTACGCTTTTAAATCTGTCAGGATA |
| | | CATCTGCATTAGCTTGTCAACATCAATATAACTTCCGTCGTCTGCATGAA |
| | | CATGCTTCTTTCTGAGTTCATTTATGTAATACTTCGCTTTTGCTTCATCA |
| | | GGATTAGTACCTCTGAGATATGCTTCGGCAAGCATCAGATACACTTCACC |
| | | ATATCTGATGACCCTTACGTTTCCAGGCTTGTTTAGATTGGGGTTTCCTA |
| | | TCATATCGTAATTTTTGAAAGGAGGATATTTCTTCTGGGCATATCCCTGG |
| | | AAATCAGGCCCGTAAGAGCCTGTCTCCCAAACAACTTTTTTTGATTCATC |
| | | CTGAATATTGGCATTAGGTTTGGTTACAAGTTCATCGTAAGTAAATATCG |
| | | CCGCATCACGACGCACATGGTCATCCGGAAGGAAATAATCATACAATTCC |
| | | TTAGTAGGCAGACAAAAGCCATATCCATTATCATAATCAGGACTATTTTT |
| | | CAACTGTCTCGGTCCGCAGAAAGTCACCCACATAGCACCTTCGCCTGCAT |
| | | CAATATTACCCCAGTTTGTATTACCAGATTTGGTAGAGGTCTGTATTTCA |
| | | AATATAGATTCCTCGTTATTCTCCTGATGAGCCGCAAACAATTTAGAATA |
| | | ATCATCCGTCAGAGTATAATTACCACTTGAAATTACATCCTCCAATAAAG |
| | | GTTTCGCTTTGTCAAAAATCTTAGCATCATCGTTGCTCCAGTCAGCCCAA |
| | | TAAAGATAGACCTTGGCCAACAGGGCTTGAGCCGCAGTCTTGGTAATACG |
| | | TCCTTTCATTGTGTCCGGGAAATTATCCTTTAGAGAAGGGATAGCTTCAA |
| | | GAAGATCTTTCTCTATTGCTTTATTTACATTTTCGCGAGTATCTCTCGTA |
| | | AACTTGAATCCTTCAGGATAAAGAGTCTCAAGACTGATAAAGCATGGACC |
| | | ATAATATCTCAACAATTCAAAATGATACCAAGCACGTAAGAACTTAGCTT |
| | | CAGCTTTATAAACTTTAGCTTCCGGACTGTCATACTCTGAATTTATTACA |
| | | AGATTACATCTATATATACCACGGTAACGAGTTTTCCACAAATTATCGGA |
| | | AATAGAATTGACACTCGTATTTGAATAATCCTCTATAGCCTGCATGTAAG |
| | | GCTGATCCTGATCAGAGCCACCACCAGTACGAGCATTATCCGAACGGATT |
| | | TCACCCATAGGTACAATGGAAGCAAGTGCATTACCCGAAGCACCACCTAT |
| | | GTGAGCTAACGGATCATAACAAGCAGTAAGCGCTTTGAACATCTGTTCAT |
| | | CGGTCCTATAAAAAGAACTTTCTGTTTCGGACATTATAGGAGCTGTATCC |
| | | AGGAAACTGTCGCTGCAAGATGATGATGCAATAGCAGCAAACATGAGGAC |
| | | AAGAATATTATTATGTATTTTCGACTTCATAATTTTCAATTTTAGAAATT |
| | | AAGACTTAAACCAAATCTGAATGTACGGGCCTGAGGGTAAGTACCATAGT |
| | | CAATACCTGTGCTAAGAATATTGCCACCTGCCATATTTCCTACTTCAGGA |
| | | TCCATAAACGGATAGCTGGTGAAAGTGGCAAGATTATCAATTGCTGCATA |
| | | AATTCTTGCTTTATTCAGCATCAACTTGTTTATTAATTTAGTTGGGAATG |
| | | AATAGCCTACCTCAAGTGAAGAAATCTTTAAATGCGAACCATCATAAAGA |
| | | TAAAAATCGGATGGTTTGCCAAAGTTTCCATTAGGATCTTTGGATGAAAG |
| | | ACGAGGCACTCCATTATCATCACCTTCTTTCCGCCATCTGTCAAGATAGA |
| | | ATGATGGAAGGTTGCTGCGTCCGTATGCTTCCTGTCGGTAAATATCAGAG |
| | | AAGACTTTATATCCAGCTTTTCCTGTTAAGAAGATTGTCATATCAATACC |
| | | TCTCCAGTCGGCACCTAAATTCAAACCGAATGTCCATTTTGGCCAAGGAT |
| | | TGCCACAATCGGTTCTATCTTCATCTGTAATCTGCCCATCGTTATTTGTA |
| | | TCTTGCCATATAAAGTCACCCGGAACGGCATCAGGTTGTATCACTTTACC |
| | | GTCTTTTGATTTATAGTTCTGTATCTGCTCTTCATTTTGGAATATTCCTA |
| | | AGTTCTTATAAAGGCGGAAATAACCCATAGCATGACCTTCCTCCATACGC |
| | | GTTACATTAACAGATGTTCTCCAGCTACCACCATCAGTATATCCATTTAC |
| | | ATTTCCTATCTTTACAACCTCATTTTTAAGATATGAGGCATTTGCGGAAA |
| | | TAGAGAAGTTGATTTCGTTCCAATTTTTATTAAATGTCATCTGCATTTCC |
| | | ACACCCTGGTTTGTTATATTACCAAGGTTTCTAAAAGCTGCATTATTACC |
| | | TCTAATGGCTTCAACTGTTGGCTGGAACAACAAATCCTTAGTACTTTTTT |
| | | TAAACCAGTCGAAACTTGCTCTAATCATACCATTATAGAATGTCATATCG |
| | | GCACCAACATTAAATTGTTCAGAAGTTTCCCATTTCACGTCTGGATTAAC |
| | | AAGGTTATTAGGAGCAGATCCCACAGTGATGGCATTACCAAACGTGTAAT |
| | | TATAATTATTGCCAATAATAGAAGTATAGGAGAATGGAGAAATTCGCTCA |
| | | TTTCCGTTCTGTCCCCAAGAGAATCTAAGTTTGAAGACATCAAAGTTCTT |
| | | AATTTTCCAGAATTTCTCATTTGAAACATTCCAACCTAATGAAACGCCCG |
| | | GGAAAGTAGCATATCTGTTATTGGGACCGAAATTTGAAGACCCATCGCGT |
| | | CTGACCACAACTTCCGCCATATATTTTTCAGCATAATTATAGCTTAGACG |
| | | AGCAAAATATGAGAACATACTATGTCTAGGATTAGCACCGCCACTATTAG |
| | | CTGATGTCATAACATCACCAGCATTAAGATACCAGTAATTCTCATTGGTC |
| | | ATTGCTTCATTTGGATATTTATTTCGTGTTCCGGCCATAAACTCATAAAC |
| | | ATCTCTTGATGCAGAAGTACCTAACAGGACAGATGTAGAATGTTCACCAA |
| | | AAGATTTTTTATATCGCAATGTATTCTCCCACTGCCAACTACTATTAGCA |
| | | TTTGTACTTTGTTCTACCCTAGAATTATCTTCTTTACATTCTGCAGAATG |
| | | AAAAAACTTTGGTGCAAACATTCTTCCACGGAAATTCCGATGATTAATAC |
| | | CAAAATCTGTGCGGAAAACAAGGTCTTTAATAAAAGTGATCTCAGCATAA |
| | | ACATTACCAAAAAATTGCTGGGTAATATTTTTATTCTTAGGTGCCTCATC |
| | | CATAAATGCAATAGGGTTCCACATACGGCTATAAGGTACAGGAGAGACTC |
| | | CATATCCGAAAGTATCGTTGCTATTCTCATCATAAACCGGAGTAGTAGGA |
| | | TCAATATTATAGGCGTATGATATCGGATTATAACCATTGATACCGGTTGC |
| | | CACTCCACTATTCTCTATATATGCATAGTTGACGTTTGCACCTACACTTA |
| | | AGAAATCATTTATAGAATAGGAACTGTTCAGCCTTGTGCTGAATCGTTTG |
| | | TAAAATGACGCATCTTCACCGATAATACCATTCTGGTCTAGATAATTCAA |
| | | TGAAAGCAAGCTTGAACCCTTATCACTGCCAAAGTTAGCAGTAATGTTAT |
| | | GCTCAGTAACAGGAGCTGTATTCAATATTTCATTAAACCAGTCTGTATTA |
| | | TAACCTGTTGGAGCAGTAGGTACACCACCGGCAAGCGGCATATCATCATT |
| | | GTCGGCAAACTCTTTCATCAGCATAATGTACTGTTCATCATTCAGCATGG |
| | | TTGGTTTCTTTGCTACTGTAGAGAAACCATAGTAACCATCATAAGCAAGC |
| | | GATGTCTTTCCTTTCTTTCCTTTCTTTGTGGTTATAAGGACTACACCATT |
| | | AGCGGCTCTGGCACCATAAATAGCAGCTGAAGTTGCATCCTTCAAGACTT |
| | | CCATGCTTTCAATGTCGTTGGGATTTACACTGTTCATGTCGTCCATAGGC |
| | | AGTCCGTCAATTACAAAAAGAGGATTAGAGTTTCCATTTGTACCAACACC |
| | | ACGAATTACCAGCTTCGGTGCTGTTCCTGGCTGACCGGAATTTGTCACAA |
| | | CGTTCACACCACTAACCCTACCGCTCAATGCATTCACGGCATTTGCTGGT |
| | | TTAGATTGCAATAAATCATCGGAATCGATGCTACTGATAGCACCTGTTAC |
| | | AACACTTTTTTTCTTAACCTCATATCCTATTGCTACAACTTCCTCGAGTG |
| | | CAATGGCAGATGTTTTTAATTGAACGTCTATCTTAGACTGACCTTTATAC |
| | | ACTATATTCTGTGTATCATATCCTACGAAGCTATAAATCAATGTCGATTC |
| | | CATTGGTACATTTTCCAAGATATAATTTCCGTCCAAATCAGAAATAATAC |
| | | CGTTTGTGGTACCTTTAACTAAAATACTTGCACCTATCACAGGTAAACCA |
| | | TCGGAGTCTGTTATACAACCGGTAACTTTCCCGTTCTGTGCATTTAATGG |
| | | TAAACTGAACGTTATAAGAATCAGCATACACATTAATGATAGTGTTCTGT |
| | | TCATAATCTAGAGTTTTTTGTAATTAGTGTTTTTCTTAAAATAAAAAGTT |
| | | TTGTTCTATCAGTTGCGCGCTACTTACTGACACTTGCAAATATATATACT |
| | | ATGTAATATAACCAAAGGGGGAAAATTTCATTTAAATAGGGGGGGGAAAT |
| | | AGATTAACTAAATATTTTAAGGAAAAATGGCTGTTAGAATCCATTCCCAG |
| | | ACTCCAACAGCCATTTTATCACTAACAATCGCCTGTTAATCAATATATTT |
| | | TTCTGCCCATTTCCTTAAGATTTGCATCCCTGCCCAGTGGAACAAAAGTA |
| | | AATCCGTATGAATAGCTTCCCTTCAGAAGACGCTTGTCTATTGAAGGACG |
| | | GGCTTTCAGACTCCAGCTATCTGTTCCGCCCACTCCAGCCTGAACCAGGT |
| | | CGATATTAAGAGTATTAGAATACAAGTCCTTTTCAAGTTCATTTATATGT |
| | | TTAGCCTTATCAATCGCATTCTGCGACATCTCCCACACTGAAACAGATAG |
| | | GGGTTCATCGCCGACAATCATCACACCTGCCTTATCCGACTGCAAGGCAA |
| | | ACCATCTCACGTCACAACGGTTTCCGTTTTCCTGCGGCATTACATAGTCA |
| | | AATCCCAGAGCGGACACCTTGCAGTTATATATAGACACCATTGCAGAGGC |
| | | TTTTCTGTCGGAATAGTTTTCCCATGGGCCACGTCCATAATATGTCACAT |
| | | CCGACAAACGATTGGTACATTCGCATTGCAATCCTACGCGCAACATTTCT |
| | | GATATTTCAGGAGACTTCATCATTGAATAATGAACGCCTATTGTTCCGTC |
| | | TGCTTTTACTTTATAATTCAAGGTAAGTCTCAGTCTTTCATCTATAGCCT |
| | | TTAGCACCTTAACCTCAAGATTGCCTTCCGATTTGCGTACATCTATAGAA |
| | | ACTGTCTTTAGCTTTAATGGAGCATCTTTCCAGAATGCAAACAGTCTATC |
| | | GACCTTCCATCCTCGCCAGTCATTGTCTGTTGACGCTCTCCAGAAGTTTG |
| | | GTTTCAGAGCAGATGTGATGATACTTTCATTATCTATCTTATACTGACTG |
| | | ATATAACCATCACTGATATTCAGATAAAAGTTCTTTCCCTTCACGCTGAT |
| | | GTCTTTCTTGTTATCTGAATCGATTTCCATATCCAATGTAGTATCAACGC |
| | | ATTCTACTATCTTTGGTAAAGAAAGATACTTAAACTGTTCCCAGGCAACC |
| | | TCGTATCCAGCTTTGGCATACAGATTGTCATTCTTGAGCCTGGCACTCAG |
| | | GAATAACCAATATTCCGCACCGTCATCGGCCTTGAAATTCTGAATAGGAA |
| | | GTTTTAGTTTACAGCTCTCACCAGCTGGTGTTGTCGGCACAATAATCTCA |
| | | CCTTCCTGCAATACACTGTCTTCGTCCTTCAATTGCCAAAAATAACGATA |
| | | CTCATCTGTTGAAAGGAAGAAGTTTCTGTTTTTTACAGTTATCTCTCCAC |
| | | TATAGACATTATCAGTTGTAAATGATACAGGAGCAAACACGTACTTGCAT |
| | | TCCTCAGTAGCAGGTTTAATGGAGCGGTCGGCACTGATAACACCATTTAT |
| | | ACAGAAGTTTTGGTCGTTGTGCTCCCCTTTCTCATAGTCACCACCATAAT |
| | | TCCATGATTTCTTATTATATTTCCGTTCATTATCCAGCAATCCCTGGTCT |
| | | ATCCAGTCCCAAATATATCCGCCGGCAAGCGCATCATGAGAACGTATTGC |
| | | ATCCCAGTATTCTTTCAGCCCGCCGGTAGAGTTTCCCATAGAATGTGCAT |
| | | ATTCACACATTATTATCGGACGGTTCATGACCGGATTCTTAGTCATTGCT |
| | | ATAAGCTCATCGACCATAGGATACATACGGCTAATGACATCGACGTATAA |
| | | AGGATCATCGGGATTGGCATACACACAAAGCTCTTTCTTTGCCGGTTTGA |
| | | CATCTTCGTTCACATTAAAATCTATCTCACTAGTAACGATTGACGCTTCC |
| | | TTACGTCCGATAGGTTTGTATAAAGGATTTTCCGGCTGTCCTTGCGCCCC |
| | | CTCGTAATGAACAGGACGGGTTGGGTCATAATCTTTCAGCCATCCTGACA |
| | | GAGCTGCATGATTAGGGCCGCATCCAGACTCGTTGCCCAACGACCACATA |
| | | AACACAGAAGGATGGTTCCTGTCTCTCACAGCCATTCTTACCACTCTCTC |
| | | CATGAACGAGTTAGCCCACTCAGGCCTATTGGACAGATACCCCCTTTGAT |
| | | GATGAGTTTCAAGATTAGCCTCATCCATTACGTATATACCATACTTATCG |
| | | CACAGTTCATAGAAATAAGGGTCGTTAGGATAGTGCGATGTACGGACTGT |
| | | ATTGAAGTTATAACGCTTCATAAGCAGAACGTCTTCGAGCATCTCATCAC |
| | | GTGTAACGGTCTTACCTCCGGTCTCGCTATGGTCATGGCGGTTTACACCA |
| | | ATGAGTTTAATAGGAGTGTCATTCACCAGAATCTGATTACCTGTTATTTT |
| | | AATATCCCTGAACCCTACCTTATTACTTCTCGCATCCACCACGTTGCCCT |
| | | TTTTGTCTGTGAGCTTTATAACCAAAGTGTATAGATAAGGGTGTTCCGAA |
| | | TTCCATAGTTTTGGCTTAGAAACAATTCCCTCCATCATTCCGTAATAAAC |
| | | ATTATCACGCTGAGGATAAGGTTCGTTCACCACATAATCGGCAGTAACGG |
| | | TAATGTCTTTTCCAAACACCGGTTTCCCATCGGCATCATATAATTGGGCT |
| | | GACAGATTCCATCCCTTCAAATCATCCATATTCTGATTTGTTATTTCCGG |
| | | ACGGATCTGTAACCGTGCTATATTCTTCCGGAAATCGATGCGTGTCCTTA |
| | | CTCCATAATCATATATTGCCACCTGCGGAATGGACATGATATATACTTCA |
| | | CGATGGATACCAGCCATTCGCCAGTGGTCGGCATCTTCCATATAACTTCC |
| | | GTCGGTCCACTTATACACTTGCACCGCCAGTTTATTCTCCCCCTTCTTAA |
| | | CGTATTCGGTAATATCAAATTCAGTAGGCAGACAACTGTCTTCGGAATAT |
| | | CCCACCTTCTGTCCGTTTATCCATACATTAAATCCCGAATAGACGCCTCC |
| | | GAAATGGAGTATAATCCTGTCGCTCTTCCACTTGTCAGGAACAACAAACT |
| | | CCTTGATATAACACCCCGTCTGATTATTCCTGTCAATATATGGCGGACGA |
| | | GCAGGGAAAGGATAAATAGTATTTGTATATATAGGATAGCCATATCCCTG |
| | | CATCTCCCAACATGAAGGAACAGGAATAGTTTTCCATGATGATGAATTGT |
| | | ACTCCACTTTATAAAAACCGGCGGGAGCCAATGCCATATCCTCGGAAAAG |
| | | TTAAACTTCCATTGGCCGTTCAACGACATATACTCCGATTTCTCTCTGTC |
| | | TCCATCCAAAGCCCAATCCACTCTCCGGAAAGAATAAGTAGTACTGCGGG |
| | | AAGGCAAACGGTTAATTCCGTTTATGGTCTGATCCTGCCATACATTCTGA |
| | | TTGTTTCTCCACTGATTGGCACCGTTGTCCGATGCAGACAGAAATTGCAT |
| | | CATGAAAAATAACACAGAAAATGAAAAAATAGATTTTAAGTTCAAGTTCA |
| | | TAAATTCGCATTTTAAGTTTCTATGCAAATATATAAGTATAACGAACAAT |
| | | GAATAGGGGGTATTTCTATCTATATAGAGTGGTATTTTTACATATGAGCT |
| | | AAAACTTAAAAAAAACTGTCAGTATTACTATGCTATGTAGCACTCTATAT |
| | | GAAAATATTATATATTCCCAAGTCAAAAGCCTTTTCAAACAATTTTTATA |
| | | TATTCTCATCCTATCCCTTCCATCAAAGATAAATTCCAATCCTGATTTGC |
| | | CAGCCGCATTTATTCCTTTTTTCAGGAGAATTTTCTTTATGGCTATCGCC |
| | | ATGAAAATTCACCTGAAAAAGAATGCGGCGGCAAACGGATTAGAATTAAA |
| | | GAAAAGATTACAGGGATTAACTGCGACCGACGTGACGCATAGCCGTAATT |
| | | CAAAGGCGGCTATCCTTATATTCCATATATGACCTCACAAATACTGTGAA |
| | | AATCCACTTTCCCCAATAACAAAACATAGCCTGCCATATCAACACCCAAA |
| | | ATAAGACAGGGATTTCAACTCCCTCCGATCTGCATAGTCTGGTGGCTTCG |
| | | CTATGCTTTTACTCCTACATCCATTTTTTTTCTTTCTTTTTTCCTCTGTT |
| | | CCCGTTCTTTCCTATCCTTCGTGTGACATTTGATGACACCTGATGACATC |
| | | TAATGTCATCTATTTGTAAATCAATTGTTTACTCAATTTATCATCTTACA |
| | | TTTGGACTGTGAAACAAATCAAGTAGTCACTCAAAACAAAAGATTATGGC |
| | | ACAAGAAAACAGTCCTGACAAGGAAAAAAGGCAAGGCCGGACAAAGAAAC |
| | | CCGAAAAGCCTTATGTGGAACAAATTGACGAGCTTCTGCTGGTACATAAC |
| | | AAGAATGACCCAAAGGAAGGTTTGGGAGTAATCAGCAAGATGGACGAGAA |
| | | AGGCAATTATCAGACGGTTACACCGGAAGAGAAGAATGAGAACTCATTCC |
| | | TGAAATTCGACAAGAATTCGAGTATTCTCGAAAACTTCATCAAGAATTTC |
| | | TGGAGCCAGCTGAAGGAGCCTACGCATTTCAGGCTTATCCGTATGACCTT |
| | | CAATGATTACAAACAGAACAAACAGGCTCTCAAGGACCTGGCCGAAGGCA |
| | | AGAAGACAGACGCGGTAAAGGAGTTTCTGAAACGCTATGAAATCAGACCG |
| | | AAAGTAAACAATCAGAAAAACAGTCAAACAAAAGAGGAGGAAACAACAAT |
| | | GGCAAAGAAGCAGGAACAGACAACGCAGGCTCAGCCTGAACAGGTATCAC |
| | | AGGTGGAAGCTGCCGCACAGGGGCGCGAACAGCAGGAACCGCAACGCCAG |
| | | CAGACACCCACGTACCGCTACAACGAGAACATGATTAATTGGGAGGAACT |
| | | GGGTAAGTTCGGTATATCCAAAGAAATGCTGGAGCAGTCCGGACAGCTTG |
| | | ACAGCATGTTGAAAGGATACAAGACCAACAGAACCATGCCGCTGACACTC |
| | | AACATTCCTGGGGTACTGACCGCAAAACTTGATGCACGCCTTTCGTTCAT |
| | | ATCCAACGGCGGGCAGGTCATGCTGGGCATCCACGGTATCAGAAAGGAAC |
| | | CTGAACTGGACCGTCCTTATTTCGGACATATCTTCACGGAAGAGGACAAG |
| | | AAAAACCTGCGTGAAAGTGGAAACATGGGACGCGTGGCTGACCTTAACCT |
| | | GCGTGGCAACACGACAGAGCCGTGTCTGATTTCCATCGACAAGAATACCA |
| | | ACGAACTGGTAGCCGTACGGCAGGAGCATGTCTATATCCCGAATGAAATC |
| | | AAAGGGATAACCTTGACTCCGGACGAAATCCAGAAACTGAAAAACGGAGA |
| | | ACAGATATTCGTAGAGGGAATGAAGTCCAATCAAGGTAAAGAGTTTAATG |
| | | CCAATCTGCAATATAGTGCGGAAAGAAGAGGCATCGAATTTATCTTCCCG |
| | | AAAGACCAGGCTTTCAACCAGCAGACGCTTGGCGGTGTACCGCTTTCCCC |
| | | CATGCAGCTCAAAGCGTTGAACGAAGGACACACCATCCTTGTAGAGGATA |
| | | TGAAACGAAAGAACGGCGAACTGTTTTCTTCCTTTGTTACCATGGACAAG |
| | | GTTACAGGCGGGCTCCAATATACGCGCCACAATCCGGAAACGGGAGAAAT |
| | | CTACATACCAAAGGAAATCTGTTCGGTACAGCTCACACCGGAGGACAAGG |
| | | AAGCGTTACGCAAAGGGCAGCCCATCTATCTTGAGAACATGATCAACCGT |
| | | AAAGGTGAGGAATTCTCGTCATTCGTCAAGCTGGACCTGGCAAGCGGAAG |
| | | ACCACAGTATTCCAGAACTCCGGACGGTTTCAACGAACGACAGGCACCAG |
| | | CCATCCCGGCTGAGGTTTACGGACACCTGCTTTCGGCACAGGAAAGAGCT |
| | | AATCTTCAGGACGGAAAGGCTATCCTCGTAACGGGTATGAAAGGTCCCAA |
| | | CGGCAAACCGTTCGATTCCTATCTGAAAGTAAACGCAAACACCGGACAGC |
| | | TGCAATATTTCCAGGAAAATCCGGATGTGCGCCGCAATACTTCACAGCGT |
| | | GCTTCACAGACTGACAATACCCAGCAGCAGGAACAGAAGAAGGGAGCAAA |
| | | ACAGGCTGTCTGACCTGAACGGGATTCAAATCATTCAAATCATCAATTAC |
| | | TAAAAAAGGAAAGAACATGAACAAGACCAATCATCATATCTACAAGACTG |
| | | AACAAATCGACTGGGAGAAACTGGAATCGGTAGGTATCAGCAGATCGCAA |
| | | ATTGAAAAGGACGGAAACATGGACCTGCTCCTTCAGGGAGAGGAAACCAA |
| | | TGTCATGTCCATTAAAATCAAGACTCCTGTATTTTCACTGACCATGGACG |
| | | CCACACTCAGTCTGATTGAAGACGAGAATGGAAATCCGGTCATCAGCGTA |
| | | AACGGTATCAACCCTTCAGGTGAATAAATAAGAAACCATAATGTATCATC |
| | | TCTCTTTCCATACGGACTTACCGTATGGAAAGAGATAAAAACAGAATTTA |
| | | TCATGATTGCCATATTAACAGACAAACCAAGTGTAGGAAAAGAAATCGGA |
| | | AGAATCATCGGTGCAACCAAAGTAAGAAACGGATATGTGGAAGGAAACGG |
| | | CTACATGGTTACATGGACTTTCGGGAACATGCTGTCACTGGCCATGCCGA |
| | | AGGACTACGGAACCCAGAAGCTGGAACGGAATGACTTTCCTTTCATCCCG |
| | | TCCGAATTCGAACTGATGGTACGGCATACACGCACCGAGAACGGATGGAT |
| | | ACCGGACATTGATGCCGTGCTCCAGCTTAAAGTAATCGAGAGAGTGTTTC |
| | | AGGCATGCGATACCATCATTGCGGCTACCGATGCCAGCCGTGACGGGGAA |
| | | ATGACATTCCGCTATGTCTATCAATACCTGAACTGTACACTGCCTTGCTT |
| | | CCGTCTGTGGATTTCCTCTCTTACCGACGAGTCTGTGCGTAAAGGCATGG |
| | | AAAACCTGAAGCCGGACAGTTGCTACGACAGCCTGTTCCTTGCTGCCGAC |
| | | AGCCGCAACAAGGCGGACTGGATTCTCGGAATCAACGCCAGCTATGCCAT |
| | | GTGCAAGGCGACGGGCCTTGGCAACAATTCTCTCGGACGGGTACAGACAC |
| | | CGGTACTGGCTACCATCAGCAGACGCTACCGTGAAAGGGAGAACCATATT |
| | | TCATCGGACAGCTGGCCCATCTACATCAGCCTGCAAAAGGACGGCATCCT |
| | | TTTCAAGATGCGCCGCACACAGGATCTTCCCGACAAAGAATCCGCTACAA |
| | | TGTTTTTCCAGGACTGCAAGCTGGCACATCAGGCACAGATTACAGGTATC |
| | | AGCCACAGCGTTAAGGAAATACTTCCACCGGACCTGCTTGACCTGACACA |
| | | ACTTCAGAAGGAAGCGAACATCCGCTATGGTTTTACCGCATCAGAGGTGT |
| | | ATGACATCGCCCAGTCTCTTTATGAAAAGAAACTGATTTCCTATCCGCGG |
| | | ACTTCCAGCCGTTATCTGACGGAGGATGTGTTTGACTCGCTTCCACCAAT |
| | | CATGGCGCGTCTGCTTTCATGGGAGCTGTTCCCTGCAGCTAAAGGAACTG |
| | | GAGGTATTGACATATCCAATTTGTCCCGCCACGTAATAAGCGCAGAAAAA |
| | | GCCAATGTACATCATGCCATCATCATTACAGGTATCCGTCCCGGAAATCT |
| | | GTCCGAAAAGGAAATACAGGTTTACAGACTTGTAGCCGGAAGGATGCTTG |
| | | AAACATTCATGGCTCCATGCCGCATAGAAACGACAAATGTTGAAGCGGTT |
| | | TGTGCGGCACAGCATTTCAAGGCCGAACAAACAAGAATCATTGAAGCCGG |
| | | CTGGCATGATGTGTTTATGCGTTCCGACATGGTTCCAAAATCAGGATATT |
| | | CTGTCAATGAACTCCCCGAAGTGGAGAAAAGTGATACTCTGAATGTATGC |
| | | GGATGCAACATGGTACACAAGAAACAGCTGCCGGTAAATCCGTTCACGGA |
| | | TGCAGAACTGGTGGAATACATGGAACAGAACGGACTGGGTACAGTATCCT |
| | | CACGTACCAATATCATCCGTACACTGGTTAACCGTAAGTATATCCGTTAT |
| | | TCAGGGAAATATATCGTTCCGACCCCGAAAGGCATGTTCACCTACGAAAC |
| | | CATCCGTGGAAAGAAAATTGCGGATACTTCACTCACCGCAGACTGGGAAA |
| | | AACAGCTGGCCGGACTTGAAAGCGGAATGATAACCGGACAGGACTTCCTG |
| | | AACAGGATCAGGACTCTCGCCAAGGAAATGACTGATGACATTTTCAACAC |
| | | CTATTCCACAAAAGAAGAATAACATCTATACCTAATCAACCAAGAGAATG |
| | | CAGGCCGGAAGGTCTGCATTTTTTTGTATCCGTACAGAAAAGAATCTGTT |
| | | TTTCCGCTTTTAAGCGGCAAAGGTCTTGGATTGCCTGCCTTTTGCCGCAA |
| | | GGCTGCCCTCATGGGCTTGGCTGGACAGGAAAAAATCATCCTCGCTGCGC |
| | | TCCGGTATTTTTTCCTGCCAGGCCTTGCGCAAAAAGGCAATCCAAGAGGC |
| | | CGGAGGCCTATAAAATCGGGAAAACACATCCCGATGGGATTATTCATTCA |
| | | TAAAATTAAGGATTATGAAACTACAGATTATCAGAAAGATCGGCAGACAT |
| | | GCAACAGCGATATTCCTGATTACCGGAATATGTCTGCTGACAAGTAAAGG |
| | | GATTGTCCCTACTGGGATGATTACGCTGCTGTTGCTTGCAGGAGGGTTCA |
| | | TCGGTTTTCTGTTCAGGATACTGGTCATTATTTTCAAGATTCTTATTCTT |
| | | CTGTTCATTGTAGGATTATTTGTCGCATAACCCAAAATATAAATATACAT |
| | | ATATGGAAACAGTTGCTATAACCTCACAAGCTCCTGTCATGCCGGCTGTA |
| | | TGGCCACAGAACGAACATATCAGACCGGTTAAAAGACGTCTGCCCAATAC |
| | | AGTTGATGAACCTAAAAATATCGGCTACTATCTGGAATCGCTACGTGATA |
| | | TTTCCAGCAATCCGGACAGAGAGAATATTCTGAAAGAATTCTTCAAGGAA |
| | | ACTTATGTATAACCATAAAATTTTTCAATTATGTTTTTTCAATCAATTTA |
| | | TCAGATGATTACAGCAGGTACGGATCTGAATATCAATATCCGTAAAGTGG |
| | | ACAACAGCCTGAGCGTAGCAGTCATGCCAAGGCGGAACAGCCTGAAAGAG |
| | | GATACGCGACAGAACATGGTGCCACTGATCGTGAACGGAACACCGGCAGA |
| | | ACTGGATATGGGCTTCCTGCAGACCATACTCCAACCGATACAGAAGGTAC |
| | | AGGGACTGCTTGTCAATGCGGAAAATTTCGAGAAACAGGCAGAAAAGGCT |
| | | ACATCACAGGCCAAATCATCCAAGGCTCCAACAATACCGGCCGAATCAAA |
| | | GGAAGCCAGGGAAAAACGGGAAAAGATGGAAAAGCTCCTCAAGAAGGCTG |
| | | ATGAAGCAACCGCCGCAAAAAGGTACTCCGAAGCAATGACATGGCTGAAA |
| | | CAGGCACGGGTACTGGCTCCTACAGAAAAACAGAAGGATATTGACGAAAA |
| | | GATGCAGGAAGTACAGAAACAGGCTAGTGCAGGAAGCCTGTTCGGTATGG |
| | | CAGAGGAACCGGCGCCGGTAATTCCCCAACCACAAGGCTATATGAACGGT |
| | | CAGTCACAACCAGGTATGCAAACAAGCATATTCCCGGAGCAACAGACCCA |
| | | TACTATGAATCCTGAACCTGTCATGCAGCCTGCTCCACAGCAGGTATCAC |
| | | AACAAATTCCACAAGGAATACCTCAACCGGCATATGGAACGAACGGGACA |
| | | TATAACCCACCTGCTCCAAACAGCCCGATAGTAAAAGGAGCAGACATACC |
| | | GCAAGGCGCAACAATGCATCCTTACCCACAGCAGCCATACTACCAGCAAG |
| | | AGGCGACTCCTTATCCAACACAACAGCCACAGCAACCGACAAACGGACAT |
| | | ATACCGAATGGGGCTGCGCAAGTACAGAATGGAAACGGACGGGAATACCA |
| | | GACTGCATCGGCTACACATGAGACATTCTGCTTCGATCCGGAAGACGAGA |
| | | ATGACAGGGAACTTCTAAGAGAGGACCCGTATGCGGAATATCCGGATTTT |
| | | CCGGCTGAGTACCGAATGAAGGACGAGGCACAGGTAGAAATGGTATACTG |
| | | CTGATATACACAATAAACGATTTGTAAAACCAATAAACTATAAACAATAT |
| | | GGCACTGGAAATTAAAGGAATGAAAAGAGTATTCAAGATGAAGAAGAACA |
| | | ATCAGGAAATCGTACTGGATGATCCGAACGTAAACATGTCTCCGGCTGAA |
| | | GTGATGGACTTCTATTCCATGAATTATCCGGAACTGACAACCGCGACCGT |
| | | ACACGGACCGGAAATCGAAGACGACCGGGCGGTATATGAATTCAAGACCA |
| | | CTATCGGAGTAAAAGGGTAAGAGCATGAAAAAAGGACAACGTAAAGACAA |
| | | GAAACCATGTACACAACTTACGGAACGGGCTTTGGAAAATTTAGCCAGAC |
| | | TTATCATATCGGAACTCGAAAATACGGACATAAGCCGGGGCATCAGGAAC |
| | | AGAAAGAAAAGAAGACTCCCTCCCGCAGAAAGCCTCATGGTTTTCTGAAC |
| | | ACGAGAATACCTTCCATCGCTCCCGATCTGTATGTTGAGAATGACAGGGA |
| | | TGTAACGGTAAATGTCACCACCAAAGAGAATCTTGATTTCCTGTACCGTT |
| | | CAGCCATGAAGTATGCGCAGCTCCTGGATGTGGAGCTGCCATACCATCCT |
| | | ACAGGCAGGACTTCCACAAGAGAGAAAATATGCCTGCTATATAATGCACT |
| | | GGATTCCATAGTATCTCATCATGTAAATCTGGAACTTATTGGTGACAGGC |
| | | TCCAGTTCTGCATCTACCATTTCCATGAATGGCCGGATTATACGCTTTTC |
| | | TTTATGCCGATAGACTTTACGGAAAGGCTGCACGGTGAAATTAAAAAGAT |
| | | TACACTGGAGTTCATCAGAAAGTTCATCAAATATCACAGGATGATGGATA |
| | | TAACCGATACCCCTTATTTTGAGATGTCGGAAGTCTGTATCGATTATGTG |
| | | GACTTTGAACAGCTCGATGAGGAAGAGAAAAAGGATTTGTACAGAAAGGA |
| | | AAAGCTTTTCAGGTCATATGAGAAAGGGAGAATCCACAGGAAGCTGTGCC |
| | | GGATGCACTCCAGGGCTTTCTGTAGGAATCTGGAAGAACATATCCGCAAC |
| | | TGTACTCCTTCCAGCGATAAGGAAAGAAGACTTTTGGAACTGATTACCGA |
| | | AGGGCTGTCCCTGATTGCAAAGGACAGCCCTTATATCTTGAATTATGATT |
| | | ATGATTTTGCAAGCGAAAAGGAACGGGATTTCGAGCCGCCACCGCTCGAA |
| | | TATCAGATTCTGCTTACATATTCCATCACGGATACGGTTACCAAAGACAT |
| | | GGAAAGCTGTTTCAGTACTGACTGTCAGGAAACATATAACCAGACTCCCG |
| | | TATCATTTACCTTCATCACGCCGGAAACAGAGGAACTTTTCAAGCCGGAC |
| | | AACTATCCGGAACGGTTTGAGAAATGGTTTGAGAAATTTGTAGAACATGT |
| | | TACCTATAATTTATAAACATCATGAATGAACTGACCAAAAATATGCAAAA |
| | | AATGATGGTACCGAAGGCTGCAATCATAGCCTACAAGTATGAAGACAGAA |
| | | GAAATCTTGATACCAGGTACTTTATAGAATTACGTCCAATCAGAAAAAGC |
| | | GGACAGATGGGGGCAGGTATCCCCGTCACATACGAATTCATGAATACCCT |
| | | GCTGGAATCCTATACGGAAGAAATGAGCGGGATACCGGCAGGCAGAGTCC |
| | | CTGAAAACATGCTGGCCTGCAATCCGAGAAAAGGACAGGAAGAATATATC |
| | | TGGTACAATCCGCCCGGAAAAAGACAGATGTTCTTTCACAAGGATCTCAA |
| | | TATACAGGACGGCATGTTCAATCTGCCGGGAATTATCTACCAAGTAAAAA |
| | | ACGGAAACATGGACGTGTTCGCTTTCAAGGGGAAACGTCCGGTGGAGACG |
| | | ACTCCGCTGTTCCGTGCCCCGTTCTTCAACGTGACCGGATCAAGTGTCTG |
| | | CCTTGGCAACAGTTCTCTGGAAAAGCCACAGAACCCGACTTTCCTTTCCC |
| | | TGCTGGAATACTGGGAAAAACGGTTCTGGCTGACTGAATTCTCCCATCTG |
| | | GGAGGAAATGTCAATCCTACCGTTTCAAATCTTGTCATCGTCACCGAAAA |
| | | TATAAGAAACAATCCGTTCGACATGAACGAACTCAAGCCCATGAATAAAA |
| | | AACTTAAAGACATACTTCCATGAAAAAGATACATTTTACCGACCGCTACC |
| | | TGCTCAATCCACGTCATCCGGTAACGGTATTCGTCATCGGAGCTGGAGGT |
| | | ACCGGCTCACAAGTGATAACCAATCTGGCACGCATGAGCATGGCACTTCA |
| | | GGCATTAGGTCATCCGGGACTGCATGTCACCGTATTCGATCCCGATACGG |
| | | TTAGCCAGGCCAATATAGGACGCCAGCTTTTCAGTGAGACGGAACTGGGA |
| | | CTGAACAAGGCCGTATCACTTGTCACACGCATCAACCGTTTCTTCGGATA |
| | | CGCATGGACTGCCGAACCGAAATGTTTCCCAACGAAGAAATTTTCAGGAT |
| | | ATGATACAGCCAACATATTTATCACCTGCACTGACAATATACGTTCACGT |
| | | CTTGAGATTTGGAAATTTCTAAAGAAAACTCGTAAAGAGAACTTCAATGA |
| | | CTATTTGGTTCCTATATATTGGATGGATTTTGGGAACAGCCAGACAAAGG |
| | | GACAGGTCATCATCGGGACGGTACGTGAGAAAGTTCTCCAACCTTCTTCA |
| | | CAAGAATATATTCCCATGCCTAAAATGAATGTCATCACCGAGGAAGTGGA |
| | | CTATGCGAAAATCAAGGAAAAAGAATCAGGACCAAGCTGTTCTCTGGCGG |
| | | AAGCCCTGGAAAAACAGGATTTGTTCATTAACTCCACACTGGCACATATC |
| | | GGATGTGACATATTATGGAGAATGTTCAAGGAAGGAAAGACACTGTATCG |
| | | CGGTGCCTATGTCAATCTGGATACATTGAAAATGACCGCAATCCCGGTGT |
| | | AATGACAGAAGTGACCGTATCATCTTTCCATCAGAATACGGTCACTTATT |
| | | CTATTTGCTACTTATTATTTACTACGTTCTTACCACGCTGGAGCAGGAAA |
| | | CTCTGTATCTCTGAGGCGAGATAGAATGATTTCCCGTTCTTTTCCACCGA |
| | | GTAATATTTAATCTTGCCCTCTTGCCTGTAACGTGCCAAAGTTCTTTGTG |
| | | ACACACCAAGGAGTTCTGCCAGATCCACATTATCAAGCAGTCTGTCTCCA |
| | | TTCATACATTCTTTCAGACGATTCATCTGGTCCAGTTTCTTTTCAATGCG |
| | | GGCAAATCCCTCTACCATTGTTCCTATAAGTCTTTCGAGTATCTCATTAT |
| | | CTATATATGACATAATTCCAATGTTATTAAGTGAATAAATCGATACTCTC |
| | | TTCGTGCGCACTCTAAGAGTATGTACTTATAGTAGTGAAAATAGTATGCC |
| | | TGAATCTAAGACAAAGATCAACAAGCTTATTAGGCGCTGATAATCAGGCG |
| | | TATAATTTTTTCTACTTAATATTTAGTGTAAACCAAAAGTGTAAACTATG |
| | | TAATACAGAATTGGGAACGGGTTAACACAGCCACCAACAATGACATCTGA |
| | | TGCTACCTGACGACACCTAATGACAACATTTTGTATCATATACATATTCA |
| | | AAATACATTTGTACAAACTCAACTTTTTTGGATATGGAAATCATTGGAAT |
| | | TGAAACAGCTACATATGAAAAGACATTAAAGGAAATTGAAAACTTCCTTG |
| | | ATACCATTGATAAATTGATTACAGCTTCTTCACAGAAAACAATAGGGGAA |
| | | TGGTTGGATAACCAAGAAGTTTGCCTGATCCTCAAAATTTCTCCAAGAAC |
| | | ATTACAGAATCTTAGAGATACAGACCAAATCTCTTATTCTCAAATTGGGA |
| | | AAAAGATTTATTATAAAAAAGAAGATATTCAGAAGTTCATTGAAAAACAC |
| | | AACAGAAAATTATGAGCAAGGTAATTACCCAAGATAATGAGCAAGTTATT |
| | | CAGATATACAATAGGTTAAAAGATACGCTAACAAGACTCGAAGATATTCT |
| | | GAAGAATAACAACCCAACACTTAATGGGCATAGATATATGAATGATGCAG |
| | | AATTGGCTAATTACCTTAAAGTATCAAGACGCACTTTACAAGAATATAGA |
| | | AATAATGGAATCTTATCTTATTAOTCAGATTGGAGGTAAAATTCTATATC |
| | | GGGAATCTGATATAGAAGAACTTCTTGAGAAAAACAGACAGGAAGCATTC |
| | | CGTTAAACATTTCTTGGAATTTTCGTTGATTTTCAAAGCAAAAATCAGTA |
| | | TCTTTGCAATACTGACAAAGAGTTGTATATCAGTGCAGAACAAAGAAGTT |
| | | CAATCGAGGTGAAATAGGTGGACTAAATGACAAACAACAAGATAAGTAAT |
| | | TGATTATTAGCGATAAAAAATATAAGGTTCCGCCCCCAGGCGGATCACTG |
| | | AAAACAAAAGAGAAAT |

1. (canceled)

2. A bacterium comprising one or more transgenes that increase or enable the bacterium's ability to metabolize one or more bile acids or bile salts, wherein the one or more transgenes comprise:
(i) a transgene encoding a 3α-hydroxysteroid dehydrogenase (3α-HSDH), or a functional fragment or variant thereof, having at least 80% identity to an amino sequence encoded by SEQ ID NO: 18; and/or
(ii) a transgene encoding a 3β-hydroxysteroid dehydrogenase (3β-HSDH), or a functional fragment or variant thereof, having at least 80% identity to an amino sequence encoded by any one of SEQ ID NOs: 28, 30, 32, or 36.

3. The bacterium of claim 2, wherein the 3α-HSDH, or the functional fragment or variant thereof, comprises an amino sequence encoded by SEQ ID NO: 18

4. The bacterium of claim 2, wherein the 3β-HSDH, or the functional fragment or variant thereof, comprises an amino sequence encoded by any one of SEQ ID NOs: 28, 30, 32, or 36.

5. The bacterium of claim 2, wherein the bacterium is capable of achieving a rate of metabolism of the one or more bile acids or bile salts of greater than 0.5 mM/hour in a subject's gut.

6. The bacterium of claim 2, wherein the bacterium is capable of achieving a rate of metabolism of the one or more bile acids or bile salts of greater than 0.8 mM/hour in a subject's gut.

7. The bacterium of claim 5, wherein the rate of metabolism is maintained for at least 6 hours, at least 12 hours, at least 24 hours, at least 36 hours, at least 48 hours, at least 60 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

8. The bacterium of claim 2, wherein the bacterium is capable of converting at least 10%, at least 20%, at least 30%, at least 40%, at least 50%, at least 60%, at least 70%, at least 80%, or at least 90% of the one or more bile acids or bile salts to one or more different bile acid or bile salt products in a subject's gut.

9. The bacterium of claim 2, wherein the bacterium converts at least 70% of the one or more bile acids or bile salts to one or more different bile acid or bile salt products in a subject's gut.

10. The bacterium of claim 8, wherein conversion is maintained for at least 6 hours, at least 12 hours, at least 24 hours, at least 36 hours, at least 48 hours, at least 60 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

11. The bacterium of claim 5, wherein the subject's gut comprises a complex-native microbiota.

12. The bacterium of claim 11, wherein the complex-native microbiota comprises at least 10 bacterial species.

13.-137. (canceled)

138. A method of reducing a level of a bile acid in a subject, the method comprising administering to the subject a bacterium comprising one or more transgenes that increase the bacterium's ability to metabolize the bile acid, wherein the one or more transgenes comprise:
(i) a transgene encoding a 3α-hydroxysteroid dehydrogenase (3α-HSDH), or a functional fragment or variant thereof, having at least 80% identity to an amino sequence encoded by SEQ ID NO: 18; and/or
(ii) a transgene encoding a 3β-hydroxysteroid dehydrogenase (3β-HSDH), or a functional fragment or variant thereof, having at least 80% identity to an amino sequence encoded by any one of SEQ ID NOs: 28, 30, 32, or 36.

139-143. (canceled)

144. A method of treating a bile acid disorder in a subject in need thereof, the method comprising administering to the subject a bacterium comprising one or more transgenes that increase the bacterium's ability to metabolize one or more bile acids, wherein the one or more transgenes comprise:
(i) a transgene encoding a 3α-hydroxysteroid dehydrogenase (3α-HSDH), or a functional fragment or variant thereof, having at least 80% identity to an amino sequence encoded by SEQ ID NO: 18; and/or
(ii) a transgene encoding a 3β-hydroxysteroid dehydrogenase (3β-HSDH), or a functional fragment or variant thereof, having at least 80% identity to an amino sequence encoded by any one of SEQ ID NOs: 28, 30, 32, or 36.

145-158. (canceled)

159. The bacterium of claim 6, wherein the rate of metabolism is maintained for at least 6 hours, at least 12 hours, at least 24 hours, at least 36 hours, at least 48 hours, at least 60 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

160. The bacterium of claim 9, wherein conversion is maintained for at least 6 hours, at least 12 hours, at least 24 hours, at least 36 hours, at least 48 hours, at least 60 hours, at least 72 hours, at least 1 week, at least 2 weeks, at least 3 weeks, at least 4 weeks, at least 2 months, at least 3 months, at least 4 months, at least 5 months, at least 6 months, or at least 1 year.

161. The bacterium of claim 6, wherein the subject's gut comprises a complex-native microbiota.

162. The bacterium of claim 7, wherein the subject's gut comprises a complex-native microbiota.

163. The bacterium of claim 8, wherein the subject's gut comprises a complex-native microbiota.

164. The bacterium of claim 9, wherein the subject's gut comprises a complex-native microbiota.

165. The bacterium of claim 10, wherein the subject's gut comprises a complex-native microbiota.


SEQUENCE LISTING (83 sequences)

SEQ ID NO: 1 - DNA, 780 bp, Bacteroides fragilis
  source 1..780: mol_type = other DNA
atgaacagatttgaaaataagataatcattatcacgggagctgccggtggaatcggcgcatcaaccacacgccgcattgtatctgaaggcggcaaagtagttattgctgactattcaagagaaaaagcagaccaatttgctgccgagcttagtaattcgggagcagatgtacgtccggtttatttttctgctacagaattgaaaagctgcaaagaactaatcacctttacaatgaaggaatacggacagatcgatgtactggtaaacaatgtaggaggtacaaatcccagacgggacacaaacatcgaaactctggatatggattattttgacgaagcctttcatctgaatttatcttgtaccatgtatttgtcccaactggttatccccattatgagcacacaaggtggtggaaatattgtaaacgtagcctcaataagtggaatcacggccgattcgaatggtactctttatggagccagcaaagcaggagtcatcaatctgaccaaatacattgccacccaaacgggaaagaaaaacatccgttgcaatgcagtagcaccaggattgatcctgaccccggccgcactgaataatcttaatgaagaggtacgcaaaatatttctcgggcaatgtgcgacaccctatttaggtgaaccgcaagacgttgccgcgaccatcgcttttttagcctccgaagatgcacgttacattaccggacagaccatagtagtagatggcggattgacaatacacaatccgacaataaacttagtataa

SEQ ID NO: 2 - DNA, 768 bp, synthetic construct
  misc_feature 1..768: note = Description of Artificial Sequence: Synthetic polynucleotide
  source 1..768: mol_type = other DNA
atgttcaacagcgacaatcttcgtctggatggtaaatgcgcaattataacaggtgcaggcgccggaatcggtaaagaaattgcaataacgtttgctacggctggagcttcagtggtagtgagcgacataaacgccgatgcagcgaatcatgtggttgatgaaatccaacaactgggtggacaggcctttgcatgtagatgcgacatcacttctgaacaagaattatccgcccttgcagattttgcaatttcgaagcttggaaaggtggacatccttgtcaataatgctggtggaggtggacctaaaccgtttgacatgccaatggcggattttcggcgggcgtatgaattgaatgtattctcattctttcatttaagtcagcttgtagctcccgaaatggaaaaaaatggaggaggagtaatactgacaattacgagtatggctgccgagaataaaaacatcaacatgacgtcctatgctagttccaaagcagctgcatcccatcttgttcgtaatatggcattcgacctgggcgaaaaaaatatccgcgtaaatggtattgcaccgggcgctatattaactgatgctcttaaatcggtcattacgccagaaatcgagcaaaagatgctgcagcatacaccaattcgtcgtcttggccaaccgcaagacattgccaacgctgcattatttttatgttcaccggccgccagttgggtatcaggtcaaatcttgacagtatctggtggtggagtacaggaactgaactaa

SEQ ID NO: 3 - DNA, 804 bp, synthetic construct
  misc_feature 1..804: note = Description of Artificial Sequence: Synthetic polynucleotide
  source 1..804: mol_type = other DNA
atgaataagttggaaaacaaagttgctctggtaacatctgccacacgtggcatcggattagcaagtgctataaaattagcgcagaatggagccatcgtttatatgggtgtacgccgtcttgaagcaacgcaggaaatatgtgacaagtataaagaagaaggcctgattcttaagcccgttttctttgatgcatataatatagacatatataaagaaatgattgatacaattataaaaaacgaaggtaagatcgacatcttggtcaataacttcggtacaggacgtccggaaaaggatctggatcttgtaaatggggatgaagacacattcttcgaattattcaactataatgtaggttctgtttatcggctgtccaaattaataattccgcatatgattgaaaataaaggcggcagcatagtaaatatcagttctgtgggtggatccattccagacattagtagaatcggttatggggtatctaaatcgggggtaaacaatataacaaaacagatagcaattcaatacgctaaatatggcataagatgtaatgctgtgctgccaggcctgatcgctacggatgcagctatgaactccatgcctgatgagtttcgtaaaagtttcttgagtcatgtcccgttaaatcgcattggcaatcctgaagacatagccaattctgtgttgttttttgtaccatcagaagatagctcgtatatcaccggaagtatactggaggtgagtggtggttataatttgggtacacctcaatatgctgaattcgtgggttcaaaagtggtagagtaa

SEQ ID NO: 4 - DNA, 789 bp, synthetic construct
  misc_feature 1..789: note = Description of Artificial Sequence: Synthetic polynucleotide
  source 1..789: mol_type = other DNA
atgaaacgtctggaaggaaaagttgccatcgtaacaagtagtacaagaggtattggtcgtgcatcggctgaagcattagctaaggaaggtgctttggtatatctggcagcccgttctgaagagttggcgaatgaagtcatcgccgatataaaaaagcaaggtggcgttgccaaatttgtgtatttcaacgcaagagaggaagaaacgtatacttcgatggtagaaaaggttgctgaagcagaaggaagaatcgatatccttgttaacaactatggtgggactaacgtgaacctggataagaatttgacagctggtgatacggacgaattttttcgcatcctgaaagacaatgtacagtctgtttatttgccagccaaagccgctataccgcatatggaaaaagtgggtggaggttcaatcgtaaatatttcgactattggatcggtggtacctgacatatctcgcatcgcctactgtgtatcaaagtcagcaattaattccttgacgcaaaatattgcattgcagtatgccagaaaaaatatccgttgtaacgccgtcttgcccggtttgattggaaccagagcagcccttgagaacatgacggatgaattccgggacagcttcttgggtcatgttccacttaaccgtgtaggacgtcctgaagacattgcgaatgcagtattatattatgcaagtgatgattccggttatgtgacgggaatgatccacgaagttgcaggtggatttgctttgggaacaccacagtattctgaatactgtcctcgttaa

SEQ ID NO: 5 - DNA, 768 bp, synthetic construct
  misc_feature 1..768: note = Description of Artificial Sequence: Synthetic polynucleotide
  source 1..768: mol_type = other DNA
atgtcgtatgaatcgcctttccacttgaatgatgcggttgccatagtaactggtgctgcagcaggtattggaagagcaattgccggcacatttgcaaaagccggagcatcagtggtcgtaaccgatttaaaatcggaaggtgctgaagctgtagctgccgcaattagacaggctggcggaaaagccattggtctggaatgcaatgtaacggatgaacagcacagagaagctgtcattaaagcagcattggatcagtttgggaagatcactgtcttggtaaataatgctggtggaggaggtccaaagccgttcgatatgccgatgtctgatttcgaatgggcattcaagttgaacttgttttcccttttccgtttgagtcaactggcggctccacatatgcaaaaagcaggcggaggtgctattctgaacataagctcaatggcaggtgaaaacacgaatgtacgtatggcaagctatggtagttcaaaagcagctgtaaaccacctgacacgcaatatcgcatttgatgtaggtcctatgggtatccgtgtaaatgcaatcgcccctggagccataaagacagacgctttggctaccgtattaacccccgaaatcgaacgtgcaatgttgaaacatacgccgcttgggagattgggagaagcgcaggatattgcgaatgctgcactgtttttatgttcccctgcagcggcctggattagtggtcaggtattgacagtatccggtggaggggtccaggaattagattaa

SEQ ID NO: 6 - DNA, 804 bp, synthetic construct
  misc_feature 1..804: note = Description of Artificial Sequence: Synthetic polynucleotide
  source 1..804: mol_type = other DNA
atgaaaaaactggaagataaggtggcaataattaccgcagcaactaagggcattggactggcttcggcagaagtacttgcagagaatggcgcactggtatacattgcagcaagatctgaagagttggcaaaagaagtaatctcgaatattgaatccaatggtggtcgcgcgaaatttgtgtactttaatgctcgtgaaccgcagacatatacaacaatggtggaaacagttgcccaaaatgaaggacggttggatatcctggtgaacaattacggcgaaacaaacgtgaagctggatcgtgatttagtgaatggcgacacggaggaattttttcgtatagtgcaggataaccttcagtctgtatacctgccttcaaaagccgctattccgcgtatggcgaaaaatggaggaggctccattgtaaacatttcaacaattggttccgttgtcccggatttgggacgtattgcttattgtgtttcgaaagccgccataaattctttaacacagaatattgctctgcagtatgctcggcaaggggtgagatgtaacgcggtattaccgggattgataggaactaaagccgcgatggaaaatatgacggacgaattcagagattcctttttgcgtcatgtcccaataaatcgcgtgggaaaacctgaagatatagccaaagccgtactgtactatgcatcggatgattctgattacgtaacaggaatgattcatgaagtcgcaggtggatacgctcttggatcccctcagtatgctgaattcagtgccatgatggaacgttcaagataa

SEQ ID NO: 7 - DNA, 792 bp, synthetic construct
  misc_feature 1..792: note = Description of Artificial Sequence: Synthetic polynucleotide
  source 1..792: mol_type = other DNA
atgactctgcgcgaaaagtatggagagtggggaattatactgggtgcaacggaaggtgtgggaaaggcattttgtgaacgtttggctaaagaaggaatgaacgtagttatggtgggaagacgggaagaaaaactgaaggaattaggtgaagaacttaaaaacacctatgagatagactataaagtggttaaagcagatttctctttgccagacgcgactgataaaatatttgcagccacagagaacttggacatgggattcatggcatatgtagcctgcctgcattcatttggaaagatccaagacacaccttgggaaaaacatgaggctatgataaacgtcaatgtggtaaccttcatgaagtgcttctatcattatatgaaaatctttgccgcccaggatcgtggagcagtcatcaatgtaagcagtatgactgggattagttcatccccgtggaatggtcaatatggagccggaaaagcctttattctgaaaatgacggaagcagtggcgtgcgaaacggaaaaaaccaatgtcgatgtagaagtgatcacactgggaacaacactgacaccgagcttactgagtaacttgcctggtggtccgcaaggtgaagctgtaatgaaaacggctcaaacacccgaggaagttgtagatgaggcattcgaaaagcttggaaaggaactttcagtaatctctggagagcgtaataaagcttcagttcatgactggaaagcgaatcacaccgaggatgattatatccggtatatgggttcgttttaccaagaataa

SEQ ID NO: 8 - DNA, 792 bp, synthetic construct
  misc_feature 1..792: note = Description of Artificial Sequence: Synthetic polynucleotide
  source 1..792: mol_type = other DNA
atgaacctgcgtgaaaaatacggagaatggggactgatacttggtgccacagaaggcgtaggtaaggcattctgtgagaaaattgccgcaggtggtatgaacgttgttatggtcgggcgccgcgaagagaagttaaatgtacttgccggggaaatccgtgaaacttatggtgtggaaacgaaagtagtaagagctgatttttcgcaacctggagccgcggaaacagtatttgcagcaacggagggccttgacatgggattcatgagttatgttgcatgcctgcactcctttggaaaaatccaggatacaccgtgggaaaagcatgaagcaatgatcaacgtgaatgtggtaaccttcttgaaatgtttccatcactacatgcgtattttcgcagcccaagatcgtggagccgtaattaatgtgagcagcatgaccggcatttcaagcagtccttggaatggacagtatggtgcagggaaagcgtttatcctgaaaatgactgaggcagtagcatgcgaatgcgaaggaaccggagttgatgtggaagtaattacattgggaacaacactgacaccgtcacttctgagcaatttgcccggtggtccgcagggtgaagctgtaatgaaaatcgcgttgacacccgaagaatgtgtggatgaagcgttcgaaaaactggggaaagaactgtctgtaatcgccggacagcgtaataaagactctgtgcatgattggaaagctaatcacaccgaggatgaatatatccggtatatggggagcttctatcgggactaa

SEQ ID NO: 9 - DNA, 786 bp, synthetic construct
  misc_feature 1..786: note = Description of Artificial Sequence: Synthetic polynucleotide
  source 1..786: mol_type = other DNA
atgaacttccgtgaaaagtatgggcagtggggtattgtattaggtgctacagaagggattggtaaggcaagtgcatttgaacttgcgaagcgtggaatggatgtgattttggttggacgtcgtaaagaggctttggaagaattggccaaggcaatccacgaagaaacgggcaaagaaatccgtgtgttgccgcaggatctttcagagtatgatgccgctgaacgtttaatcgaagctaccaaagacctggacatgggcgtcattgaatatgttgcatgccttcatgcaatgggtcagtataacaaggttgactatgccaagtacgaacaaatgtacagagtgaacattcggacgttcagcaagctgcttcaccattatattggggaattcaaggaaagagatagaggtgcatttattacgattggatccctttctggctggacttcgcttcctttttgtgctgaatatgcagcagaaaaagcctacatgatgaccgtaactgagggtgtcgcttatgaatgcgcaaatactaatgtggacgtaatgttgttgtcggcaggatccacaatcaccccaacatggctgaaaaataaaccctctgaccctaaagcagttgcggctgccatgtatcctgaagatgtcataaaagatgggtttgaacaactggggaaaaagtttacgtatcttgccggagaattaaatcgcgagaaaatgaaagaaaataacgctatggatcggaatgatctgatcgccaaactgggcaaaatgtttgatcatatggcataa

SEQ ID NO: 10 - DNA, 795 bp, synthetic construct
  misc_feature 1..795: note = Description of Artificial Sequence: Synthetic polynucleotide
  source 1..795: mol_type = other DNA
atgaatcttcgggaaaaatacggagagtggggaataatcctgggtgctacagaaggtgtgggaaaagcattcgccgaaaaaatcgcatctgaaggaatgagcgtggttctggttggaagacgtgaggagaaactgcaagaactgggcaagtctatttccgaaacatacggcgttgaccatatggtaatccgtgctgattttgcacagagtgattgcaccgataaaatatttgaagctacgaaggacttggatatggggtttatgtcgtatgttgcatgtttccatactttcggtaaacttcaagacaccccttgggaaaaacatgaacagatgatcaatgttaatgttatgacgttcctgaaatgcttctaccactacatgggcatttttgcaaagcaagatcggggagcagtaataaatgtaagtagtctgacagccatttctagtagtccttataatgctcaatatggtgctggtaaatcatatattaaaaaattgacagaagctgtagcagctgaatgcgaatctacgaacgtggatgtggaagtcattactttgggaacaacaatcacgccgagtttgttgtccaatctgccgggtggacctgcaggtgaagcggtaatgaaaactgccatgacgcccgaagcatgtgttgaagaagcttttgataacctgggtaaaagcttgagtgtaatcgctggtgaacataacaaagctaacgtgcacaattggcaagcaaacaagacggatgacgagtatatccgttatatgggctcattctattcaaacaactaa

SEQ ID NO: 11 - DNA, 798 bp, synthetic construct
  misc_feature 1..798: note = Description of Artificial Sequence: Synthetic polynucleotide
  source 1..798: mol_type = other DNA
atgaacatgaatcttcgcgagaagtatggtgaatggggtattatactgggggcaacagaaggggttggcaaagccttctgtgaaaaaatagctgctggtggcatgaatgttgtgatggttggacgccgtgaagaaatgcttaaagatctggggcgggagatcagcaacaaatatggggtggaacatctggtaatcaaggctgattttgctgacccatcgtccgtagataaaatttttgagcagaccaaggaattggacatgggtttcatgtcatatgtggcatgttttcataccttcggaaagttgcaagatacgccatgggagaaacatgaacagatgattaatgtgaacgtgattacgttcttcaaatgcttctaccattatatgggaattttcgctaaacaagatcgtggggcaattattaatgtatctagcttgacaggaatctcttcatctccttataatgcacaatatggcgctggcaaaagttacatcttgaaattaacagaagctgttgcgtgtgaagcagccaaaactaatgtggacgttgaagtgataacactgggaacaaccattacacctagcctgttgaaaaatttgcctggaggaccggctggagaagccgtaatgaaaagtgcattaacacctgaagcatgtgttgatgaagcgtttgaaaatttgggtaagacattcagtgtaatcgctggagaacacaacaaaaaaaacgttcataactggaaggcgaatcatacagcggatgaatacatcacatacatgggcagcttttacgagaaataa

SEQ ID NO: 12 - DNA, 753 bp, synthetic construct
  misc_feature 1..753: note = Description of Artificial Sequence: Synthetic polynucleotide
  source 1..753: mol_type = other DNA
atgttcatgatgcttaaaaataaagttgccatagtaacaggaggaacacgtgggataggcttcgcagttgtgaaaaaattcatagaaaatggagcagccgtttctttgtggggatccagacaggaaactgttgaccaggcattagagcaactgaaagaattgtatcccgatgcaaagatcagtgggaaatacccttcattaaaagatactgcgcaagtgacaacaatgattaatcaagtgaaagaagagtttggtgcagtcgacattttggtaaataatgcaggtatatcccaatccacatcattctataattatcaaccggaagagtttcaaaaaattgtggatttgaatgtgaccgctgtatttaactgtagtcaagcagccgcaaaaataatgaaagaacagggaggtggtgtaatcttgaacacctcaagcatggtgagcatttatggccaaccgtcaggatgtggctatcctgcatctaaattcgctgtgaatggactgactaagtcactggccagagaattgggttgtgacaatataagagttaatgccgtggcaccgggcataacaagaactgatatggttgctgccctgcccgaagcagtaataaagcccttgattgcaaccattcctcttggacgcgtgggcgagcctgaagatatagcaaacgcatttttgtttttggcctctgatatggcatcttatgtaactggagagattctttctgtagatggcgccgcaagaagctaa

SEQ ID NO: 13 - DNA, 780 bp, synthetic construct
  misc_feature 1..780: note = Description of Artificial Sequence: Synthetic polynucleotide
  source 1..780: mol_type = other DNA
atgggcatatatgttataactggagctacatctgggatcggtgctaaaacagccgaaatccttagagaacgtggccatgaggtcgtaaatattgatctgaatggaggagatattaatgcaaatcttgcgacaaaggaaggaagagctggagcaattgctgaattgcatgaacgtttccctgagggtatcgatgctatgatttgtaatgctggggtaagcggaggaaaagtgccgatttcgcttatcatatccctgaactactttggagcaactgaaatggcacgcggcgtatttgacctgctggagaaaaaagggggatcttgtgtggtaacaagttcgaattcaattgcccaaggtgccgcaagaatggatgtggctggaatgttgaataaccacgcggatgaagacagaatcctggagctggtcaaagatgttgatccagccatcgggcatgtatactatgccagtactaaatatgccttagctcgttgggtaagacggatgtcgccggattggggatcaagaggcgtccgtctgaatgcgattgctcctggaaacgtgagaacagctatgaccgcaaacatgctgccggaacaacgtgctgcaatggaagccattcctgtgcccacacatttcggtgaagagccgttgatggatcctgtggaaattgctaatgcaatggcgtttattgcttctcctgaagcgtccgggatcaatggagttgtgctgtttgttgatggtggcactgacgcactgctgaatagcgagaaagtatactaa

SEQ ID NO: 14 - DNA, 753 bp, synthetic construct
  misc_feature 1..753: note = Description of Artificial Sequence: Synthetic polynucleotide
  source 1..753: mol_type = other DNA
atgggaaagctggaaggtaaagtagcaatagtaaccggcggtacgcgtggaatcggattcggaattgttgaaaaattcttggcagaaggtgcaaaggtcgctttatttggaagtagacaggagacagtggacgcggcgcttgaaaagattaaacagaatgatcctgaggcgccagttatgggattgcatcctgctttaactgacccggatgaaatagcagccgcctttaagtccgtggtagatactttcggaagtttggacatcttagccaataatgctggaacagactcaagaaccaaacttgtggattatacacttgaggaatttcagaaagtaatgcgccttaatgtggaagctacattcgtatgtagtcaggcagcggctcgtataatgatcgagcaaggaactggcggtgccataatcaacacatcctctatggttagcatctatggacaacctgccggatgtgcttaccccacgtctaaatttgccgtcaatggattaacgaaaagtcttgcccgtgaattggctcctcataaaattcgtgtgaacgctgttgccccaggtgttacgcatacagatatggtggatgccctgcctcgtgaagtcatcgaaccattgattaaaaccatcccattgggacgcatgggagaaccggaagacattgcaaatgcttttgcattcttagcttcggatgaagcctcgtacataagtggtgatgtgctgtctgttgatgggttaagccgtagctaa

SEQ ID NO: 15 - DNA, 783 bp, synthetic construct
  misc_feature 1..783: note = Description of Artificial Sequence: Synthetic polynucleotide
  source 1..783: mol_type = other DNA
atgggaatatatgttattactggcgcatccagtggaattggagcaaaaactgccgaaatacttcgggaacgtggccatgaggtggtaaacatcgaccttaaagatggagacatcgaagcgaatcttgcaaccaaagaaggacgggctggagcattagcggaattgcatgaacggtatccggaaggaatcgatgccatgatctgcaacgccggcgtgtctggaggtaaagtgcctatctccttaataatctccctgaattatttcggtgcaactgaaatggctcgtggagtcttcgatctgttggaaaaaaaaggtggtagttgtgttgttacatcgagtaatagtattgcacaaggtgcagctcggatggacgttgccggaatgttaaataaccaggccgatgaagatcgcattgtggaactggtaaaggatgtagacccggctgtgggacatgtctattatgcctccactaaatatgcccttgccagatgggtaagaagaatgagccctgattggggtagccgtggagtgcggttaaatgcaatcgcgcctggtaatgtccgcaccgcgatgacaagcaatatgctgccggaacaacgggccgcgatggaagcaatacctgtaccgacccattttggagaagaaccgcttatggacccggaagaaattgccaatgcgatggcattcgttgcttcacctgaagcatcgggtcttaatggtgtggttctgttcgttgacggtggaacagacgccctgttaaacagcgaaaaggtgtattaataa

SEQ ID NO: 16 - DNA, 783 bp, synthetic construct
  misc_feature 1..783: note = Description of Artificial Sequence: Synthetic polynucleotide
  source 1..783: mol_type = other DNA
atggggatttatgtgattacgggagccacatctggtattggcgctaaaactgcctcgattcttaaagaacaagggcatgaagttgtcaatattgacttaaagggaggcgacatcaatgctaatttagcaagcaaggaaggaagagctgccgccattgacgaattacataaacgctacccggatgggatcgatgcgatgatttgtaatgccggtgttagtgccgcgaatggatctattcctctgattatatcgcttaattatttcggtgcgacagaaatggcaataggtgtacgcgacttgttggaaaagcgtgggggtaactgtgtcgtaatatcatccaataccattgctcaaggagcagcgcgtatggacgtggtcggtatgttgaataatcaagcagatgaagaccgtatattggatttggtaaaggattacgaccctgcgacagcacatgctttttatgcagccactaaatatgcacttgcgcgctgggcacgtagaatgtctgcagattggggtgcacgtggagtgcgcgtgaatgctgtggcacctggaaatgtccggaccgcaatgacagaccagctgacagacgaaatgcttgtagctgtgcgtgctcttccagttcctacaaattatggaggtgaccctttgatggacccgaccgaaatcgctaatgcaattgcttttttaagctcccctgaagcgcgtggtattaatggagttgtcttgtttgtagatggaggtaccgacgctctgctgcacagtgaaaaagtttattaataa

SEQ ID NO: 17 - DNA, 792 bp, synthetic construct
  misc_feature 1..792: note = Description of Artificial Sequence: Synthetic polynucleotide
  source 1..792: mol_type = other DNA
atgggtgtgtatgcaattacaggagcttcttccggaattggagccaagactaaagaattgctggaactggaaggacataaagtaatcaacatcgatttgaaaggaggtgacatttgtgtgaatttagcttcggtggaaggccgtgaagaagcaatcgctaaactgcatgagatgtgtcctgatggattggacggcatgatctgtaatgctggagtaagtggtgcttgtggcaacctggaacttataatcagcttgaactatttcggaactatagcagtagcgaaaggggtgtacgacttactggaaaagaagcatggatcatgtgtggtaaccgcatccaacaccataagccaaggagctggccgtatggatattgcggatttgctgaataacattggtgacgaaaaacgggtgttggaactggtaagtagcctggattcttcaaacttatcggtgggcaattctatgtatgtaagcactaaatatgccctggcaagatgggtaagaagagtatccgcaacgtgggcagccaatggtgtcagaatcaacgcgattgcacctggaaatgtaaacacagctatgaccgctactatgtcaacctctgcaaaaatggcacttaatgcccttcccatcccaactaaattcggactggaaactttgatggacccagaagagattgcagaagtaatgatcttcttggtgtcgaaaaaagcctctggcatcaatggaaatatcatgtttgtcgacggtggaacagatgctcttcttaactcggagaaggtatattaataa

SEQ ID NO: 18 - DNA, 768 bp, synthetic construct
  misc_feature 1..768: note = Description of Artificial Sequence: Synthetic polynucleotide
  source 1..768: mol_type = other DNA
atgcctgtaactgctgtaacaggttctgcgtctggaatcggtgcagctgtatgtgatgtattaagagcagcaggacaccagataattggtatcgaccgtgtgaatgctgaggtgattgcagatctttccacgccggaagggagacaagctgctgttgaagaagttcttgaaagatgtgacggagtattagatgggttagtatgttgtgctggagtgggtgtcactgtaccgtcttccggactgatcgtagctgtaaaccattttggcgttactgctttggtggaagggctggcttcggcgctggaacgtggtgaacgcggagcagccctgatcgtgggatcggtcgccgcgactcatgcggacgattctcagcccatggtcgaagctatgcttgctggtgatgaagcacgggctattgcactggctaatgaattagatcaggcacatatcgcatatgcatcttcgaaatatgcggtaacccgttacgcccgtcaacaagctgttgcatggggaggaagaggattacgtctgaatgtggtggcccctggtgccgtggaaaccccgttacatcaggcttccctggaggatccgcgctttggacaagcagtacgtgattttgttgctccgttgggacgggctggacaaccggccgaaatagcagcccttgttgcattcttacaatctccgcaagcttcgtttatccatggctctgtaatgttcgtagacggtgggatggatgcaatggttcgccctacaaaattctaataa

SEQ ID NO: 19 - DNA, 783 bp, synthetic construct
  misc_feature 1..783: note = Description of Artificial Sequence: Synthetic polynucleotide
  source 1..783: mol_type = other DNA
atgggaatctatgtgataactggagcaacctccggaatcggtgccaaaacagctgaaattttgcgtggacgtggccatgaagtagtaaatattgatctgaatggcggggacatcaatgccaaccttgcaactaaagatggaagagctcatgctattgctgaattgcatgaaagatatccggaaggaattgatgcgatgatctgtaatgctggtattagcggaggaaaagctccgatatctctgatcgtgtcgttaaattatttcggggctacagagatggcacgtggcgtattcgatcttcttgaaaagcgtggtggatcctgcactgtgacatcatccaattcaattgcgcaaggggctgctcgtatggacgttgctggtatgttgaataaccatgcagacgaggatcgcattctggaactggtaaaagatgtcgatccggccatcggacacgtatattacgcatcaaccaaatatgctttggcaagatgggtgcggagaatgtcccctgaatggggaagtcgtggtgtacggttgaatgccgttgcgccaggaaatgtacgtacagcgatgaccgacaatatgcttccggaacaacgtgcagcaatggaagcgattcccgttccaacacattttggagaagaaccgcttatggaacccatcgaaatcgcaaacgctatggccttcattgcatcaccagaggcctgtggaattaatggtgtcgtcctttttgtggatggaggtaccgatgctctgctgaactctgaaaaagtctattaataa

SEQ ID NO: 20 - DNA, 783 bp, synthetic construct
  misc_feature 1..783: note = Description of Artificial Sequence: Synthetic polynucleotide
  source 1..783: mol_type = other DNA
atgggcatttatgtgatcactggtgcctcttctggaattggtgcaaaaactgcggaaatattaagagagcgtgggcacgaagtggtgaacatcgaccttaatggaggtgatattaatgcgaatttggcaacaaaagaaggccgtgccggcgcaatcgcggaattgcatgagagatacccggaaggcatagatgcaatgatttgtaacgccggtgtaagtggaggaaaagtcccgatctctctgataatttccttgaattactttggtgctactgaaatggcccggggtgttttcgaccttttggaaaaaaaaggtggtagctgtgtggtaacttcaagtaattcgattgcgcaaggtgccgcacgcatggatgtagcaggcatgttaaacaatcatgcagatgaagatcgcatcctggaacttgtaaaggatgtggacccagccattggtcacgtttattatgcctccactaaatatgcattggctcgttgggtcagacgtatgtcaccagattgggctagccgtggcgtaagactgaatgcggtagcacctggtaatgtccgtacagcaatgacagcaaacatgcttccggaacagcgtgcagcgatggaagccattccagtacctacgcattttggtgaagaaccgttaatggatccggttgaaatcgctaacgtaatggcatttgtcgcaagcccggaagcaagtggtattaacggagtggttttatttgtggatggtggaactgatgcgcttttgaatagcgaaaaagtttattaataa

SEQ ID NO: 21 - DNA, 783 bp, synthetic construct
  misc_feature 1..783: note = Description of Artificial Sequence: Synthetic polynucleotide
  source 1..783: mol_type = other DNA
atgggtatctatgtgattacaggagctagttccggaattggcgccaagacggcagaaattctgcgtgaacgtggtcacgaagtggtcaacattgacctgaatggcggagacatcaatgcaaacctggccacgaaagaaggaagagcgtctgctttggcggaactgcatgaacgtttcccggaaggaatagatgcgatgatctgcaacgccggtgtatctggtggtaaagtgccgatatcactgatcatatccctgaattattttggtgctacagaaatggcccgtggagtcttcgatctgcttgaaaaaaaaggaggttcgtgcgtagtaacatcttctaatagtattgcacaaggagccgctcggatggatgttgcaggaatgttaaataaccatgcggatgaagatcgtatactggaactggtaaaggacgttgatccagctattggccatgtatactacgcatcaacaaaatatgctctggcacgttgggtaagacggatgtctccggactggggaagtcgtggtgtcagactgaatgcaatagcacctggaaatgtacgtacagccatgacatcaaacatgttgcccgaacaacgtgcagcaatggaagcaattcctgttcctacacactttggagaagagcccttaatggatccgatcgaaattgcaaattcgatggcatttattgcatctccggaagccagtggaattaacggcgtggttctttttgtagatggtggaaccgacgctttattgaactcagaaaaggtatactaataa

SEQ ID NO: 22 - DNA, 783 bp, synthetic construct
  misc_feature 1..783: note = Description of Artificial Sequence: Synthetic polynucleotide
  source 1..783: mol_type = other DNA
atgggtatttatgtcatcactggagcctcatcaggaatcggtgccaaaacggccgaaatattgcgtgaacgtggacatgaggtggttaatattgacttaaaagatggcgatatcgaggcaaatttggcaaccaaagaagggcgtgctggtgcaatagcggaattgcatgagagatatccggaaggtatcgatgcaatgatatgtaatgctggcgtttctgggggaaaggttccgattagtttgataatatctttgaactatttcggtgctacagaaatggcgaaaggagctcgtgacctgctggaaaaaaagggtggaagttgcgtaataacatcatctaattcgatcgcacaaggagcagcccgcatggacgtcgcaggaatgttgaataaccaagctgacgaggatcggattttggaactggtgaaggatgttgatccggctgtcggacacgtgtattacgcatcgacaaaatatgcccttgcaagatgggtccgccgcatgtctccagattggggttcccgtggtgtaagactgaatgccatcgcaccgggaaatgttcgtacggcaatgacagcgaatatgttaccagaacagcgggctgctatggaagccattcctgtaccgacacactttggagaagaaccattgatggaaccgattgaaattgctaatgcaatggcttttatcgcatcaccagaggcttctggtatcaatggtgttgttctgttcgtggatggagggacagacgcattgttaaatagcgaaaaagtttattaataa

SEQ ID NO: 23 - DNA, 783 bp, synthetic construct
  misc_feature 1..783: note = Description of Artificial Sequence: Synthetic polynucleotide
  source 1..783: mol_type = other DNA
atgggaatttatgtaatcacgggagccagctctggaataggagcgaaaacggcatcgatattgaaagaacacggtcatgaggtggtgaatattgacttaaaagggggagatatagatgccaacctggcatctaaagaaggacgtgctgccgcgattgctgaactgcatgaaagatacccggaaggaatcgatgcaattatctgtaatgctggagtgtccgctgccaatggcagtatccctctgataatctcattgaactacttcggtgcaacagaaatggctattggagtacgtgatctgctggaaaaaaaaggaggaaattgcgtcgtaatctcaagtaatacaattgcccaaggagccgctcgtatggacgttgtcggaatgctgaataatcaggcagatgaagatcgtattttggagctggttaagaactatgatcctgcaagtgcccatgcgttctatgccgcgactaaatatgctttggcgagatgggccagacgcatgtctgccgattggggtgccagaggtgttcgtgtaaatgcagtggcacccggtaatgtacggacagcaatgacggaccaactgacggatgaaatgctggttgctgtaagagctttgccggttccgactaattatggtggggatcccctgatggatcccgccgagatagctaatgccattgcctttttatcttcacccgaagctcgtggagtcaatggcgtagtgttgttcgtggatggaggaacggatgccttgctgcattcagaaaaggtctactaataa

SEQ ID NO: 24 - DNA, 831 bp, synthetic construct
  misc_feature 1..831: note = Description of Artificial Sequence: Synthetic polynucleotide
  source 1..831: mol_type = other DNA
atgaatttcggcggattcattatggggcgttttgacgaaaaaatcatgctggtgaccggagccacaagcggtataggacgcgctgtggctatacgtgcagccaaggaaggtgccacagtagtggcagttggccgcaacgaagaacgtggagctgcagtagtagctgcgatggaggaggctgggggtaaaggtgaatttatgaaatgtgacgtttccaacaaagatgctgtaaaagcgttgttcgcggaaatccaggaaaagtacggtaaacttgatgtcgctgtgaataatgccggaattgtaggcgcaagtaagaccgtggaggaattggaggatgatgactggttccaagttattgatgcgaacctgaattcctgttttttctgttgtagagaagaagtaaagcttatgcagccctctggtggtgcaattgtaaatgtcagcagtgtagctggtatgcggggtttcccgtctgccgctgcttatgttgctagtaaacacgcagtatctggcttgacaaaagccgtcgctgttgactacgccacaaaagggatcacctgtaatgctatttgtcctgctggaactgatacgccgctgactgaacgttcctcagcagacatcaaaacacgtatggctgagattgcagcccagggtaaagatcccatggagtggttgaagaactctatgctttccggaaagactgaaacactgcaaaaaaaaaatgcaacacccgaggagcaagcggcaacaatactgtattttgcatcagatgaagcccgtcatatcacaggaagcatagtagcatctgatggaggtttcacgacctattaa

SEQ ID NO: 25 - DNA, 786 bp, synthetic construct
  misc_feature 1..786: note = Description of Artificial Sequence: Synthetic polynucleotide
  source 1..786: mol_type = other DNA
atgtatgacgacttgaaaggtaaaaccgtagtggtgaccggatcatccaaaggactgggtgctgctatggctcgtcggtttggagctgaagggatgaacgtggttgcgaattatcgttcggatgaggaaggtgcaagagaaaccgtcagagcaatagaagaggctggaggtgctgctgctgcagtacaggcggacgtgtcaaagaacgaatgtgttgatgcactttttgatgccgcaatgttctcgtttggaggtgtggacatatgggtgaataatgccggtattgaggtggcttctccaagcgaccgtaaatcgatagaagaatggcaacgtgtgatcgatgtgaacctgacaggagtatttgccggttgccgccgcgcaatagaccactttttagatcgtaaaatgcccggcgtaataatcaatctgtctagtgtgcatgaaatcatcccgtggccgcattttgctgattatgccgcgtcaaaagccggtgtaggtatgttaaccaaaacgcttgctttggagtatgcagatcgtggtatacgtgtaaatgcaattgcaccaggagccatgaataccccaatcaatgcagaaaaattcgccgacccggaagcccgtgccgcaacagaaagattgatccctatgggatacgttggagcgcctgaagatgttgctgctgcagctgcgtggcttgcatcggatcaggccagttatgtaactggaacaacccttttcgtagacggaggaatgactctgtatccaggatttcaatttggacagggataa

SEQ ID NO: 26 - DNA, 783 bp, synthetic construct
  misc_feature 1..783: note = Description of Artificial Sequence: Synthetic polynucleotide
  source 1..783: mol_type = other DNA
atgagcgaagcacgtcacaatcctgttttagctggacaaactgcagtgataactggaggtgcctccggaatcggcaaaagcatcgtacaaagattcttggaggctggtgcttcgtgtctggcagccgatttgaatgaggaagctctggcggcattgaaacaggaactggcggaatatggcgataagttagacgtggtcaaagtggatgtatcaaatcgtgatgatgtcgaaggcatggtagaccgtgcagttcagacctttgggcaaatggacatcatagtgaacaatgcaggcatcatggacaacctgttacctatcgccgaaatggatgatgacgtgtgggaaagattaatgaaagtgaatctgaatagcgtaatgtatggaacccgtaaagcagtacgttactttatggaacgtggagaaggcggcgtgataataaatacagcctctctttctggtctgtgtgcaggaagaggtggatgcgcctatacagcatctaaatttgcagtagtgggactgactaaaaatgtcgcatttatgtatgcagacactggaatccgttgcaatgccatatgccctggaaacacccaaactaacattggggtgggtatgcgtcagccttctgaaagaggaatggctaaagcgacgacgggatatgctggtgcaacaagatcgggaacgcctgaggaaattagtgctgcggcagccttccttgccagtgatcaagcaggtttcattaatggcgaaacattaactattgatgggggttggtcagcttattaa

SEQ ID NO: 27 - DNA, 843 bp, synthetic construct
  misc_feature 1..843: note = Description of Artificial Sequence: Synthetic polynucleotide
  source 1..843: mol_type = other DNA
atgcaggatgtattcaccttaaaaaacggagtaaccatgcccagaatcggatttggaacttacaataccagtgacgatgaagcatgtcgtgtagtctgtgatgcggtggaggtgggatatagattgatcgatacggctgcaatttacgagaacgaagcaggtattggccgtgctttggccacttgtggagttccgcgtgaagaactgttcatcactagtaaagtatggaatactcaccgtggctacgacaaaacgatggaatcgtttaatgcgagctgcgaacgtttaggtgtggattatctggacctttttcttatacattggccggccaatgaaaaacagtttggagctgaagccgaggcaattaacgctgacacatggcgcgcattagaggatctgtacaaaaatggcgccgtccgtgctattggcttgtcaaatttcaaaccgcatcatatagaagctttgctgaaacatgccgaaatcgagccgatggtggatcaaatcgaattttatcctggacgtatacaagctgaaactctggaatattgcctggaacgggatttggtagtagaggcatggtcaccgctgggtagaggtaaaactttgactaatgaagctatcgcagaaataggtgcacggtatgggaagtccaatgcacaagtatgcttacgctggctgatccagctgggaatgttgccacttcctaagtcgggaaacattgagcgcatgaaacaaaacttggaagttttcgattttgaactgacacccgaggagatggctgtaatatctgcacaggagaatccgactggacggttttgggacgcggatgaaatcgacttttaa

SEQ ID NO: 28 - DNA, 834 bp, synthetic construct
  misc_feature 1..834: note = Description of Artificial Sequence: Synthetic polynucleotide
  source 1..834: mol_type = other DNA
atgaattttggcggttttatcatgggccgcttcgatgaaaaaattatgttggttactggagctacatctggaataggacgcgcagtggcgattcgtgctgccaaagaaggtgcaaccgtgatcgctgtaggacggaatgaagaacgtggtgcagctgtagtagcagcaatggaggaagcgggtggcaaaggtgaattcatgaaatgcgacgtgtcgaataaagatgcggtgaaagccctttttgccgaaatacagggcaagtatggtaaactggatgtagcagtaaacaatgctggaatcgttggcgcctccaaaacagtcgaggaactggaggatgatgattggttccaggtaattgacgcaaacttgaactcctgttttttttgctgccgtgaagaagtaaaacttatgcagccgtccggaggagcaatcgttaatgtgtcatcagttgcaggaatgcgtggttttccgtcagcggctgcgtatgtggcctcgaagcatgcagtttccggattgaccaaagccgttgcagtagactatgccaccaagggaatcacatgcaatgctatttgtcctgctggaactgatacgccgcttacggaaagaagttccgctgatataaagacccgtatggcagaaattgctgcacagggaaaggatccgatggaatggctgaaaaactccatgttatcggggaaaactgaaacactgcagaaaaaaaatgccacaccggaagaacaagccgcgaccatcctgtattttgcttctgatgaagcacgccacataactggtagcattgtggcttcagatggtggtttcacgacttactaataa

SEQ ID NO: 29 - DNA, 873 bp, synthetic construct
  misc_feature 1..873: note = Description of Artificial Sequence: Synthetic polynucleotide
  source 1..873: mol_type = other DNA
atggattttctggcgttgctgtgctataataccatcaaaagcaataaagaagtaataaaccgtggacgcttcagcggtaaaatcatgttggtaactggtgccacgagtggaataggccgtgcagtggctctgcgtggagcgaaagaaggagctaccgtaatcgcggtaggcagaaacgaagaaagaggtaatgctgtagtggaagccattgaaaataaggagggaaaggcagtattcaaaaagtgcgatgtatcggataaggaggcagttaaaaaactgttcgcggaaatcaaggaagaatttggcaagttagatgtggcagtaaataatgctggtatagtgggagcatcgaaaactgtggaagaactggaggatgatgattggtcgaaggttattgatgcaaacttgaattcatgtttttactgctgcagagaagaagtgaaactgatgaaagagaatggaggtgcaattgttaatgtttcgtcggtagcgggaatgcgtggatttccaagtgcggcagcttatgtcgccagcaaacatgcagttagtggattaacaaaagcggtagccgtagactatgcgacgaaagggattacatgtaacgctgtatgtcctgctggaacggacacaccattaacggaacgtagctcggctgatataaagactcggatggccgaaattgcagcacaaggtaaggaccctatggaatggctgaaaaattctatgctttcaggaaaaacagagactttgcagaaacgtaatgccactcctgaagaacaagctgctacgatattgttttttgcatcagatgaggccaaacatattacaggatcgatagttgcttcagatggaggattcaccacctactaataa

SEQ ID NO: 30 - DNA, 813 bp, synthetic construct
  misc_feature 1..813: note = Description of Artificial Sequence: Synthetic polynucleotide
  source 1..813: mol_type = other DNA
atggaccggttcgagaataagataatgttggtgacaggtgcaacctctggtatcggaaaagctgtggctttgcgtgccgcatctgaaggtgccactgtaattgcagtgggaagaaatgaagaaagaggtcatggtgtggttgaagcgattacttcagcaaacggaaaagccgaattcatgaagtgcgatgtatccgataaagaacaggtcaaagagctttttgcaaaaatcaaggaaagttatggacggttagatgtagccattaacaacgctggaattgtcggtgcaagcaaaacggtagaggaactggaggatgaagattggtcgaacgtaatagatgccaatctgaacagctgtttttactgctgccgtgaagaggtaaagctgatgaaagaaacagggggtgctattgtaaacgtatccagtgtagctggaatgcgtggattcccttctgcagctgcctatgtggcatccaaacatggcgtatctggtttgactaaagcagttgctgttgactatgcaacaaagggaataacctgtaatgccgtatgtcctgccggaacaaacacccctcttaccgaaagaagtagtgcagacattcaggaacatatggcagctctggctgctcagggtaaagatccaatggaatggcttaagaattccatgatgtccggaaagaccgaaactctgcaaaaacgcaatgccacaccggaagaacaggctgcaaccatattgtattttgcctctgatgaagctaagcacatcactggaagcatcgtggcttcagatgggggtttcacaacatattaataa

SEQ ID NO: 31 - DNA, 825 bp, synthetic construct
  misc_feature 1..825: note = Description of Artificial Sequence: Synthetic polynucleotide
  source 1..825: mol_type = other DNA
atgttcaaggatcgtttcaatggccaaacaattattgttactggaggtacgtccggtatcggacgtgctgtatgtattcgtgcagcactggaaggagctaacgtggtagtaagtggacggaacaaagaacgtggccaggcagtcgttgatgaaatattaaagcaaggtggtgaggcaatattcgtacagggcgatataactaaaaaagaagacgtcgtgcatctgtataaggaggcagagcagaaatatggcgaaattcatatcgccatcaataatgctggcatcgtcggagcatcaaagattctggatgaggtaacggacgaggattggggatccgtaattaatgcaaatcttaacagcatgttttattgttgcagagaagccgttaaatacatgttaaagcatggaaaaggtggtgccattgtaaataccagttcagtagctggcatgcgtgggtttccgtctgctgcagcttacgtggcaagcaagcacggcgtaaatggcttgacaaaagccgtggcggtagattatgccacgaaaggaattcgttgcaactctgtaaatcctgccggaacggatacgcctctgacagaaaatgccgcagctggtattaaggctaaaattgctgaactggtaaaacagggaattgacccacagacttttctgaaagaaagcatgacatcaggaaaaactcagactcttcagaagagaaatgcatcaccggaagaacaagccagtaccattttgtatttcgcaagtgatgaagcaaaacatatcactggaagcatcatagcgagtgatggaggattcactgtatattaataa

SEQ ID NO: 32 - DNA, 825 bp, synthetic construct
  misc_feature 1..825: note = Description of Artificial Sequence: Synthetic polynucleotide
  source 1..825: mol_type = other DNA
atgattcaagatcgtttcgctggaaaagttatggtagtaacaggcggaacatctggaattggtaaggcagtgtgcctgcgtgctggagctgaaggagcaaaagtggtgattgctgggcgtaatcaagcacgtggtcaagcaatagaaaaggagattcgcgaagcaggtggagaagctacatttatccagtgtgatgtgacgcagaaagaggacatcataaatttgtatgcaaaaaccatcgagatttacggtcagttggacatcgcaattaacaatgccggcattgttggagactctaaaaaaatagaagatttgacggatgatgattggttctctgtggttaacgccaatctgaacgcaatgttttactgtatccgggaggaaattaaatacatgttaaaaaacgagaatggaggagctatcgtaaacacagcaagtgtcgctggaattcgtgccacgccggccggtcctgcatacgtcgcatcgaaacatggtgtggtaggcctgacaaagtccacagccatggactacgcgaaaaacaacatcatctgcaatgcagtttgtcctgctggaacggacacacctttgacagaagcagctaaagaaaaaatctatgcgaaaatcgccgaattgaaagcgcaagggatcgacccttctgaatttatgaaaaattccatgatcgcaggaaaaacgcagaccttacaggggagaaatgccacatcagaggagcaagcctcgacaattctgtattttgcatccgatgaggcccgccatatcaccggaagcatagttgttgctgatggaggctttaccgtgtactaataa

SEQ ID NO: 33 - DNA, 834 bp, synthetic construct
  misc_feature 1..834: note = Description of Artificial Sequence: Synthetic polynucleotide
  source 1..834: mol_type = other DNA
atgcgtgattattttgagggcagattcgagggaaagaacatgttagtgactggtggaacttccggaatcggcaaagcagtttgtataagagccgcaaaggaaggagcgtttgtaatcattgttggaagaaatgaagaacgggcacaagctgttttgtccgagatagtgcaaaatggtggaaaggcgcgcttcatcaaagcggatgttagccgtgaagatgaggtaacatcgctgtttaacataatcaacaatgaagttggtgaattgcacgtagccataaataacgccggagtagttggacatggtgaacgtatcgatgagcttagtactgaagaatggagccgcgtgatcaatacaaacttaaattccgctttttattgctgtagagaggaagtgaaaaatatgttaaatcataaacaaggtggttcgatcgtaaatgtatccagcgtagccggaacaactgggttttatcgcgcaagtgcgtatgtaacgtctaaacatgcactgaatggcttaacaaaagctgtggcaaatgatttggcaaaatttaatatccggtgcaactctgtttcccctgctgtaactgctacgccccttaatgatcgtagtgcacaagagataaaggttaaacttggaacagctatggcgcagggaaaatcacttgaagaagcaaaatcagaaactatgataggaggtaaaaccgaaacattgcagaaacgtagcgcaacaccggaagaacaggcagcaaccattttgtacattgcatccgaggaggctgcccatataacaggctcaatcattatgtcagacggtggatataccgcttactaataa

SEQ ID NO: 34 - DNA, 834 bp, synthetic construct
  misc_feature 1..834: note = Description of Artificial Sequence: Synthetic polynucleotide
  source 1..834: mol_type = other DNA
atgaagggatattttgaaggaagatttgagggtaagaatgtattggtgacgggtggtactagtggaattggtagagcggtatgtattcgtgcaggtaaagaaggtgcatatgtaattgtggttggtcgtaatgaagctcgtgggcaggcagtcgtttccgagatcattaacaatggtggaaacgctatgtttttccaagcagacgtgagcaaagaaaatgatgtgatcaagctgttcgaggttgtatctgacaaggtgggaaagattcatgtagtgatcaataatgcgggaattgttggacacggtgaacgcatcgatgaactgagcacagacgaatggttaaatgtggtaaatacaaatctgaatagcgcattttattgttgccgggaagctgcgaaaaatatgatcaatcacaaaatcggtggaagcattgtgaatgtatcgtccatcgctggatcaacaggtttctatcgcagttccgcttatgtggccagtaagcatggcctgaatggtttaactaaagctgtggcgaacgatctggctatgtttaacattcgctgtaactcggtatcccctgctggaacagctacacctctgagtgacagaagttccatggaagtgaaaacgaaattgggagcagcaatggcagctggtaaaagcctggaagaagcaaaatcggaaacgatgataggaggaaagacggaaactcttcagaaaagatctgctacatctgaagaacaggcagccaccattttgtacgttgcaagtgatgaggcctcacacatcacgggatcaatcatcatgtccgatggtggttataccgcgtactaataa

SEQ ID NO: 35 - DNA, 819 bp, synthetic construct
  misc_feature 1..819: note = Description of Artificial Sequence: Synthetic polynucleotide
  source 1..819: mol_type = other DNA
atgcatatgaaccggttcggaaataaagttatgttgattactggagcaacgtctggtattggaaaagctgtagcgttacgtgctgcaatggaaggcgctacagtgatagcagttggacgtaatgaagaacggggaaacgcagttgtcgatgaaattgcaaaagaagaaggtaaggctgtgttcatgaaatgtgatgtaagcgatgtggaacaggtgaaacagcttttcacaaatatccaagagaaatatgggaagattgatgtcgcaattaataatgccggggtagtaggtgcctcaaaaactgttgaggaattggcggacgatgactggctgaacgtaattaatgccaacctgaattcctgtttttattgctgccgtgaggaagtaaaattgatgaaagaaaatggtggcgcgatcgtgaatgtttcgtctgttgctggaatgcgtggattccctagtgctgcagcctatgttgcgtcgaaacatggcgtgtctggactgacaaaagcggtcgcagtggattatgccacgaaaggcatcacatgtaatgcaatttgtccagctggaactgatacacccctgaaagaaagatcgtcggcgggaatcaaagaacgtatggcggaattagctgcgcaaggcaaagaccccatggaatggctgaaaaattccatgctgagcggaaaaacagaaactcttcaaaaacgtaatgcgacaccggaagaacaggcagcaactatcttgtattttgcaagtgatgaagcccgtcacattaccggatccattgttgcctccgatggtggttttaccacatattaataa

SEQ ID NO: 36 - DNA, 825 bp, synthetic construct
  misc_feature 1..825: note = Description of Artificial Sequence: Synthetic polynucleotide
  source 1..825: mol_type = other DNA
atgattcaagacagattcgcgggcaaggtgatggtagtaactggtggtacatcaggtataggcaaagctgtttgccttcgtgctggcgccgaaggagcaaaggtagtgattgcaggtcgcaatcaagcacgtggagaagcaattgagaaggaaattcgtgaagcaggtggagaagcggtattcatccaatgtgatgttacacgcaaggaagatatcataaatttgtatgcccgcactgtcgaaatctacggtcgtctggatatcgcagtgaacaatgccggtatcgtgggcgactccaaaaaaatcgaggatttgactgacgatgactggtttagtgtcgtaaatgcaaacctgaatgccatgttctattgtattcgtgaagagataaaatacatgctgaagaatgagaatggtggagcaatcgtgaataccgcatctgtagctggaatacgtgcaacacctgctggtcccgcttatgtcgcatcaaaacatggcgtggtgggtctgactaagagtactgcgatggattatgccaagaacaacataatttgtaatgcagtatgccctgctggaacggacactcccctgacagaagcagctaaggaaaaaatctatgccaaaatagcggaattaaaggcacagggaattgatccctcggaattcatgaaaaattcgatgatagctggtaaaacgcaaacgctgcagggtagaaatgcaacatcggaagaacaagccagcactattttgtattttgctagtgatgaagcgagacatattacgggaagtattgtcgtcgctgatggtggattcacagtgtactaataa

SEQ ID NO: 37 - DNA, 825 bp, synthetic construct
  misc_feature 1..825: note = Description of Artificial Sequence: Synthetic polynucleotide
  source 1..825: mol_type = other DNA
atgttcacagagcgctttaaagacaaagtgatggtggtaacgggaggaacatccggaatagggaaagcagtatgtatccgggctggtgccgaaggtgcaaccgtcgttatcgccggtagaaatgaagaacgtggaaaagcgattgaacaaaccataaccgataatggcggaaaggctctgttcgtacgttgtgatgtaaccaaaaaagaggatataattgctttatacgctaagacaatggaagtctacggacgtatcgatatcgcaattaataatgcagggatcgtgggtgatagtaagaaaatagaggacctgacagatgatgattggttcagtgtagtaaatgcaaacctgaacgcgatgttttattgtatacgtgaagaggtcaaatatatgatgaaaaatgaaaatggaggtagcattgtaaacaccgcgtccgtggcaggaattcgtgctacaccagctggacctgcttatgtggcctcaaaacatggcgtggtaggactgacaaaatctactgcaatggactacgctgggaagaatattacgtgcaatgccatttgcccagctgggacggatacacctttgacagaagccgctaaggaaaagatctatgcaataatagctgatctgaaagcccaggggaaagatccacaggaattcatgaaaaattctatgatagctggtaaaacagaaactctgcagcatcgtaatgccacttctgaagagcaagcagcgaccattctgtattttgccagtgatgaagccagacatatcacaggttccattgttgcctcagacggtggatttacagtctactaataa

SEQ ID NO: 38 - DNA, 825 bp, synthetic construct
  misc_feature 1..825: note = Description of Artificial Sequence: Synthetic polynucleotide
  source 1..825: mol_type = other DNA
atgtttacacaacgcttcaaagataaagttatggttgttaccggagggacctccggaattggaaaagccgtatgcattcgtgctggtgctgagggagctgcagtggttattgctggaagaaatgaaaatcgcggaaaagcgattgaaaaaacaataaccgacaacggtggtacggcccttttcgtacgctgtgacgtaaccaagaaagaagacattcttgctctgtacgccaaaacaatggaagtgtacggaagattggatatcgcggtcaacaatgccggaatagtgggcgactcaaaaaagatagaggacctgactgatgatgactggttcagtgtggtaaacgcaaatttgacggcaatgttttactgcatccgtgaagaagtcaaatatatgatgcaaaacgaaaatggaggatgtatcgtgaatacagcatctgtcgctggaattcgtgctacacctgccggtccggcgtatgtagcgtcaaaacatggagtggtaggattgacaaagtctaccgcaatggactatgctaatcggaacatcacgtgtaatgcagtttgccctgctggtactgatacacctttgactgaagccgcaaaggaaaagatttacgcaaaaattgcagaattgaaagcgcagggaaaggatccgcaagaattcatgaaaaatagcatgatcgcaggtaaaactgaaacattgcagcaccgtaatgctacaagtgaagagcaagctgcaactattctgtattttgcctctgatgaagccagacacattacaggtagcattgtagcttctgatggcgggtttaccgtatattaataa

SEQ ID NO: 39 - DNA, 873 bp, synthetic construct
  misc_feature 1..873: note = Description of Artificial Sequence: Synthetic polynucleotide
  source 1..873: mol_type = other DNA
atggatttcctggcacttctgtgctacaacaccattaaatcgaacaaagaagttataaacagaggtcgtttttctggcaaaattatgctggtaactggtgctacttcaggaatcggaagagcagttgcgttgcgtggagcaaaagaaggtgcaacagtaatcgctgtgggccgcaatgaagaacgtggaaacgcagtggtcgaagccattgaaaataaagagggtaaggccgtgttcaaaaaatgcgacgtaagtgataaagaggcagtcaagaaattgttcgccgaaatcaaggaagagttcggaaaattggatgttgcagtaaataatgcgggtatcgtgggagcatctaagactgtggaggagttggaggacgacgattggagtaaagtgatcgatgctaatctgaattcttgtttttattgctgcagagaagaagtcaagctgatgaaagaaaacggaggagccattgtcaatgtatccagcgtagccggaatgagaggatttccgagtgctgctgcatacgtagcttccaaacatgcagttagtggtctgacaaaagccgtggcggtggattatgcaaccaagggtattacatgtaatgcagtatgtcccgctggaacagatacaccattgactgaacgttcctccgctgacattaaaactcgtatggccgaaattgctgctcagggaaaagatcccatggaatggcttaagaactcgatgttgtcaggaaaaaccgaaacattgcagaaacgtaatgcaacgccggaagaacaagcagctactatactgttctttgcatcggatgaagccaaacacatcacaggatctattgtggcatccgatgggggtttcactacttattaataa

SEQ ID NO: 40 - DNA, 834 bp, synthetic construct
  misc_feature 1..834: note = Description of Artificial Sequence: Synthetic polynucleotide
  source 1..834: mol_type = other DNA
atgcgtgattattttgaaggcagattcgagggaaaaaacatgcttgtgacaggcggaactagcggtatcggcaaatcagtatgtatacgggccgcgaaagaaggagcttttgtaattgtagttggacgtaatgaagaacgtgggcaaagcgtggttaatgagatcgtcgaaaatgggggtaatgcccggtttatcaaagtggatgtatctcgcgaagatgaggtcataaatctgtttaatgtcataaataacgaaattggagacattcacgtagctataaacaatgcaggaatcgtcggacatggagaacgcattgatgaacttagcacagaagaatggctgagagtaataaatacgaatttgaacagtgctttttactgctgccgtgaggaagccaaaaatatgctgaaacacaaacaaggtggaagtatagtaaatatttcttctatcgcaggatcaactgggttctatcgctccagtgcctatgtatcatcgaaacatgcccttaatggtttgactaaagctgtagcaaatgacttggcagcgttcaacatccgctgtaacagtgtaagtcctgcaggaactgcaacacctttgaacgatcgttcagccgaggaaatcaaaggtaagatcggagcagctatggcgcagggaaaatcgatcgaagaggcaaaaagcgaaacaatgatcggtgggaagaccgaaactctgcaaaaacgtagcgctactgcagaggaacaagcagcaaccatcctgtatgtggcatcagaagaggctgcacacattaccggctcaataatcatgtcagatggcggttatactgcgtattaataa

SEQ ID NO: 41 - DNA, 852 bp, Parabacteroides merdae
  source 1..852: mol_type = other DNA
atggaaaaaggattagccatcataaccggtgccgacggaggcatgggacaagtaatcacggcagccctcgcaaaagaaggctatccggtgattatggcttgcctcgatccggaaaaagccgtccccgtatgcacccggatccagcaagaaaccggtaacactcagatcgaagtgcgggaaatcaatctcgcttccctctcatccgtaaacaattttaccggtcaattattaaaagaaggacgtcccgtcagcctcctgatgaacaatgccggaatcctgacgactccggtacgcaaaactgaagatgggttggaaacaatcgtaagcgtcaattatgtggctccctacatgctcacccgccagttattaccattgatgcaaccaggatgccgtattgtaaatacagtgtcctgcacgtatgccatcggccggatcgaaccggacttctttgaaaaaggaaggaacggacgttttttccgcattccggtctatagcaataccaaactggcgctgttattgttcacccaagagtttgccgaacggctgcaagacaaagacatcaccataaatgcctcggacccgggaatcgtcagtacgaacatgatcacgatgcaagcttggttcgacccgcttaccgatatcttgtttcgcccctttatcaaaacaccggcccaaggcgcggcgaccgccatccatctggccctttcggatgaggcgaaagatagaaacggttgttgctatgccaattgcaaaaagaggaatgtgtcagaacgcatccggcatcatgcacagcaaaaacaactttgggacgatacggaaatcttgctccggcaaaaaggaatccggttctga

SEQ ID NO: 42 - DNA, 861 bp, Bacteroides dorei
  source 1..861: mol_type = other DNA
atgagtaaattagccataataaccggtgccgatggaggaatgggtactgaaataacccgtgcggtagcacaggccggctatcatgtaataatgttgtgttacactctttttaaaggagaggagcgtaagaaccagttgattttggaaactggcaataaagagatagaagtcagacaagttgacctttcttccatggcttctgtgactaatatcgcggacgacttgttggggcgcggaaagcatatcgatttactgatgaacaatgcgggaacaatgagttccggcggtttgattacaacggaggatggtttggaatatacggtagctgtgaattatgtggccccttttttactgactttgaaattattgcctctgatgggacaaggaacccggattgtgaacatggtttcttgcacgtattccatagggaagattactcctgaatttttgattcgtggaaaaagaggcagtttttggcgtatccctgtttacagtaatacaaaattggctttgtggctttttacccgtgaactttctgaaaggctgaaaacagaaggaattactgtcaatgctgccgatcctggtattgtttctaccaatatcatccgtatggatatgtggtttgacccgttgactgacatactgttccgtccttgtatccgtactccgaagcaaggagccgcgacagctgtcagtttgcttttggatgatcgatggaaagaagttacagggcaaatgttcgcttcttgcaagcctaaaaaagtaaaggataagtttatgaatcatccacaggcaagacagctttgggcggatacgaaagcatatttggagaaactgaaattggaggagccaattgtctga

SEQ ID NO: 43 - DNA, 861 bp, Bacteroides vulgatus
  source 1..861: mol_type = other DNA
atgagtaaattagccataataaccggtgccgatggaggaatgggaaccgaaataacccgtgcggtagcacaggccggttatcatgtaattatgttgtgctacactcttttcaaaggagaggagcgtaagaaccagttgattttggaaacaggcaataaagagatagaagtcagacaggttgacctttcttctatggcttctgtgactaatatcgcagaagatttgttggggcgtggaaaacatatcgacttactgatgaacaatgcgggaaccatgagttccggaggtttgattacaacggaggatggtttggaatatacggtagccgtgaactatgtggccccttttttactgaccttgaaattattacctctgatggggcagggaacccggattgtgaacatggtttcttgtacgtattccatagggaagattactcctgaattttttgttcgtggaaagagaggaagtttttggcgtatccctgtttacagcaatacaaagttggctttgtggctttttacccgtgaactttctgaaaggctgaaagcagagggaatcaccgtcaatgctgccgatcccggtattgtttctaccaatatcatccgtatggatatgtggtttgatccgttgaccgacatactgttccgtccttgtatccgtactccgaagcaaggagctgcgacagctgtcagtttgcttttggatgagcgatggaaagaagttacaggacagatgtttgcttcttgcaagcctaaaaaagtaaaggataagtttatgaatcatccgcaggcaagacagctttgggcggatacgaaagcatatttggagaaactgaaattggaggagccaattggctga

SEQ ID NO: 44 - DNA, 852 bp, Bacteroides thetaiotaomicron
  source 1..852: mol_type = other DNA
atgagtgaagagaaatgggcaatcatcaccggtgccgacggcggcatgggaacggaaataacccgtgccgtagccgaagccggttaccatattattatggcttgctatcgtccgtccaaagcggaaccgatacggcagcgtctagtgaacgagacaggaaacgcaaacatggaagtcatggcagtcgatctgtcttctatggcatcgacagcttcttttgccgatcggattgtggagcgtcatctccccgtttccctgctgatgaataacgccggaacaatggaaaccggacttcacatcaccgacgacggctttgaacgaacggtcagtgtgaactatctggggccgtacctgcttacccggaaactccttccggcattgacatgcggagcccgtattgtaaacatggtttcttgcacgtatgcgatcggacacctcgattttcccgatttcttccggcagggaagaaagggaagtttttggcgaatccctgtttacagcaataccaaactggctttgatgctgtttacgatcgaactttcggaacgcctccgtgaaaaaggaatcactgtcaatgccgccgatcccggcattgtttctaccgacatcatcactatgcaccagtggtttgaccctctgacggatatctttttccgcccctttatccgcacgccgaagaaaggggcttccactgccgtcggcctcttgctggatgaggcagtggccggagtcagcggacagctttatgcgagcagccacaggaagcagctgtccgaaaaatacctctgccatgtgcagcaaaaacaactgtggcaggaaacggaacaggctttggaacgctggttgaaataa

SEQ ID NO: 45 - DNA, 852 bp, Bacteroides caccae
  source 1..852: mol_type = other DNA
atgaatgaagtgaaatgggcgatcattaccggagctgacggaggcatgggaacggagataacccgtgccgtggccacagccggctatcatgtcataatggcttgttataacccgcaaaaagcggaaaacgtgtgccaacgtttaatgaaagaaaccggaaatccgaatttggaagtactcgctattgatctgtcttcgatgcactccgtagcctcttttactgatcggattttggaacgtaaactttccatttccttgttgatgaataatgccgggacaatggaaaccggattttctattacgaacgatggatttgaacggacggtcagtgtaaattatgttggtccttacctgctgactcgtaaattagttccgactatggcatccggagcacgtattgtaaatatggtttcgtgtacgtatgcaatcggccgtcttgattttcctgatttctttcacagggggaaaacgggaaacttttggagaatacctgtttatagtaacacaaaattggctttgttattatttactttcgaactatccgagcaacttcgggagaaaggaatcaccgtcaatgctgccgatccgggaattgtctctactgatatcatcacgatgcataagtggttcgaccctctgacagatatattcttccgtccttttattcgtaagccgaagaaaggagcttctacggcaattggtctgttgctggacaaaaaagaagccggtgtgacagggcaactctatgtcaataatcaccggaaaagcttatccgataagtatgtgaaccatgtacagaaagagcagttgtgggaaataacggagcgtttgctggcgcaatggttggagtag

SEQ ID NO: 46 - DNA, 855 bp, Bacteroides finegoldii
  source 1..855: mol_type = other DNA
atgaatgatataaaatgggctatcattaccggtgcggacggagggatgggaacggaaatcacacgtgccgttgccaaagctggttatcaggtaataatggcttgttacaacccccaaaaggcggagactgtccgcgcttgtttgattgaagaaaccggaaacccgaatctggaagttatggctcttgatttggcttccatgcaatccgtagcttcttttgccgaccggatattagaacgtaaccttcctgtttctctgctgatgaataatgcgggaacgatggaaacgggacttcatattaccgtagatgggtttgagcgaacggttagcgtgaattatgtaggaccttatctgcttacccggaaactgattcctgcgatggtgcgcggtgcgcgaattgtaaacatggtgtcttgcacttatgcgatcgggcgtattgaacttcccgatttctttcacagaggcaaggtcggagaattttggagaattcccgtttacagcaatacgaaactggctttattgttgtttaccattgaactgtccaagctactccgtgataaaggaattaccgtcaatgctgccgatccgggcattgtctctactaatattattactatgcataagtggtttgacccgctgacggacatttttttccggccttttattcgcaagcccgcacaaggggcttccaccgctatcggtttgttgttggatgaaaaagaagccggagtgacggggcaactgtatgctagtaatcgtcggaaagaattatcggataaatacgttcatcacgtgcagagggagctactgtgggaagtcacggaacgttcgttggcacgatggatttcttcctaa

SEQ ID NO: 47 - DNA, 840 bp, Bacteroides uniformis
  source 1..840: mol_type = other DNA
gtgaatttagctgttataactggggcagacggtggcatgggcatggaaattacccgcgcagtggcaactgccggctatcaggtcatcatggcatgccgtgacccccaagctgccgaacccaagcggcaactactgatgcgtgaaaccggtaatccgcgtattgagactgctcccattgatttggcatctctggcttcagtggccgcatttgcagagcatctgttgaagcggggagagccgttggcgttgctgatgaacaatgccggaaccatggaaacggaacgccgcattaccgaagacggactggaacggacggtgagtgtcaattatgtagggccttacctgctgacccgcaagctgctaccattgatgggagaggggagccgtattgtgaatatggtatcttgtacgtatgccatcggtcatcttgactttccggattttttcctccggggaaggaagggtggcttttggcgcattcctatatatagcaacacgaagctggcattgactctgttcaccatcgacttggccagtcgcgtcaaacacaaaggtattgttgtgaatgcggccgacccggggattgtgtctaccaatatcatcaccatgcatatgtggtttgacccgctgacagatatacttttcaggccttttatccgtactccccgtaagggagctgcaacagctgtcggcttattgctggatgaggatgccggtaaacgtacggggacattgaatgccagttgccgtcccaagtctctttcggagaagtacacccggcatgtacagatggaagaactgtgggagaggacggaaagtatagtgaaaaaatggttgtaa

SEQ ID NO: 48 - DNA, 768 bp, synthetic construct
  misc_feature 1..768: note = Description of Artificial Sequence: Synthetic polynucleotide
  source 1..768: mol_type = other DNA
atggacatgggattgaaagataaggtagtcttaataacaggtggaggaggcggaatcgcacgtggcatcgaacgtgcatttgcaacagaaggagcaaagtttattctgacggacctgttccctggaggcttggaagccgctaaggaggaattggaacgcgattttggatccgaagtctttacgatactggcaaatggaagtgtagaagaagaggtgcgtgcttctgtcgaagcaggtgccgaacattttggtggccgtattgatgttctgatcaataatgctcaggcttccgcatccggattgactttggtacaacattcagaggaggatttcgatcttgcagtgcgctctggactttatgctacgttcttttatatgaagcatgcctatccatatttgaaggaaactgcagggagtgtcattaatttcgcaagtggtgccgggatcggaggtaatcccggacaatcatcatatgcagctgctaaggaaggtattcgtggcatgagtcgtgttgcagcttcagaatggggaccggataatattaacgtaaacatcgtgtgccccatagtaatgaccaaagcactggaagaatggcgtgaaagagaacccgaaatgtacgaaaaaaacgtgaaagcaatacccctgggtcgctttggagatgcggaaaaggatgttggacgcgtatgtgtatttcttgcaagcccagatgccagttttgtaactggagatacaattatggttcagggcggttccggcatgaaaccataa

SEQ ID NO: 49 - DNA, 768 bp, synthetic construct
  misc_feature 1..768: note = Description of Artificial Sequence: Synthetic polynucleotide
  source 1..768: mol_type = other DNA
atggacatgggattaaaggataaagtagttttaattaccggaggtggaggaggtatagcacgtggtattgaacgtgctttcgcaactgaaggtgccaaattcattctgactgacttatttcctggaggattggaagcagctaaagaagaactggagagagactttggttccgaagtcttcacaatcctggcaaatggatctgtagaagaagaggtccgtgcagccgtcgaagctggagccgaacatttcggtggccgtatcgatgtgctgataaataacgcgcaagcatccgcttccggattgactttggtgcagcattctgaggaagattttgatttggcagtccggagtggattgtatgcaacgttcttctatatgaaacacgcctacccgtatcttaaagagacagccggatcagtaatcaattttgcttcaggagctgggatcggtggtaatcccggtcaatcatcatatgctgccgctaaagaaggcattcgtggtatgtcacgtgtggcagcttctgaatggggaccggataatatcaatgtaaacatcgtgtgccctatagtaatgactaaagcgttagaagaatggagagaacgtgaaccggagatgtatgagaaaaatgtcaaagctattccgttgggtcgttttggtgacgcagaaaaggacgttgggagagtttgtgtattcttggcatcacctgatgcatccttcgtaacaggagacactatcatggtacaaggcggatcaggtatgaaaccgtaa

SEQ ID NO: 50 - DNA, 801 bp, synthetic construct
  misc_feature 1..801: note = Description of Artificial Sequence: Synthetic polynucleotide
  source 1..801: mol_type = other DNA
atgggattcttagaaggaaaaactgccataattaccggcggtggacgtgcagtcttaaaagatggctcttgcggttcaattggatatggtatagcaaccgcgtacgcaaaagaaggcgcaaacttagttattacaggacgtaatgtgcaaaaattggaagatgccaaggaagaattggagcgcttatacggaatcaaagtattgccgattcaagccgatgtatctgcaggcaatgataacgccgcaaccgttcaaaatgtgatcgataaaactattgaagaattcggacggattgatgtgttgatcaataatgcccaagctagtgcttctggagtatccctggcggagcatacaacggaccaattcgatttggcaatctattctggtttgtatgctgccttctattacatgcaagcatgttaccctcacttgaaagaaacgaaaggtaccgtgataaactttgcaagtggagcaggattgtttggcaatgtgggacaatgttcgtatgcagctgcgaaagaaggaatcagaggtttgacacgtgttgccgcaaatgaatggggtgccgatgacattaacgtgaatgtaatctgcccgttggcttggacggcacaattggagaattttgcggaggcatatccggacgcattcgaaacgaatgttcatatgccgccgatgggacattatggtaatgtagagactgaaattggacgtccgtgtgtacagttggcttcacctgattttcgtttcatgtccggggaaacaatcacgttggaaggcggtatgggattacgtccataa

SEQ ID NO: 51 - DNA, 813 bp, synthetic construct
  misc_feature 1..813: note = Description of Artificial Sequence: Synthetic polynucleotide
  source 1..813: mol_type = other DNA
atggatttcattgacttcaaagaaatgggacggatgggaatttttgacggaaaagtggcaataatcactggtggagggaaggcgaaaagtatcggatatgggatagctgtagcgtacgcaaaagaaggcgcaaacttggtcttaacgggacgtaacgaacaaaaactgttagacgctaaggaagaactggaacgcctgtatggaatcaaagtccttcctctggctgtggacgttacgccttcggatgaatcagaagatcgcgtaaaagaagccgtacagaaagtcattgccgaattcggacggatcgatgttttaatcaacaacgcacaggcatcagccagcggtattccgttgtcgatgcaaacgaaggatcatttcgatttgggaatctatagtggattgtatgctacgttctactacatgagagaatgttacccgtatttgaaagaaacccagggctccgttataaattttgcatcaggcgcgggtttgttcggtaatgtcggacagtgttcttacgcagctgccaaagaaggaattcgcggattatcccgtgtcgctgcaacagaatggggcaaggacaatattaatgttaatgtcgtgtgccctttggctatgacggcccagttggaaaattttaaattatcgtacccggaagcatatgaaaaaaatctgagaggtgtccctatgggacggtttggtgaccctgaattggacattggccgtgtatgtgtgcagcttggatctccggattttaaatatatgtctggtgagacactgacccttgaaggtggaatgggacaacgtccgtaa

SEQ ID NO: 52 - DNA, 780 bp, synthetic construct
  misc_feature 1..780: note = Description of Artificial Sequence: Synthetic polynucleotide
  source 1..780: mol_type = other DNA
atgggcatttttgacggtaagacggcaatcataacaggtggtgggaaagcgcgtagtattggttatggaatcgccgtcgcatatgcgaaagaaggtgctaacctggccttgacaggcagaaacgaacagaagctgttggatgccaaggaagagctggaacgcctgtacggaataaaggttttgccgttacaagctgatgtgacacctgatgaaaaaagcgaggaagtcgtgaaagaaacggtgcagaaagtggtagacacctttggccggattgacgttttgatcaataatgcgcaggcttcagcctcaggtattccgctgagcatgcacatgaaagaccatttcgacctgggtatttattctggcctgtacgcggtattttattatatgcgtgcgtgctacccgtacttaaaggaaacccagggatccgtgataaattttgcttctggtgctggattgtttggaaatgccggtcagagcagttatgccgcggcaaaagagggaatacgtggcatctctcgtgtggccgctacagaatggggtaaagataacattaatgtgaacgtggtctgtccgctggccatgactgcgcagctggaaaattttaaggaagcgtacccggaagcgtacgaaaaaaacctgaaagcggtgccgatgggtcggtttggtgatcctgagaaagatatcggaagagtctgcgtgcatttgggaagtcctgatttgaagtacatgtctggtgaaactcttactctggaaggtggtatgggccaacggccttaa

SEQ ID NO: 53 - DNA, 801 bp, synthetic construct
  misc_feature 1..801: note = Description of Artificial Sequence: Synthetic polynucleotide
  source 1..801: mol_type = other DNA
atgggttttctgacaggtaagacagcaatcataaccggtggtggacgtgctaccttaagtgatggaagctgcggaagtatcggatacggcattgctaccgcatatgccaaggaaggagcaaatttgacgctgacaggacgtaacgtgaaaaaattggaagacgcgaaagaagaactggaacgtctgtatggaataaaagtgttggctgtccaagctgatgtttcagctggagccgataacaaagccgtagtggaacaggtaatcaagcaaactgtcgaggaattcggaagaatcgatgtacttataaataatgcacaggcttccgctagtggagtatcaatagcagatcataccacggaacaattcgatcttgcgatatattccggtttatatgcagcgtactattacatgcaggcatgttatccatatttggccgaagctaaaggaagtgttattaactttgcaagtggtgctggacttttcggccattatggacagtgttcgtatgcagctgcaaaagaaggtattcgtggtcttacacgtgttgctgcaacagagtggggcaaggatggaatcaacgtaaatgtcgtttgtcccttggcatggactgtccagctggaaaatttcgaaaaagcctatcctgatgcattcaaggcaaatgtaaaaatgcctccggcaggacactatggagatgtagagaaggaaattggtagagtatgcgttcagctggccagccctgacttcaaatttatgtctggagaaacgatcactttggaaggtggcatgggacttcgtccataa

SEQ ID NO: 54 - DNA, 801 bp, synthetic construct
  misc_feature 1..801: note = Description of Artificial Sequence: Synthetic polynucleotide
  source 1..801: mol_type = other DNA
atgggctttctgaacggaaaaacagtaatagttactggaggtggacgttctgtactttctgacggccgctgtggatctattggttatggaatagtaactgccttcgcaaaagaaggagctaatattgtaatcacgggacggaacgtaaaaaagcttgaggacgcaaaagaggaaatcgaacgcctgtatggagtaaaagtactgcctgtaagagccgatgtgtcggcaggtggagacaataaggccgtggtcgacgaagtgataaaacaaacaatagatacgtttggaagaatagacgtcttagtaaataatgcccaggcttcggcatctggtgtaactctggaagaccatacaacggaacagtttgacttggcaatatacagcggtttatatgcaacattctattacatgcaagcatgtcttccctacttaaaggaaacaaagggttctgtgataaactttgcttcaggcgcgggtctttttggtaactatggtcaatgtgcctatgcagcagcaaaggaaggagtccgtggtttaacacgcgtggctgcaactgaatggggccagttcggcattaatgttaatataatctgtccccttgcttggacagctcaactggagaattttgaaaaggcttatccagaagcatttaaagaaaatgtaaaaatgccaccagctggccattatggggatgcggaaaaagaaattggtagagtatgtgtccagttggcttcaccggacttcaaatacatgtcgggagaaactatcacattagaaggtggtatgggacttcgtccctaa

SEQ ID NO: 55 - DNA, 762 bp, synthetic construct
  misc_feature 1..762: note = Description of Artificial Sequence: Synthetic polynucleotide
  source 1..762: mol_type = other DNA
atgaaagaactgaatgagaaagtggccattatcacaggtgctggacaaggtatcggcaaagggatagccttacatctgggtaaacgtggcgtgaaagttgtttgcgttggacgccgtttggatccgattgtccaaaccgtgaaagaagttgaagaagctggtggccaaggattcgctataacttgcgatgtgggtaatcgggaggatgttaaaaaagtggtcaaagcgaccgtagaaaaatacggaacggtcgatgtagttgtaaataatgcacagagtttgcctgggtccgccaaagtggaagatacaacgtacgaacagatgcttactgcatggcaaagcggaacaatcggttcactgaacatgatgcaagaatgtttcccatatatgaaagaccagaacgaaggtagaatcatcaactttgcttccgcaacaggcatgtttggctatgcaggacagcttgcctatggctgcaacaaagaatcgattcgtggactgaccaaaattgcggcaaaagaatgggctcaatacaacattatcgtaaattgtgtgcttcctggggccgaaagcccagcagcaaaggtatgggccgagaaattcccggaaaagtataaggaaataatggaagcccaaccaatgaaacgttttggggatggtgaagatgacataggtcgtgtaatcgcttttcttgcaggtcctgattctaaatattacactggacagtgcctgttggttgatgggggatatagtatagccccgtaa

SEQ ID NO: 56 - DNA, 762 bp, synthetic construct
  misc_feature 1..762: note = Description of Artificial Sequence: Synthetic polynucleotide
  source 1..762: mol_type = other DNA
atgaagcagctgaatgaaaaagttgctattgtaaccggtgctggtcaaggtattggacaagggatcgctttgtgtctgggcaaacgtggggtaaaggtggtatgcgtaggaagaagacctgaaccgatcgaagcaaccgccaaagaaatccgtgatttgggaggtgaatcgtttgctatgacctgtgacacagcggatcgtgacagagtaaaggaagtcgtggcaaaaactgtagagacatataaaacagttgatgtaatgatcaataatgcccagagtttgcctggaagcgctcctgtggaagaggtaacatatgaaatgatgtacactgcctggtctactggtactctgggaagcttgaattttatgcaagaatgcttcccttatatgaaagaacaaggtgaaggacgtgttataaattttgcaagtgccacgggtatgttcggttatgctggaaatcttgcctatggctgcaacaaggaagcgattcgtggattaaccaaaatcgcggcaaaggaatggggaaagtatggcatttgcgtcaactgcgtgttgcctggagctgaaagtccagcagctaaaatctgggccgaaaagtttcccgaaaaatatgcagagattctggaacagcagcctatgaaaagactgggagatgccgaaaaagacatcgcaccagtcattgccttcctttccggacctgattcttgttattattctggccagtgtcttctggttgatggtgcctattccataatgccctaa

SEQ ID NO: 57 - DNA, 762 bp, synthetic construct
  misc_feature 1..762: note = Description of Artificial Sequence: Synthetic polynucleotide
  source 1..762: mol_type = other DNA
atgaaacagctgaatgaaaaggtggccatcgtaaccggcgccggacagggaattggaaagggaatcgcattgtgtcttgcgaagcgtggcgtaaaagtagtatgtaccggaagacgggaagctccaatccaacagactgtggctgagattgaagaattgggtggacagggactggccatgacatgtgattcggcagatcgtgcccgcgtggaagaggtagtaaaagcagccgtggatacattcggctctattgatgttatcgtgaataatggacaggctattgtgccgtccgcccctgtagaagacacgacatacgaaaacatgttagccgcatggcagtctggtactataggatcattaaattacatgcaagctgcattcccgcatatgaaggaacaacatgaaggccgtataataaatttcgcctctgctacgggaatgtttggaatcgctggccagcttgcttatgggtccaataaagaagctttgcgtgggctgacaaaaatagcagccaaagaatggggacaatatggtatctgcgtaaatgttgtattacctggagcggaatcacctgcagcgaaggcatgggctgaaaaatttccggaggaataccagaagcaagtaatgctgaacccaatgcatagatttggtgaccctgaggatgatatcgcaccggtagttgcatttttagcaggccctgattcatgttattattccggccagtctgtaatcgtcgatggcgcgaattccattatgccttaa

SEQ ID NO: 58 - DNA, 762 bp, synthetic construct
  misc_feature 1..762: note = Description of Artificial Sequence: Synthetic polynucleotide
  source 1..762: mol_type = other DNA
atgaaacaattgaatgaaaaagttgcaattgtcacaggagctggacaaggaatcgggaaaggcattgccctgtgtcttgctaaacgtggagtaaagattgtggccactggtcgtcgtttggaaccgatcgaagcgacaatagcagaaataaaggaacttggtggtgatggactggcgatgagttgcgattctgcagacagagaacgcgttttcgaagtagtaaaaaccgccattgacacctttggtagtattgatgtcatcgtaaacaatggacaagccattgtaccaagccaaccggtggaggatactgaatacgaaaacatgttaaaagcatggcaatcgggagttattggaagtttgaattacatgcaagcagcttttccatatatgaaggaacagcatgaaggtcgcatcataaattttgcatccgcgactggtatgtttggaattgcgggtcagttagcctatggtagtaacaaggaagctctgcgcggattaacaaaaatcgcggctaaagaatggggacaatacggaatttgtgtgaatattgtgctgcctggagctgaatctcctgccgcaaaggcctgggccgaaaaattccctgaagaatatgcgaaacaagtaaatttaaacccgatgaaacggtttggagatccggaagctgacatcgcgcctgtggtagcttttcttgcaggacctgacagctgctattttagcggacaatccgtgatagtagacggtgcaaattcaattatgccgtaa

SEQ ID NO: 59 - DNA, 762 bp, synthetic construct
  misc_feature 1..762: note = Description of Artificial Sequence: Synthetic polynucleotide
  source 1..762: mol_type = other DNA
atgaaacaattgaatgaaaaggtggctattgtgactggtgctggacaagggattggaaaaggaattgccttatgcctggcgaaaagaggagtcaaaattattgcaacgggacgtagactggaacccattgaacaaacaatagcggagataaaggagctggattctgatggactggcaattacatgtgactcagcggatcgtgcccgtgttgaagaagttgtgaaaactgctgccgatacatttggaacagtggatatcgtggttaataatgcacaagctatcgtgccgtctgcgcctgtggaggaaactagctatgacaacatgttcaaagcatggcagagtggagtaattggcagcctgaactatatgcagtccgtgtttccttacatgaaggaacaacacgaaggtcggatcataaattttgcaagcgctaccggtatgtttggtatcgcgggacagttggcctatggatcgaataaagaagctatccgtggaatgaccaaaattgcagcaaaggagtggggacagtatggtatctgcgtcaatgttgttttgccgggtgctgaatcccctgctgcaaaggcttgggcagagaaatttcctgaggagtatgcgaaacaagtgaatttaaacccaatgaaacgttttggtagtcccgagaatgacatagctccagtgattgcttttttggccggaccggattcttgctatttttctggacaatcagtagtggtagatggagcgaatagcattatgccgtaa

SEQ ID NO: 60 - DNA, 762 bp, synthetic construct
  misc_feature 1..762: note = Description of Artificial Sequence: Synthetic polynucleotide
  source 1..762: mol_type = other DNA
atgaaacaactgaatgaaaaagtggctatagtaactggggccggacaaggtatcggcaagggaattgcattatgcctggctaagcgcggcgtaaaaattgttgccactggacgtcgtttggaaccgattgaacaaacgatcgctgaaattaaggagctgggtggcgatggatttgctatgtcctgtgattctgctgatcgtgctaaagttgaagaggtggtaaaagcaacagtggatacctacggaattgtcgacgtcgtggtaaataatgctcaagcaatcgttccgagtgcccctgtggaagaaacgacgtatgagaatatgttgaaggcttgggaatcaggggtaatcggcagcttgaattatatgcaggccgcttttccatacatgaaagagcagcatgaaggtcggatcatcaattttgcaagcgcaactggaatgtttggcattgctggtcagctggcctatggcagtaacaaggaagccttacgtggtttaactaaaattgctgccaaagaatggggacagtacggaatatgcgtaaatatagtccttccgggtgcggaaagtcctgcagccaaagcatgggcagccaaattcccggaagagtatgcgaaacaagtaaatttaaatccgatgaaaagattcggtgatccggaaaatgacattgcacctgtcatcgcgtttttagctggcccggactcatgctattacagtggacaaagtgttattgtggatggagctaattcaattatgccgtaa

SEQ ID NO: 61 - DNA, 762 bp, Parabacteroides merdae
  source 1..762: mol_type = other DNA
atgacaactgaacatttcaccttatttctaattgttatggcagctatcgccgccatagtcttcatagccctttatttcgtcgaagccggttatggaatgttgttcgataaaaaatggggacttccgataccgaacaagattgcttggatttgcatggaagcgccggtttttatcgtcatgtttttgttatggaacggatcggaacgacagttcgagacagtaccgttcctgatattcttattcttcgaactgcattatttccaacgatcttttatttttcctctgttgataaaaggcaaaagtaaaatgccggcaggcatcatgcttatgggaatcacctttaacctcctgaacggttatatgcagggagaatggattttctacttagcaccgcaggatatgtatacgaaaagctggctgcacagccctcaatttatagtcgggacaatcttgttcttcaccggcatggcaatcaatatccagtcagaccatattgtccgccacctcagaaagcctggcgacacgaaccattatctgcctaaaaaaggcctgttcaaatatgtgacatcagccaactactttggcgaaatcgtggaatggtgcggatttgcaatcctgacctggagtgcaagcggagctgttttcgcttggtggacatttgcaaaccttgtacctcgcgcaaacaccatctaccataaatacaaagcgatgtttggtaacgaactggaaaaccgtaaacgggttattccttttatatattga

SEQ ID NO: 62 - DNA, 765 bp, Bacteroides dorei
  source 1..765: mol_type = other DNA
atgggacaacagacttttgaatttttgctattggcaatgtccgcacttgcggtgattgtatttgtagccctctattatgtacgtgccggttatggtatattccacaccccgaaatggggactttcagtgaacaataaattaggttgggtgctgatggaagcgcctgtattccttgtaatgctttatctgtggtggaacagcagcgtgcgttttgatgccgctcctttcctcttttttcttctttttgaattacattatttccagcgctcttttatcttccctttcctgatgaaaggaaagagccggatgccccttgccattatgttgatgggagtggtctttaatgtcctgaacggactgatgcagggcgaatggttgttctatctggctccggaaggactctatacagatgcctggctcagtactccttctttttggtttgggatcattttgttctttatagggatgggcattaatctacattccgacagtgtgatccgccatttacgtaaaccgggcgatacacgtcattatttgccgcagaagggaatgtaccgatatgtcacttcgggcaactattttggcgagttggtggaatggatagggtttgccgtactcacttgttcgcctgctgcatgggtgtttgtactgtggacgtttgctaatctggctccacgtgctaattccatccgtaaccgttaccgggaagagtttggtaaggatgcggtaggaaaaaagaaaagaatgattccttttatttattga

SEQ ID NO: 63 - DNA, 765 bp, Bacteroides vulgatus
  source 1..765: mol_type = other DNA
atgggacaacagacttttgaatttttgctattggcaatgtccgcacttgcggtgattgtatttgtagccctctattatgtacgtgccggttatggtatgttccacaccccgaaatggggactttcagtgaacaataaattaggttgggtactgatggaagcgcctgtattccttgtaatgctttatctgtggtggaacagcagcgtgcgttttgatgccgctcctttcctcttttttcttctttttgaattacattatttccagcgctcttttatcttccctttcctgatgaaaggaaagagccggatgccccttgccattatgttgatgggagtggtctttaatgtcctgaacggactgatgcagggcgaatggttgttctatctggctccggaaggactctatacagatgcctggctcagtactccttctttttggcttggggttattctgttctttatagggatgggcattaatctacattccgacagtgtgatccgccatttacgtaaaccgggcgatacacgccattatttgccgcagaagggaatgtaccgatatgtcacttcgggcaactattttggcgagttggtggaatggatagggtttgccgtactcacttgttcgcccgctgcatgggtgtttgtgctgtggacgtttgctaatctggctccacgtgccaattccatccgtaaccgttatcgggaagagtttggtaaggatgcggtaggaaaaaagaaaagaatgattccttttatttattga

SEQ ID NO: 64 - DNA, 762 bp, Bacteroides thetaiotaomicron
  source 1..762: mol_type = other DNA
atgagtatagctgcctttaatctatttttgggcgtcatgagtctgaccgctctgattgttttcatcgccctctactttgtgaaagccggttacgggatatttcgcaccgcctcctggggagttgccatttccaacaagctggcgtggatactgatggaagcccccgtatttctggtcatgtgctggatgtggatacactcggaacgtcgttttgatccggtcatactgacattctttgtcttctttcagattcattattttcagcgcgccttcgtctttcccctgctactgaccggaaagagtaaaatgccgctggcaatcatgtcgatgggaatcctgttcaatctattgaacggctatatgcagggtgaatggatattttatctctcacccgagggaatgtatcattccggctggttcacttccgcatggtttattgcgggcagtctgcttttctttgcgggcatgttgatgaactggcattcggactatatcatccgccatttgcgcaaaccgggggatacccgtcattatctgccacaaaaagggatgtaccgctatgtcacttccgccaattatctgggcgaaatcattgaatgggcaggctgggcaatactgacttgttcactatccggacttgtattcttctggtggacagtggccaatctcgtcccccgtgccaatgcaatctggcatcgctaccgtgaagaatttggctcggaagtaggcgaacgcaaacgtgtatttccttttatctattga

SEQ ID NO: 65 - DNA, 762 bp, Bacteroides caccae
  source 1..762: mol_type = other DNA
atgactatgaatgcatttaatctgtttttgggcataatgagcctgatcgctctgattgtttttattgccctttactttgtgaaagccggatatggtatttttcgtactgcttcgtggggtgtggctatttccaataagttagcttggatattaatggaggcccctgtatttttagttatgtgttggatgtgggtgcattcggaacgccgttttgatcccgtcatactgatgttcttcatattcttccagattcattatttccagcgtgcattcgtttttcctctattgctgaccggaaagagtaaaatgccgttagctattatgtcaatgggcattctttttaatttgttgaacggatatatgcaaggacaatggatatttcatcttgcgcctgaaggaatgtacggcattgattggtttatgtcaccatggtttattctcggaactctgcttttttttactggtatgctggtgaactggcactcggattatatcatccggcatttgcgaaagccgggagatacccgccactatctgcctcaaaaagggatgtaccgctacgttacttccgccaattacttcggcgaaatagtagagtgggcaggctgggcgatactcacttgttcactttccggacttgtgtttctttggtggacgatcgctaaccttgtcccgcgtgccaacgcaatctggcaccgttaccgcgaggaattcggtgatgaggtgggaaataggaaacgtgtattcccttttctgtattaa

SEQ ID NO: 66 - DNA, 762 bp, Bacteroides finegoldii
  source 1..762: mol_type = other DNA
atgaatttagcagcttttaatctgtttttgggtgtaatgagcttgattgccctgattgtttttgtcgctctctacttcgtgaaagcaggatacggaatcttccgcacgtcttcgtggggagcggctatttcaaacaagctggcttggatactgatggaagctccggtcttcctcgtgatgtgcgtgatgtggatgtattcggaacgccgctttgagccggtgatattgaccttctttttattcttccaactgcattattttcaacgggctttcattttccctttgttattgaaaggaaaaagtaaaatgccgttggccatcatgtcaatgggaatccttttcaatctgttgaatggatatatgcaaggagaatggattttctaccttgcccccgcaacgatgtaccagtccgattggttcacctccccgtactttataataggtactttgctgttttttacgggtatgctggtgaactggtcgtccgattatatcatccgccatttgcgtaaaccgggagacacacggcattatctgccacagaaaggtatgtaccgctatgtgacttccgccaattatttcggtgaaatcgtagaatgggcaggctgggcaattcttacctgttcgctttccggacttgttttcctttggtggacgattgcaaacctcgttccgcgagccgacgccatctggaaacgttatcgcgaggaattcggcgacgcggtaggcacacggaagcgggtgtttccttttctctactag

SEQ ID NO: 67 - DNA, 771 bp, Bacteroides uniformis
  source 1..771: mol_type = other DNA
atgaatcaggaaacttttcagatatttctgtgggtaatgagtgctgtggcattggttgtctttattgcactctattttgtcaaagcgggttatggcatgttccgtactgcctcgtggggaatctccatcaataataaactggcgtgggtgcttatggaagcgccggtattcatcgtcatgtttgggttgtgggggaagagtggagcgggatttgccgtgccggtatatttcttcttcctgctgtttcagttgcactatcttcagcgggcctttatttttccgttcctgctgaaaggtaaaagccggatgccggtagctattatggcgatgggtatcgtcttcaaccttttgaacgggatgatgcaggcgggcggtttgttctatttcgctcccgaaggcttgtatgccgatggctgggcctatttgctgaaacctcatgccttgttgggaatcattctgttttttgcaggtatgttcgtcaatttgcattccgactatgtgatacgtcatctgcgcaggccaggtgatacgaagcattatcttcccggaaaagggctttaccgatacgtcacttctgccaattacttcggtgaactggtggaatggacggggtttgccatactcacagcttctcccgccgcctgggtgttcgtctggtggacgtttgccaaccttgttccccgtgccgatgccattcaccgccgttatcgggaggagtttggtgatgaggcggtaggaaagcgcaaacgcatcattccatttctttattaa

SEQ ID NO: 68 - DNA, 1227 bp, Parabacteroides merdae
  source 1..1227: mol_type = other DNA
atggaaaaagaatctatactgttcactcccggtaaaatcgggccgttgaccctgagaaaccggacgatacgggcggctgcatttgaaagcatgtgcccaggaaacgcgccttccgacatgttgtatgactaccataaatcggttgcctccggcgggatcggtatgacgactttggcctatgcggctgttacgcaaagcggactttctttcgaacgtcagctctggatgcgcccagatatcatccccggattaaaacgcatcaccgatgccatccacaaagaaggagcggccgcctccgtacaactcggacattgcggaaacatgtctcacaaaaacatctgcgggtgcaggcctatctccgcatccagcggtttcaatatctattcccctacccttgtccgtggaatgaaaccttccgaaatcacagctatggcaaaagcgttcggacaagcagttcatctggcgcgcgaagccggaatggatgcagtggaaatacatgccggtcacggctatctgatcagccagtttctttctccctataccaatcatcggaaagacgaatatggcggtagcttgcaaaaccggatgcgctttatgaaaatgtgcatggacgaagtgatgaaagctgccggtcaggatatggcagtgttggtaaagatgaatatgcgcgatggcttcaaaggaggaatggagcttgatgagacacttgaagtggctcgtaccctgcagaacgaatgcggagcacacgctttgatccttagcggtggcttcgtcagccgtgccccgatgtatgtgatgcggggttccatgccgattcatacgatgacgcattatatgcctttcggctggctaccgctcggagtcaaaatggccggacggttcatgatcccgtctgagccgttcaaagaggcttacttcctggaagatgccctaaaattcagggcggcattgaaaatgccacttgtctatgtaggcggtctgatctcacgcgagaagatagacgaggtcttgaacgacggtttcgaattcgtgagtatggcacgtgccttgctgaacgatccgtcattcgtaaacaaaatgaaggaagacgaacatgcccgttgtgactgcggacatagcaactattgcatcgcccgcatgtattccatcgaaatggcatgccacaaacatattcagaacttgcccaaaagcattgtcaaagaaatagagaaattagaatataagtaa

SEQ ID NO: 69 - DNA, 1227 bp, Bacteroides dorei
  source 1..1227: mol_type = other DNA
atgatgaactctaaattatttactcccgcctctatcgggccgctgactttgcgtaaccgtacgattcgttcggctgcttttgagagcatgtgtccgggcaatgcgccgtcccggcaattgaaggattatcactgttcggtggcagcaggtggagtgggaatgactactattgcttatgcagctgttacacagagtggcctttctttcgacaggcaattgtggatgcgccctgaaattataccgggattaagggaaataaccgatgcggttcataaagaaggagctgctgcaagtattcagttgggacattgtggaaatatgtcgcacaaaagtatttgtggggtaacacccgtaggagcttcttccggttttaatctttattcgcctactttcgtgcgtggcttgcgcaaggaggaactgccgcagatggcaaaggcatacggtcagtcggtcaactgggcacgtgaggccggatttgacgcggtggagatacatgcggggcatggctatcttatcagtcagtttctttcaccttacaccaatcatcgtaaggacgagttcggtggctcgttggagaaccgtatgcgctttatggatatggtaatggaggaagtgatgcgtgccgcaggtaatgacatggccgttctggtaaaaaccaatatgcgtgacggttttaaaggcggcatggaaatagatgaagctgtgcaggtagcgaaacggttggtacaagatggggctcatgcgttggtgctgagcggaggctttgtaagcaaagcgcctatgtatgtcatgcggggagcgatgcctataaaaagtatgacacattatatgagctgctggtggctgaaatatggggtacgtatggtaggtaaatggatgattccgacagtaccttttaaagaggcttatttcttggaagatgcgttaagattcagaacagaaataaaggaaattccgttagtatatgtgggagggctggtatctcgtgaaaagatagatgaggtattggatgatggttttgaatttgtacagatgggaagggcgttgctgaatgaacctggttttgtgaatcggttgcggactgaagaaaaggctcgttgcaattgcggtcatagtaattattgtattgcgagaatgtacactattgatatggcatgtcacaaacatctggaagagaaattacccctttgcttggaacgtgaaatagaaaaattagagaaccaatga

SEQ ID NO: 70 - DNA, 1224 bp, Bacteroides vulgatus
  source 1..1224: mol_type = other DNA
atgaactctaaattatttactcccgcttctatcggaccgctgactttgcgtaaccgtacgattcgttcggctgcttttgagagtatgtgtccgggcaatgctccgtcccggcaattgaaggattatcactgctcggtggcggcaggtggagtgggaatgactactattgcttatgcagcagttacacagagtggcctttctttcgacaggcaattgtggatgcgccctgaaattataccgggattaagggaaataaccgatgcggttcataaagaaggggctgccgtaagcattcagttgggacattgtggaaatatgtcgcacaaaagtatttgtggggtaacacccataggagcttcttccggttttaatctttattcgcctactttcgtgcgtggcttgcgcaaggaggaactgccgcagatggcaaaggcatacggtcagtcggtcaactgggcgcgtgaggccggatttgacgcggtggagatacatgcggggcatggctatcttatcagtcagtttctttcaccttacaccaatcatcgtaaagacgagttcggcggctcgttggagaatcgtatgcgctttatggatatggtgatggaggaagtgatgcgtgccgcaggtaatgacatggccgttctggtaaaaaccaatatgcgtgacggttttaaaggaggaatggaaatagatgaagctgtgcaggtagcgaaacggttggtacaggacggggctcatgcgttggtgctgagtggaggttttgtaagcaaagcgcctatgtatgtcatgcggggagcgatgcctataaaaagtatgacacattatatgaactgttggtggctgaaatatggggtgcgtatggtaggcaaatggatgattccgacagtacctttcaaagaggcttattttttggaagatgcattgagattcagaacagaaataaaggaaattccgttagtatatgtgggggggctggtatctcgtgaaaagatagatgaggtattggatgatggttttgaatttgtacagatgggaagggcgttgctgaatgaacctggttttgtgaatcggttgcgcactgaagaaaaggcgcgttgcaattgcggtcatagtaattattgcattgcgagaatgtacactattgatatggcatgccacaaacatttggaagagaaattgcccctttgcttggaacgtgaaatagaaaaattagagaaccaatga

SEQ ID NO: 71 - DNA, 1224 bp, Bacteroides thetaiotaomicron
  source 1..1224: mol_type = other DNA
atggaatctaaacttttcacccctgttactttcggtccattgacgctgcggaatcgtacgatccgctctgccgcttttgaaagcatgtgtcccggtaacgccccttcacaaatgctgctcgattaccaccgctcggtggctgcgggcggagtcgggatgacgactgtagcctatgcagccgtgacacaaagcggactttccttcgaccgtcagttgtggctgcgtccggaaatcatttccggtttgcgtgaagtgaccggagctatacacacggaaggtgcggcagcaggcatccagatagggcactgcggaaatatgtcccataaaaagatttgcggaaccactcccatttcagcttctaccggtttcaatctctattctcctacattcgtgcgtggcatgaagagagaagagttgccggagatggccagagcctacggacgggctgtccacttggcacgggaagccggcttcgacgccgtcgaggtacacgccggacacggatatctgatcagccagttcctgtctccctacaccaatcaccggaaagacgactacggcggcttgctccaaaaccggatgcgctttatggaaatggtgatgaacgaggtgatgacagcggcgggaagcgacatggcagtcattgtaaaaatgaatatgcgcgatggcttcaaaggcggcatggaaaccgatgaatctctgcaagtggccaaacgcctgctggcattgggcgcgcacgcattggtactgagcggaggattcgtcagcaaggccccgatgtacgtcatgcggggagcgatgccgattcgttcgatggcttactacatggactgctggtggctgaaatacggagtccggatgtttggaaagtggatgattccgaccgttcctttccgggaagcctatttcctggaggatgcactgaaatttcgggcggcacttccggaagccccgttgatttatgtgggcggactggtgtcccgcgaaaagatagatgaagtattggatgccggcttcgatgccgtccagatggcacgtgcgttgctcaacgaaccggagtttgtcaaccggatgaggcgggaagagcaggcgcgctgtaactgcggacacagcaactactgcatcgggcggatgtacacaatcgaaatggcctgccaccaacatctgaaagaaaaactgcctccctgcctgcaacgggagattgaaaaactggaaaagcaatga

SEQ ID NO: 72 - DNA, 1224 bp, Bacteroides caccae
  source 1..1224: mol_type = other DNA
atgaaatcaaagttatttaccccggtcacttttggacctttgacacttcgcaaccggaccattcgttcggcagcttttgaaagtatgtgtccgggaaacgccccttcacaaatgttgttggattatcaccgttcggtagccgcaggcggggttggcatgacgacggttgcttatgctgccgtgacacaaagcgggctttcttttgaccgccaattatggttacgatcttccataattcccggtttacgcgaagtgaccgacgccatacacgatgagggtgccgcagcaggtatacaaatcggtcattgcggtaatatgtcccacaagaacatttgtggagtaactcctatctctgcttcttccggtttcaatctttactctccgacctttgtgcgcggaatgaggaaagaagaacttcctgaaatggcgcgtgcttatggaaaggctgtcaatctggctagagaagctggtttcgatgctgttgaggttcacgccggacatggatatctgattagccagttcctttccccttacacgaatcaccggaaggatgaatacggaggttcgttggaaaaccggatgcgttttatggatatagtgatgaaagaagtgatgaaagctgccggtagcgatatggcggttcttgtgaaaatgaatatgcgcgacggtttcaaaggtggaatggagtcggaggaaacgatacaagtagcccgacgcctgctcgaactgggtgctcacgggctggtattgagcggtggtttcgtcagtcgtgcgccgatgtatgtgatgcgaggggcgatgcctatccgttctatggcttattatatgaattgctggtggctgaaatatggcgtccggatgttcggaaaatggatgattccgactgttcctttcaaagaggcatattttctcgaagatgcgctgaagttccgtgaggcccttccgggtgctccgttaatttatgtaggcggccttgtttctcgtgagaaaattgatgaagtgctggatgccggctttgatgctgttcaaatggcacgtgccctgctgaatgaaccgggatttgtgaaccgtatggcacgtgaagaccgtgcacgttgtaattgcgggcatagcaattattgcattggccgtatgtacacgaacgaaatggcttgtcataaacatttgaatgaagaactgcctccttgtctgcaaaaggagattgaaaaattggaaaaacaatga

SEQ ID NO: 73 - DNA, 1224 bp, Bacteroides finegoldii
  source 1..1224: mol_type = other DNA
atgaaatctaaacttttcactccggtcactttcggccctttgacgttgcggaacaggacgatacgttcggcagcgtttgaaagtatgtgtccgggcaatactccctcgcagatgctattggattaccaccgctcggttgctgcgggaggagtggggatgacgactgtagcttatgccgccgtgacgcaaagcgggctctctttcgatcgtcagctatggatgcgtccgtccataatttcccgtttgaatgaattgacaaaagccgtgcacgacgagggtgctgcggtaggtatccagatagggcattgcggaaatatgtcccacaaaaagatttgcggtgtcacgcccatatccgcttcttcaggttttaacctctattcgcctacgtttgtgcgtggaatggaacgggaagaacttccggaaatggcacaggcttacggaaacgctgtcaacttggcacgggaagcgggatttgatgcggtggaagtgcacgctggacacggttatttaatcagccaattcctttcaccttacaccaaccatcgcaaagacgagttcggaggctcgctggagaaccggatgcgttttatggatctggtgatggaagaggtgatgaaagcggcgggcaatgacatggcggtgattgtcaaaatgaatatgcgcgacggtttcaaaggcggaatggaaatagacgaatccattcaggtggcgaaacggcttctggagctgggtgctcacggactggtgctgagtggaggctttgtcagcaaagccccgatgtatgtgatgcggggagcgatgccgattcgctcgatggcgcactatatggactgttggtggctgaaatacggtgtccggatgttcggaaaatggatgattccgtccgtgccttttgaggaggcttatttcctgaaagacgccttaaagttccgggaagctcttccggaagctcccttgatttatgttggcggactcgttgcccgtcagaagatagatgaagtgctcgatgcgggctttgaagctgtacagatgggacgtgccttactcaacgaacctggatttgtgaaccggatgaagcaggaggagcaggcccgttgcaactgtggacacagtaattactgcataggccgtatgtattcgatagaaatggcctgccatcaacatttaaaagaaacgttacctccttgtctgcaaaaggagattgaaaaattggaaaagaaatga

SEQ ID NO: 74 - DNA, 1224 bp, Bacteroides uniformis
  source 1..1224: mol_type = other DNA
atggaatccaagttattcacccccgtcactttcggaccgttgactttgcgtaaccgtaccatccgttcggctgctttcgagagcatgtgtcccggaaacgctccgtcggacatgttgctcgactatcatcggtcggtagcggcgggcggtataggtatgactactgttgcttatgcggcagttactcagagcggcttgtcttttgaccgccagttgtggatgcgtcctgagattatccccggactccgtaggctgactgatgccatacatgcggagggggctgcggcaggcatccagttgggacactgtggcaatatgtcccataagagtatttgcggttgtatgcctgtcggcgcatcttccggtttcaatctttattctcctacttttgtgcgcggacttcgtgccgatgagctgccggagatggcgcgcgcttatggacgttcggtgaatctggcacgggaggccggatttgattctgtagagattcatgcagggcatggctatctcatcagccagttcttgtccccgtccaccaaccatcggaaagacgagttcggaggctcgttgcagaaccgtatgcgttttatggatatggtgatggaagaagtgatgaaggctgccggcagtgatatggctgtgctggtgaagatgaacatgcgtgacggtttccgtggagggatggaactggatgagacgatgcaggtggcccggaggctggagcagtcgggagcacatgcgctggtcttgagcggtgggttcgtcagtaaggcgccgatgtatgtcatgcggggcgaaatgcctatccgcagcatgacgcattacatgacctgctggtggttgaaatacggtgtacgcatggtaggcaaatggatgataccgagcgtaccgtttaaggaagcctatttcctggaagatgccttgaaattccgtgccgccttgaagataccgttggtttatgtgggtggcctggtttccagggacaagatagatgaggtgctggatgacggtttcgaggctgtgcaaatggcacgtgctctgctcaatgagccggggttcgtcaaccgcatgcgtgccgaagagaatgcacgttgcaactgccggcatagtaattattgcattgcccggatgtattccatcgagatggtatgccatcagcatttgaaggaggagcttccaccttgtctgaaaaaggagatagagaaaatcgaagcgaaaggctga

SEQ ID NO: 75 - DNA, 1014 bp, Homo sapiens
  source 1..1014: mol_type = other DNA
tgtaccggtatttcattcctcgctgaagatggcggatatgtgcaggcacgtactatagagtgggggaacagttatcttccgagtgaatatgttattgttcccagaggacaggatttggtatcttatactccaacgggtgtaaatggcttgagatttcgggctaaatatggtctggtaggactggctatcattcagaaagagtttgtggctgaaggactgaatgaagtagggctttcggctggattgttttattttccccattatgggaagtatgaagaatatgatgaggctcaaaatgcaattactttgtcggatttgcaggtggtgaactggatgctttcccaatttgctactatagacgaagtgagagaagctatagaaggggtgaaggtggtgtctcttgataaacctggtaaaagttctacggtacattggcgcattggcgatgctaaaggaaatcaaatggtgttggaatttgtaggtggtgttccttatttttatgaaaataaagtaggagtactcaccaattctcccgattttccatggcaggtgattaacttgaataattatgtaaatctatatccgggagctgtcactccacagcaatggggtggggtgactattttcccttttggcgcaggtgccggatttcatggtattccgggggatgtaactcctccatcccgttttgttcgtgtagcgttttataaggcaacagctccggtgtgtcctacagcgtatgacgctatattacaaagctttcatatcctgaataattttgatattcctattggtatagaatatgcgttagggaaagcacctgatattcctagtgccacacaatggacttcggctattgatttgacaaacaggaaagtgtattataaaacagcatacaataacaatattcgttgtattagtatgaagaagattgattttgataaagtgaagtatcagtcgtatccattggataaggagttgaaacagcctgtagaagagattattgtgaaataa

SEQ ID NO: 76 - DNA, 846 bp, Mus musculus
  source 1..846: mol_type = other DNA
acggacgagttcctgtggattgaaggcataccctttcccactgtatattactctcaggaaattatacgtgaggtgcgtgatcgttttgttgtaagagatgaagacaccatcattgtcacataccccaagtcgggaactcattggctgaatgagatcgtgtgcttgatattaacaaaaggcgatccgacctgggtacagagcacaattgcaaatgaacgtactccttggattgaattcgaaaacaactatcgcatcctgaattcaaaggaaggtcctcgtttgatggcaagcttgttgcctatacagctgtttcctaaaagcttttttagtagcaaagccaaagtcatatacttaatccgtaatccacgcgacgtactggtatccggttatcactatttcaatgcccttaagcagggcaaggagcaagttccatggaaaatctacttcgaaaattttctgcaaggaaagagttactttgggtcatggttcgagcatgcgtgcggctggatctcgcttcgtaagcgtgaaaatatactggtgttgtcgtacgaacaattgaaaaaggatacacgtaacactataaagaaaatttgtgaatttcttggggagaacttagaatcaggagaactggagctggtattaaagaatataagcttccaaatcatgaaagaacggatgatctcacagtcatgcttaagtaatattgagaagcatgaattcattatgcgtaaaggcattacaggggattggaaaaatcattttaccgtagctcaggccgaagcctttgataaagctttccaagaaaaagcagcagacttcccacaggagttattttcttgggaataa

SEQ ID NO: 77 - DNA, 164 bp, synthetic construct
  misc_feature 1..164: note = Description of Artificial Sequence: Synthetic polynucleotide
  source 1..164: mol_type = other DNA
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtag

SEQ ID NO: 78 - DNA, 164 bp, synthetic construct
  misc_feature 1..164: note = Description of Artificial Sequence: Synthetic polynucleotide
  source 1..164: mol_type = other DNA
gataaaacgaaaggctcagtcgaaagactgggcctttcgttttacaattgggctaccttttttttgttttgtttgcaatggttaatctattgttgaaatttaaagtttcacttgaactttcaaataatgttcttatatttgcagtgtcgaaagaaacaaagtag

SEQ ID NO: 79 - DNA, 63 bp, synthetic construct
  misc_feature 1..63: note = Description of Artificial Sequence: Synthetic oligonucleotide
  misc_feature 6..12: note = This sequence may encompass 4-7 nucleotides
  variation 6..12: note = a, c, t, g, unknown or other
  misc_feature 18..55: note = This sequence may encompass 34-38 nucleotides
  variation 18..55: note = a, c, t, g, unknown or other
  variation 58..59: note = a, c, t, g, unknown or other
  source 1..63: mol_type = other DNA
gttaannnnnnngttaannnnnnnnnnnnnnnnnnnnnnnnnnnnnnnnnnnnnntanntttg

SEQ ID NO: 80 - DNA, 62 bp, synthetic construct
  misc_feature 1..62: note = Description of Artificial Sequence: Synthetic oligonucleotide
  variation 6..12: note = a, c, t, g, unknown or other
  variation 18..54: note = a, c, t, g, unknown or other
  variation 57..58: note = a, c, t, g, unknown or other
  source 1..62: mol_type = other DNA
gttaannnnnnngttaannnnnnnnnnnnnnnnnnnnnnnnnnnnnnnnnnnnntanntttg

SEQ ID NO: 81 - DNA, 8503 bp, synthetic construct
  misc_feature 1..8503: note = Description of Artificial Sequence: Synthetic polynucleotide
  source 1..8503: mol_type = other DNA
tccgtctcagactgctatgacttgataccggctattacgagcgcttaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgggattaaaagtcggggattggtgaacaaaaaggtgtttctctctttaagagaaatatcgttttgctaaacagttgatattgaggtatcattttatcgtaaaagacatttttgctcaacaattgcttgacggaaatcaacaaattttagcattttgtaaaaaagtcgctatataatttggtgaattggagttattttcatatttttgcatcccgaagagtttctcttaaagagagaaacatcttttgcataccttttccgaccgaatttttatgtcgtaaagaggggctttgcagggggtggactcagaaagatgagaatagatgactattgtagttgaaacacatagaaagttgctgatatacagaccgatacgcatatcgggatgaaccatgagtacgttcttttctcaaaaaacataaatattcgaaaagagatgcaataaattaaggagaggttataatgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaaaaacaataggccacatgcaactgtaaatgtttacgcgggtaccgacaccgcggtggaggggaattgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccatgagacgtcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtccgtctcagccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcaggtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtatctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcgggtgtttttgttttatttaagataatacaaccactacataataaaagagtagcgatattaaaagaatccgatgagaaaagactaatatttatctatccattcagtttgatttctcaggactttacatcgtcctgaaagtatttgttgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatggggcggccgcacgacgtaccggactcagtagggagagctgtatgtgggtagtgagacgtcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtag

SEQ ID NO: 82 - DNA, 7722 bp, synthetic construct
  misc_feature 1..7722: note = Description of Artificial Sequence: Synthetic polynucleotide
  source 1..7722: mol_type = other DNA
tccgtctcaaaacggcgcgcctgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtagcctggatcacacaacatttaaaaaataacattatgaaagcactcgaaaagagattcaaaggtaatattaacaataatttattttcaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtttcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaggttcctagctgattagaaggccatcctgacggatggccttttttttgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccatgagacgtcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggcgtctcagccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagcaaaaaaacacccgttagggtgttttttcgaaaaaaaagggggaaactccccctttcgcattaatatgccgcttcgaattcttttaggaagcgtgtatcgttttcagagaacatacggaggtctttcacctgatatttcaggtttgtgatacgctcgatacccataccgagtccataaccgctgtatattttgctgtctataccatttgattcaagtacgttcgggtctaccataccgcaaccgaggatttctacccagccggtgtgtttacagaacggacatcctttaccgccgcagatattacagctgatatccatttccgcacttggttcagcaaacgggaagtaagacggacgcagacggatctttgtatcagcaccgaacatttctttggcaaagagcagcaatacctgcttcaagtcggtgaatgatacgtttttatctacatacagcgcttctacctgatggaagaaacagtgtgcgcgatagctgatagcttcgttacgatatacacgtcccggacagatgatgcggataggaggctgtgaagtttccatcacacgagtctgtacagaagaagtatgtgtacgcaatactacgtccgggtgagcttcgataaagaaagtgtcctgcatatcgcgtgccggatgatcttcggcaaagttcagtgccgagaacacgtgccagtcatcttcaatttccggaccttcggcaatgctgaatcccagacgggcaaagatatcaatgatttcgttctttacaatggtgagcgggtggcgtgtaccgagttctacaggataagccgaacgcgtcaaatccagtccgtcacaatcgttgtcctgactttcaaacatttctttcagcgcgttgattttgtcctgcgcttttgttttcagttcattcagtctcatgccgacttcttttttctgttcggcagctacattacggaaatctgccattaagtcgttaatggctcccttcttacttaggtatttgatgcggagagcttcgagttcttcggcattggaggcgtgtaaggcttccacctctttcagaagttgttcaatcttagctatcattttcttatatttttttggttggtgatgccaggctactttgtttctttcgacactgcaaatataagaacattatttgaaagttcaagtgaaactttaaattttaacaatagattaaccattgcaaacaaaacaaaaaaaaggtagcccaattgtaaaacgaaaggcccagtctttcgactgagcctttcgttttatcctaggatcagctgtacgtactcgcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatggggcggccgcacgatgagacggacctcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtag

SEQ ID NO: 83 - DNA, 78665 bp, Bacteroides sp.
  source 1..78665: mol_type = other DNA
taaggattgattcgctagctcagcaggtagagcacaacacttttaatgttggggtcctgggttcgagccccaggcggatcactgaaacaaaaagcaaaacaatgaaaaccgctgataatcaatcattatcagcggtttttctttttatccatactgcaaattgaagcagaataccgcattttactggaggtgaaataggtggacttaatttccacataaaaacaagtccacctgattggattatatttcactgattctctgcgttttgcataaaacaaactcttttcaaaacatgtatttttacaccatcaaaaaaagaagagtatggcaatgcaaagaaactattttacggtattgtttttcctgaagaaatcaaagctgcttaaaaatggagaagcaccaatctgtatgcgtatcacaataaacggaaaacgtgcagaggtacaaatcaagcgaagtatagatgttacaaaatggaatacgcaaaaagaatgcgcgattggcagggaaaagaagtatcaagaaataaaccactatcttgatacgataagaactaaaatccttcaaattcaccgtgaacttgagcaggacggtaaacctattacagcagatattataaaaaatatctattatggagaacactctactcccaaaatgctgcttgaagtattccaggaacacaattcggaatatcgggaattaatgaacaaggaatatgccgaaggtactgtacttcgatacgaacgtacagcaagatatttgaaggagtttatcagtgaacaatataaactggctgatattccattaaaatcaatcaactatgaatttataaccaaattcgaacatttcattaaaatacagaaaaactgtgcgcaaaatgcgacagtgaaatatctgaaaaatttaaagaaaatcatcaaaactgcattgataaagaagtggataactgatgatccgtttgcagaaatacacttcaaacagaccaagtgtaaccgtgaattcttaaacgaaatggaacttcgcaaaatcatcaataaagattttgatattcaacgattacaaaccgtaagggacatattcatcttctgttgtttcaccggtttggctttcacagacgtaaagaatctgaaaaaggaacaccttgtacaggctgataatggtgaatggtggataagaaaagcaagggaaaagaccgataatatgtgcgacattccattgttggatataccaagacttattttagagaaatatcagtcaaatccaatctgcaatgaaaaaggattattacttcctgttcccagcaaccaacgaatgaacagttatttgaaagaaatagctgatgtatgtggtattcagaagaatctttccacacatattgcaagacatacatttgcatcactggctattgcaaataaggtttccttggaatccattgccaaaatgttaggacacacggacattcgtacaactcgtatttatgccaaaataatgaattctaccattgccaatgaaatgaaagtactgcaaaacaagttcgcaatataattttcaaccattatttcatttcttacagcaaatatcgcactttgccactgactgtgcaaggcggccctgtcgggctggttggcggaaaaaaatcatcctcgcttcgctccggtatttttttccgccaagccttgcaccggtcattggcaaagaacagccgggccagtaagaaattgaaatactggctccacggagccggtcatgtctaatttaaataaaagaatatgactgaagaagttggaaagaaggtatgtgaaggtacagtagcagacctcatgaaggacaagaccggaaaacagacggttgtcacgttgacaagaaagaatgcttaccgagtgaagaaaatcagagaacaagggacggatgacgaagctgtcctttttcatttccgtgaacgctgtacgggaatgggctcctatgtacacacaatcgaagcggcagacggagaaacagaacttcatccgtctgaatttgaaaaatgggaagctgtggaattcctgtatcccggctatctggaagacctgcttgatgctgcatacaacgcatacagatggagttccttcgaacctgaagcaagggcggaaacagacatcatgcaatatgaaaaacaacttgtagaggatctgaaacagattccggaagaaaaacagaacgagtataccagtgcataccatagcaagttctctgccttgctgggctgtctctcacgatgtgccagtccgatggtgacagggcctgccaaattcaactgccagcgcaacaacaaagccttggatgcataccagaacagatttgatgaatttcatgattggcgtaaccgcttcaaggctgccatggaaaggatgaaagaggctgccaaaccggaagaacagaagcaagaggaggcatggaaccgcctgaagcgtgacattgcaagcagcgcacagaccattcatgatattgataccggtaaagcaagaggatacagccgtgccttgtttgtcagcagtatccttaataaagtaagcacctatgcaggaaaaggagaagtggaaatcgtacagaaagcggtggacttcattacagacttcaatgcacaatgcaaaaaaccggttatcactccgcggaaccgtttcttccaactgccggaaatggcacgccaggccagactgaaacttcaggaaatcagagaacgggaaaaccgtgaactgaaatttgaaggcggaacgctggtatggaactatgaggcagaccgcctgcaaatccagtttgacaatattccggatgaccagaggcgcaaggaactgaaatcatacggtttcaaatggtcgccgagataccaggcatggcaacggcaacttacacagaatgccgtatatgcagtcaaaagagtgttgaaccttcaaaacctataagacatgaaagaccgattgaaatatgtaatcgattcccgctacttcgacggaacatgcctgacaagtatgagtgacggattccataatgactatggtggggaaacaatcgaagaactgcgcatacgggaaaacaatccctatctgaaagcagtaacaccttctgatatagacaagaagctgcggctatacaatcagtccctgtccgaaccgttcaaggaaatcactgaagaagaatactatgacctgctggatgtactgccacccttgcgcatgagacaaaactcgttctttgtaggagaaccgtattacggaaatatgtactctttctgctttactcgtcaaggaagatatttcaagggcctacgctccgtacttactccgcaatccgaactggacagtcagatagaccgtcacatggaaatcatcaaccggaaagccgtgatctcaaaagaggaaacaagtaaaacggtcacaaccggaaccagactcattccctattatttttcactggacggaaaacagcccgtattcatctgcaaccttgtcatccaatcagattccagtcaagcaaggacggacatggcgaataccctgaaaagtcttcgccggaaccattatcagttctataaaggaaaagggcattacgaaactccggacgaactgatagaccatgtatcaggaaagaagctcacccttgtttccgacggacatttctttcaatatcctcccggcagggaatccgcaactttcatcggacacatcaaggagacatcagaggaatttcttttccggatctatgaccgtgaatatttcctgtatcttcttaaaagactgaggaccgtgaaaaaggaatcggcacaggaacaaataaatatcaaatcataacattcgggggaatgcggtaaaatgactgccgtattccctcataaaaacaatacaagtatgaacaaatcaaacactctatactggaaaacagccacagatccggctgaacgcattgaggtcagactcgtcctgaacagttatatcgacaatgacaatctgtatgtaggacttgaatcccggtctaaggagaatccggaatgctgggaatcctacacggacatcaccgtcaacctcaattctcttcccccgttccatgcctatgtggacaaccgggactgcaacagacatgtgcatgattttctgaccagtaacagaatagcagaacctgccggatttgaatatcagggattcagaatgttccgcttcaatcctgacaggttgaaggaactcgcacccgaacagttcaagacaatcagcgccaaactgccaccacaggatgacatgataaaggacatcatctatcaggaaagacgtttccctttgagaactgttcaagacattcacggaatatatcttgtttcaagcaaggaactggaagaatctctgatcgaaggagtacggaacctggatgctgcggcatatgaactgctggatggcatctgcctgttctgctccacacaggaactgcgctatcttacggatgcagaactgatagaaacaatctacgcacaataaaaaggaggaacaaatatgaaaaccggagacattgtatttctgagacgtccctataagggataccgtgccgtcgaactgatggaaagactggaatgccgctggctggtcaggattgtcgagagcggtcttgaactggaggtatatgaagatgaacttatatcagaattttaatacagacaaagtgttatggaaaaatatcagtttgcattccattcggaaataatcggctatacctctcctcatatcggtgaggtcagaaaagccatacacagaaaagtggaaaaggaaaagtctgccgccataaagaatgatattgagctgcacatgtacaaagtgcatgacggcataccggttctccttaacacctgctacctgtacgatgaaaaaggatgtatggtacacggaagtatcaagggaaccaaggattatctgcttgagacatggagataccatacaaacagacattctaaaggcatcagttccacaagaatcaggccttgcacgacaagcagggctttttcatttgtataactcttaaaatcagaaatcatgaaccagacattacaacttacagactatattccacagaatgtaagcctctactacgtggactaccgggatgatcttgatgagcatgaagacatccaggaggaatgcatccgttccaacaaaatggaaaaactctatgaaaaggcatacgaatggtatgaggaacaggaaagttcaaacatgcacgactatctggaggagacaagaaagaatatggaaacggacaatttagccggagagtttgaagagcatgaagatgaaatcagggaacttatctacgaccggaacgattccgacccggtaaaggatatgatacgcaactcgtccgtcactaatttcttctattcgctcggagtggaaatcagcggatatctgaccggttgttcactgcggggagaatcagtcgccatggcctgccataaggtacgtcgcgcactgcatctgaaaaaggggcagtttgacgagaagattgaagaactggtagagaatgccacatacggtggagaactgcgcatctacttcaacgccatgtttgacaggctcatcagcaaaggccctgagaacgatttcaagagcatccgtttccacgggaatgtagtggtggtcattgccgacagccggaacggttccggacatcatgtacggattccgctggacatcactttccctttccgaagggagaacctgtttgtcgattcacaggtacactattcctatgccaatgaagtctgcggcatgaccaatgactggtgtgattccacaaaatgggaaacaggcatgataccttttaccggatctgtccgaaaaagccggatggctgaatacaagaaacaggaagccgcttatgagcagacattccgagacgggaaatgcaccttcggtgacatgaactacaaacgccaccgtgacgtgcggtattcgaatgaatatcctgccggatgcaggtgccctcattgcggtacattctggattgactgaaaaaacatttaccaaccaataaattcaaacgatatgaaaatctgctgttcacaagagcattacgacaaggtcgtacagtatgcaaaatcaatcaatgacaagacactggaaaactgtcttgaacgtctaaaacaatgggagaagaacgagaaccgtccatgcgaaatcgaactctattacgatcatgcgccgtattcgttcggattctgcgaacgttatccggacggaaatacaggcattgtcggaggactgctgtatcatggaaatccggacgaatcctttgccgtcaccatggaacgtttccacggatggagcatacatacctgacatatatgcgacagtctgtattggggagcctcatgcaatatggggttccctttttttatgccgcagacatgatgacagcatcctcatttcttgctgcaaaaatagctgtttgccgcgcaactcccgcaaggcggccctgccgggctggttgtctggaaaaaaatcatcctcgcttcgctccggtatttttttccgccaagccttgcagggatgcgggcaaacagacaacagggacaacaagaaataagaatgcctgtaccttacaggcagacaatgtataacaataaatatcagaagtcatgattacagaccagaagacacagaacaggcttcacgcggataccggaacggaactgttctccatcagacaaaggaaggaagccgtcacaaggatgctggacattctgaaagagactccggaatacctgcaggttatgaaccatataccggcttatgccatggatgacgatacgtcagaatggtggaaatcggaagaatcggaaaatttcatgaactcactcctggaagtgatggaaagctatactccggacggatacaggttcggaccgaaatccggcacgactgacctttacggctactgggaaagcaagaccgggcggacaaccctcttccatctgcttttcagtctggaaagcggatatgaatggggaaaaggtctttcccatgagaaaacggacgcattctacaaggaaataaaagagaaatttcatggagaaggattcgacacggacagaaccggctgtacatcacaggccatgtatcttgtaaaaggaaaaacacgcctgtacgtgcatccgatggaaataagcggctactgtgaaacactgcatattccacagattacagccatactgaaaaaaggaggccgtacattccgtcttgtaaaggatacgatagcggaagaggtgtattccttcaccgatgaagaagaactggaatattaccgtgccagatacggaacgtgcatccaccggaatatactggatgccttcagcaaccgccacgcagggaaagaggacatactttccatgatggcatcacggataaatgtggctacgacatcacatctttacggtatcggatatgattcgcctgcatacaggtttgtgcatgaggcatacgacagactggtaaacaatggaaagctgaaggagaatgtccgggaaatcggttgctgcaacatcataatggccatttcaaataccaacgcaatatgagactgaattacaatgacatgctgcttctggcaatatgggaatacaacaggagacaggacgaggatctgaccctggaactgtttcaggaaacattcggacaggttcccggcgcacatttccatgacaaatgggtgcattattacaacaagaacctgctgatgatggccgcctatttcaggggtgaggaagaaaacggccagaaattctgtgatatgatcacccgacaggttgaacgctatacacaaaacaggaggagaacaggatgaatacaaagatacgatatgaccttgacagtcttgaactggcaaacggtgacttcgggtatcccattacagaaaaggaagtacggaaagtgaaccgtatgctggaactgatggagaatgtccgaagcaggcagatgtgcccgacagaaggagactgcgtggaatttgtctcacgttctggtgactatttcggaaaagctcatatagaacggataacaggaaaatatgcggatatatgcctgataccggaaacggtattctgttttgatgacatgggaaaagccgcctatgataccaccggaagtccctggacgcaggtcaatatccggaacatgaaacccgcaggttctgaaatccgcatattcagaacatggggattcgggaagcgcagcaatacgggcagtctcaggttcgatgctccggtcaggaaatgggaatacagagaaccgaatccgttatatgacggttacaccacccgtaactggttccgctatcatatcatgaaacaccgggacagggaaaggacaggcgaatacaccttccgcagcgattcattcacgctgtacagccggagcgagctggacgagctggccgcaatcctgaaaggcagactctacaagggaatcctgcctgactctcttgtactttggggataccgcatggatattaaggaaatatcacgtgaacagtggaacggtatgggacagcacggacaaatccgcatgaaattcatgggatacggtccggtcagaatccacacggacaatgaaaaccataccgtaacagtatacagaatcaacgacatattgtcttcaactatcagaattttcatattttttcagttctttttttgtttcttctattaatattttaagccactccatgatttgtattgcatgttcatgaacagtttcattttggctatcactgtcgtgtagtagcctttgaaaatcacgtaaaatattgtctttcccaagcatctcccatacaggcatcatccggtggattatttttctcatggtctcacggtcggttatcctgtcagcagattccatctcctccagttctttttcagattccataacaacgagagaaagcatatgactataatcatccgtattctccagtaaactggaaaaatcgaattctccggaaactgaaacttgtgtacgagatataatggtggataaaaaagcaagcagtccgtggatattgaacggtttatgaatacagcctacaaatccttctttttcataaattccggaatttccgtcaccacgggcagtcatgactgctactggaacagttctagaattgccgatgtccgaattgcgaagcaatcttaacaaaccgaatccgtcagtatcaggcatttgtacatctgtcaagatcaaatcatattcagaattttcaagagcggccactacttcacgtgcattcttacaggttttacaggatatacctttgcgcccgagcatatcttccgctattttcagttgtataggatcatcgtccactacaagaacattcttaggcaatatagttattgtattatggtccgatttgtcttcctcaactaactcatccgtttcaggcaaagaaagttccagtctgaacatgcttcctttaccgagtacactttctacatccatttttccttccaaaaccttaattaatcctttggtaaggaaaagtcccaaaccaaacccttcagaattgacattctgtgcggcacgctcaaatggagcaaatattcttttcagtgtttcctcatccataccgataccagtatcccttatttcaatacgaagttttccttctgaatattctgaatggaaattgacgttacccctggaagtaaacttaatagcgtttgtaagtagattggctaaaacctgttcaagtttgtccgcatcaccttttactattacatttgatcctttatgttcagaatataaaatcagaccttttgaagtcgctttacgagaaaactcatctgaaattcgttgcaagaaacggtcaagataaaatggtgtgtcgttacgcaaattaccggcttcattgattcggtaagcatccatcaaatcattaaccagatgtaaaacgtgtcgacaagaatgacggatgtcatctaaatatttttcgcgcttcctcttttcacgcgtttcagataccaaatctgcacagttatggatattaccaagtggacctctaatatcatgagaaactgtcaggatgattttcttacgcatatcaagcaaattctcgttttcttgaatagcttgttgtaatttaaatttaattatttcttccttacgtaaatctgattgtataattaaaaatgaaattaatattataaaaaccgcaatactcatcattacgataaataatcgaaaggattcttgtttgacttccgttacctctaagtttcgttctataaatgacagctgtacctgattatctaaaaaagatacaaaatcatataatttttgatttaacagcctattctgcaaacgcaagctatccacataagtttctatctgattgtttcgcatatctatgacagaaaccaatctattattaaaattctgtatttcattagttatataggggacttgtatcgtctccttctttccgaataatccggcaattcctttctttttctgagttattgtcttcacttttactgtttgagtagctattacaggcaattcattagtaagaatactatcagatttattcgcaaattggactgctttcattatttgaaacaagtgcatttctttcgttttaagcaattcccgtaaagaatcaatttgaactggacataaaaaatcacaactccttaattttatttcaagtagaacactatctgttttaaaacgttgattatgaaatatgttataatcagactcatcccatactataactgattcgcctaaagttgccaacttagtaatatacaaatgaactttattagtattctcataagcttcattaatttgaattatcagattctcaagttctttcaaccggcaacgttcatttatcattacagtaaccatacttaagactataaatcctgtaataaaatatccaataaatagtcttttgcgtaataatgaagtcatcaggaacattctattgatttatttgacatcataattctatatatttaactagtcatagtatatatcattctcaaatatttatttcaaattcaagcaataaaataaaaaaacacttcatattacaactgaactcttttatgaaaaagttgaatatatgaagtgtttttttattacgatataaactataaaatcctattcttcgggaactggtgtataaacccttatccagtccaccaggaaggtgtggtcttccacatttttcagttcctcatccgtagggcttaaacctttaacggctctccagctttggtcttccatatttattatgatgtccatgtcttttaccagacctgtaccaccagtgtagttgttggggtcgataatatccttgccgcttacggttctgacaagttctccatctacataatattcaagtgtgaaagggtctttccagaacactcctacacgatgaaaatcgtcgcgccacaatgttcccttgtcatccttataccatgagccaagatctttcggctgataatccttgaatggctggcggatgaatatgtgatggctcaggtgaagtctgtcggcaccgtaacctccgccgtctctgtcgccgccgtatgcttctatgatgtcgatttcctgagtatcgtcagggctgagcatccatacatcggatgccatggttgaatttgaaagttttgcgtatgcctctacataaaccggatactttacacgtgtcttcgatgtgatacatcccgtataggttcccggcagttcctttgtgttgggtccgcttacaactttcttcatggggacatcttcaggacggctggctcttattttaaggtatccgtcggaaacggaaacatggtctctctgccatattgtaggagcaggtcctgtccaatgattatgatagaaatcggtccatttggcatagaactcttttcctttatccttttcgtcggcaacataattaaagtcgtccgactgtggatggagtttccacaccataccgtcgccggcatcagcgggtacaggatagatatcccactcgtacgatttattattgaaatcttctgctgcacaggctatttgcagcgatgctaaacaaatggtaaacagttttctcatcgtggtatcttagtttaagttataataattattttcgttcttttgattcacctttagcggtatgtgtctgcaatgtccaggtagaaaatctcattatgctctgatagtctgaactgttgtatatatgagtaagaccccatctcaatatttcggtaggttctttttcggcatctgcactgcggttcaggccaatggcgtgtggcgcgccttttactactgacattatttcaaagtttattccgtcaggcgaccactggagtgtgttcttttcaggaccgtcggtggtgataagtgaagctatacctcctttgtaaggccatacgcaaacttcatgcccgctgtttgaaataggattatattccgatttcacatacggacccataggattttccgcaatagccactccgtgtttgatttcacggccgccccatgttatttcttctcccatacgttcgcctttgtagtacatatagaacttacctttataaggtattatacacgggtcgtgtaccttatgactgtcgaaatcacctttcgacactaccttgaatctgttatcctcatcgccttcccattcgccggtattagaaggttccagtacaggcttgtctgtcttgatccacggtccttcaggggaatcagcacatgccataccgatagtattctttacacggactgtgtaaggggattttaccgcctgatagcaaagataatactttcctttccattccatcacctcaggagtgaagactgaacggtcgtcgtaagcacctttttcaccacgtttcactgcaattccctgttccttccatgtccatccgtcttttgatgtggcataccatatatcacatctgtcccatgggaaaaccttatctttctctatatctccagcaaatccttgggtaggtccatagctctttgaataccatacataatatgtattacctattttcagcattgcactcgggtctcttcttactacgccctcttcataagcaagatcacctttaagtggttccatcttatactcaaagaaccatttattgtcgtgattttcccatttcatggcacgtttcatagctgcacttaacttatttcccttaggtattcccaatgaatcggccttacgctcatcataattctgagtgtcgtcaacggcaatagtctgtgtattgcctgtatttccgcatgctgccaatagcgacatcatgccggctgcaagaataatttttctcatactagactttattttatattaattgttagtttattcgagtgtaattcacttgtttctgcactgatattcagtaccgatgatttttctgtcgactgaagcatcagcatacatcttccctgatatgtcataatatccttactttgatatggagaaacgttcttcacgtttccattgtctataccaagcagacggtactctccatcaatgttgaacttaagcatctgttctgttgtctttacaggattaccttttttgtctgttagctgagctgtgacatgcagaacatcctttccatttgctgcgatactttgtttgtcaaccgtcagcaatatcgaatgttctttgcctgaagtccttatagctgtagtggtattacctaacttattttttccttttgcggtaatagtgccaggcttgtactgaactgcccatttatagatatgatcctcaaaatcgtctatatacttctttcccatcgacttaccgttaacgaaaagttccacttcatcacaattggaatatatctctactattaccgagtcacctttctgataattccagtgagagtttacatcatcccaaacccataattttctatcccattcatgtcctttcttatcagtaaatccatcttttacatggagatacgaagatttgtctgtagtctgtgaatatatagcaataaaaggcttgtctgtccacaatgatttcatcatgtcgtacgaaggcttcacatagccgcacatatccaggagaccacatcctatcgacttttgaggccattttgaaagacggctttcactttctcccagataatcgactcctgtccatataaacatacccggaacgaaatccctttcaatcaccgccttccattcgtgccactgaccgagattttctgtacccattataggcttgtcaggataattcttcttagcataatcatacatcacgcgacggtagctgaagcctgccacatcgagcgcgtcgatatatcctgactcaaagcttatggaaggcaggatgcagttggcggtaactacacgtgtggtgtccatctggcgtgtccatgcagctaatttttgcgctgtacggccaatgtcgtatgcatgtttaggctggattttccacatttctctgattttttctttagagtatggaggctgattccagaaataattaccgttggaatcggcaccgaagaaacctgtcgcctcgcggcatccggtataagtccattctatttcattacctatactccactggaagatacaggcatgattacggcttctcctcattacgtttttcaaatctctttctgcccattcctggaaatgctcgcaatagccatgcgtaggatagtcttctacagtttccttcatattgagtcttttatctttgggataatcccactcatcgaagaattcttcctgaaccagaagacctatctcatcgcacaaagacagaaactcttccgctcccggattgtgcgagaggcggatggcattgcatcctccttcctttagggttttcagacgccggtaccacacatcgcgtatcattgccgcgccaaccattccggcatcatggtgcaggcatactccttttatcttcatgtttttcccgttaaggaagaaacctttgtctgcatcaaaacggaatgtccgtatgccgaacctgacagtgttttcagaaattacttcatcgccattcttgatgcgtgtctcggctgtatagaggacaggtgtatcgacgctccacaaatcaggctgtttaatctcagatacgatgtcgataattttctcctcaccagcattcagttttatactgaagacctcaaaggctgcgatattgcctttattatccttatatactacctcaacaactgcagctctgggttcggagtagctgttgcacacggtaacctggttgtttactttagcatatttatcagtaaccacgggagtagtgacaaatgttccccaaaccggaatatgcagtctgtcggttacaatcattttcacatccctgtatatacctgaaccggtgtaccatctgctgtcggcataatggctgtggtcgacccttacagtcatacggttatcctcattgggattgagatagtctgtgacatcaaaataaaaaggagcatatcccgaaggatgatatccaagctttttgccatttatccaatactcagaattattatatactccatcgaacactatatagcatttctgatttgcactgattgttgtgggaaatgatttgctataccatcctattcctccctgaaggaaagctacacatccttcacccgaaatggaatcgtaaggtaaaccaacactccagtcatgtggcaggttcactttcttccattcatcaccagggacataagaagtatatgaataatgagcagaatctttcagtacgaatttccaatctttattgaaatcaacatttgaatcagatgctgaaacctttagggttgataataggattattaaagctaaaagatttttatttctcataatcttaggttttacatgttttttgatgtcacaaaactatatctttcacttataatatatgagggggatattaatgtgatatagggtgggaaatcagaattttacatctgccctgtattccaccgtcacctacaaccttgacaaaggatgttcctttcttccctcttatggttctcaggacaaacagacactttccgttatatgtccttacactattgtttatgacgttgatgttcaaatcttctatcgaaggcgatccattgtcgagtccggcaagttcaagcttgtcgtcgaggattatcctcacatccgaaggtatatcgactactgtgtttccttctttatcttcaatggatacttctacatggataaggtcataaccgttgtcggtagctgttttgcggtcgcagttcagtgccagacggcacggcttgccgcttgtggacaaagtgtctttcgacaatattctgtcgccgtccttgcctaccgcaaggagtgttccttccttgtatgccaccttccacatcagtatattatgctccatgaaatcgctgcgtttctttgttcccaacgatttgccgttcagaaacagttccacttctggggcgttggtatatacctgcaccagtatgtcctcgtccctgcggtacttccatttatcgcgtgtgtcgtaccactcccagcgtctgatccatcccgggcgtggagtgtaggtgaaacttccgtcagtatccatcttgaactcgctttccttttcaggtattgttacaatatgggttttcggtgtgtctttccacagacattcaaagaaatggccacgcgctgtcttgttgcccacgaaatcgaagaaagaacagtctccaccccttgcaggccatgggccgttctcgccaagatagtcgaatcctgtccacacgaagatgcccgctatgtacttcttgtcggccacggctgtccattcaaagagctgaccaacattctccgaaccgataataggctgatatggatatagcttatggtcgatttcataatatttgtctttatagttatatcccactacatcaagaacgtctgtatatccggagagacgcgaaactgacggaacaacgactcctgaagagacgggacgggtagtgtccacatccttaacccaaccggcaaggacagcggctgtttcagccaaatcgtcttttcctcctgacagacggttgaactctttcagtatagacttgttgtctgtttccgggtcgcccgtatggataagacccttgaaccctttattgtctttgctcgatgcccagtaatatggataggtccattctatttcattgcctatactccagagtatcacgcaaggatgatttctgtctcgcctgatgaacgacttgaggtcgtgctcggcatgcgtatcgaagtatctggtatatcctattgatatgctgtcgggcgcatcttccttagctcgctcagtaatccactttttctttgccaccttccattcgtcgataaattcattcattacaagaagtcccagactgtcgcacatttccagcagactttccgaatgcggattatgggctgtacgtatggcattgcagcctatggaacgaagtttcagaaggcgtcgcaacagggcatcatcgtatgcggcaacacccatacatcccaagtcgtggtgtatgttcactccttttatttttactgattttccgtttagaaggaagccttcatccgcatcgaatttaatgtcgcggataccaaattttgttgttttcttatccatcacatatccgtcagaagcaatcagagtagtatgaagctcatacatcgaaggcgtttcaagactccagagatgacaattctccagttcaacagatgcagtgaactcattgaaatcgcctttcagggcaacaaaatcatcggaaacagaagctattgtcttgccgtcgtacactacttcgtgcttcacggtgactccttttacacctgttccagcattcttcacctcgcataccacattcaccatcgaacggttgcctacctgtggtgtggtaacgaatattccgtctgaaggaatatagagctcgtttcttagaataagactcacattcctgtatataccggcaccgacataccatctgctatcggcatacgctcttctgtcaacgcagacagttattgtattcatcgaaccttttggtttcagatattgagtaagttcatattcaaatcccacatatccgttaggacggaatcccaacatatgcccgtttatccaaacctttgagttattatatacaccttcgaaatgaatgaacacttttttcccattcatatcatccgaggtgagaaaattcttcatgtaaatccccacaccgccagacagaaaaccattgcttccggctgtctgagtcttggtatatccttcgctgatactccagtcatgaggcagacacacatcctcccactttatatctggactcaggaacaaagtgtcctgaggcacgaaacctgctggtttgctgaatttccaatcgaagttgaaatccactttagtggaggttccggcataacagaatccggacagaaagatagttaagactgtgataatgttttttatggtcatatcgattttcagattaatattaatgacaaaaataatttcaaaagtgtaaaaacaaaaaaactctccatttatatttcagatatcaacggagagtttcatcattaaaaaaaataaaacattttataaagttactccttgcttaaggatagctatttcccggtatcccttcttttcgttcagtgcctgctttccgcttgccacttccaccacaaagtctataaaacgtctgcttaaagattccatgctttctccctctaccagagttccggcattgaaatcaatccacgtatgtttctgttcataaagcggagtgttggtcgaaaccttcacggttggaacgaatgttccgaacggtgttccgcggcctgttgtgaacagcacgatatggcatccggcagaagcaagagccgtacttgccactaggtcgttgcctggtgcgctcaacaggttaagtccgtgtgttgtgacacggtcgccatatttcagaacatcctccaccatcgagcttcccgacttctgtgtacatcccaatgatttctcctcaagcgtggaaatacctcccgccttgtttcccggtgaaggattttcatatattggctggtcgttgcggatgaagtagttcttgaagtcgtttatcatggccactgtgtcgtcgaatatctccttcgtgcggcaacggttcatgagcagtgtctcggctccgaacatttcaggtacctccgtgaggactgttgtcccaccctgggcaacaagatagtcagagaacaccccaagcatcggattggccgtgataccggacagtccatcagacccgccgcacttgagtcctatacgcagttttgacagggggacatcagtccgcttgtcttccctggctatggcatacatctcacggagaagtttcataccctcttctatctcatcatctactttctgagaaacaaggaaacggatcctttgggtatcatagtcacctataaactcacgaaaggcatcaggctggttgttctcacagccaagacctacgacaaggacagctccggcattgggatgaaggaccatgtcacgcaatatcttacgggtgttctcatggtcgtcacccaactgcgagcatccgtagttatgagggaaagatataatggagtcaaccccctcgcaacctgtttccttgcgaagctgctcggccaactggtttactattccgttcacgcaacccaccgtagggataatccatatctcattacgtatgccggcttctccgttagcacgcaaataccctttgaatgtatggttctcgttcgtgaatgtctgtttctcgaacttcggagtgtaagtgtatgtactcagaccggaaaggttcgtcttgacggttttctcgttcagcagatgtcctttcctgacttcctttacagcgtgcgatatggggaaaccgtattttatcaccatatcaccttctgcaaaatccttcagggcaatcttatgaccggcaggtatatcctccattaattctatggaattgccgttcacctctattacagtccctttggacaatgggtgcagtgccacagccacattgtccgcagggtttatctggatatattcagtcataacaaactaacatttataaattgaagaatacaggtagaagtatcaacctacaaggtcttttactgtctgaagcattccttcgctctggattttgttgatatagtaaattacacggtctgccagtcccgagatagtattaaggtcttcaccccaaatggaagtatcggcgagaactgtcttcacaagattttctaccgagccatcgttccacaaacttgtaagcatcgccatgatttcctgtgcatcgttaggaactatctctacaccatcggcacgctttccacctttgtagtatactatgatggctgcaagaccgagtacaagtccttcaggaagcacacccttacgtttcagatattccttcactcctggaaggtcgcgtgtggcatacttagggaatgagttaagcatgattgatgttacctgatggtctacgaaaggattattgaaacgttccaggacatcatcggcaaacttcttgagttcctctttcggcaggttgagggtctccatcagctcgtcgaacatcacacgtttgatgaacttgcctatcacctcatgttggcatgcgtctctcacgatattgacgcccgaaaggaatgccaccggcgacaatacagtgtgaggaccgttcagcagagtaaccttgcgttcatgataaggctcctccgacgggacgaacagaacgttcagtcccgccttgtttgcaggaaattcttcggcaaccgattccggtgcttcgataacccacagatgaaaagcctcgccctgtacaactaaattgtcatcaaagtatagtttagtttttatgttgtctatgtctttacgagggaaacccggtacgatacggtccaccagtgtggcatatacaccacatgcagtttcaaaccatgacttgaactcttcgccaaggttccacaattcaatatactgatagattgtttccttcagtttgtgaccgttgaggaagataagctcgcatgggaagatgatgagtcctttcgacttgtcaccgttgaaatgtttgaatctgtgataaagcaactgtgtcagcttgcccggataagagcttgcaggagcatcctcaagcttgcacgacggatcgaagttgataccggcctcagtagtgttcgagattacgaatctcatatcaggctgttccgccagtgccatgaagtcattatactggctgtatggattcagcgcgcggctgatgacatcaatcattctgaatgagttcaccacctcgccattgttcagtccctgaagattgacatgatacagacagtcctgggcattgagggcatcaaccatacctttttctataggctgcaccacaacaacactgctgttgaaatctgtcttttcattcatattcgagataatccagtcgacaaacgcacgaaggaaattaccttcgccaaactgtatgatacgttccggacgtactgcctttactgcagtcttactatttaaagctttcattgtaatgccaaaaaattaaaattgataagattaaaattcaaccaacattctgaataccttacctggattttccgaccatttctgcagagcctcgcctgcctcttcaggtttcactacggcagagataagttcgttcatcgggcagttgccattctgaagataatgtatcacggcacggaaatcctcaggcattgcattgcgcgaaccgcgtatgtcgagttccttctggacaaaatattttgtctggaaagccacttcactcttggcatagccgatacatgccacacggcctgtgaaacctacaatgtcgatggcagtaacatatgtgataggactacccacagcctctatcaccacatcagccatatagccgtcagtaagttcccttactctttccaccacattttcagtcttcgaattgataaccatcgaagcacccaggcgttttgccagttcaagcttctcatcgtcaatatccaatgctattacccttgcgccacgaagcgatgctcttactatggcgccaagtccaatcattccgcaaccaatcacggccacagtatcaatgtcagttacctgagctctcgacacggcatggaaacctacgctcataggctcaatcagcgcacattccttatccgaaagaccggcagccggaataacctttgtccaagggaggacaaggaactcctgcatagaaccgttacgctgaacacccaaagtctcgttgtgttcgcaggcattcacacgtccgttgcggcatgaagcacactttccgcagttggtatatggatttactgtcacgttcattcccttctcgaaaccgacaggaacgccttcgcctatttcctctatcacagcacccacttcatgtcccgggatgacaggcatcttcaccataggatttcttcccaggtaagtattaaggtcggaaccacagaatccgacatatttgatacgaagtaaaatttctccggctccaagtgttggtttaactatatcagctacttgaacctttccggcttcagtaatttgtacagctttcataatctatgtatttatttaaatttgttattgtattattttgatgttgcattaattcaatgttgttttttctctatcttatatcctctccagccataatatgccgtaaagaagaaacatatcagaggtattacatatgccacctgatagaagtccgcgttatgattcatcacaaatgcggtgaactgagggatgcacgcattacctataatagccatcacaaggaatgccgaaccactctttgtgtcctcgccaaggtcgcgtagtgcaagtgagaactgggttggatacattatcgacatgaagaacgacactgcaagcatggcataaagtcctgtcataccaccgaacatgataattactccacacagtatgatatttactatagcgtatgtaagcagcatatcctgaggtctgaatttcgacattagcatagtacctatccatctgccgccaaggaaagccagcatatacagtccgaagaatgtggtcgcctcatcctccgacagacctgcatacatgcagcagtaaactaggaacaggctgttgatggctgtctgccctccgttatagaagaactgtgcgataactccccatctcaggtgtttgcgtttcaacactgcaaaattgataagcttgcccttctcgccgtgcgattcctccttgtcaatatcaggcaacttatacagtgcaaacaccacagcaagaataatcagcaggactgcaagaaccagataaggcatcttcatggagtctgtctccatctgaataaatccgtcccaacctccgggaaagtcggcaggcagagtctcgcgagtatagttctgtccggtaagtataagcttactcagaaacattgcggatatgaaagcaccaagaccgttgaacgactgtgcaagattcagtcttcttgaagccgtatcgtgtgtacccagagctgtcacatacggattggcagcagtttcgaggaagcacattcccgttgccatgatgaagaagattacaagatatgcccagtattcctttatctcggctgcagggaagaaaagcagaccaccgatggctgcaagaatgagaccgacaattatacccgacttatagctgaaacgtttcatgaacattgctatcggtatgggaaacaggaagtaggccagccaataggcagcttcagtgaacgaggcctcaaaagcattcagttcacaggttttcatcaactgcctgatcattgtaggcaatagattactgctgatagcccacatgaagaacaagctgaatatcagtaaaagcggtataaaatatttgtttttcattctgacatgtttttaatataaggtaactcaggcagattcttgaaaccgtaaaaggctttcgcgttctcgcccaagaaaagttttttgcttctctcttccaattcttttgatttaatcacaaagtcgtacgacatcttgtaggtaatggctgtgattgtgcgtggatagtcggaaccccacatcagtttctcgaagccaacaaggtcggcagcttcgttgatggctctgacagcgctgcggaacggatagaactcgtcattgaacagccaagtgataccgcccgactcaatcatcacattcttatgacgggcaagcattatctgcttcttccaatccggtttagtcaccataccgaaatgcccgatggcaatcttcaagtacggacattctgaaatgatttcttccatctcgcccacctggaggtctccctctgccatatctatggaaagaatcacccccttgtcttccattagatgaaacatcctcatcatctcgtccgagttgagcatcaccctaccgtccttcagttgcaggcggtgtcccggaatctttatggccttgaaccctttgtctataagttcaaccgcctggttatagaaacccggttttctgaattcacacataccacacacgaagaacctgtccggatatttcgtcatcacctccatcagatagtcattctgaatgccgtcgatatactcctgtgtgacaacagccgcgccaatcagggcataattcatattagccaggaaaacctcagccgtgtttcttccgtcaatcataaagggggggggagcatttgtctcacctcccccataaacaatgattgaccgttctctgtagtcttgattttcaggccatctacttcagtgtcctgataaagccacagatgcgaatgggcgtcaattattgtataatccatagaaacagtatttatgaatttgcccaacttactctttgctgatcgcctattatctccttaaccttttccacaaggctccagtctatcggttcctcaatgtattttatgttctgaagcacagactctgttcttgccgagctgaacaatgttgtaggtattctcggattgcttacagagaactgcaccgcaagtttctcgatagggtatccctgttcagcacaatacttggcagcctttgcacacacctcaatcaatggttttggagccggatgccattcaggaacacctctatgtgtgagaagtcccataccgaacggcgaagcgtttatcactcccacaccattttcgtcaaaatagtcgaggaagtccaccagcttgtcgtcgttcaatgaatagtgacagaagttaagcaccgcctctactgtacccggagcggcatggtcgataatccatttcaggttttcgagctgcaggtcggtgatacccacgtggcccaccacgcctttcttcttcagttccaccagagcaggcaatgtctcgttcaccacctggttcatatccgagaactcaacgtcgtgaacgttgataaggtcgatatagtcgatgttcagacgttccatactttcgtaaacactctcctgagcgcgtttgtccgagtagtcccacgtattcacaccgtccttgccatagcgtcccacctttgtagaaaggatgaacgattctcttggcaattccttcagagccttacccaatacggtttcggctttataatgtccgtaatatggagaaacatcaataaagttcagtccgcgttccactgctgtaaaaacagactgtatagcgtcactttctttgatagaatgaaaaactccgcccaatgaagatgcgccataactcaatacaggaaccttaagtcctgtctttcccaattcacgatattccatttttgataaataatttaaaggttaatattttttactctgtttattcttattcatacagatagaacatacgttccatcatcttccatttctcgtccgatgtggccccctcggcacactgctggaatttggctacgtattcttcccattcggcctgacgcggcagagtggcaagctttgccatagctgtatcccagtcaaaatccagaggtgtttccactatcataaagagttttgaccccaatatgtatatttccatttccaggattcccacctcgcgtattccggcgcgtatctcaggccatgcctcttccttactgtgagcctttctgtaggcttcaatcaattccggattctcacgcagactcaatgtctgacagtatctcttcacaggcagggaataacttttcactttatatccttctgtcttcatgatattattgatattaatatgttagtattacatgtcactgtctttatcttttcgacgatgctaaagtatgaagtatccatcaaaacaatagaggagattttcaaaaaagaaagaggggatattataccccctctttttcgacatttttacccctcataaaggagataaaaagtcaccccaaactctataaaaaatcaaaacagattgaactgcattcctgtgtagaaaaatccctggttggatttcggattccaatacgtcatcaccgtcaacgggatttcatattccataatccgaagtttataaatcacattcagggacacctgagtaattcctgccgattcggcatacatggttctgttcaccatttccccgctttcatttcttgaatttctcaatgcgaaagctgttccaataccaggaccgacccttagcttttcgttctgatagatggtatagcccacatatacgaaactggagtagatgttcttgctgttgtccagatccctgtcgcgaccgtaaacaagtgtagagaagctcaactccagcggaaatttcctgtcgcccgtataattgaccatgagatcaacgaaacgtccagtttcatcaggcttatagttgaagaactccttattattatatgtagccccgggcgagaaattatatgtatctatagcctttatctgaaacctgccatgagtatatgctatatactggctcagctccttataactccccctggtgttcgatccgccaaggaaaccggcggtaaacctccccgatgggtcggaaaccgacaaatcggacgagagaatcagtccgtcggccacttcaatgccacgccatagaatcatgttctgtagagtagtactgaaatgaagctgagcctgaacatttgctgacaaaaatataaatacaggaattaacagtcgctttttatacttacaggtatccaatgataatatatgtatcatactcagagcagtagaaaatcggttttaaattattattatggatttatttgtcgaaatactctataagattataaacattccagttaatatccgacatgtatttggtcaatgatgtataaggtttatagttataatcgagcatacctttattgcaatcctcatcatccagatacttgaagaaaacccatcctacacaattcttggcttcgagcagtcccaaggtaaaatgctggtaagcgaatccacggttttgctggtcgcgtaccacgaaaccagctccacttgaattgtcaagcttagtatcctcacccttggtatagaattccgttaccatgaaaggagtaccgcccgcctggttcttccagccatccatgtagcctttttcaggcgaccatttactataataatttatggaaatgacatcacaatattttcccgctgccttaattatataactgttgtatttaggaaggctgtgcaggcgtgaacccagataaagcaattcaggatccttcgatgccttaaccgcattctttatggcagaataatatttttccgcacaaataccggcaaactcattgttcagttcatccgttacatcagaaacatttgcactcttgtccttatccgtcataaacttggcggctgcaatataagcaggatcctgcttgtttgaaattttcaggaatctgtcgagcagcctgtttccccatgtagagaagtctatctcattatccgagaagaatcccaacacatccgggttgtttctgaacatgccgaaagcatccgaattgagatactccttgcaccattcatcccatccatcataaaacacaagacctatcttaagattcacgttctgccccggatagctaattcccttgctattcttgaactctgcaaggaatgaaaaggaaggagcctgtgtcagaggacttgaagccgatttattataatcatttacagccttgtcgccttcttccttaccgaaagcgcagacactatgaaatcctatttcagagaattgtttctgcgactttgccacccagtcatctactgaactgtaaagcttgccgaaagctgagctgttgccatccattctgaatgaggcgataccccttacataatatggataaccttcggggtcgactatccaacttcttccatttgagtttttctcaaccctgaaccgtccagtagccttggatttttgcccttttgcgtatgagccatatttattcacgctttgcaaatactcatcctgtgtttttgtctgctgttcataaccaaccaggtatggcaatatccttgtctttgcctctataaaagccttgtcaggtttttccgcatactcgacaattatcggttgatactgcttggtgctattaggataggtttcagcaggaccgggaacaggcagttgcagttctacatcatcatcgtcattatcgcctgcattgtcgccgggagtattatagtcctccacattccccggttgtgagtaaataacctcaggcggaatatatgagaactcctcctgagggtcttcacatgacaaagcgaagaacggaacactcaagcaaatggttttagtaataatagtagaatatttcattgttgcaaatatttagtaaattaatataaatcccatgtcctgattgtatccccccatcggtggtctatcgggaactccatttctccccatgccttaacagaagtccaaggttggtcggcatcagtccagaatgggtcagaggcaggcaatcccaacggaaggaatgcaagtgtagtcatatacaggctgccattgtttgtataatgattcgaaatgccagtctgatgtccgcagaatcctatggtgaggaatccgccctcattgaagttattgcccgacttgaacatacgtttcatacacgctgtcagcgcacatctcacctgtgctttcgatactcccgccggcaactcattataccatgctataagagccagtggctgcattgttgccatacggtaaggtatagagcgtccgaaaacagggaatgttccttcaggagatatgaaacgctccagaatcatggcgaacctctgtgccctcatcaatgccctgtcatagtacttgcgatagtcgaaacgtgtcctcacgcccgattccattattgcatgtatagattcgagatacataggatggaacacataactgctataataatcgaatgcaaagtgctgtccgtctgcgtaccatccgtcgcctacataccattcctccaccttgcggaaagtagaatttatacgatatgtatcctgtccggcatcaattttggcaaggaagctttcaatggtggccgagaacagcagccagttagtgtaaggagggtcaatgcgtcggagacctttgaactcttttatgtagcgttcctttgttgtctggtccagcggtttccacagctggtcgaacgcgcgcaggaaactttccgcaatataggcagcatcaaccagtgcctgaccatgaccgttccacaacagataatccggactattagggtccaccgcatttgcataactcttcaatgcccattctttcagttgcttgcgctgctgtccttctgctgtatcatcgtcaggcaggctcaaccatggagctataccggccatgagacgtccgaaagtttccatatatgcaaccttcttgttacggttatcccagtttggacttacctcaagaatcatatttttctgcagttcccctttcgccatattgctcaacacaggagcagccatcctgtaagccatatccgtccagtattttcttgtctcgttgttgtttgcctcgagataacgcacatactcgcaagcggcaagaaggaatgcgcctaccccaaagttggcagtcgacttggcgtcaaccacctgtcccggaatagccttttcaccgattggctggacataacccaccgaccagtctttctgcagtgcagtcttggtaagatatttccatgctttccccactacaggcataaattcatccttgtcaagataaccgttgtttatcccccaaagcataccgtaagtgaagaaagcggtaccgcttgtttccggtcccggagcatgttccggatccatcatacttcttgtccagtagccctccggctgctgcagacatgcaaccgcctttgccatacgcacaaacttatcctcgaaaaaagacagatgctcataaccctccggcaggtccttcagcacctttgccagagcggcaagcacccatccgtcgcctcttgcccagaaatccttctttccgttcagactcttatgcttgggataaacatattttgcgtcgcgataatagagtccttcctcctcatcatacattattgagtccgacgtacaaagatattcatacagtttcttaagataccggtgattatgcgtaatcttatacatcttcgtcattaccggcatcaccatataaagtccgtcgctccaccaccagtaatccttacgcggtgtgctcatctggtactccatgacttcgcgtgcacgcttgattttataattctccggcatgacgttatacaagtccgcataagtctggaagcacacctgataatcgccgaacagcacataatcatcctttaccccgtatttatacttccattcagatttgttgttgcttttcgcacccatccactggttatactcagcccatgcctccgaatactttctgtattcttctttcccagtaaggaaataggcttccatattaccggtgtgatatgccgcataatcccagaaagaccttgcttcgggggcatgatttttctgccaggcatcgttcactttttcaatcatctccctaacttgctgagcctcagtttttttttgcgaaggaaaatgaaggtaaaacagctataaggatgtataacatccagtagtatctataacagttcatctttgtgatattgtttacattttctaaaacgaaatggggaagaatatatattcctccctcatttcacgaataattgtattattatatttatttgttaggagtccattctgctccgttgttgaaaccttctgttgtagagtcaaaacttgcatctgctcctgtacttggtctttctgtaatttcttcaatcttaaaagaagtgattttagcggttccagtagcatcagtaccaccagggacattagtctgtacagttaaaataacgttctcaagaaccggccacacaagtgaaccatctgctcttgaagctggagtttcagcagaagtagaactactgattgtgaatgtatttgtataggttccacttccggtatttcttccaatccagaatttatatttatcagatgctcccaatctgaatgttgttgcacagtcgttagatgcgtatgtataagtaaatttgtaagtacaaccatcacggaatgacattgatttagtaactgggaattgattatctgctggaacaatttccaattctccacttgcattaattttttcggcaactccttctgcaagatattccttaactgcatctatgttagcgaagttaaaatcaaaagcatctgcatgagtcaaagcaacattggcagattcaatcttgatattagcatcttcgttgttttcgtttttagcagtcaaagcactaacagcataatcagtgttataacttacgctaatatttgcgtcattactataaatcttatcaccaagaataagagtcatagtagttccattcacagaaccggaagcaacaggaattgtttttcctgctactgttatggtaaatgctttgttaacagcatcagtgaatgttccagaaacttccttatcgagtgtaagttcaattcggtcattacctgttgtctgatcaggaacaatttctttagctgaagaaacggcaacagtagtttgtttttccaaatccacaggaggttcaccgcctccttgatcatcattcaatactatcgttacaatctgtcctttagtaactataaggttttcaccactgaagttataagttttagtaccagaatttcttgtaagttctaaagtaaatccatcggtaaatgtcaccggagctacaaccattgagtattccttggcatttttattttgttcattaggaccaacaaatgttccctctttagcggttagagttataacattagaaccggattccactgtcaggtttgctgaagcatcaatttttacgttccctgcaatctttacatcaccaccagcagtaagtttaatacctgtaaggtcagtaagattatttttaaacttaaccaatccacaagtattctggaaagttaaagatttgttattatctgttgcagtagcataagatatatttgcatttgcatcgaatccccaagccggagctgtctgttcagatggcagtgtagtagttacgacaccttcaagacacacagcttcggcattataaggataaagagctgtatatgaattgttaggtgtagccttacctgtaaacgttgtaactgtgctaccacctgtagcggtagtaaacttgttattttcttggcctgaaaagatattgattgcatctcctgttgtccaccacaccgttgttccattctgcaacgaactacggcttgaaggcgtaccggcaacaaaagtcatatcctgaggaccactgactgcatttacattcgacagttcgtcttttgtacaagactggagcattgcaatactcatcaaagccgctccacaaaatagcatcgtatttttcatgacataaattatttgttaaacagtttcaataataaaaaatcacatcacttgttattcatattcttattctttaggatcaggtttccattcagtaccgtcatcttcaaaatcatcatgaccgccatctacaattccgggaggtattgatattcggcataccgcactttttattccattacccgtatctacagaagcaccgatattagaatctctgcccccgtcgattgccacgaccgtacatctcattttatcgtccgatggtgtaatcatcaacacatcagggaaagaagttccccaaactattgacttgtaaccggtataagggagattatccttggttatattaatacccaactccacagtgccactatatggtaattctatataactgacaggtttgttgtcagtctgcccatccttgaacactacatattcaatttttatctcctcagctggtgttccatcacctccacctacgccatcatcatccttatcacacgagattgccgtaaactgtataaaaagaagtatgaaaaggttgtatactgacagaatccgtggttttatatcaaccataataaaatgttatttaagcgccaaacaaaattttcaatattcaaaaggcataagaggaaaccctgaatatgccttattaccatgaaaacaaatcaatctacctttttcaatccggaatcagaaaaatatgttatttatttagaacatatttttccgatttgccagattacaatcacaataaataaatcaacaactaaatctaattacctaatcttataactaaaccctcaaacaatgttatttaaccttttctatcttgacatcatcaagcaggaagcatccaccattacctgaacccggaacagctgtgaaacgatatacaaaaccattttcctgcaatttgaatttaactgttgtaagattgtaattcttacggtctttcttgacctcagcagtggcaatttcttccagtttctttgaatccggattatagtactcaatcctgaagttaggtttgtcaccccaactgtatttggtataagctgaaatctgatattctgctccagtttcatagctgatgtttacagcctgccacataccaaccttcacctcaacagcatagttgcctgaatgtgcctttttcgcatcaactattttgttatctttcttttcccagacattccatgatgtcaagtcacctgactcaaaatcaccgttcttaatttcctgagcgtatgcagaagtcatcatcattccgcaagccatcattgctaaaatttcttttttcattttttctaaggtttttaatttaagtattatgttgtatctattaaaatcactcttctattggaaccaacttataagccctgacccagtcataataagtagtacttttgtccttatccttcaagtcctcagctgtaggtacttgtttttcccaatcgtatgtttcagtaactatatgtatgaacataggtcggtcaaacggagtatctgtatattttgttgtaggcttgatagtgtacatatactttccgtcataatagaatttcacggtatttgcatccacccaccaacaaccgtaagtatggaaatcttctgccgatgggtccgtcatatacgaaaccacatccgaacgtttcgccgtattgtcagtacgtttgcctccttgttcctgataccaatagtgagtattactgttcatctgcatattccatgtcttgttccacggattatcagggttgacacttcttattatacccattgtttctataatatcaagttcctgactgctccatgtctttatcttcttgccgcctttcattatttccttcattaccgggcggttggaaagccaaaaagtagacgacatggtagtgagcgaagccttcatccttgtttcataatacccataatgtgcctggttctttgcagaagcaaccgctccaccggcaagacgatatttatcgcccggctttccatcaagtccttctgttggcgacaaaacggtattgattatacgaagacaacctttcttgacactaacattctctgccttgaaagttgcaggcggccgaccgttagtccaataaggacttttagcatgccatttagcggcattaagacgtttaccattgaattcatcagtataatcttcgttaactacccatttataaccctcaggagcctcaggcaaattttttatatgctcttcagccaaagaatattccttatcattttttaatgtataagatgacaggaataaagatgcagcagataaatacaatactgtttttctcataaactttgtcgttttagattttttgttacacgacaaaagtatataagtttcatgaaagcattaagggggatttacatcgtaaaaggtggggtaaaattctaccactccctgaaacacaattatttcactcatgaaaccatgtgtttttacgatatataaaacccgacagaagaataataccgtattaccggctaatttacataagaataacttttcaaaccgccatataccccactttacgtccgtaccctcagtcctcgactccggcaatatgttttccatatcgagatctatggttttctgcctcggattcaaccactaactgtcgagcatgtggattgcgtatctgtcatagaatctctttccgaaccatattatctcgtctgtgctaagtatgttgttcagacggataatctttccggtattttaccacctacttctcttgcaaatcctgatctgatataaccggatactctcaattcattgatttccgacttgtatacagtctgcgaagaggcattgaaactactgcacagactgaacagcagcaggggaataatttaactgattttaatagtagacattctgtgttcataatatttcattttaatgattacgtttctgactttcgtctgatgcaaaattatgaggtatcggacggggttgtatctttcagtaaaaatcagtaaagtcttggcaaggggtaaaaaacttaacatcttgtatataaatatattacaaacaaggtgcaaagattttcagtaaacgatggcgaatacagaacctatatatttacacgccataaaatgaagaaaaagcagtaggaaaaaaatgcgggcaagttccggataaaatgtgggcaagtttaaggtaaaacttgcccgcattttagatagaatgcgatcgcatttaaaacaagtaaaaaacgaagaaaaaaaatatgtgttcttcacagaacacatatttcaaaaataggtataaacacgctaaacaatgttaacaaaatctatttataaaaaaagctcacatcaataatatctgcaacatttttacaatactccataaatgaagagaccttgggatgatttatacacagagctatctgtgatgtaggcgaaaaacgtcctgtcccgtcaagaaacgctgtaagctcagatgggaggagtatactgccaatacctggatttacgtcagtcagaacgactgtatttacagcttccaccgctgacacatcaagataatcgagtgccggaagatctgcgaagtgcaattttcctatcatattgccgcctttgctgccctgaagagagacactctccaatgaagaacaaccggatatatggatttcactgtcgaatattgaagtttcggaaacatcatcattaagtataacagaaggaacaactaccaattgaagcgaactgttattctccaccctaagtactttcaatgatgatgcggaacttaaatccattcccaaaggtgtatcaatattagagattgaaaatactgaaactcccgaagacggcttgacatacgacatggaataatgcttggacttcactcccgaaatgtctacttttcctctgaaaccgggatttgacaatatatactccacaccctcaaggttagctgtctgcgacaggaaaatgaggtcgttcccttcggttatcctcttcgtgacatcaatctccaacgatgagacaaacaccgacgggaagtttctgtaaagatatgaacggagcaaaggatccggtactcttcggtttactgtatattcagtgtaatttccatcctcgtccgacatcacgacaagacatttgtccgtcatggctttatagaatgcaggtatgacatctgtattccattttgcaaagtaaggaagtttcagatttgtaagacttttacaaagcaccgtagtgccatcggttgaaataagattgagatacgaagttatgccgtcattgccgcgtaaagctaccgattttattccttcgggcaggtcagcaaagtcgaatatagaaaaactgttacactcaagattgacatctgcgagcgaaggaaaactcctcaaaccgctaatagatgtaagttcgcatctactcaagtccaaagaagtggtattgagaacttgattgtcacaaatcagctctccgttttcgctgaaattaaatcctttccgggtcaagacatcgcgtaactttgtatcaaaagtcacttcagacacttcaaagtcggaaatttctgtttcatccttacacgagattattgtgaaacagagaactatcagtacataaaagctaataaaattcctcataacaatcagttttgtggtaataagactatattatcaatccaagccgcgtcgttctgtctttcgcacacaatggcacacactacttttttcactgtagaattaaaatcgaaagatacggctttataattgccgggagaagaaaattcctctgtatataccgttcctgtagacatatcctgtagcatgactttcaacttacatgctccttcggtctttacatcagcagagaagcgataagtcctgccactctccatgtcaaccctctgcatgagtcctgcatgaccagatatacaggctacattattgcctgcattgtcagtctgtacgcaaaccgtaccatagttacccaatggctgccatgctgaaagtccttcgctgaaggttccattctgcaaggtagagacagtatatttctcaacctgcagtatcatggacgatacgtgacctcctccgtcggaaaaggtgatgtcgacattattatcgccattcttcagcagctgtatgtcgaacggtacttctatcataccgaaaaatatattgcggttgctctggccgtagcctttccagttgtcgggaacactcacagcggtaccattaatctttaccaccggtttcttggaagcagagacaggacggcctatcgacatacgcaagcttgctctgcccgaaccggactcgattcctgtgaaggggaacgaaagggatgatccggcggaaatcggtttcagatactcactgctgtaatatttattgcggattatggagttcgtgaatgctgacgaagacacatctgctacaaggactatggtctgatttgggacaattgagatgctttcaggcatggacgggacattctgttccgtatattctatacctgcgttataattgacatatagagaacgctttgtgacattcgatacatccttccagctattcttattgttcagatatacagtctgcgggttatcatcaagattatcaagggcgatatagagtctgcctccatccttgaatgcctgtacctgaatatcaggattactgctggttatatcaacacgttcgccttttacattcttccagagttcgaagaaatattttttgtcattaagcctccatgtggtattcttcagattctgaggattgtcgggaataaacagtgccgcactatatgaagtataattgtttgcagcggtgatatgccactcagccttatctgagacaaaaggtattgagataaacaaattgtcctgacgttccatcagattaaacagaaaatgattaaacgacgaaacactccgcacactgcttatgtcatcatagctgtcgtcgggcttgctgttgtcaatacctccaaactcggaaatggcaagaggcttgacatgtccgaacttaatataggaatacgcctcaaccatatcaagaactgcttcggagttacttcctgaacgtttcgtatcggtgccggttacatttattccatcataaagatgtacagagaatccatccatatatgcacctgcccgatcgatgaacattttcatgcgggtgttccagtaattgaagttcccatcctcccaggcggggtaggctgcggcatagcctatcaccttcatctttccgttaagacgcggattattgtgtatatgtttacctattgaagcataaaaatcgaccatcagttcgcgcatagcctgtccctgaacggtaaaaccggcatcatttgcatgaacgaacggttcattgaggggttcaaaaaactcaggtaccagctcgctgttggaataatactcagccgaccatgcacctgcagcctgaacgtctatgccgccctgtatgtgctgtacatagggatgctctgtggcaatatatctttttacggaaatatttccgctgtatggtttcatctgaggatatttgcctacctcatgcgtcttgttatacgcatacgagtatggtccccagaactttcttccaagaccgacctgatagtcggcaagaaacttgcctacatccttatcatcatcggaggtggaatgaatattgaaatatttagaacggtcgagttctgaaacaccgctcaaaaagcgacgggtattatagtcgacaaccacctcgttcctttcctgacaataaataccgggaggaacacctagggtaaatgccgataacagaaaaatatatttatagctcataatttctttccttttagacacagaaacttgtcagtcctgatgtggatacattattttctcactttcttatcgtagcgttcagtctgaagaatcatagtagccacacggcctccattatccgggaatgttactgacaccgaattttttccttttctgattaaccggtagtcgaaaggtatttctatcataccgaagaaatcgtctctgccggtctggtcatatcctctccaattgtcgggcatgtcgactttcttgccattaaccattatttcaggtttcttcgacatctcgtgcttcctgcctattgacatacgcagaacagctcttcctgtacccggtttcagaccatcgaaatcaaacacaattggttttccggcttccaccggctgaagataagtgttgctataatatttagtacgaactattctgtttgaatactttttacggatgatgtcggcacacaatattattgtctcatcttttataatgtcaatactttgaggcatcgagttcagcgtcttttcatcataaactatacctttatcgaaaatcatcttcaaagagcgcacagaaacattatctacacccttccaattcagtacgtttttcaagtttaccttatgtgtatagtcatcaagattgtcgacagctatgtaaagcctgtcatcgtccttaaaagctgccacctgtatgtccggattgtcggaaacaatatctacacgttcgcctttcacatccttccataacttgaagaaatatttcttgtcgttcagtttccatgcggtattcttcaagtcgtgaggattgttggcaacaaataaagcagctccgtatggttcgaaattatattgtttcgttatatgccattcggccttgtcagaaacaaagggtattgagatgagcatcttgtcttcgcgttcaagaagattgaacagtatatgattgaacgaagcgacagttcgtacagaggctatcggattatatcctttggaagtgttgtctattcctccatattcggttacggcaagaggaagaactttccccaagcggatgaacgagtagttttccataaggtcgagaatagcttcggaattacttcccgaacggcgggaactcttgcctactatgtttattccatcgtaaagatgtaccgacaagccatccatgtactccccggcacggtcaatgaacatcttcatagtattattccaatggtcgaaatcgcgcaactccatagccggatatgccgcggcatatccaatgattttcatttttttcagacttggctcagcgtgaatatgctttcctgtctgtgcataaaaatctgccatgagcatcctcatttcctgaccatgcatattgaaacatttgtcgcgtgcatggacaaagggttcgttaatgggttcgaaaaattcaggaactgcccctttcacatgcttggaatagtattcggcagcccatgcacccgccttcactgggtctatgccccattgtatggtacgcgcgttggcatgttccgtagcgacatatcgttttgtttccttcaaatcagtgtagttcaaaggcttttctgaaaaaggatattcgccaaccttttttgtcttgccatatgaataagagaacggtccccagaaagagcggccgattcctacaccgtaatctgcaagaaatttcctgacatctggatcagaatctttagatgtgtgtatattgaaatatttacctctgtcaagtgccgatacatcattcaggtatctctgagtggcataatccactgtgacagtagtgttataagtcttattctcggaagatgataaaggaaaaaccgagaaagacaaacacacagacaaagctgtaagaattatgttattcattgtattatcaaaatttaaaaggcagagaacactccgatagttcaattaaagtattccctgccattaagattatcacttctgtttaaacactaatatcagaaatcggccggtttgagtacatcgttcagcaccacttcatattcaacttctgttccgtcgttttcagtaacagtaagatggccgtaaccgccacttgagttattttctttcttaccttcaaacatgaacattctcttcttcgtcacttcctgttcttctttatcgcctgtttcaggattgataacttcttccttttcagtatagacttcattgaaagagaatgagagatgtttttctgtatccgaattgattttcagccactcgggcaattccgaaggagcctcagacgactcggcaaagaactcgatcttattcattctcaaagtctgatagtcattcttccaggcaatgaggtcgaacaattcgcgataaacagaaaacttggaaacctcgcctgtctttcctgtttccacattcttgagataggtaagttcatacacgggagtagaatcaagttcgacctcagcccatttgtcattatcacatgctccgaacaaaaccaaagcacataagaatgtaattgtcttataaattttatctattagcttcattgttactataatttattatggtcttacttcaatatatccgaaaaatatatcgtcaaaataaatattatccttaaaggcattaaagcgcatactgagcaatatattgtccatttcagcctttgaagtcacagtggttgtggccgacatccatttgctgtcggagccattcacaatgccgcaccatggtctatcgctctgccatgtcatatcttcagctccttctttacctgccggaacgaaatacggactcatacccttaccctgtttataccccggtgtataatatttgtagctgaaagtatatgtacctttaccaccagtaaatgtcttggagagtaatgccctgcatcggtcaaatgcttcgacaaacatacattttgcactgttgtttattccatccttcagaggattgtccacaacctgtgaaggaactacaggatgtgttttggtatcggcatcaataactttccagtcggcatatgtgtcagaattttcaaaatcttcatccaggaacgcaccaaaagtagtcgctacgtttggagctgtagcctttatctcaaggttctgatatccaaccaacgcttcagttaaagttcctgtaagggtcagttcatctgtgttatagattttctcaaccaaagtaagaatcagttcatatctgctttgcttgtttacttctgctgctgtgatgtttacgctacccctgacagctgacggtctgttatacgagttggagtaagtaagctttagagatgatggatttatctctttatatccaaactcagaattatccaaatctatagcaatgtgtgtttggtcaatctgacggatgttataagtaataggatcatcactaggtactactgtaatagccaaaggcacaacaagagtttttggcgaagcttttggagtgtacttacctttaccctcactggcagaagttctttctattgtcatggaaagaagcaatggcttatcgctgaatttctttgcagtgaactggtatggagtgtcaaaactggttaattcgtcatttacgccagtatccgcacatttgaaagtccatttgttaggcaatccgtatgaatcgtccttaatatagacagacttaccatattcaagttcgtatttttcgtattcgggagcttcctcagttccaccgactattccggtctttatttcctgtgtacactccggatcactgtatacctttacggccggtacgaggttaggatcatacacgcggatatggaaagttgtatccatcacatatacatcaccctcctgcttagcataacaatattttttgatatatcctccggtattgtcgtcatacaccgaatatggatatacaacctgtctgcggaaagtattgcacaaacgtaccgtatggtcaccgggtttagtgaaatacacatgtatggttttcaaatcgttggtatgagggatggattcatcaatcaggtttgtatagtctgtctgtccccactccatcttaccattaaggaactttgtaccatcatccgacacaacccactgatgcgacaacatgccttgggataagtccattatacttatatagttattaagattcagctgaataggtgaaacgttttcctgatctgtactcacatgccaggtacattcagccacgttattcaacggttcaaactcatcatccttacaagatgtcagaaccgagattaatgaaagagcaatatataaaaatctatttttcatcgtatttatttattaatatcaggatttgatgtaatttctatatttggaataggccagtatgccacttgcggaccgtagttcaatgatgcttggaaataatccacaaaagcgtttcctctcttttctggcggcagctcataaaatctgtactgctttccaaagttgaatgctgataccaaagcattagggtcatcaggattaggcttaagatatttggtctgaatcatacagtacttatattcgtcggatgccaactgatcaaacctttccttagttatattccagcgtctcaaatcaatgacacgtatggcatgtccttccatacacagttcaagaggacgttccacatacatcagatgattcattacatcacttgcagcatattccttctcatcgtatgtatatctcttgaattctccctgttccgattttccgataagcacaactccagcacggtgacgtaccttgttgatggcattgatagctgactgaacatttccatcgcttgcaccgcctttaatcagacattctgcatacatcagatatatatctgccaaacggataagacgatagtttattcctgaggccatagcaggcttaaattcagtttcactcttacgtgtatcccaatttgataattttctgaaatacgctgaagagccacggttgaattttgatacctgttgtgggagagactgataatatatcagactttcatcgccgtttattgcaagagaggcagatgcacgcatggaatagcttctgaggcgatatgcctgaccgtcttcccatttaaattccggaactatgtcatcgtagccggtaatcttattgtataaaactttattatctccgacagttgagacgagtcgttcgcgtactccaacatattttcctgctgttgcatcccacgtataaacgtacgttctgttatatacgacaccctgacggtccacctgcgagctgaaagttgttcccaactggtcgtatataatatccctatgttcaggatcaccataattgtcggactgcatttttatccagttacgttcatcaagtctgtccaccggctctgtttcgaatgcttcaacaagccaaaaagcaggaacagtgttaagccaggcatcgcccaagccatttacattcattccccatatattatataaggtagactccgaccatgtaccgaattctgtattatactgtgtagaataggaaacctcgagaatagattccgaattgaattcattggcagcagtaaaattatcgactatgtcatcaaccaaagcaaaacctccattatcaataatatccttaaaatattcggcagctttattatactctttatcataaaggtagcttttgcctaatattgcctttacagcccaagaggtgatacgtcccaaatcggttttctcccatttgtcattcaagccaaggtcaagagctttctgtaaatcttctctgtaatatttcttgatttcatcacttggtgtaacctttttatagtaatcttcttctacctctgcaatttcattaatataaggaacattaccattattgaatgaattattgagataaaaataaaacaagccacgcaaagaatatgcctgtgcctcaatctgagcaagcttggttatttgaggttcatctgtaacatttggacggattttctctatactggccagaacctgattcgcacggaacacaccagtatacagtgcagaccatttaccacggactgttccgtatgaatcattaaaggtttgcttataggcttcgttatcaaactgctttctgtccttattaccttcaactgctatatcacttctacggttctcatcgagcggatgataaatattggtatttttcaaagcattatatacagcagccagtcctttctcgcagtcgcctattgttttataaaaattctgtgttgtcagctgatgtatgttttcctgcgtaaggaaatcgtcgcatgaaaccaatgtcatgcccgacatcaacagactgaatactattgttttatatctgaagttcatatatttatattattaaaagttagaaattaatctggaatccgccacgcatctggatacttataggatatgttccatagtccaaaccacgacgtgacaatccattactaccgacctcagggtcgtatccgtcgtattttgtcagtgtaagaagattatcggctgcaacgtataaacggaacttgcccaatccaagctttgatacccaactcttggggaatgaatatcctaacataatatttttaagtctgacaaatgaaccgtcctcaatccacatatcagtatgagcacgatagttgttatgcccctctgtacgataagaaggaatggtagaggtatagttggtaggggtccacatgtatatcagttccttattggttcttctttgatatgtatatatcttcgtaccgtttattatttcatttccaactgaagcataccagttcatagagaaatcgaagcctctatagtcggccgagaagttcaaaccaagttcataatccggcataccactaccggcataaacacggtcgtcatcattaagaacaccatcattattggtatcgatatacataaggtcacccatacgggcacttgactgtaatttctgatattctgcaagcttctgttcagtattgattacccctgcggttggcataacaaagaaagcaccggcttcatatcctttcttgattgcagttacataatcacttcctgatgaaacaggtttaccgtcggggaagaaatataactcattttttcctgccatagacacaatctcattcacgtttttggtaaatgtaccagtcaagctgtaattaacaccacgtattttgttgcggtgagtaagtgaaaactcaacaccacggttttccatatctccggcattcaatgtaacagttgaactctggccccctccatttgacggtggcacgaccatcgggaaaagcatattcttcttgttactcttgtacaaatcaagacctaagataagcttgttattatataaagccatgtcgataccggcattaagctgctgggttgtttcccatttcacattcggattggcaaatcccaattgggtaaaaccatttgcaagaatttcggaagttccggtaccaaaagtatagtcgtagtttttgtatatagctggtgcgtatgaataatcagggaagttctgattaccggtagtaccatagctgaatcttaattttaacgaatttactagccacctgaatctgtcgaagaatgattcctcagaaatattccatcctacagacaatgacgggaacaatccccaacgattttcttcggagaacttagatgaaccgtcgcgcctgatactggcacttgccatgtatttgtctgcatagctatattgtagacgacccaacataccaaccattgtactgatacggtcctgtccccactggccactgcctgtacccacagtcatatcggatgttcccgcatttaggttcggaatctcgttagtaaccaaatccattatactggcatagaacatctcgtatgtatatttctccatactgaaaactccggtaaatttaatatcatgcttttttatcttcttattataatttaccattgtttcccaagtgagactggtattctttgaatgagtatcttttaattgcgaacggtaattagagctggttaccttttcgcctttctgattatatacctcaaactcaggtcgaattgagacagctttctgattgttatatccaaagcccaaacgtgtggaaacattcagtccgggaattacattataagcaagataaaaattaccgttaaatgattctgtgtccttatgattttcctctttcaatcttcccaatgtataacttacgccctgtaaatctgcaggatcgccagctgcatttactatacttgcctgtggataaatctgagaacgagtaggcgagtagtcataacattcgttcaataacccccaagccggagataactggttttctatcttcatagcgatgttagtgttgatagtccattttccgcgctgaaaatgtgtattcgaacgaatattatatcttttgtaatcggaatttatcaacacacctttctggtcgaaatagttcgcggtaaggttatatgtcaaatctttcttgccgccattcgcagtaacagaataattctgtattggtgcgttattattgactacatattcatataaactagagttgttgaagaaattcacaggatatgttttcagattagaccaggccaggtcgtctgtattctggtttccttccatcattctgttagacatcacttttacaaatatactctcgttggcatcaagcaaatgaatattcgaagtaatgtgctgtacaccataatatccgtcgacagctatcttcatttctccttccttacccttctttgtggtaataaggataacaccggaagcaccgcgagtaccataaatggcagccgaagcagcatccttaagaatatctatacttgctatttcgctactactcaatcccgggtcgccctcgaacgggacaccatcgacaacatataaaggagaactgtcgcctgagatagaacttaaaccacgaatctggatgttggatttggctccaggctcaccagaacttgcctgaacgttaactccggcaaccataccctgaagagctgtacccaagtcggaagtactgatcttagtaatctcatctgagtttacacgtgccactgcacctgtcacctcttttttacgcattgagccataacctacaacaaccacttcatccaacacttttgtgtcttcctgaagcttgatattataaatctgaccattcttgattgcagcttttacagttttatacccaacaaaactgaacactaagttacctttagtcggtaccccttgaagaacgaaattaccatccatatcagtaatagttccaagagaagtaccttcaacttgaacagctgcgcctataacttcaaggttattggcagcatcaatcacctttcctttaactgttatcttctgtgaatacatagacaatgtatagaagataagcatcacgaacaacatgtacctgccatggtaccattttttctgatttctcatttgtaaaaattttaatttagcaataggttatgaaattccttttataactgacgctaaattatttatttataatggtacaaaaggggagaattatatatttaaaaagggggtaaaattttacccccacttatattaagaatccaaatcggtctgtatactctgttctttgtactgttgcggcaatacaccgaattctttcttgaaacattctctgaaatacttcaaatcattgaaccctacatcgtatgtcacctctgatacagaataccgtcctgtcttcaacagttctgccgctctcttcattcttattgaacgtacaaaagcattggctgttactcccataagtgctttcagcttcttgttcagaaccaaggccgtcacgccaagacctttacatatatcctctatctggaacgaagagtctgtaatgttgtcctctattatctttacaagtttctcaaggaacttatcgtcggtagatgtagtgcttacctcggaaatctttattgccggaactttcttgtgttgaagaatccgcttcctgttggttataatggaattaagcagctctttcattatcttgttgtcgaaaggtttagggcaataagcatctgcatggaatttatatccgatgaaataatcctgcaatgtagtcttggctgaaagcaatactacaggaatatgagatgtccttacatcctgcttgattctctcacacagttccagaccattcatgcccggcatcattatatcggataaaacaagatccggttgcaaatctggaatcatgttccatgccatctccccatcatgggctatcattatcttatacttatccgacaacagtaatgacaacatattacatatatccttattgtcatcaacaatcaatatagccggagattctccgtccacttctatgtctatcatctcttcatgctcgcacgattcacttcttaacacatcagcaaacttttcatcctccccactgttggcagagatattctccgtaaccatgtccccctcagttatcataggaattacaacatggaaaacagtgcctttaccttcctctgatacaaacgtaatatttccattatgtatctctacaagccgcttggtcagaaacagacctataccggtacctccttcagcagagtttttattctgactgtagaaacgctcgaagaggtgtgttttcaggttgtcggatattccgtttcccgagtctgccacagagatgtttattttgttatcctgttcattgacagtaaacgatacaaatcctccggcaggagtatgcttaatggcattcgatacgagattatagattatctgttccataagatgagggtcgaacagaaagcttatatcactgcgtgagacagaatattccagccctacacctttctgttttgcccaatacgtgaactgctgaaatacttcttttgagaaagacgagaagttgccatatttgagattcagactaagcattcctttctcgctctttgagaagttcatcagctggttgacaagacttaacaggaacttactgttatgctccattgtctgcagcatgccggcaagatacttgtcggacgaatacttgcccgattcaataatcatactaagtggagaatgaataagtgtgagtggtgtcctcaattcatgcgatatgttggtaaaaaatgtagtctccttttcaagaagttcttcagtcttgcgtttttccatgtttgctatatatagagcatttctgcgctgcacccgtgaggtataatacaccttgaaccggtataaagacaagacaagcaatataaaatagagtgtataggcataccatgtacgccagaaaggagggttaataatgacaggtatggaaagttcattcaaactgtagactccatcgctattcctgaccctcagtctgaacatatattcgcctgaaggaagctttgtgtagaaagcctcacgatgaaaagcggaggtggaaatccatgaatcatctacgccttcgagcatatattcgtaaccaaccttataaggacttctgtaatccagggagctgaactggaatgagaaagtgtttaaattataaggcaattcaatgtgctctgtaaaacttacacttttgtcgaaataagctgaatatgtggaatctgcctcaacgctgtgattgaagattttaaaatcaacgagtgtaggactaccgttgaaatctatcacatcaaagtcattaggtctaaagacgttaattccgtttacgccaccgaatatcattgttccatccgtcattactccagcagaaagttccataaattcataatcctgaagaccatcgaaaatatcataagatcttattctctgtgtgttgatattcaacgaattaattcctttattggtagaaatccataatgttccatccgtgccattaacaattgattttattgtattgctgctcaacccgtctgcagagctaaaattttcaacgcaggcattatggttttcatccaaatccacgattttccttaacccacgtccaagtgttccataccagatattatgattcaagtcttcacatacaggcactatatagtcgagttcatcaagtcccttgactgagttcaaaacaggattatctatatacaaatctgcagattccaatactttaagaccgaagctggaagctacccatatattacccttatgatctttaatgatgtttcttactatcttaagttctttattgtcagatgttttgatttccttcatcacacctgtggacaaatcatatctgaaaagacctttattatatgtgccaatccacaaatattttccatcggcaagcattgcgcgcacatttctcaaacctgagatctttttataatcattatcagaagtgaaactgtaaataccatcgtacatcagagacacatacatgcagtcggtgtagtttgagtatgctgttgagtatactatcctgtttgccgtgaaaggaataagtctggcattaccggtaatggaattaaaatgatatagccctgagccttctgtgcctaaatatatatcagatttggcaaatgtataaacggacgatatatgatcatttcctattcctctgaataaatctataggtttattattttcgcgtatactcataaagccactcttgaaaaatcctatccaaagaatatcgtttttatcaagaactacagtttgcggatagctgtaagaatatgtagcaataacctgtggttttgactcgatggcatgcaatacatcaaaagtcaacacattcacagtgcttgtagtggcataaaataatcttttgtttttatataccatttttcgtatatcacagttttccaacagggtacttaccttgcaggtatgcttgtcgtataaacataattgatgattttccagatttgagtacaatatttgagaagatgagatgactatggctgaagctatagggcatcccaatagtttgttaagcagtaattcatctccatcgacgttacattcgtacaggccgtcttcggaggagagcattatcgtattatctatttctatgatgtcggaaatgtatggtaattttaatgttgatcttaagacagtatttattttgccattttgaaaatcataatttacaaggtatatactttcatcagaggaatgaaaccagactctgtctttagagtcgacaagaatcttatcgcaagtgaaatttttatcaataccgctgtgaccaagatttaatgaaacgaattcgttctttacagaattgaacaggaacactcctctatcggctgtacctatccacagatttccatgtgaatcttcgtcaatacatactatcagattactgttaagaccgtttgactgatatccgtaaaccttaaattcatatccgtcaaacctgttcagtccgtcgttcgtggccaaccatataaagccttttgagtcttgataaatacattgcacatcattttgggaaagtccatcaagagtagtgtactttcttgtgacaaactcattggatgcaaaggatttgcaaactataatcagaactgatattaaacttaagattaatctaaacatataactattattctttatatttcatcaagattacaaagttattgattttatctaaaacatcaagtatttacagtagttaatagataattatagatattttccactttagaatgcgtatcaaaatcaatcaagaaaaaaataaatctttaacttcatttcatagtataaaacaaaaaaagcatcgtaccattacactcaataatagatacgatgcccgaaagaaattacagtaacagactgtattgggattgttcttaaaaagacttatctgtatgactttatatatatgtcgagtatttcggtatccgacagttcatgagggtccagactgaacaatgcacccatggcagttcgcgcattatcaatcatcttagggaaatcttcctttactattccccagtcgctaagcttcaaatcgcggacattgcattccttctgcattctcaccaaagcatctataaaatgttcgggattaaggttcttgcatccggtcataacatctgccatgcgcatatatctctttgtcctgtcataaataaaagtagagaaataggcctcgcttatagctatcaggccaacaccatgaggaagagcgggatagtatgcgctgagagcgtgctcgagagaatgttcggaagtacaactggatgtggattcaaccattcccgccagcgtacttgcccaagccacctttgccctcgctttcaggttatttccatccttcaccgcaacaggtaaatatttatacagcagtctgatggcctcaagagcgaaaatatcacttattggggttgcacaattggcaatatagccttcggctgcatgaaagaatgcgtcgaatccctgataggcagtcagatgtggcggaactgaaaccatcagttccgggtcgattatcgacagacatgggaaagttaaagtggagccgatacctatcttttcgtttgtttccagattggttatgacagtccatgggtcagcctcggttccggttccggctgttgtaggaatggctatgatgggcaatgctttgctgtaaggaagccccttgccggtacctccttcaacatattcccaataatcgccatcattacatgccatgattgcaatggatttggccgtatctatcgaacttccgcctcccaaacctataatcatatcgcaattttcctcacgacagattgccgtaccttccattacatggtcttttattgggttaggcaatatcttgtcgtacaccacggcatcaacattattttctttcagcagaccaatcaccttatccagataaccatatttacgcattgatgttccggatgaaatgactatcaaagcctttttgccgggcaatgtctctgttgaaagacgtttaagttcgccacatccgaagagaatcttcgtcggaatattataaccaaaaacaaaattattgtccataaatattatcagtcagtcaacttactatcttaaagcctcatcaatcactttcttgagttcaggataagcctcatctgtatcgcccacctgttttctcaactcacgcagtttctttttcatgtccttaagaactttggcgtatttaggattatcagccaggtttaccatttcgtaagggtcgttcttcacatcgtagagttcgaaagaaaccggagtaggaacaatcttgtggctgttcttcaaccatgacattgatttctgtccgtaacgtttgtcgtcgtaatgacggccatagaaaagtatcagcttatagttttccgtgcggatacctatgtgtgccggaacgtcgtgatgaatcatgtgcatccagtatctgtagtaaacagcatccttccagttttctggctttttgccttcgaacacagaggcaaagctctttccatccatgtatgaaggttctttgccaccgaccatctctataagagttggagcaaaatcaatgttgttaatcatcaggtccgacttggctcccttgtaaggacatctcgggtcgcggactatgaaaggcattctttgagattcttcatacatccatctcttatcctgcagatcgtgttcgccaagcatcataccctggtcgcctgtatatacgataatggtattttcccagagtccttccttcttgagatagtcgaaaagacgtttcaggttgtcatccacaccctttacgcaacgcagatacgatttcaggtaatgctggtaggcaaggtatgtattctccatttcatcacctgtattgcacttatattccattacataattgcggatttcatgacggcttgagacagaagttccgatgaagtgacgaagtgaatcgttcttgcctcttgtgccttcggagccccatttgtctgtatcgaacaatgacaatggaacaggcacttccacatcgtcaagataatattcatagcgcggtgcgtactcgaacatatcgtgcggtgccttgtaatgatgcatcatgaagaaaggtttggacttgtcgcgtctgttcttcaaccagtcaatagcaaggttggtcacgatatccgaggagtaacccattttctttatctggttattaggccatttcttgtcagttacgtcacttgtaaggaaaatagggtcgaagtattcgccctgtccgccatgaccgttgaatacagaataatagtcgaagtgcgacggttcgcatcccaaatgccatttaccgatcatggcagtctgatatcccatattatggaactcatcaaccagatattcctggtccggctgaagcacttcatccaaagtgagcaccttgttacgatgggaatactgtccggtcatgatacatgcacggcttggggtactgatggagtttgtacagaaacagttctcgaagagcataccgtcccttgccagttcatcaattgtaggagtagggttcagtactgcaagacgacttccgtatgcgccgatagcctgcgaagtatggtcgtccgacatgatgtagatgacattcatctgtttctgctgtgctgcgacaccaacacatacagacaggaatggcataacagccattcccttcattatattattttttaaattcgttttcataagtcagattatcattgaaatagaacttgcaagacatatcatcgaatgattttacgtccttattctgcattttaacccattgttctgatttagccttgacagcgacctgagttgaaacctcattaccgtcgactacacttttaagagtgacatttgcatcctctgcattatggtttgccacacgtacagtgataaggcatccgttatcaaccttatcgtatagcggtttggaaaccaccgcccctttaagcttaatcttgaacacatgtgcatattcagtaggtttgttcttagggaagtttactacaagaccctcgtcagtcatcttatagtcaatcttctctgagcttccaagcatttcaaccgactcaatttccacgttctggcaatacttaggagcaaatgacttgatagtaacactaccatctgtccaagccagagacacggcatagaggttattgtcgcgtgtagtaaagcgaatgtcgtccgctgtatattcagtttttgtattgtctgtcatataacctgcggtgcctgcgttatgtccttcgaaagcaatcacccatggtcgtgagccataaatagcctcaccgttagtcttcaaccatttacctatctcggcaagtacgttcttctgttcgtctgtaatagtaccgtcggccttaggacctatattcagcaataagttaccgttcttgctgacaatatcaacaaagtcgtcgatgatatggtcaggactcttgttttcctcgcccacacaatagctccacgatttcttgcctacagaagtatcagtctgccatggatattcacggattctgtcgctcttacctctttctatatcgaacacctggatattgtcgccatatccgaatttagtgttaaccacaacttctttattccaatcaagagccgaattgtaataataagccatgaatttatagaaagtaggctggaacggatattttcccacagtccagtcgaaccatatcaattcaggctgatatttgtcgataagctcgtatgtatgcataaggaactgacggcgtgaacgttcgttcgagccttcatacttaccacaataaggtgtcataccctgaccttcgggctcatgcagtctttcgccatacagagtgattgtagtgtcctgaacatcagaaggagtttccattccatattcatagaaccatgcattctcgcatctgtgagaagaaagtccgaaacgcagaccggctttcttggtagcttccttcaattcgccgattatatcccttttcggtcccatatccacagcattccacttattgaaagtactgctgtacatggcaaatccgtcgtgatgctcggccaccggaacaatgtattgtgctccagatgattttaccactgccagccactcgtcggcattgaaattttcggctttgaacatagggatgaaatccttatatccgaatttggtcaaaggaccgtaagtctgtacgtgatacttattaataggatgaccttccttgtacatccagcgggaataccattcactgccgtatgcaggaacggaataaactccccagtggataaagataccgaacttggcatccttaaaccattcaggaatagtgtaattttgagcaatcgatgccgaatcggccttgaacacatcagtaccttttaaagatacagtagaatctacattaggagcgtatgtagaattgcacgacgccaacaggcttaatgccgcaactcctaaaaccgttttcatggatttcttattcataataatcttattacattaaataatgacattaattttttctgtaagcaaagatacacttgagttccatttacaataaataatttaattactatagtaaggggtaaaatatttaccacctattattgaacaaatttaccccctctcatatatgataataaactgccaatatcgaattacaagtaaatatatatttcaacaaaaaaggtttagcctattattacacaacaatttcaccctaagaataaaatatatatagagtaaatttgccaatataacaaactgtaaaaacaaatttatgaaaaactatttgatttacttactcgcagcagtatcgtgtacaactgtagcagacctaaatgctcaagtcagtacaaaaacaggtaatgaaaccacagaacttacaattccgaaaaagttctacaaggacagcattgatttcagcaatgctccgaaaagacttaacaacaagtaccctctttccgaccagaagaacgaaggcggatgggttctaaacaaaaaggcctctgacgagttcaaaggaaagaagctgaatgaggaaagatggttcccgaacaaccctaaatggaaaggaagacaacctactttctttgcaaaggagaatactacatttgaagacggctgttgcgtgatgagaacttacaagccagcaggatcactgcccgaaggatatactcacactgccggtttcctggtaagcaaagaacttttcctttacggatatttcgaagcaagactgagaccaaacgactcgccatgggttttcggtttctggatgtcgaacaatgaaagaaactggtggactgaaatagacatttgcgagaactgccccggcaatcctgccaacagacatgacctgaactcgaacgtgcatgtatttaaagctccagcagataagggtgatataaagaaacatatcaacttccctgccaaatactatataccattcgaattgcagaaagactttcacgtatggggacttgactggagcaaggaatatatccgactatatatagacggagtactgtacagagaaatagagaacaagtactggcaccagccattacgcatcaatcttaacaacgaatcgaacaaatggttcggagccttgccggacgacaacaatatggattctgaatatctgatagattatgtaagggtgtggtacaagaaataagaaataacataatctgaaattataaaaggcagtcttcattatcagtatgctgatgataaagtctgcctttttaacaagaagataaagattttaatctgccctatcactcatttacttcatccggatactctgtaagcgagtttcccgaattgcttatttcaatagagccgataggaagataattgaacttcttgctccatgcagagataccataatctcttctaagaataggcatcatgacctcctcggcacgtcctgagcggacgaggtcaaaccatctgtcaccctcgcatgccagttcacaacgacgctcataccatagaacatcaattacgcttttaaatctgtcaggatacatctgcattagcttgtcaacatcaatataacttccgtcgtctgcatgaacatgcttctttctgagttcatttatgtaatacttcgcttttgcttcatcaggattagtacctctgagatatgcttcggcaagcatcagatacacttcaccatatctgatgacccttacgtttccaggcttgtttagattggggtttcctatcatatcgtaatttttgaaaggaggatatttcttctgggcatatccctggaaatcaggcccgtaagagcctgtctcccaaacaactttttttgattcatcctgaatattggcattaggtttggttacaagttcatcgtaagtaaatatcgccgcatcacgacgcacatggtcatccggaaggaaataatcatacaattccttagtaggcagacaaaagccatatccattatcataatcaggactatttttcaactgtctcggtccgcagaaagtcacccacatagcaccttcgcctgcatcaatattaccccagtttgtattaccagatttggtagaggtctgtatttcaaatatagattcctcgttattctcctgatgagccgcaaacaatttagaataatcatccgtcagagtataattaccacttgaaattacatcctccaataaaggtttcgctttgtcaaaaatcttagcatcatcgttgctccagtcagcccaataaagatagaccttggccaacagggcttgagccgcagtcttggtaatacgtcctttcattgtgtccgggaaattatcctttagagaagggatagcttcaagaagatctttctctattgctttatttacattttcgcgagtatctctcgtaaacttgaatccttcaggataaagagtctcaagactgataaagcatggaccataatatctcaacaattcaaaatgataccaagcacgtaagaacttagcttcagctttataaactttagcttccggactgtcatactctgaatttattacaagattacatctatatataccacggtaacgagttttccacaaattatcggaaatagaattgacactcgtatttgaataatcctctatagcctgcatgtaaggctgatcctgatcagagccaccaccagtacgagcattatccgaacggatttcacccataggtacaatggaagcaagtgcattacccgaagcaccacctatgtgagctaacggatcataacaagcagtaagcgctttgaacatctgttcatcggtcctataaaaagaactttctgtttcggacattataggagctgtatccaggaaactgtcgctgcaagatgatgatgcaatagcagcaaacatgaggacaagaatattattatgtattttcgacttcataattttcaattttagaaattaagacttaaaccaaatctgaatgtacgggcctgagggtaagtaccatagtcaatacctgtgctaagaatattgccacctgccatatttcctacttcaggatccataaacggatagctggtgaaagtggcaagattatcaattgctgcataaattcttgctttattcagcatcaacttgtttattaatttagttgggaatgaatagcctacctcaagtgaagaaatctttaaatgcgaaccatcataaagataaaaatcggatggtttgccaaagtttccattaggatctttggatgaaagacgaggcactccattatcatcaccttctttccgccatctgtcaagatagaatgatggaaggttgctgcgtccgtatgcttcctgtcggtaaatatcagagaagactttatatccagcttttcctgttaagaagattgtcatatcaatacctctccagtcggcacctaaattcaaaccgaatgtccattttggccaaggattgccacaatcggttctatcttcatctgtaatctgcccatcgttatttgtatcttgccatataaagtcacccggaacggcatcaggttgtatcactttaccgtcttttgatttatagttctgtatctgctcttcattttggaatattcctaagttcttataaaggcggaaataacccatagcatgaccttcctccatacgcgttacattaacagatgttctccagctaccaccatcagtatatccatttacatttcctatctttacaacctcatttttaagatatgaggcatttgcggaaatagagaagttgatttcgttccaatttttattaaatgtcatctgcatttccacaccctggtttgttatattaccaaggtttctaaaagctgcattattacctctaatggcttcaactgttggctggaacaacaaatccttagtactttttttaaaccagtcgaaacttgctctaatcataccattatagaatgtcatatcggcaccaacattaaattgttcagaagtttcccatttcacgtctggattaacaaggttattaggagcagatcccacagtgatggcattaccaaacgtgtaattataattattgccaataatagaagtataggagaatggagaaattcgctcatttccgttctgtccccaagagaatctaagtttgaagacatcaaagttcttaattttccagaatttctcatttgaaacattccaacctaatgaaacgcccgggaaagtagcatatctgttattgggaccgaaatttgaagacccatcgcgtctgaccacaacttccgccatatatttttcagcataattatagcttagacgagcaaaatatgagaacatactatgtctaggattagcaccgccactattagctgatgtcataacatcaccagcattaagataccagtaattctcattggtcattgcttcatttggatatttatttcgtgttccggccataaactcataaacatctcttgatgcagaagtacctaacaggacagatgtagaatgttcaccaaaagattttttatatcgcaatgtattctcccactgccaactactattagcatttgtactttgttctaccctagaattatcttctttacattctgcagaatgaaaaaactttggtgcaaacattcttccacggaaattccgatgattaataccaaaatctgtgcggaaaacaaggtctttaataaaagtgatctcagcataaacattaccaaaaaattgctgggtaatatttttattcttaggtgcctcatccataaatgcaatagggttccacatacggctataaggtacaggagagactccatatccgaaagtatcgttgctattctcatcataaaccggagtagtaggatcaatattataggcgtatgatatcggattataaccattgataccggttgccactccactattctctatatatgcatagttgacgtttgcacctacacttaagaaatcatttatagaataggaactgttcagccttgtgctgaatcgtttgtaaaatgacgcatcttcaccgataataccattctggtctagataattcaatgaaagcaagcttgaacccttatcactgccaaagttagcagtaatgttatgctcagtaacaggagctgtattcaatatttcattaaaccagtctgtattataacctgttggagcagtaggtacaccaccggcaagcggcatatcatcattgtcggcaaactctttcatcagcataatgtactgttcatcattcagcatggttggtttctttgctactgtagagaaaccatagtaaccatcataagcaagcgatgtctttcctttctttcctttctttgtggttataaggactacaccattagcggctctggcaccataaatagcagctgaagttgcatccttcaagacttccatgctttcaatgtcgttgggatttacactgttcatgtcgtccataggcagtccgtcaattacaaaaagaggattagagtttccatttgtaccaacaccacgaattaccagcttcggtgctgttcctggctgaccggaatttgtcacaacgttcacaccactaaccctaccgctcaatgcattcacggcatttgctggtttagattgcaataaatcatcggaatcgatgctactgatagcacctgttacaacactttttttcttaacctcatatcctattgctacaacttcctcgagtgcaatggcagatgtttttaattgaacgtctatcttagactgacctttatacactatattctgtgtatcatatcctacgaagctataaatcaatgtcgattccattggtacattttccaagatataatttccgtccaaatcagaaataataccgtttgtggtacctttaactaaaatacttgcacctatcacaggtaaaccatcggagtctgttatacaaccggtaactttcccgttctgtgcatttaatggtaaactgaacgttataagaatcagcatacacattaatgatagtgttctgttcataatctagagttttttgtaattagtgtttttcttaaaataaaaagttttgttctatcagttgcgcgctacttactgacacttgcaaatatatatactatgtaatataaccaaagggggaaaatttcatttaaataggggggggaaatagattaactaaatattttaaggaaaaatggctgttagaatccattcccagactccaacagccattttatcactaacaatcgcctgttaatcaatatatttttctgcccatttccttaagatttgcatccctgcccagtggaacaaaagtaaatccgtatgaatagcttcccttcagaagacgcttgtctattgaaggacgggctttcagactccagctatctgttccgcccactccagcctgaaccaggtcgatattaagagtattagaatacaagtccttttcaagttcatttatatgtttagccttatcaatcgcattctgcgacatctcccacactgaaacagataggggttcatcgccgacaatcatcacacctgccttatccgactgcaaggcaaaccatctcacgtcacaacggtttccgttttcctgcggcattacatagtcaaatcccagagcggacaccttgcagttatatatagacaccattgcagaggcttttctgtcggaatagttttcccatgggccacgtccataatatgtcacatccgacaaacgattggtacattcgcattgcaatcctacgcgcaacatttctgatatttcaggagacttcatcattgaataatgaacgcctattgttccgtctgcttttactttataattcaaggtaagtctcagtctttcatctatagcctttagcaccttaacctcaagattgccttccgatttgcgtacatctatagaaactgtctttagctttaatggagcatctttccagaatgcaaacagtctatcgaccttccatcctcgccagtcattgtctgttgacgctctccagaagtttggtttcagagcagatgtgatgatactttcattatctatcttatactgactgatataaccatcactgatattcagataaaagttctttcccttcacgctgatgtctttcttgttatctgaatcgatttccatatccaatgtagtatcaacgcattctactatctttggtaaagaaagatacttaaactgttcccaggcaacctcgtatccagctttggcatacagattgtcattcttgagcctggcactcaggaataaccaatattccgcaccgtcatcggccttgaaattctgaataggaagttttagtttacagctctcaccagctggtgttgtcggcacaataatctcaccttcctgcaatacactgtcttcgtccttcaattgccaaaaataacgatactcatctgttgaaaggaagaagtttctgttttttacagttatctctccactatagacattatcagttgtaaatgatacaggagcaaacacgtacttgcattcctcagtagcaggtttaatggagcggtcggcactgataacaccatttatacagaagttttggtcgttgtgctcccctttctcatagtcaccaccataattccatgatttcttattatatttccgttcattatccagcaatccctggtctatccagtcccaaatatatccgccggcaagcgcatcatgagaacgtattgcatcccagtattctttcagcccgccggtagagtttcccatagaatgtgcatattcacacattattatcggacggttcatgaccggattcttagtcattgctataagctcatcgaccataggatacatacggctaatgacatcgacgtataaaggatcatcgggattggcatacacacaaagctctttctttgccggtttgacatcttcgttcacattaaaatctatctcactagtaacgattgacgcttccttacgtccgataggtttgtataaaggattttccggctgtccttgcgccccctcgtaatgaacaggacgggttgggtcataatctttcagccatcctgacagagctgcatgattagggccgcatccagactcgttgcccaacgaccacataaacacagaaggatggttcctgtctctcacagccattcttaccactctctccatgaacgagttagcccactcaggcctattggacagataccccctttgatgatgagtttcaagattagcctcatccattacgtatataccatacttatcgcacagttcatagaaataagggtcgttaggatagtgcgatgtacggactgtattgaagttataacgcttcataagcagaacgtcttcgagcatctcatcacgtgtaacggtcttacctccggtctcgctatggtcatggcggtttacaccaatgagtttaataggagtgtcattcaccagaatctgattacctgttattttaatatccctgaaccctaccttattacttctcgcatccaccacgttgccctttttgtctgtgagctttataaccaaagtgtatagataagggtgttccgaattccatagttttggcttagaaacaattccctccatcattccgtaataaacattatcacgctgaggataaggttcgttcaccacataatcggcagtaacggtaatgtcttttccaaacaccggtttcccatcggcatcatataattgggctgacagattccatcccttcaaatcatccatattctgatttgttatttccggacggatctgtaaccgtgctatattcttccggaaatcgatgcgtgtccttactccataatcatatattgccacctgcggaatggacatgatatatacttcacgatggataccagccattcgccagtggtcggcatcttccatataacttccgtcggtccacttatacacttgcaccgccagtttattctcccccttcttaacgtattcggtaatatcaaattcagtaggcagacaactgtcttcggaatatcccaccttctgtccgtttatccatacattaaatcccgaatagacgcctccgaaatggagtataatcctgtcgctcttccacttgtcaggaacaacaaactccttgatataacaccccgtctgattattcctgtcaatatatggcggacgagcagggaaaggataaatagtatttgtatatataggatagccatatccctgcatctcccaacatgaaggaacaggaatagttttccatgatgatgaattgtactccactttataaaaaccggcgggagccaatgccatatcctcggaaaagttaaacttccattggccgttcaacgacatatactccgatttctctctgtctccatccaaagcccaatccactctccggaaagaataagtagtactgcgggaaggcaaacggttaattccgtttatggtctgatcctgccatacattctgattgtttctccactgattggcaccgttgtccgatgcagacagaaattgcatcatgaaaaataacacagaaaatgaaaaaatagattttaagttcaagttcataaattcgcattttaagtttctatgcaaatatataagtataacgaacaatgaatagggggtatttctatctatatagagtggtatttttacatatgagctaaaacttaaaaaaaactgtcagtattactatgctatgtagcactctatatgaaaatattatatattcccaagtcaaaagccttttcaaacaatttttatatattctcatcctatcccttccatcaaagataaattccaatcctgatttgccagccgcatttattccttttttcaggagaattttctttatggctatcgccatgaaaattcacctgaaaaagaatgcggcggcaaacggattagaattaaagaaaagattacagggattaactgcgaccgacgtgacgcatagccgtaattcaaaggcggctatccttatattccatatatgacctcacaaatactgtgaaaatccactttccccaataacaaaacatagcctgccatatcaacacccaaaataagacagggatttcaactccctccgatctgcatagtctggtggcttcgctatgcttttactcctacatccattttttttctttcttttttcctctgttcccgttctttcctatccttcgtgtgacatttgatgacacctgatgacatctaatgtcatctatttgtaaatcaattgtttactcaatttatcatcttacatttggactgtgaaacaaatcaagtagtcactcaaaacaaaagattatggcacaagaaaacagtcctgacaaggaaaaaaggcaaggccggacaaagaaacccgaaaagccttatgtggaacaaattgacgagcttctgctggtacataacaagaatgacccaaaggaaggtttgggagtaatcagcaagatggacgagaaaggcaattatcagacggttacaccggaagagaagaatgagaactcattcctgaaattcgacaagaattcgagtattctcgaaaacttcatcaagaatttctggagccagctgaaggagcctacgcatttcaggcttatccgtatgaccttcaatgattacaaacagaacaaacaggctctcaaggacctggccgaaggcaagaagacagacgcggtaaaggagtttctgaaacgctatgaaatcagaccgaaagtaaacaatcagaaaaacagtcaaacaaaagaggaggaaacaacaatggcaaagaagcaggaacagacaacgcaggctcagcctgaacaggtatcacaggtggaagctgccgcacaggggcgcgaacagcaggaaccgcaacgccagcagacacccacgtaccgctacaacgagaacatgattaattgggaggaactgggtaagttcggtatatccaaagaaatgctggagcagtccggacagcttgacagcatgttgaaaggatacaagaccaacagaaccatgccgctgacactcaacattcctggggtactgaccgcaaaacttgatgcacgcctttcgttcatatccaacggcgggcaggtcatgctgggcatccacggtatcagaaaggaacctgaactggaccgtccttatttcggacatatcttcacggaagaggacaagaaaaacctgcgtgaaagtggaaacatgggacgcgtggctgaccttaacctgcgtggcaacacgacagagccgtgtctgatttccatcgacaagaataccaacgaactggtagccgtacggcaggagcatgtctatatcccgaatgaaatcaaagggataaccttgactccggacgaaatccagaaactgaaaaacggagaacagatattcgtagagggaatgaagtccaatcaaggtaaagagtttaatgccaatctgcaatatagtgcggaaagaagaggcatcgaatttatcttcccgaaagaccaggctttcaaccagcagacgcttggcggtgtaccgctttcccccatgcagctcaaagcgttgaacgaaggacacaccatccttgtagaggatatgaaacgaaagaacggcgaactgttttcttcctttgttaccatggacaaggttacaggcgggctccaatatacgcgccacaatccggaaacgggagaaatctacataccaaaggaaatctgttcggtacagctcacaccggaggacaaggaagcgttacgcaaagggcagcccatctatcttgagaacatgatcaaccgtaaaggtgaggaattctcgtcattcgtcaagctggacctggcaagcggaagaccacagtattccagaactccggacggtttcaacgaacgacaggcaccagccatcccggctgaggtttacggacacctgctttcggcacaggaaagagctaatcttcaggacggaaaggctatcctcgtaacgggtatgaaaggtcccaacggcaaaccgttcgattcctatctgaaagtaaacgcaaacaccggacagctgcaatatttccaggaaaatccggatgtgcgccgcaatacttcacagcgtgcttcacagactgacaatacccagcagcaggaacagaagaagggagcaaaacaggctgtctgacctgaacgggattcaaatcattcaaatcatcaattactaaaaaaggaaagaacatgaacaagaccaatcatcatatctacaagactgaacaaatcgactgggagaaactggaatcggtaggtatcagcagatcgcaaattgaaaaggacggaaacatggacctgctccttcagggagaggaaaccaatgtcatgtccattaaaatcaagactcctgtattttcactgaccatggacgccacactcagtctgattgaagacgagaatggaaatccggtcatcagcgtaaacggtatcaacccttcaggtgaataaataagaaaccataatgtatcatctctctttccatacggacttaccgtatggaaagagataaaaacagaatttatcatgattgccatattaacagacaaaccaagtgtaggaaaagaaatcggaagaatcatcggtgcaaccaaagtaagaaacggatatgtggaaggaaacggctacatggttacatggactttcgggaacatgctgtcactggccatgccgaaggactacggaacccagaagctggaacggaatgactttcctttcatcccgtccgaattcgaactgatggtacggcatacacgcaccgagaacggatggataccggacattgatgccgtgctccagcttaaagtaatcgagagagtgtttcaggcatgcgataccatcattgcggctaccgatgccagccgtgacggggaaatgacattccgctatgtctatcaatacctgaactgtacactgccttgcttccgtctgtggatttcctctcttaccgacgagtctgtgcgtaaaggcatggaaaacctgaagccggacagttgctacgacagcctgttccttgctgccgacagccgcaacaaggcggactggattctcggaatcaacgccagctatgccatgtgcaaggcgacgggccttggcaacaattctctcggacgggtacagacaccggtactggctaccatcagcagacgctaccgtgaaagggagaaccatatttcatcggacagctggcccatctacatcagcctgcaaaaggacggcatccttttcaagatgcgccgcacacaggatcttcccgacaaagaatccgctacaatgtttttccaggactgcaagctggcacatcaggcacagattacaggtatcagccacagcgttaaggaaatacttccaccggacctgcttgacctgacacaacttcagaaggaagcgaacatccgctatggttttaccgcatcagaggtgtatgacatcgcccagtctctttatgaaaagaaactgatttcctatccgcggacttccagccgttatctgacggaggatgtgtttgactcgcttccaccaatcatggcgcgtctgctttcatgggagctgttccctgcagctaaaggaactggaggtattgacatatccaatttgtcccgccacgtaataagcgcagaaaaagccaatgtacatcatgccatcatcattacaggtatccgtcccggaaatctgtccgaaaaggaaatacaggtttacagacttgtagccggaaggatgcttgaaacattcatggctccatgccgcatagaaacgacaaatgttgaagcggtttgtgcggcacagcatttcaaggccgaacaaacaagaatcattgaagccggctggcatgatgtgtttatgcgttccgacatggttccaaaatcaggatattctgtcaatgaactccccgaagtggagaaaagtgatactctgaatgtatgcggatgcaacatggtacacaagaaacagctgccggtaaatccgttcacggatgcagaactggtggaatacatggaacagaacggactgggtacagtatcctcacgtaccaatatcatccgtacactggttaaccgtaagtatatccgttattcagggaaatatatcgttccgaccccgaaaggcatgttcacctacgaaaccatccgtggaaagaaaattgcggatacttcactcaccgcagactgggaaaaacagctggccggacttgaaagcggaatgataaccggacaggacttcctgaacaggatcaggactctcgccaaggaaatgactgatgacattttcaacacctattccacaaaagaagaataacatctatacctaatcaaccaagagaatgcaggccggaaggtctgcatttttttgtatccgtacagaaaagaatctgtttttccgcttttaagcggcaaaggtcttggattgcctgccttttgccgcaaggctgccctcatgggcttggctggacaggaaaaaatcatcctcgctgcgctccggtattttttcctgccaggccttgcgcaaaaaggcaatccaagaggccggaggcctataaaatcgggaaaacacatcccgatgggattattcattcataaaattaaggattatgaaactacagattatcagaaagatcggcagacatgcaacagcgatattcctgattaccggaatatgtctgctgacaagtaaagggattgtccctactgggatgattacgctgctgttgcttgcaggagggttcatcggttttctgttcaggatactggtcattattttcaagattcttattcttctgttcattgtaggattatttgtcgcataacccaaaatataaatatacatatatggaaacagttgctataacctcacaagctcctgtcatgccggctgtatggccacagaacgaacatatcagaccggttaaaagacgtctgcccaatacagttgatgaacctaaaaatatcggctactatctggaatcgctacgtgatatttccagcaatccggacagagagaatattctgaaagaattcttcaaggaaacttatgtataaccataaaatttttcaattatgttttttcaatcaatttatcagatgattacagcaggtacggatctgaatatcaatatccgtaaagtggacaacagcctgagcgtagcagtcatgccaaggcggaacagcctgaaagaggatacgcgacagaacatggtgccactgatcgtgaacggaacaccggcagaactggatatgggcttcctgcagaccatactccaaccgatacagaaggtacagggactgcttgtcaatgcggaaaatttcgagaaacaggcagaaaaggctacatcacaggccaaatcatccaaggctccaacaataccggccgaatcaaaggaagccagggaaaaacgggaaaagatggaaaagctcctcaagaaggctgatgaagcaaccgccgcaaaaaggtactccgaagcaatgacatggctgaaacaggcacgggtactggctcctacagaaaaacagaaggatattgacgaaaagatgcaggaagtacagaaacaggctagtgcaggaagcctgttcggtatggcagaggaaccggcgccggtaattccccaaccacaaggctatatgaacggtcagtcacaaccaggtatgcaaacaagcatattcccggagcaacagacccatactatgaatcctgaacctgtcatgcagcctgctccacagcaggtatcacaacaaattccacaaggaatacctcaaccggcatatggaacgaacgggacatataacccacctgctccaaacagcccgatagtaaaaggagcagacataccgcaaggcgcaacaatgcatccttacccacagcagccatactaccagcaagaggcgactccttatccaacacaacagccacagcaaccgacaaacggacatataccgaatggggctgcgcaagtacagaatggaaacggacgggaataccagactgcatcggctacacatgagacattctgcttcgatccggaagacgagaatgacagggaacttctaagagaggacccgtatgcggaatatccggattttccggctgagtaccgaatgaaggacgaggcacaggtagaaatggtatactgctgatatacacaataaacgatttgtaaaaccaataaactataaacaatatggcactggaaattaaaggaatgaaaagagtattcaagatgaagaagaacaatcaggaaatcgtactggatgatccgaacgtaaacatgtctccggctgaagtgatggacttctattccatgaattatccggaactgacaaccgcgaccgtacacggaccggaaatcgaagacgaccgggcggtatatgaattcaagaccactatcggagtaaaagggtaagagcatgaaaaaaggacaacgtaaagacaagaaaccatgtacacaacttacggaacgggctttggaaaatttagccagacttatcatatcggaactcgaaaatacggacataagccggggcatcaggaacagaaagaaaagaagactccctcccgcagaaagcctcatggttttctgaacacgagaataccttccatcgctcccgatctgtatgttgagaatgacagggatgtaacggtaaatgtcaccaccaaagagaatcttgatttcctgtaccgttcagccatgaagtatgcgcagctcctggatgtggagctgccataccatcctacaggcaggacttccacaagagagaaaatatgcctgctatataatgcactggattccatagtatctcatcatgtaaatctggaacttattggtgacaggctccagttctgcatctaccatttccatgaatggccggattatacgcttttctttatgccgatagactttacggaaaggctgcacggtgaaattaaaaagattacactggagttcatcagaaagttcatcaaatatcacaggatgatggatataaccgataccccttattttgagatgtcggaagtctgtatcgattatgtggactttgaacagctcgatgaggaagagaaaaaggatttgtacagaaaggaaaagcttttcaggtcatatgagaaagggagaatccacaggaagctgtgccggatgcactccagggctttctgtaggaatctggaagaacatatccgcaactgtactccttccagcgataaggaaagaagacttttggaactgattaccgaagggctgtccctgattgcaaaggacagcccttatatcttgaattatgattatgattttgcaagcgaaaaggaacgggatttcgagccgccaccgctcgaatatcagattctgcttacatattccatcacggatacggttaccaaagacatggaaagctgtttcagtactgactgtcaggaaacatataaccagactcccgtatcatttaccttcatcacgccggaaacagaggaacttttcaagccggacaactatccggaacggtttgagaaatggtttgagaaatttgtagaacatgttacctataatttataaacatcatgaatgaactgaccaaaaatatgcaaaaaatgatggtaccgaaggctgcaatcatagcctacaagtatgaagacagaagaaatcttgataccaggtactttatagaattacgtccaatcagaaaaagcggacagatgggggcaggtatccccgtcacatacgaattcatgaataccctgctggaatcctatacggaagaaatgagcgggataccggcaggcagagtccctgaaaacatgctggcctgcaatccgagaaaaggacaggaagaatatatctggtacaatccgcccggaaaaagacagatgttctttcacaaggatctcaatatacaggacggcatgttcaatctgccgggaattatctaccaagtaaaaaacggaaacatggacgtgttcgctttcaaggggaaacgtccggtggagacgactccgctgttccgtgccccgttcttcaacgtgaccggatcaagtgtctgccttggcaacagttctctggaaaagccacagaacccgactttcctttccctgctggaatactgggaaaaacggttctggctgactgaattctcccatctgggaggaaatgtcaatcctaccgtttcaaatcttgtcatcgtcaccgaaaatataagaaacaatccgttcgacatgaacgaactcaagcccatgaataaaaaacttaaagacatacttccatgaaaaagatacattttaccgaccgctacctgctcaatccacgtcatccggtaacggtattcgtcatcggagctggaggtaccggctcacaagtgataaccaatctggcacgcatgagcatggcacttcaggcattaggtcatccgggactgcatgtcaccgtattcgatcccgatacggttagccaggccaatataggacgccagcttttcagtgagacggaactgggactgaacaaggccgtatcacttgtcacacgcatcaaccgtttcttcggatacgcatggactgccgaaccgaaatgtttcccaacgaagaaattttcaggatatgatacagccaacatatttatcacctgcactgacaatatacgttcacgtcttgagatttggaaatttctaaagaaaactcgtaaagagaacttcaatgactatttggttcctatatattggatggattttgggaacagccagacaaagggacaggtcatcatcgggacggtacgtgagaaagttctccaaccttcttcacaagaatatattcccatgcctaaaatgaatgtcatcaccgaggaagtggactatgcgaaaatcaaggaaaaagaatcaggaccaagctgttctctggcggaagccctggaaaaacaggatttgttcattaactccacactggcacatatcggatgtgacatattatggagaatgttcaaggaaggaaagacactgtatcgcggtgcctatgtcaatctggatacattgaaaatgaccgcaatcccggtgtaatgacagaagtgaccgtatcatctttccatcagaatacggtcacttattctatttgctacttattatttactacgttcttaccacgctggagcaggaaactctgtatctctgaggcgagatagaatgatttcccgttcttttccaccgagtaatatttaatcttgccctcttgcctgtaacgtgccaaagttctttgtgacacaccaaggagttctgccagatccacattatcaagcagtctgtctccattcatacattctttcagacgattcatctggtccagtttcttttcaatgcgggcaaatccctctaccattgttcctataagtctttcgagtatctcattatctatatatgacataattccaatgttattaagtgaataaatcgatactctcttcgtgcgcactctaagagtatgtacttatagtagtgaaaatagtatgcctgaatctaagacaaagatcaacaagcttattaggcgctgataatcaggcgtataattttttctacttaatatttagtgtaaaccaaaagtgtaaactatgtaatacagaattgggaacgggttaacacagccaccaacaatgacatctgatgctacctgacgacacctaatgacaacattttgtatcatatacatattcaaaatacatttgtacaaactcaacttttttggatatggaaatcattggaattgaaacagctacatatgaaaagacattaaaggaaattgaaaacttccttgataccattgataaattgattacagcttcttcacagaaaacaataggggaatggttggataaccaagaagtttgcctgatcctcaaaatttctccaagaacattacagaatcttagagatacagaccaaatctcttattctcaaattgggaaaaagatttattataaaaaagaagatattcagaagttcattgaaaaacacaacagaaaattatgagcaaggtaattacccaagataatgagcaagttattcagatatacaataggttaaaagatacgctaacaagactcgaagatattctgaagaataacaacccaacacttaatgggcatagatatatgaatgatgcagaattggctaattaccttaaagtatcaagacgcactttacaagaatatagaaataatggaatcttatcttattatcagattggaggtaaaattctatatcgggaatctgatatagaagaacttcttgagaaaaacagacaggaagcattccgttaaacatttcttggaattttcgttgattttcaaagcaaaaatcagtatctttgcaatactgacaaagagttgtatatcagtgcagaacaaagaagttcaatcgaggtgaaataggtggactaaatgacaaacaacaagataagtaattgattattagcgataaaaaatataaggttccgcccccaggcggatcactgaaaacaaaagagaaat